(19) United States
(12) Patent Application Publication
(10) Pub. No.: US 2023/0147015 A1
(43) Pub. Date: May 11, 2023

(54) HETEROCYCLIC COMPOUND, ORGANIC LIGHT-EMITTING DEVICE COMPRISING SAME, MANUFACTURING METHOD THEREFOR, AND COMPOSITION FOR ORGANIC LAYER

(71) Applicant: LT MATERIALS CO., LTD., Yongin-si, Gyeonggi-do (KR)

(72) Inventors: Eui-Jeong CHOI, Yongin-si (KR); Geon-Yu PARK, Yongin-si (KR); Young-Seok NO, Yongin-si (KR); Dong-Jun KIM, Yongin-si (KR)

(73) Assignee: LT MATERIALS CO., LTD., Yongin-si, Gyeonggi-do (KR)

(21) Appl. No.: 17/785,803

(22) PCT Filed: Dec. 29, 2020
(86) PCT No.: PCT/KR2020/019248

§ 371 Date: Jun. 15, 2022

(30) Foreign Application Priority Data
Dec. 30, 2019 (KR) 10-2019-0177328

(51) Int. Cl. H01L 51/00; C07D 405/04; C09K 11/06; C07D 471/04; C07D 405/14; C07D 409/14; C07D 495/04; C07D 487/04; C07D 491/048; C07D 405/10; C07D 209/86

(52) U.S. Cl. CPC H01L 51/0073; C07D 405/04; C09K 11/06; H01L 51/0067; H01L 51/0072; C07D 471/04; C07D 405/14; C07D 409/14; H01L 51/0074; C07D 495/04; H01L 51/0071; C07D 487/04; C07D 491/048; C07D 405/10; H01L 51/0052; C07D 209/86; H01L 51/5012

(57) ABSTRACT

The present specification relates to a heterocyclic compound represented by Chemical Formula 1, an organic light emitting device comprising the same, a method for manufacturing the same, and a composition for an organic material layer.

### TECHNICAL FIELD

This application claims priority to and the benefits of Korean Patent Application No. 10-2019-0177328, filed with the Korean Intellectual Property Office on Dec. 30, 2019, the entire contents of which are incorporated herein by reference.

The present specification relates to a heterocyclic compound, an organic light emitting device comprising the same, a method for manufacturing the same, and a composition for an organic material layer.

### BACKGROUND ART

An organic electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

Studies on an organic light emitting device comprising a compound capable of satisfying conditions required for materials usable in an organic light emitting device, for example, satisfying proper energy level, electrochemical stability, thermal stability and the like, and having a chemical structure capable of performing various roles required in an organic light emitting device depending on substituents have been required.

### PRIOR ART DOCUMENTS

### Patent Documents

U.S. Pat. No. 4,356,429

### DISCLOSURE

### Technical Problem

The present disclosure is directed to providing a heterocyclic compound, an organic light emitting device comprising the same, a method for manufacturing the same, and a composition for an organic material layer.

### Technical Solution

One embodiment of the present application provides a heterocyclic compound represented by the following Chemical Formula 1.

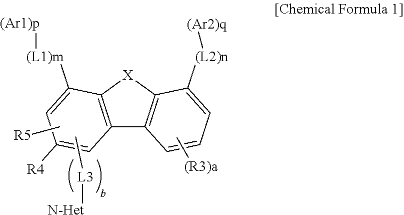

In Chemical Formula 1,

X is O; or S,

N-Het is a monocyclic or polycyclic heterocyclic group substituted or unsubstituted, and comprising one or more Ns,

Ar1 and Ar2 are the same as or different from each other, and each independently a substituted or unsubstituted C1 to C60 alkyl group; a substituted or unsubstituted C6 to C60 aryl group; a substituted or unsubstituted C2 to C60 heteroaryl group; —P(═O)RR′; or —SiRR′R″,

R3 to R5 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; —SiRR′R″ and —NRR′, or two or more groups adjacent to each other bond to each other to form a substituted or unsubstituted C6 to C60 aliphatic or aromatic hydrocarbon ring or a substituted or unsubstituted C2 to C60 aliphatic or aromatic heteroring,

L1 to L3 are the same as or different from each other, and each independently a direct bond; a substituted or unsubstituted C6 to C60 arylene group; or a substituted or unsubstituted C2 to C60 heteroarylene group,

R, R′ and R″ are the same as or different from each other, and each independently a substituted or unsubstituted C1 to C60 alkyl group; a substituted or unsubstituted C6 to C60 aryl group; or a substituted or unsubstituted C2 to C60 heteroaryl group,

p and q are an integer of 1 to 4,

b, m and n are an integer of 0 to 4,

a is an integer of 0 to 3, and

when p, q, a, b, m and n are 2 or greater, substituents in the parentheses are the same as or different from each other.

In addition, one embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound represented by Chemical Formula 1.

In addition, in the organic light emitting device provided in one embodiment of the present application, the organic material layer comprising the heterocyclic compound of Chemical Formula 1 further comprises a heterocyclic compound represented by the following Chemical Formula A.

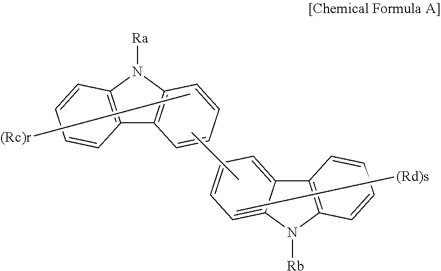

In Chemical Formula A,

Rc and Rd are the same as or different from each other, and each independently selected from the group consisting of hydrogen; deuterium; a halogen group; —CN; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiR201R202R203; —P(═O)R201R202; and —NR201R202, or two or more groups adjacent to each other bond to each other to form a substituted or unsubstituted C6 to C60 aromatic hydrocarbon ring or a substituted or unsubstituted C2 to C60 heteroring,

Ra and Rb are the same as or different from each other, and each independently a substituted or unsubstituted C6 to C60 aryl group; —SiR201R202R203; —P(═O)R201R202; or a substituted or unsubstituted C2 to C60 heteroaryl group,

R201, R202 and R203 are the same as or different from each other, and each independently hydrogen; deuterium; —CN; a substituted or unsubstituted C1 to C60 alkyl group; a substituted or unsubstituted C6 to C60 aryl group; or a substituted or unsubstituted C2 to C60 heteroaryl group,

r and s are an integer of 0 to 7, and

when r and s are 2 or greater, substituents in the parentheses are the same as or different from each other.

In addition, another embodiment of the present application provides a composition for an organic material layer of an organic light emitting device, the composition comprising the heterocyclic compound represented by Chemical Formula 1, and the heterocyclic compound represented by Chemical Formula A.

Lastly, one embodiment of the present application provides a method for manufacturing an organic light emitting device, the method comprising preparing a substrate; forming a first electrode on the substrate; forming one or more organic material layers on the first electrode; and forming a second electrode on the organic material layer, wherein the forming of organic material layers comprises forming one or more organic material layers using the composition for an organic material layer according to one embodiment of the present application.

### Advantageous Effects

A compound described in the present specification can be used as a material of an organic material layer of an organic light emitting device. In the organic light emitting device, the compound is capable of performing a role of a hole injection material, a hole transport material, a light emitting material, an electron transport material, an electron injection material or the like. Particularly, the compound can be used as a light emitting material of the organic light emitting device. For example, the compound can be used alone as a light emitting material, or two of the compounds can be used together as a light emitting material, and can be used as a host material of a light emitting layer.

Particularly, the heterocyclic compound according to the present application has each of substituents of Ar1 and Ar2 at a No. 4 position of each benzene ring of dibenzofuran, and by increasing thermal stability through blocking an electronically weak position of the dibenzofuran with the substituent, an organic light emitting device comprising the same has properties of a particularly improved lifetime.

Particularly, the heterocyclic compound represented by Chemical Formula 1 and the heterocyclic compound represented by Chemical Formula A can be used as a material of a light emitting layer of an organic light emitting device at the same time. In this case, a driving voltage of the device can be lowered, light efficiency can be enhanced, and lifetime properties of the device can be particularly enhanced by thermal stability of the compound.

### DESCRIPTION OF DRAWINGS

FIG. 1 to FIG. 3 are diagrams each schematically illustrating a lamination structure of an organic light emitting device according to one embodiment of the present application.

### REFERENCE NUMERAL

100: Substrate
200: Anode
300: Organic Material Layer
301: Hole Injection Layer
302: Hole Transport Layer
303: Light Emitting Layer
304: Hole Blocking Layer
305: Electron Transport Layer
306: Electron Injection Layer
400: Cathode MODE FOR DISCLOSURE

Hereinafter, the present application will be described in detail.

In the present specification, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present application, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present application, in a “case of a substituent being not indicated in a chemical formula or compound structure”, hydrogen and deuterium may be mixed in compounds when deuterium is not explicitly excluded such as a deuterium content being 0% or a hydrogen content being 100%. In other words, an expression of “substituent X is hydrogen” does not exclude deuterium such as a hydrogen content being 100% or a deuterium content being 0%, and therefore, may mean a state in which hydrogen and deuterium are mixed.

In one embodiment of the present application, deuterium is one of isotopes of hydrogen, is an element having deuteron formed with one proton and one neutron as a nucleus, and may be expressed as hydrogen-2, and the elemental symbol may also be written as D or 2H.

In one embodiment of the present application, an isotope means an atom with the same atomic number (Z) but with a different mass number (A), and may also be interpreted as an element with the same number of protons but with a different number of neutrons.

In one embodiment of the present application, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

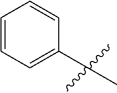 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

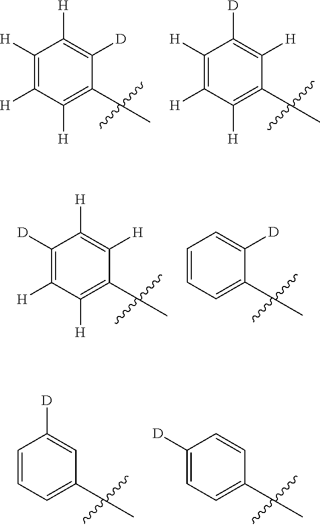 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present application, “a phenyl group having a deuterium content of 0%” may mean a phenyl group that does not comprise a deuterium atom, that is, a phenyl group that has 5 hydrogen atoms.

In the present specification, the halogen may be fluorine, chlorine, bromine or iodine.

In the present specification, the alkyl group comprises linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may comprise a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethylbutyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group comprises linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may comprise a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group comprises linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may comprise methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the cycloalkyl group comprises monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may comprise a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group comprises O, S, Se, N or Si as a heteroatom, comprises monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group comprises monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The number of carbon atoms of the aryl group may be from 6 to 60, specifically from 6 to 40 and more specifically from 6 to 25. Specific examples of the aryl group may comprise a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, an indenyl group, an acenaphthylenyl group, a 2,3-dihydro-1H-indenyl group, a fused ring group thereof, and the like, but are not limited thereto.

In the present specification, a fluorenyl group may be substituted, and adjacent substituents may bond to each other to form a ring.

When the fluorenyl group is substituted, the following structural formulae and the like may be included, however, the structure is not limited thereto.

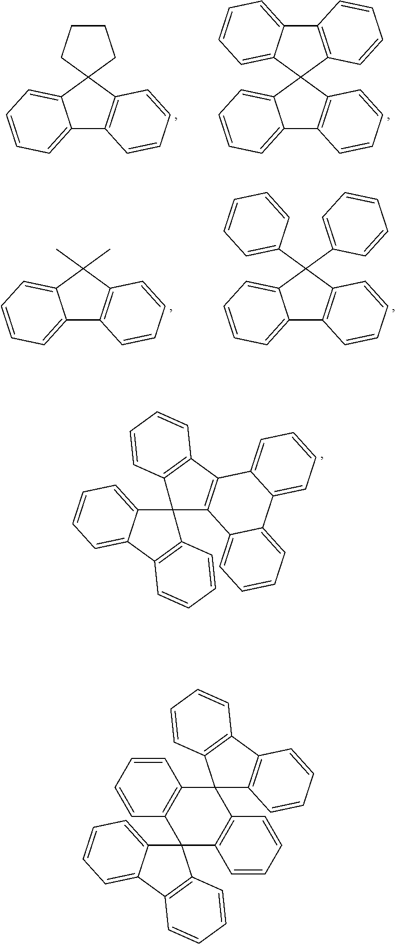

In the present specification, the heteroaryl group comprises S, O, Se, N or Si as a heteroatom, comprises monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may comprise a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrobenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may comprise a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the phosphine oxide may comprise a diphenylphosphine oxide group, a dinaphthylphosphine oxide group and the like, but are not limited thereto.

In the present specification, the silyl group is a substituent comprising Si, having the Si atom directly linked as a radical, and is represented by —SiR104R105R106. R104 to R106 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may comprise a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

As the aliphatic or aromatic hydrocarbon ring or heteroring that adjacent groups may form, the structures illustrated as the cycloalkyl group, the cycloheteroalkyl group, the aryl group and the heteroaryl group described above may be used except for those that are not a monovalent group.

In the present specification, the term “substitution” means a hydrogen atom bonding to a carbon atom of a compound being changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification, “substituted or unsubstituted” means being substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine, or being unsubstituted, or being substituted with a substituent linking two or more substituents selected from among the substituents illustrated above, or being unsubstituted, and

R, R′ and R″ are the same as or different from each other, and each independently a substituted or unsubstituted C1 to C60 alkyl group; a substituted or unsubstituted C6 to C60 aryl group; or a substituted or unsubstituted C2 to C60 heteroaryl group.

One embodiment of the present application provides a compound represented by Chemical Formula 1.

Particularly, Chemical Formula 1 is a tri-substituted compound in which N-Het substituting one side benzene ring of the dibenzofuran is a substituent having electron transport characteristics (electron transport character moiety), and in addition, substituents of Ar1 and Ar2 are present at a No. 4 position of each benzene ring of the dibenzofuran. By increasing thermal stability through blocking the No. 4 position of the benzene ring, an electronically weak position of the dibenzofuran, with the substituents of Ar1 and Ar2, an organic light emitting device comprising the same has properties of a particularly improved lifetime.

In one embodiment of the present application, the heterocyclic compound represented by Chemical Formula 1 may have a deuterium content of greater than or equal to 0% and less than or equal to 100%, preferably greater than or equal to 20% and less than or equal to 100%, and more preferably greater than or equal to 40% and less than or equal to 100%.

In one embodiment of the present application, the heterocyclic compound represented by Chemical Formula 1 may have a deuterium content of 0% or 100%.

In one embodiment of the present application, Chemical Formula 1 may be represented by the following Chemical Formula 2 or 3.

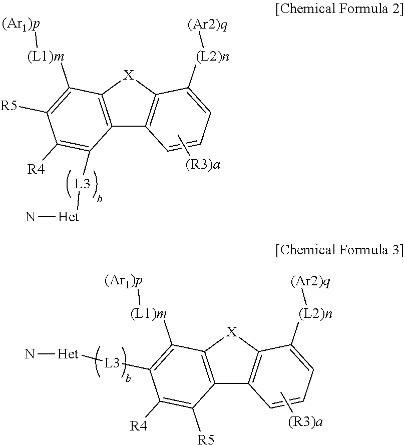

In Chemical Formulae 2 and 3,

X, R3 to R5, N-Het, Ar1, Ar2, L1 to L3, a, b, m, n, p and q have the same definitions as in Chemical Formula 1.

In one embodiment of the present application, Chemical Formula 1 may be represented by any one of the following Chemical Formula 4 to Chemical Formula 10.

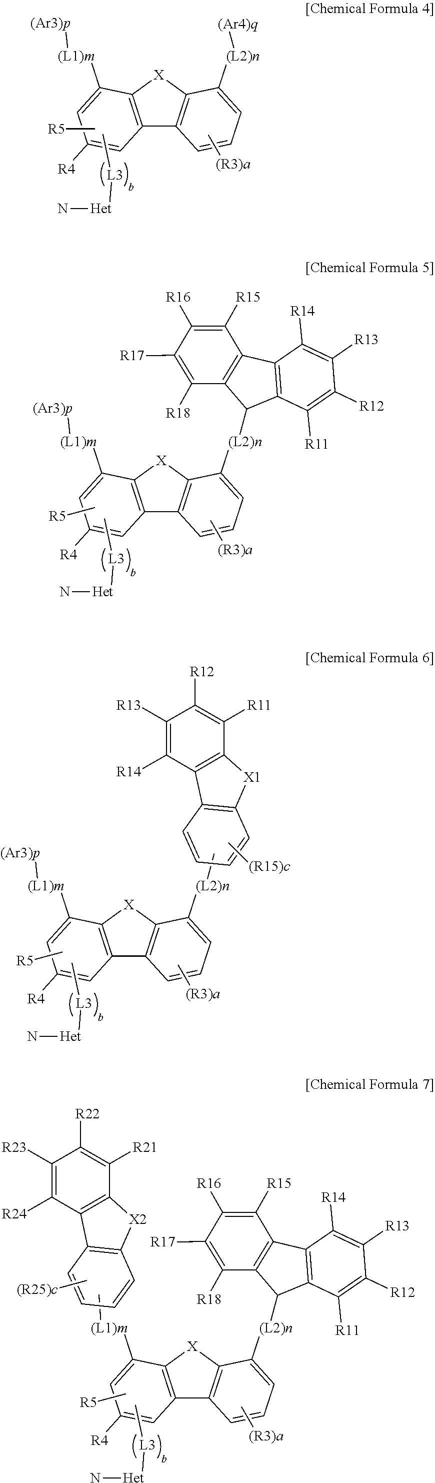

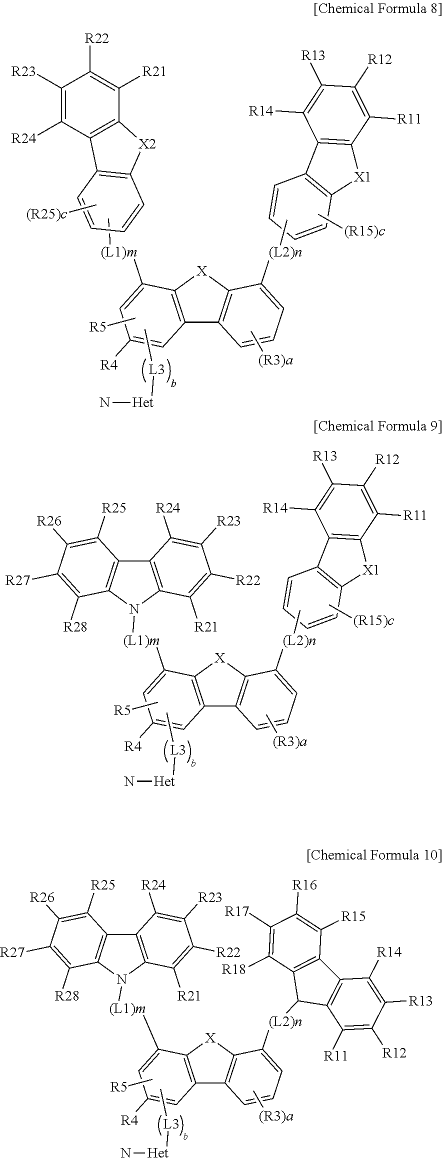

In Chemical Formulae 4 to 10,

X, N-Het, R3 to R5, L1 to L3, m, n, p, q, a and b have the same definitions as in Chemical Formula 1,

X1 and X2 are the same as or different from each other, and each independently O; S; or NR31,

Ar3 and Ar4 are the same as or different from each other, and each independently a substituted or unsubstituted C6 to C60 aryl group,

R11 to R18 and R21 to R28 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; —SiRR′R″ and —NRR′, or two or more groups adjacent to each other bond to each other to form a substituted or unsubstituted C6 to C60 aliphatic or aromatic hydrocarbon ring or a substituted or unsubstituted C2 to C60 aliphatic or aromatic heteroring,

R31, R, R′ and R″ are the same as or different from each other, and each independently a substituted or unsubstituted C1 to C60 alkyl group; a substituted or unsubstituted C6 to C60 aryl group; or a substituted or unsubstituted C2 to C60 heteroaryl group, and

c is an integer of 0 to 3, and when c is 2 or greater, substituents in the parentheses are the same as or different from each other.

In one embodiment of the present application, L1 to L3 are the same as or different from each other, and may be each independently a direct bond; a substituted or unsubstituted C6 to C60 arylene group; or a substituted or unsubstituted C2 to C60 heteroarylene group.

In another embodiment, L1 to L3 are the same as or different from each other, and may be each independently a direct bond; a substituted or unsubstituted C6 to C40 arylene group; or a substituted or unsubstituted C2 to C40 heteroarylene group.

In another embodiment, L1 to L3 are the same as or different from each other, and may be each independently a direct bond; or a substituted or unsubstituted C6 to C40 monocyclic or polycyclic arylene group.

In another embodiment, L1 to L3 are the same as or different from each other, and may be each independently a direct bond; or a substituted or unsubstituted C6 to C20 monocyclic arylene group.

In another embodiment, L1 to L3 are the same as or different from each other, and may be each independently a direct bond; or a C6 to C20 monocyclic arylene group.

In another embodiment, L1 to L3 are the same as or different from each other, and may be each independently a direct bond; or a phenylene group.

In one embodiment of the present application, L1 to L3 may have a deuterium content of greater than or equal to 0% and less than or equal to 100%, preferably greater than or equal to 20% and less than or equal to 100%, and more preferably greater than or equal to 40% and less than or equal to 100%.

In one embodiment of the present application, L1 to L3 may have a deuterium content of 0% or 100%.

In one embodiment of the present application, R3 to R5 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; —SiRR′R″ and —NRR′, or two or more groups adjacent to each other may bond to each other to form a substituted or unsubstituted C6 to C60 aliphatic or aromatic hydrocarbon ring or a substituted or unsubstituted C2 to C60 aliphatic or aromatic heteroring.

In another embodiment, R3 to R5 are the same as or different from each other, and each independently selected from the group consisting of hydrogen; deuterium; a substituted or unsubstituted C6 to C40 aryl group; and a substituted or unsubstituted C2 to C40 heteroaryl group, or two or more groups adjacent to each other may bond to each other to form a substituted or unsubstituted C6 to C40 aromatic hydrocarbon ring or a substituted or unsubstituted C2 to C40 aromatic heteroring.

In another embodiment, R3 to R5 may be hydrogen; or deuterium.

In one embodiment of the present application, X may be 0.

In one embodiment of the present application, X may be S.

In one embodiment of the present application, N-Het may be a monocyclic or polycyclic heterocyclic group substituted or unsubstituted, and comprising one or more Ns.

In another embodiment, N-Het may be a monocyclic or polycyclic C2 to C60 heterocyclic group substituted or unsubstituted, and comprising one or more and three or less Ns. In another embodiment, N-Het may be a monocyclic or polycyclic C2 to C40 heterocyclic group substituted or unsubstituted, and comprising one or more and three or less Ns.

In another embodiment, N-Het may be a monocyclic or polycyclic C2 to C40 heterocyclic group unsubstituted or substituted with one or more substituents selected from the group consisting of a C6 to C40 aryl group and a C2 to C40 heteroaryl group, and comprising one or more and three or less Ns.

In another embodiment, N-Het may be a substituted or unsubstituted triazine group; a substituted or unsubstituted pyrimidine group; a substituted or unsubstituted pyridine group; a substituted or unsubstituted quinoline group; a substituted or unsubstituted 1,10-phenanthroline group; a substituted or unsubstituted 1,7-phenanthroline group; a substituted or unsubstituted quinazoline group; substituted or unsubstituted pyrido[3,2-d]pyrimidine; or a substituted or unsubstituted benzimidazole group.

In another embodiment, N-Het may be a triazine group unsubstituted or substituted with one or more substituents selected from the group consisting of a phenyl group, a biphenyl group and a naphthyl group; a pyrimidine group unsubstituted or substituted with one or more substituents selected from the group consisting of a phenyl group, a biphenyl group, a quinoline group and a naphthyl group; a pyridine group unsubstituted or substituted with one or more substituents selected from the group consisting of a phenyl group, a biphenyl group, a quinoline group and a naphthyl group; a quinoline group unsubstituted or substituted with a phenyl group; a 1,10-phenanthroline group unsubstituted or substituted with a phenyl group; a 1,7-phenanthroline group unsubstituted or substituted with a phenyl group; a quinazoline group unsubstituted or substituted with a phenyl group or a biphenyl group; or a benzimidazole group unsubstituted or substituted with a phenyl group or a biphenyl group.

In one embodiment of the present application, the 1,10-phenanthroline group may be represented by the following Chemical Formula 1-1, the 1,7-phenanthroline group may be represented by the following Chemical Formula 1-2, and the pyrido[3,2-d]pyrimidine may be represented by the following Chemical Formula 1-3.

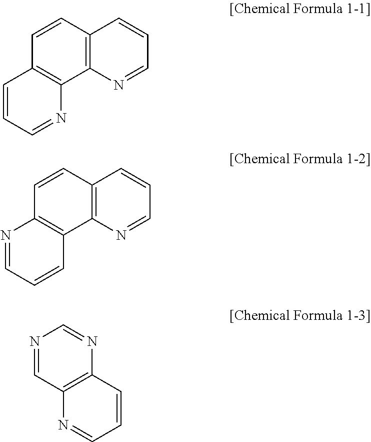

In one embodiment of the present application, N-Het may have a deuterium content of greater than or equal to 0% and less than or equal to 100%, preferably greater than or equal to 20% and less than or equal to 100%, and more preferably greater than or equal to 40% and less than or equal to 100%.

In one embodiment of the present application, N-Het may have a deuterium content of 0% or 100%.

In one embodiment of the present application, Ar1 and Ar2 are the same as or different from each other, and may be each independently a substituted or unsubstituted C1 to C60 alkyl group; a substituted or unsubstituted C6 to C60 aryl group; a substituted or unsubstituted C2 to C60 heteroaryl group; —P(═O)RR′; or —SiRR′R″.

In another embodiment, Ar1 and Ar2 are the same as or different from each other, and may be each independently a substituted or unsubstituted C6 to C60 aryl group; or a substituted or unsubstituted C2 to C60 heteroaryl group.

In another embodiment, Ar1 and Ar2 are the same as or different from each other, and may be each independently a C6 to C60 aryl group; or a C2 to C60 heteroaryl group unsubstituted or substituted with one or more substituents selected from the group consisting of a C6 to C60 aryl group and a C1 to C20 alkyl group.

In another embodiment, Ar1 and Ar2 are the same as or different from each other, and may be each independently a C6 to C40 aryl group; or a C2 to C40 heteroaryl group unsubstituted or substituted with one or more substituents selected from the group consisting of a C6 to C40 aryl group and a C1 to C10 alkyl group.

In one embodiment of the present application, Ar1 and Ar2 may have a deuterium content of greater than or equal to 0% and less than or equal to 100%, preferably greater than or equal to 20% and less than or equal to 100%, and more preferably greater than or equal to 40% and less than or equal to 100%.

In one embodiment of the present application, Ar1 and Ar2 may have a deuterium content of 0% or 100%.

In one embodiment of the present application, X1 and X2 are the same as or different from each other, and may be each independently O; S; or NR31.

In one embodiment of the present application, Ar3 and Ar4 are the same as or different from each other, and may be each independently a substituted or unsubstituted C6 to C60 aryl group.

In another embodiment, Ar3 and Ar4 may be a substituted or unsubstituted C6 to C40 monocyclic or polycyclic aryl group.

In another embodiment, Ar3 and Ar4 may be a C6 to C40 monocyclic or polycyclic aryl group.

In another embodiment, Ar3 and Ar4 may be a C6 to C40 monocyclic aryl group.

In another embodiment, Ar3 and Ar4 may be a C6 to C40 polycyclic aryl group.

In another embodiment, Ar3 and Ar4 may be a phenyl group; a biphenyl group; or a naphthyl group.

In one embodiment of the present application, Ar3 and Ar4 may have a deuterium content of greater than or equal to 0% and less than or equal to 100%, preferably greater than or equal to 20% and less than or equal to 100%, and more preferably greater than or equal to 40% and less than or equal to 100%.

In one embodiment of the present application, Ar3 and Ar4 may have a deuterium content of 0% or 100%.

In one embodiment of the present application, R11 to R18 and R21 to R28 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; —SiRR′R″ and —NRR′, or two or more groups adjacent to each other may bond to each other to form a substituted or unsubstituted C6 to C60 aliphatic or aromatic hydrocarbon ring or a substituted or unsubstituted C2 to C60 aliphatic or aromatic heteroring.

In another embodiment, R11 to R18 and R21 to R28 are the same as or different from each other, and each independently selected from the group consisting of hydrogen; deuterium; a substituted or unsubstituted C6 to C60 aryl group; and a substituted or unsubstituted C2 to C60 heteroaryl group, or two or more groups adjacent to each other may bond to each other to form a substituted or unsubstituted C6 to C60 aliphatic or aromatic hydrocarbon ring or a substituted or unsubstituted C2 to C60 aliphatic or aromatic heteroring.

In another embodiment, R11 to R18 and R21 to R28 are the same as or different from each other, and each independently selected from the group consisting of hydrogen; deuterium; a substituted or unsubstituted C6 to C40 aryl group; and a substituted or unsubstituted C2 to C40 heteroaryl group, or two or more groups adjacent to each other may bond to each other to form a substituted or unsubstituted C6 to C40 aromatic hydrocarbon ring or a substituted or unsubstituted C2 to C40 aromatic heteroring.

In another embodiment, R11 to R18 and R21 to R28 are the same as or different from each other, and each independently selected from the group consisting of hydrogen; deuterium; a C6 to C40 aryl group; and a C2 to C40 heteroaryl group unsubstituted or substituted with a C6 to C40 aryl group, or two or more groups adjacent to each other may bond to each other to form a C6 to C40 aromatic hydrocarbon ring unsubstituted or substituted with a C1 to C20 alkyl group, or a C2 to C40 aromatic heteroring unsubstituted or substituted with a C6 to C40 aryl group.

In another embodiment, R11 to R18 and R21 to R28 are the same as or different from each other, and each independently hydrogen; deuterium; a phenyl group; a biphenyl group; or a carbazole group unsubstituted or substituted with a phenyl group, or two or more groups adjacent to each other may bond to each other to form a benzothiophene ring; a benzofuran ring; an indole ring unsubstituted or substituted with a phenyl group; or an indene ring unsubstituted or substituted with a methyl group.

In one embodiment of the present application, R, R′ and R″ are the same as or different from each other, and may be each independently a substituted or unsubstituted C1 to C60 alkyl group; a substituted or unsubstituted C6 to C60 aryl group; or a substituted or unsubstituted C2 to C60 heteroaryl group.

In another embodiment, R, R′ and R″ are the same as or different from each other, and may be each independently a substituted or unsubstituted C6 to C60 aryl group.

In another embodiment, R, R′ and R″ are the same as or different from each other, and may be each independently a substituted or unsubstituted C6 to C60 monocyclic or polycyclic aryl group.

In another embodiment, R, R′ and R″ are the same as or different from each other, and may be each independently a substituted or unsubstituted C6 to C40 monocyclic aryl group.

In another embodiment, R, R′ and R″ are the same as or different from each other, and may be each independently a C6 to C20 monocyclic aryl group.

In another embodiment, R, R′ and R″ may be a phenyl group.

In one embodiment of the present application, R31 has the same definition as R.

According to one embodiment of the present application, Chemical Formula 1 may be represented by any one of the following compounds, but is not limited thereto.

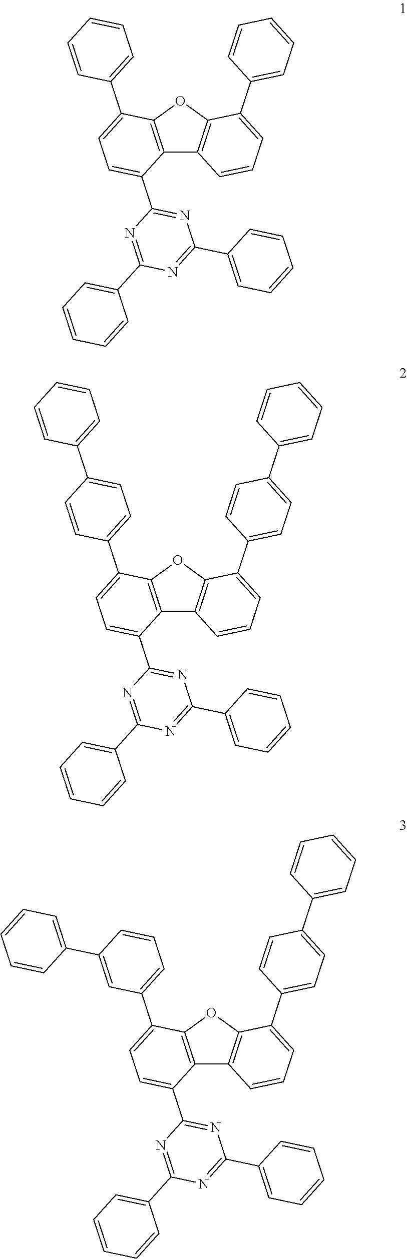

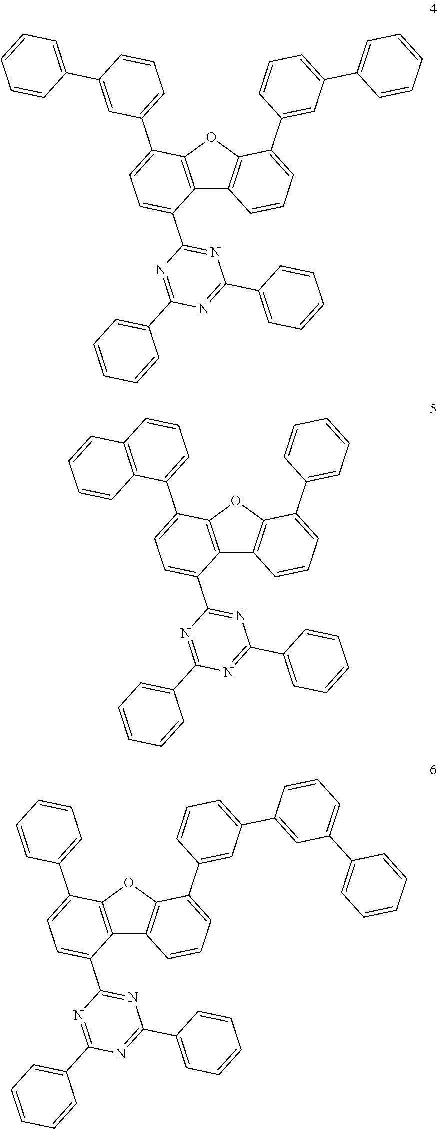

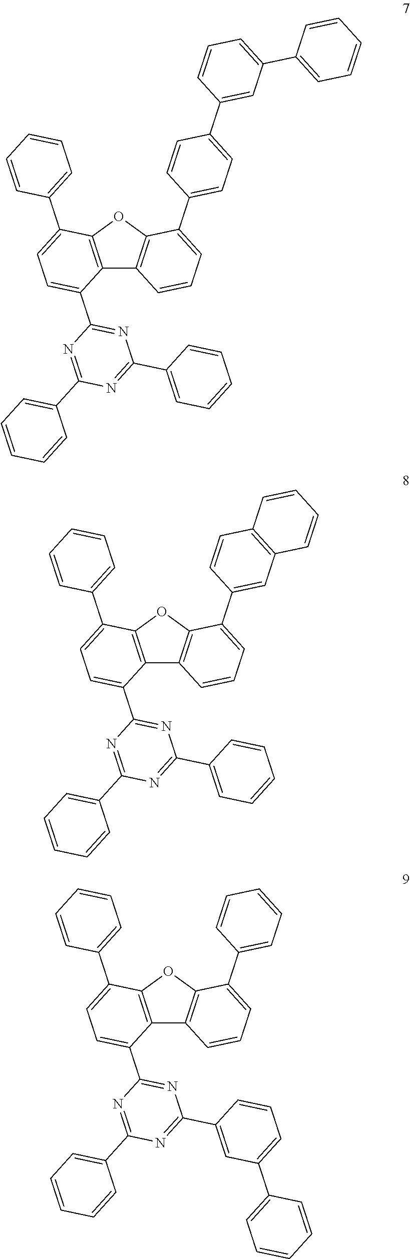

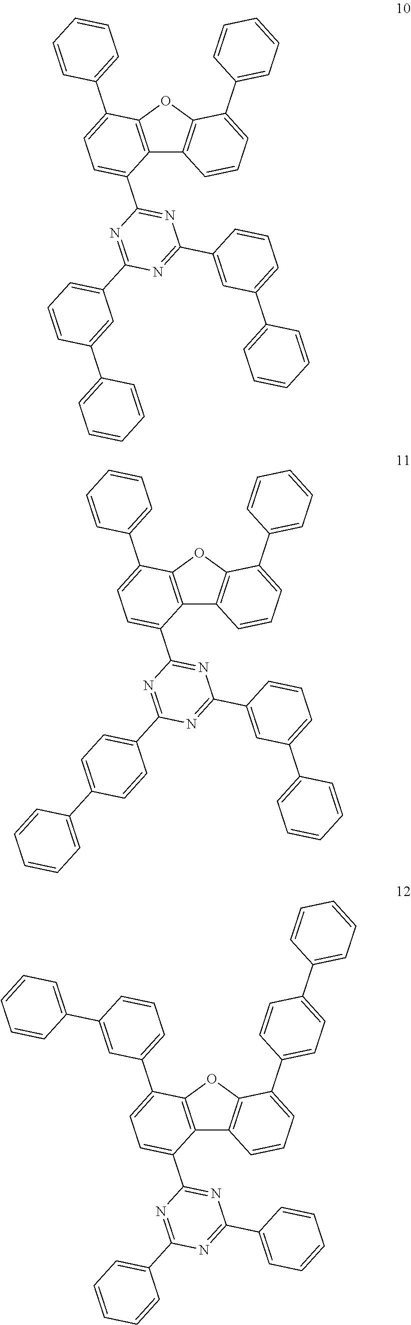

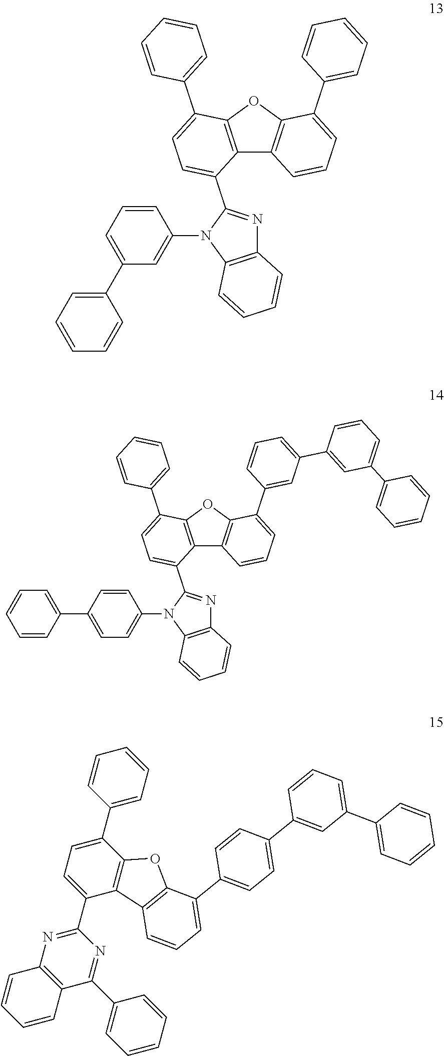 [SMILES: c1ccc(-c2cccc(-n3c(-c4ccc(-c5ccccc5)c5oc6c(-c7ccccc7)cccc6c45)nc4ccccc43)c2)cc1 ; c1ccc(-c2ccc(-n3c(-c4ccc(-c5ccccc5)c5oc6c(-c7cccc(-c8cccc(-c9ccccc9)c8)c7)cccc6c45)nc4ccccc43)cc2)cc1 ; c1ccc(-c2cccc(-c3ccc(-c4cccc5c4oc4c(-c6ccccc6)ccc(-c6nc(-c7ccccc7)c7ccccc7n6)c45)cc3)c2)cc1]

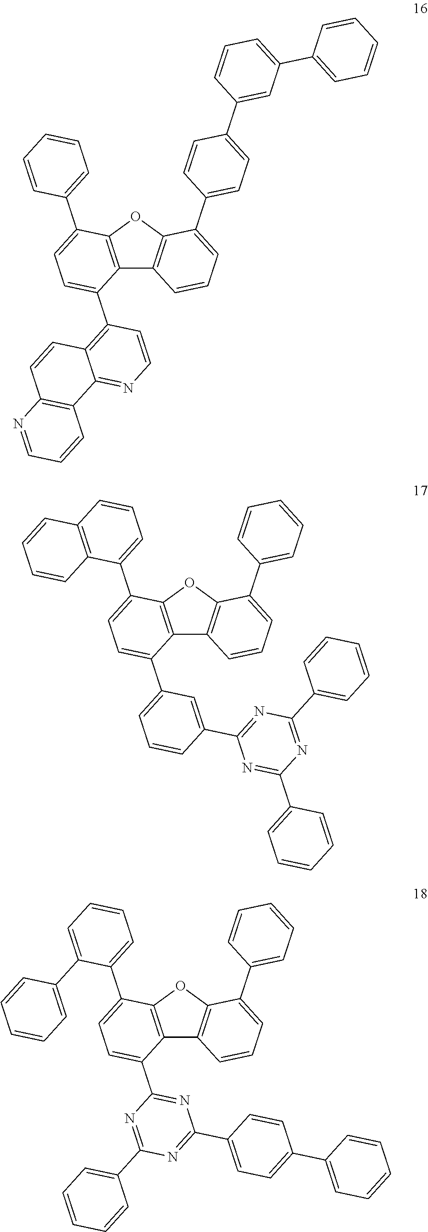

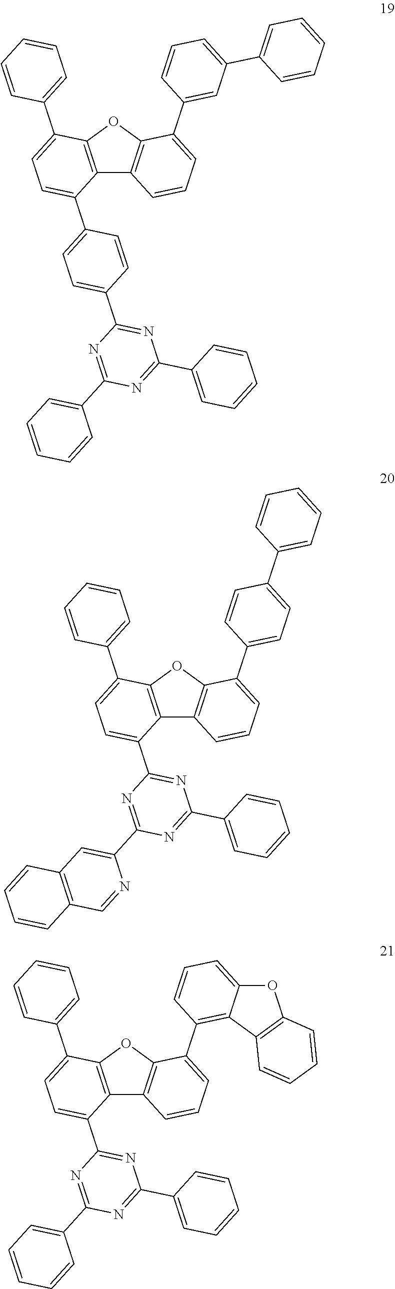

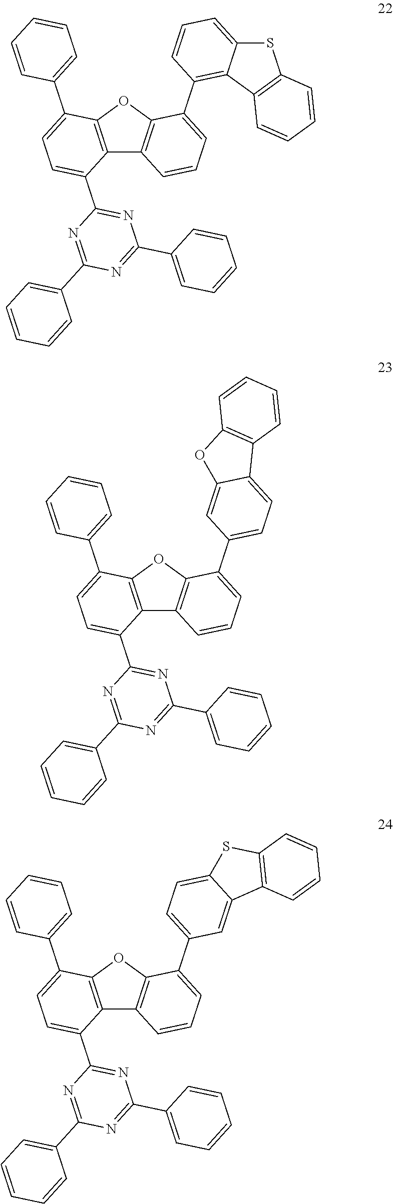 [SMILES: c1ccc(-c2nc(-c3ccccc3)nc(-c3ccc(-c4ccccc4)c4oc5c(-c6cccc7sc8ccccc8c67)cccc5c34)n2)cc1 ; c1ccc(-c2nc(-c3ccccc3)nc(-c3ccc(-c4ccccc4)c4oc5c(-c6ccc7c(c6)oc6ccccc67)cccc5c34)n2)cc1 ; c1ccc(-c2nc(-c3ccccc3)nc(-c3ccc(-c4ccccc4)c4oc5c(-c6ccc7sc8ccccc8c7c6)cccc5c34)n2)cc1]

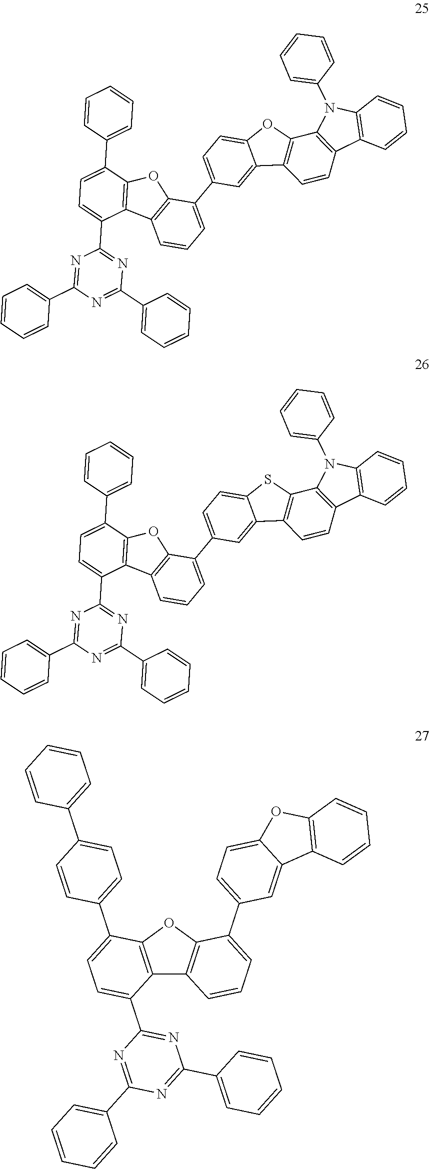 [SMILES: c1ccc(-c2nc(-c3ccccc3)nc(-c3ccc(-c4ccccc4)c4oc5c(-c6ccc7oc8c(ccc9c%10ccccc%10n(-c%10ccccc%10)c98)c7c6)cccc5c34)n2)cc1 ; c1ccc(-c2nc(-c3ccccc3)nc(-c3ccc(-c4ccccc4)c4oc5c(-c6ccc7sc8c(ccc9c%10ccccc%10n(-c%10ccccc%10)c98)c7c6)cccc5c34)n2)cc1 ; c1ccc(-c2ccc(-c3ccc(-c4nc(-c5ccccc5)nc(-c5ccccc5)n4)c4c3oc3c(-c5ccc6oc7ccccc7c6c5)cccc34)cc2)cc1]

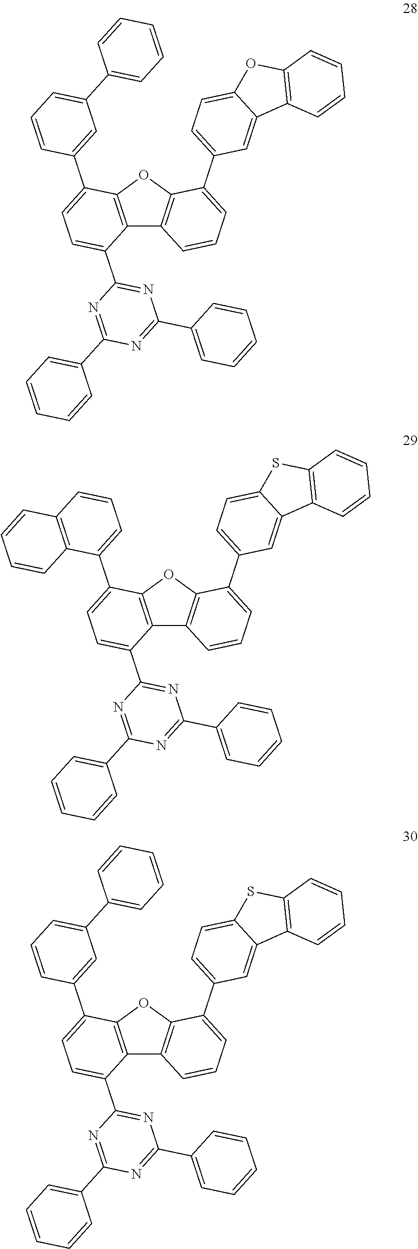

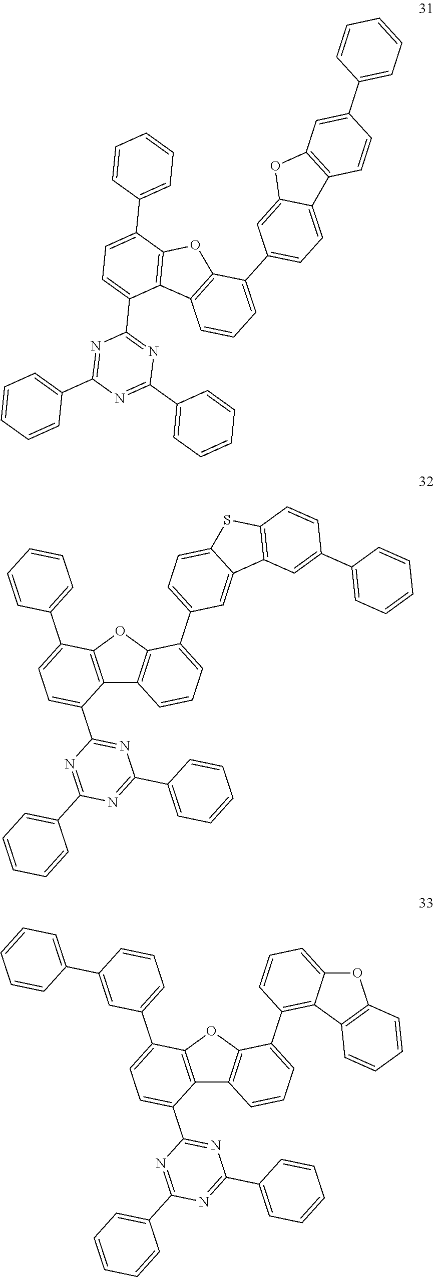 [SMILES: c1ccc(-c2ccc3c(c2)oc2cc(-c4cccc5c4oc4c(-c6ccccc6)ccc(-c6nc(-c7ccccc7)nc(-c7ccccc7)n6)c45)ccc23)cc1 ; c1ccc(-c2ccc3sc4ccc(-c5cccc6c5oc5c(-c7ccccc7)ccc(-c7nc(-c8ccccc8)nc(-c8ccccc8)n7)c56)cc4c3c2)cc1 ; c1ccc(-c2cccc(-c3ccc(-c4nc(-c5ccccc5)nc(-c5ccccc5)n4)c4c3oc3c(-c5cccc6oc7ccccc7c56)cccc34)c2)cc1]

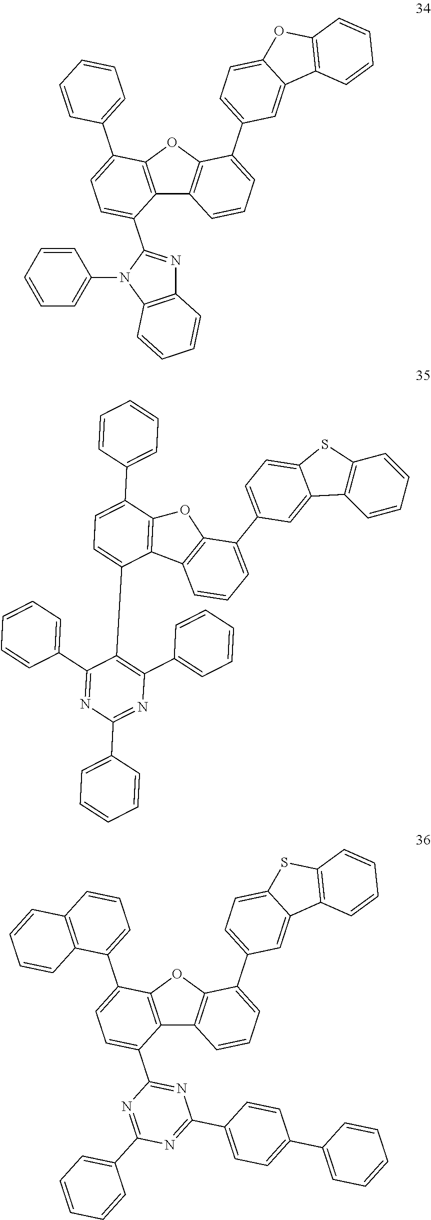 [SMILES: c1ccc(-c2ccc(-c3nc4ccccc4n3-c3ccccc3)c3c2oc2c(-c4ccc5oc6ccccc6c5c4)cccc23)cc1 ; c1ccc(-c2nc(-c3ccccc3)c(-c3ccc(-c4ccccc4)c4oc5c(-c6ccc7sc8ccccc8c7c6)cccc5c34)c(-c3ccccc3)n2)cc1 ; c1ccc(-c2ccc(-c3nc(-c4ccccc4)nc(-c4ccc(-c5cccc6ccccc56)c5oc6c(-c7ccc8sc9ccccc9c8c7)cccc6c45)n3)cc2)cc1]

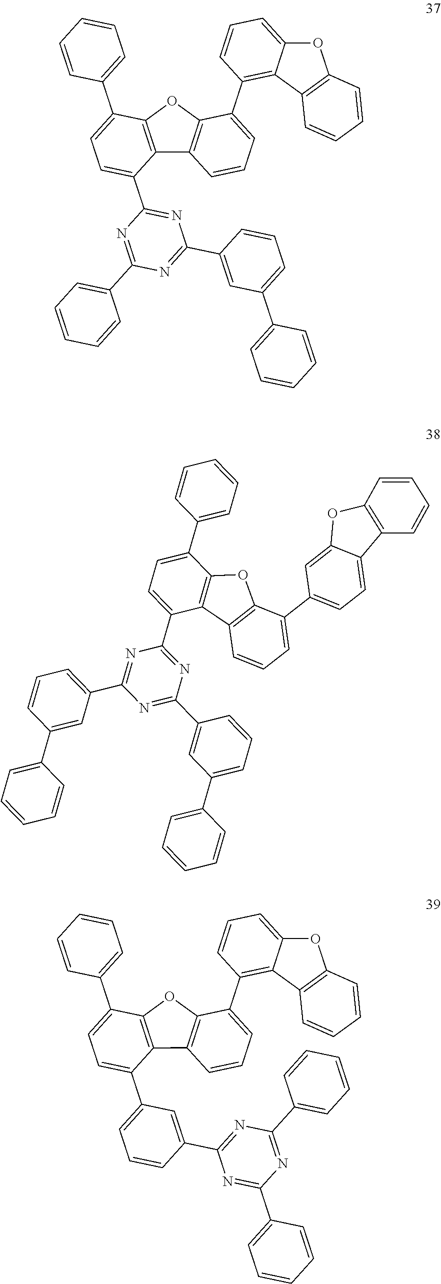 [SMILES: c1ccc(-c2cccc(-c3nc(-c4ccccc4)nc(-c4ccc(-c5ccccc5)c5oc6c(-c7cccc8oc9ccccc9c78)cccc6c45)n3)c2)cc1 ; c1ccc(-c2cccc(-c3nc(-c4cccc(-c5ccccc5)c4)nc(-c4ccc(-c5ccccc5)c5oc6c(-c7ccc8c(c7)oc7ccccc78)cccc6c45)n3)c2)cc1 ; c1ccc(-c2nc(-c3ccccc3)nc(-c3cccc(-c4ccc(-c5ccccc5)c5oc6c(-c7cccc8oc9ccccc9c78)cccc6c45)c3)n2)cc1]

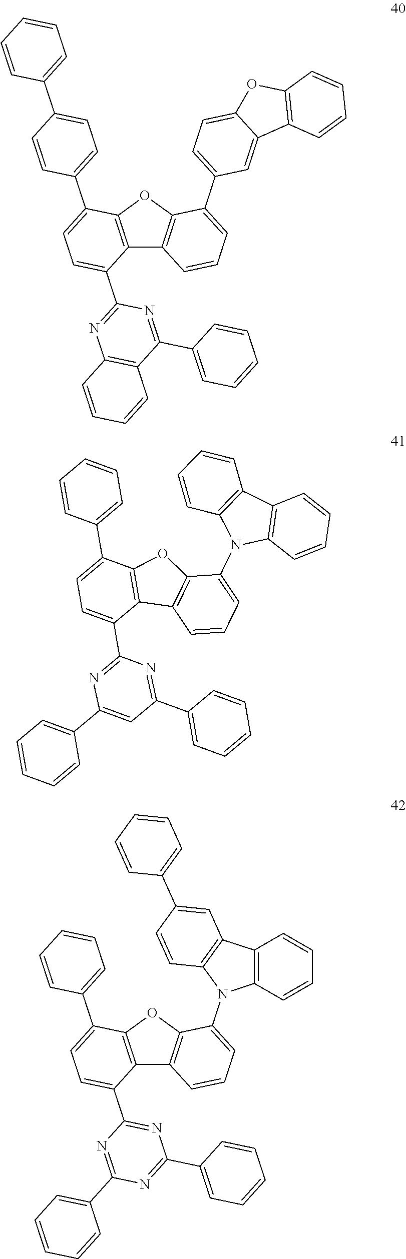

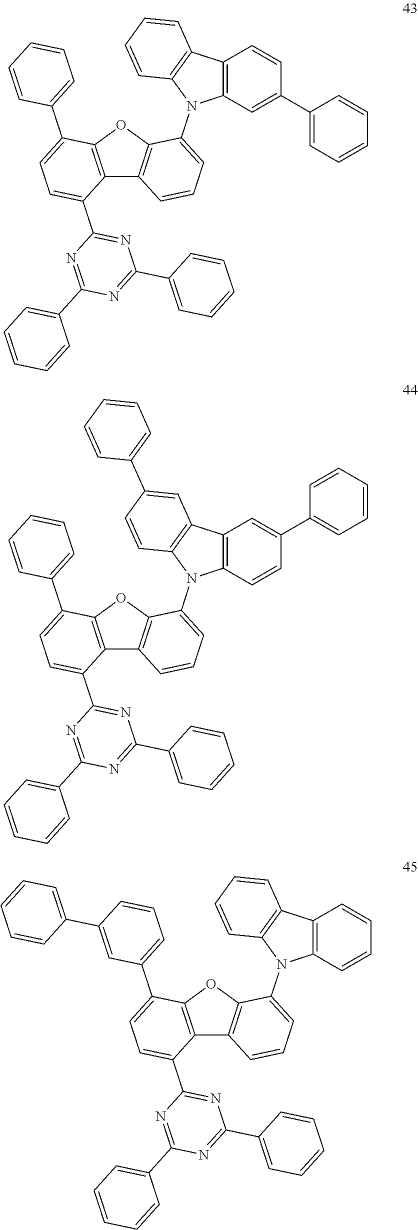

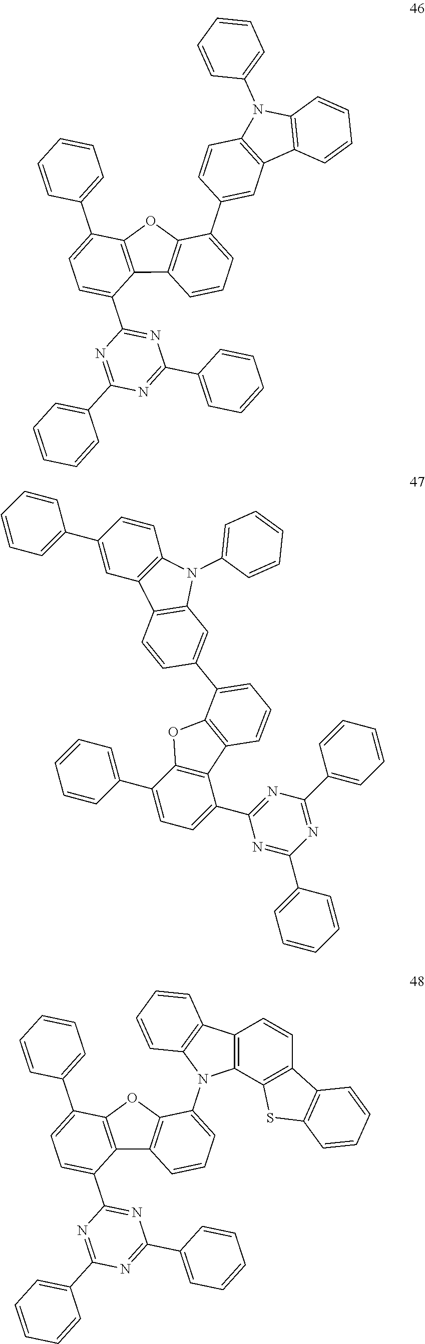

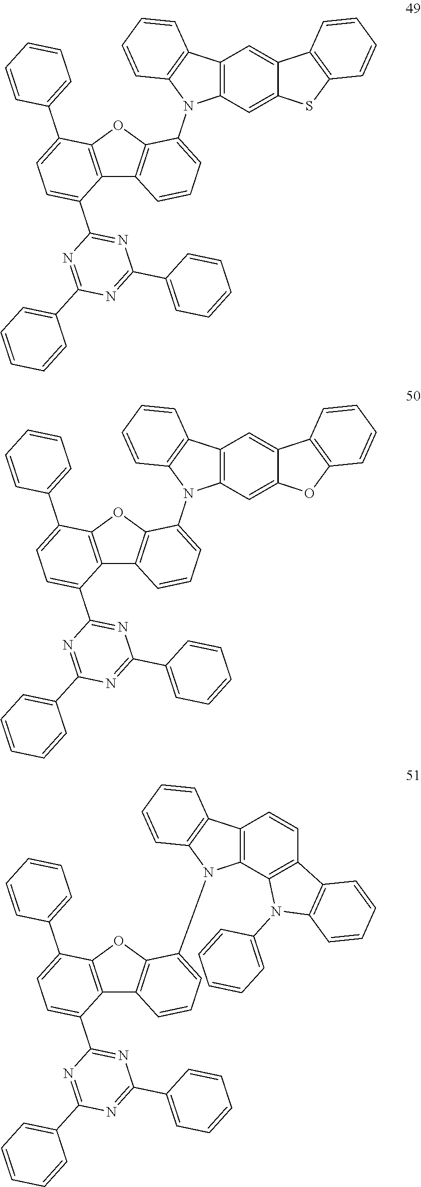 [SMILES: c1ccc(-c2nc(-c3ccccc3)nc(-c3ccc(-c4ccccc4)c4oc5c(-n6c7ccccc7c7cc8c(cc76)sc6ccccc68)cccc5c34)n2)cc1 ; c1ccc(-c2nc(-c3ccccc3)nc(-c3ccc(-c4ccccc4)c4oc5c(-n6c7ccccc7c7cc8c(cc76)oc6ccccc68)cccc5c34)n2)cc1 ; c1ccc(-c2nc(-c3ccccc3)nc(-c3ccc(-c4ccccc4)c4oc5c(-n6c7ccccc7c7ccc8c9ccccc9n(-c9ccccc9)c8c76)cccc5c34)n2)cc1]

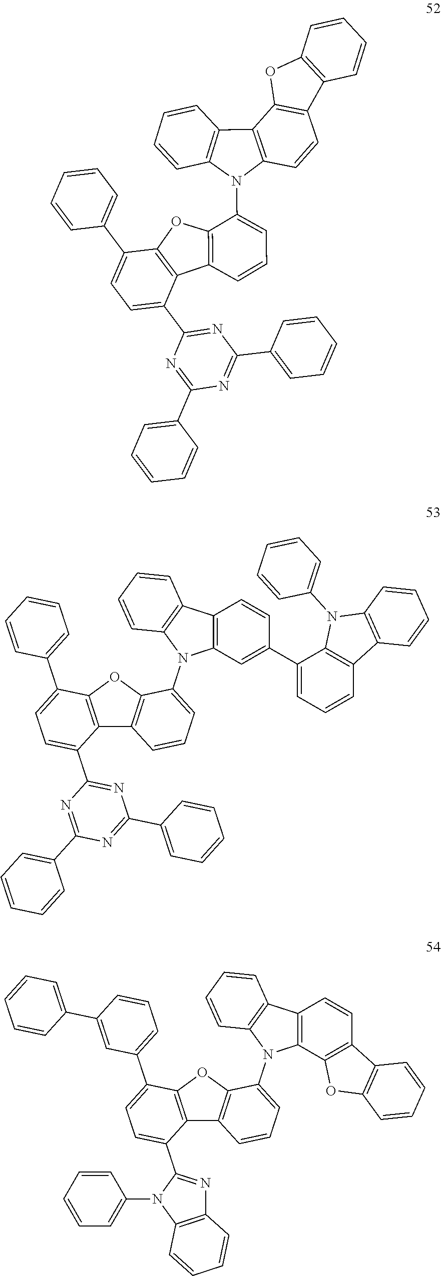 [SMILES: c1ccc(-c2nc(-c3ccccc3)nc(-c3ccc(-c4ccccc4)c4oc5c(-n6c7ccccc7c7c8oc9ccccc9c8ccc76)cccc5c34)n2)cc1 ; c1ccc(-c2nc(-c3ccccc3)nc(-c3ccc(-c4ccccc4)c4oc5c(-n6c7ccccc7c7ccc(-c8cccc9c%10ccccc%10n(-c%10ccccc%10)c89)cc76)cccc5c34)n2)cc1 ; c1ccc(-c2cccc(-c3ccc(-c4nc5ccccc5n4-c4ccccc4)c4c3oc3c(-n5c6ccccc6c6ccc7c8ccccc8oc7c65)cccc34)c2)cc1]

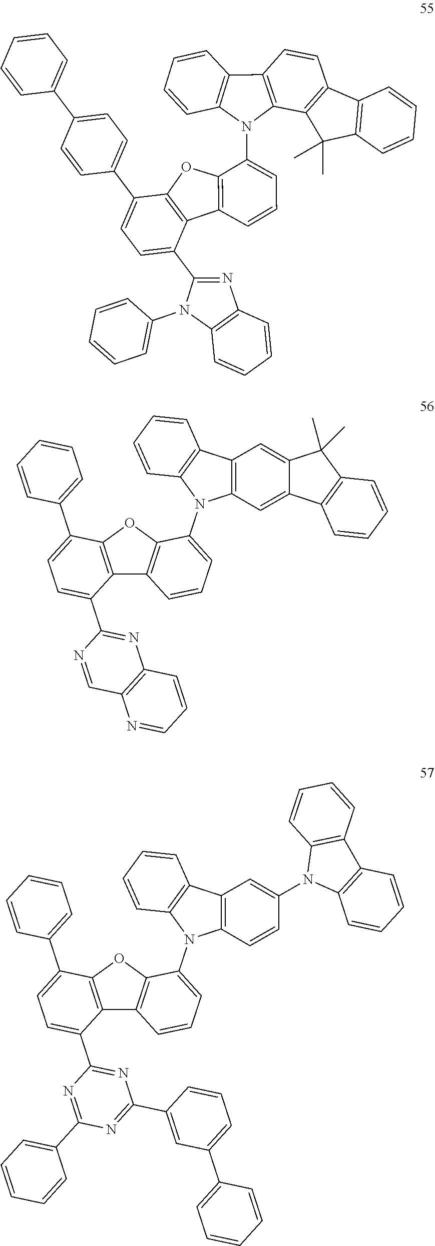 [SMILES: CC1(C)c2ccccc2-c2ccc3c4ccccc4n(-c4cccc5c4oc4c(-c6ccc(-c7ccccc7)cc6)ccc(-c6nc7ccccc7n6-c6ccccc6)c45)c3c21 ; CC1(C)c2ccccc2-c2cc3c(cc21)c1ccccc1n3-c1cccc2c1oc1c(-c3ccccc3)ccc(-c3ncc4ncccc4n3)c12 ; c1ccc(-c2cccc(-c3nc(-c4ccccc4)nc(-c4ccc(-c5ccccc5)c5oc6c(-n7c8ccccc8c8cc(-n9c%10ccccc%10c%10ccccc%109)ccc87)cccc6c45)n3)c2)cc1]

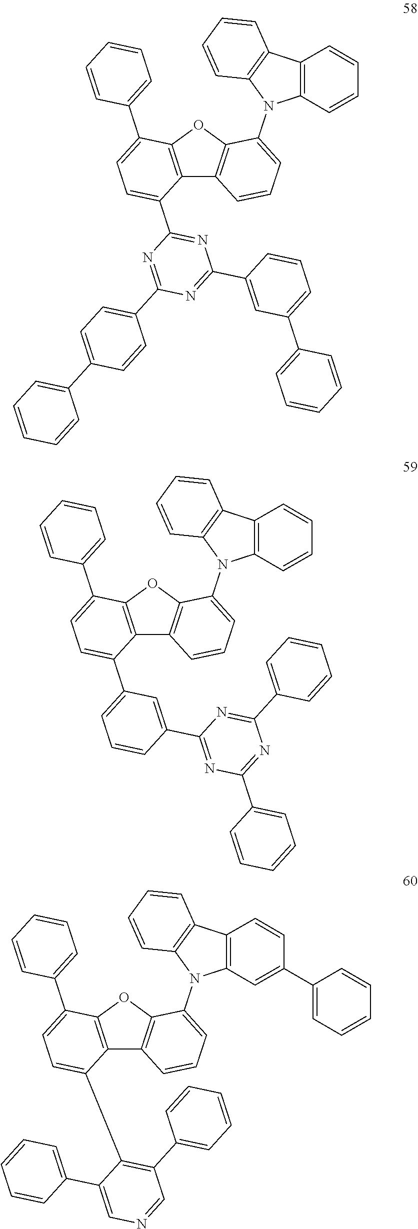 [SMILES: c1ccc(-c2ccc(-c3nc(-c4cccc(-c5ccccc5)c4)nc(-c4ccc(-c5ccccc5)c5oc6c(-n7c8ccccc8c8ccccc87)cccc6c45)n3)cc2)cc1 ; c1ccc(-c2nc(-c3ccccc3)nc(-c3cccc(-c4ccc(-c5ccccc5)c5oc6c(-n7c8ccccc8c8ccccc87)cccc6c45)c3)n2)cc1 ; c1ccc(-c2ccc3c4ccccc4n(-c4cccc5c4oc4c(-c6ccccc6)ccc(-c6c(-c7ccccc7)cncc6-c6ccccc6)c45)c3c2)cc1]

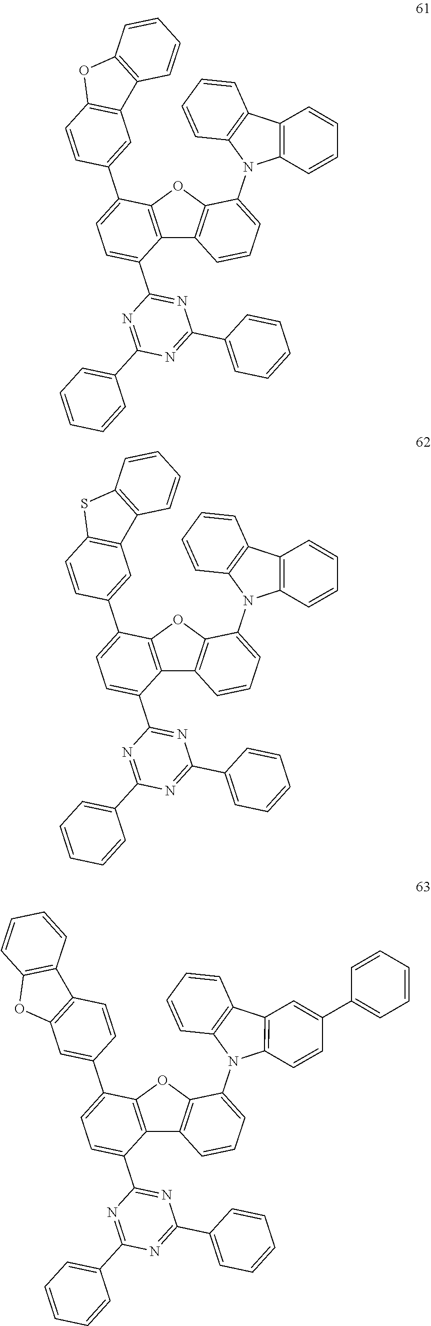

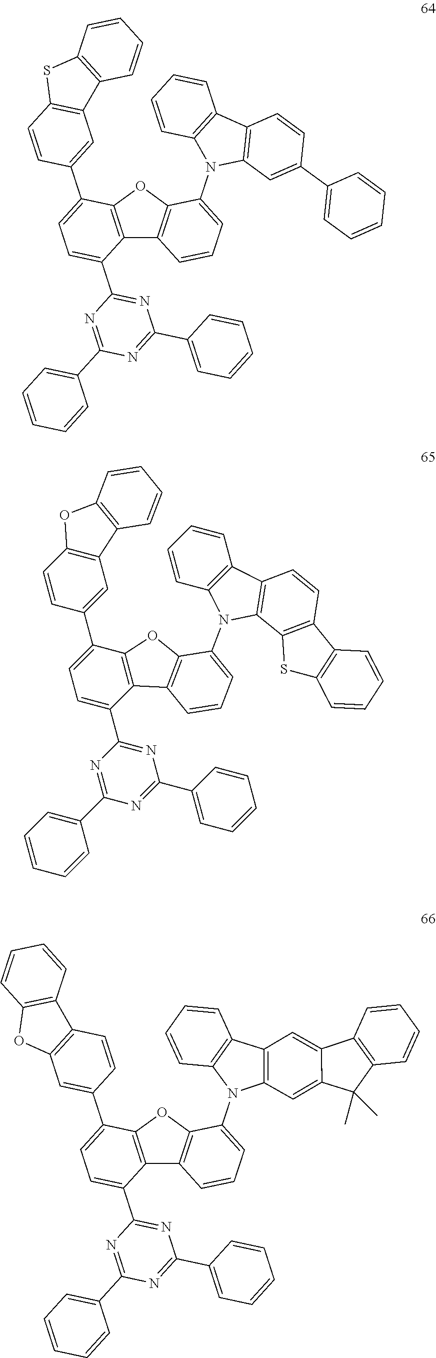 [SMILES: c1ccc(-c2ccc3c4ccccc4n(-c4cccc5c4oc4c(-c6ccc7sc8ccccc8c7c6)ccc(-c6nc(-c7ccccc7)nc(-c7ccccc7)n6)c45)c3c2)cc1 ; c1ccc(-c2nc(-c3ccccc3)nc(-c3ccc(-c4ccc5oc6ccccc6c5c4)c4oc5c(-n6c7ccccc7c7ccc8c9ccccc9sc8c76)cccc5c34)n2)cc1 ; CC1(C)c2ccccc2-c2cc3c4ccccc4n(-c4cccc5c4oc4c(-c6ccc7c(c6)oc6ccccc67)ccc(-c6nc(-c7ccccc7)nc(-c7ccccc7)n6)c45)c3cc21]

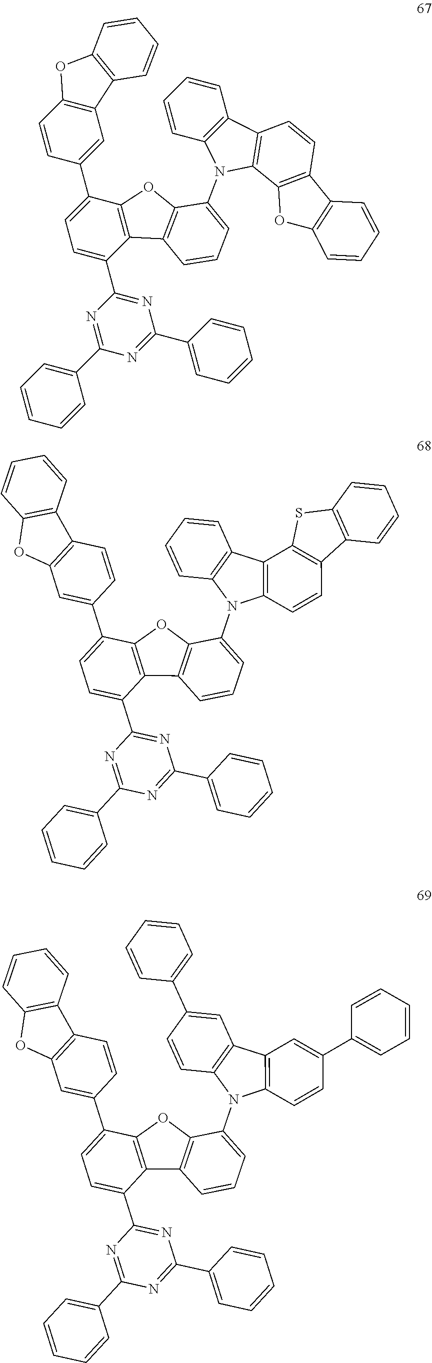

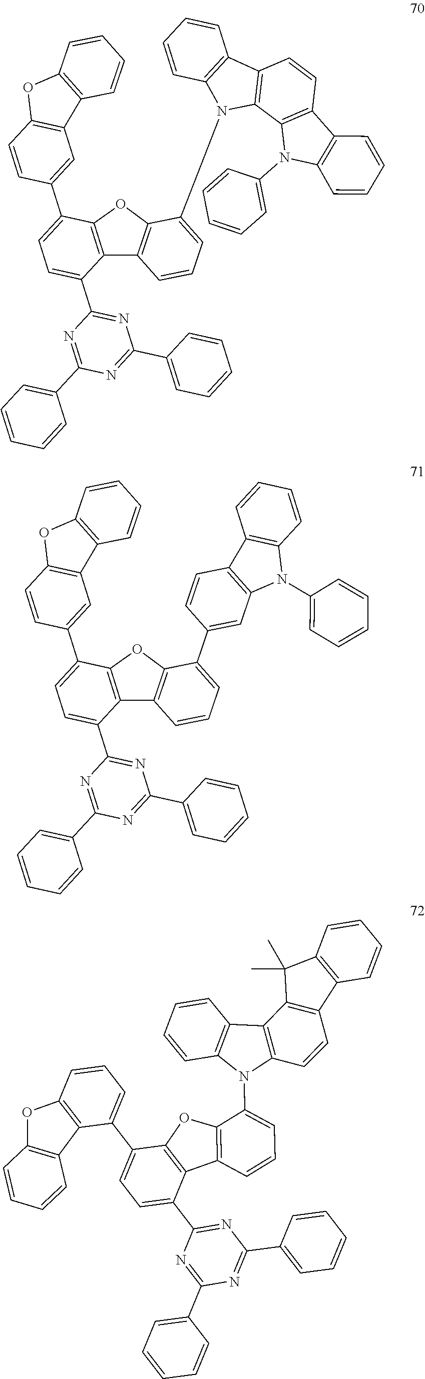 [SMILES: c1ccc(-c2nc(-c3ccccc3)nc(-c3ccc(-c4ccc5oc6ccccc6c5c4)c4oc5c(-n6c7ccccc7c7ccc8c9ccccc9n(-c9ccccc9)c8c76)cccc5c34)n2)cc1 ; c1ccc(-c2nc(-c3ccccc3)nc(-c3ccc(-c4ccc5oc6ccccc6c5c4)c4oc5c(-c6ccc7c8ccccc8n(-c8ccccc8)c7c6)cccc5c34)n2)cc1 ; CC1(C)c2ccccc2-c2ccc3c(c21)c1ccccc1n3-c1cccc2c1oc1c(-c3cccc4oc5ccccc5c34)ccc(-c3nc(-c4ccccc4)nc(-c4ccccc4)n3)c12]

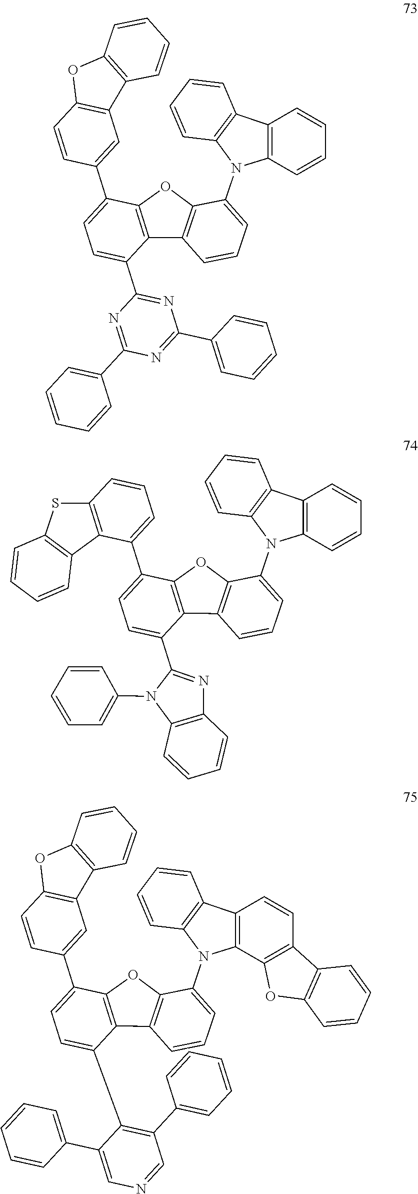 [SMILES: c1ccc(-c2nc(-c3ccccc3)nc(-c3ccc(-c4ccc5oc6ccccc6c5c4)c4oc5c(-n6c7ccccc7c7ccccc76)cccc5c34)n2)cc1 ; c1ccc(-n2c(-c3ccc(-c4cccc5sc6ccccc6c45)c4oc5c(-n6c7ccccc7c7ccccc76)cccc5c34)nc3ccccc32)cc1 ; c1ccc(-c2cncc(-c3ccccc3)c2-c2ccc(-c3ccc4oc5ccccc5c4c3)c3oc4c(-n5c6ccccc6c6ccc7c8ccccc8oc7c65)cccc4c23)cc1]

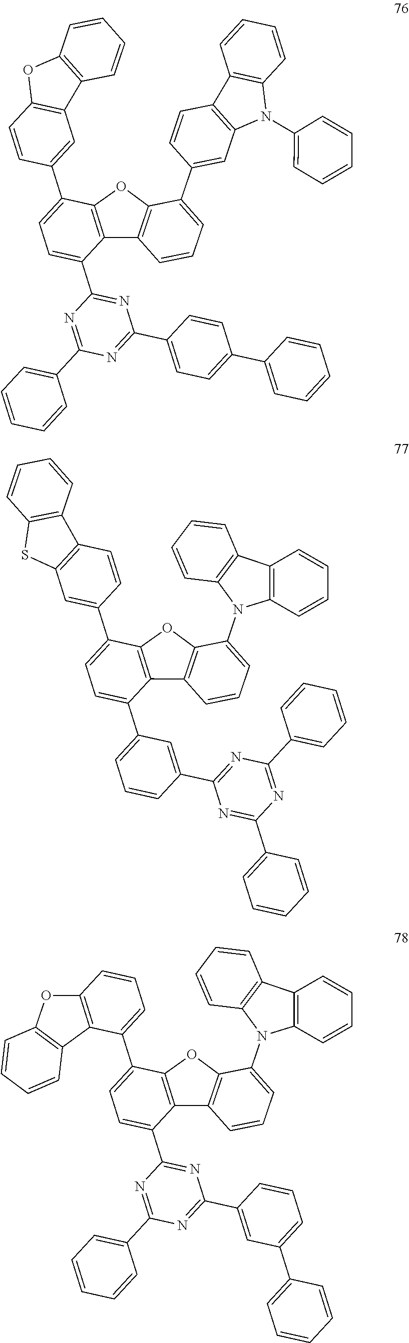

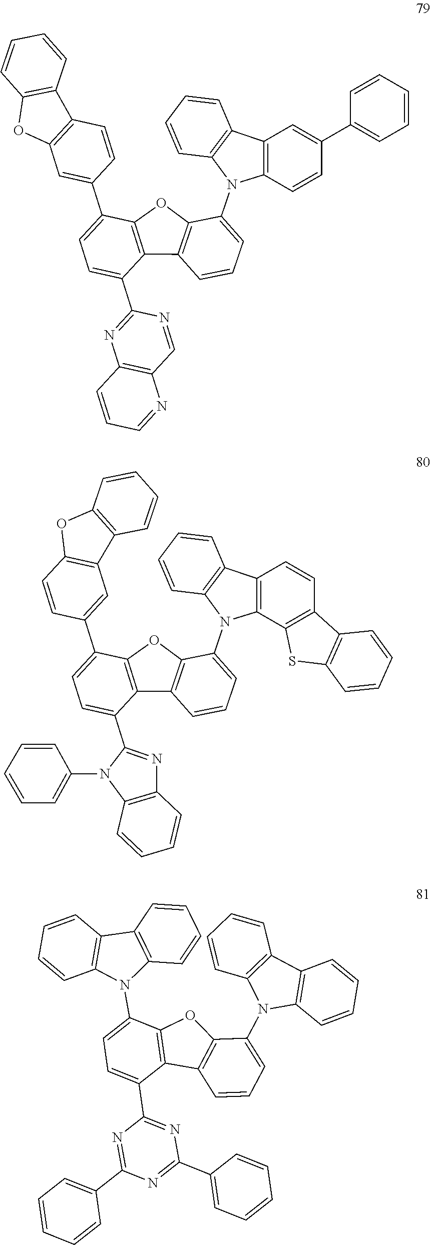

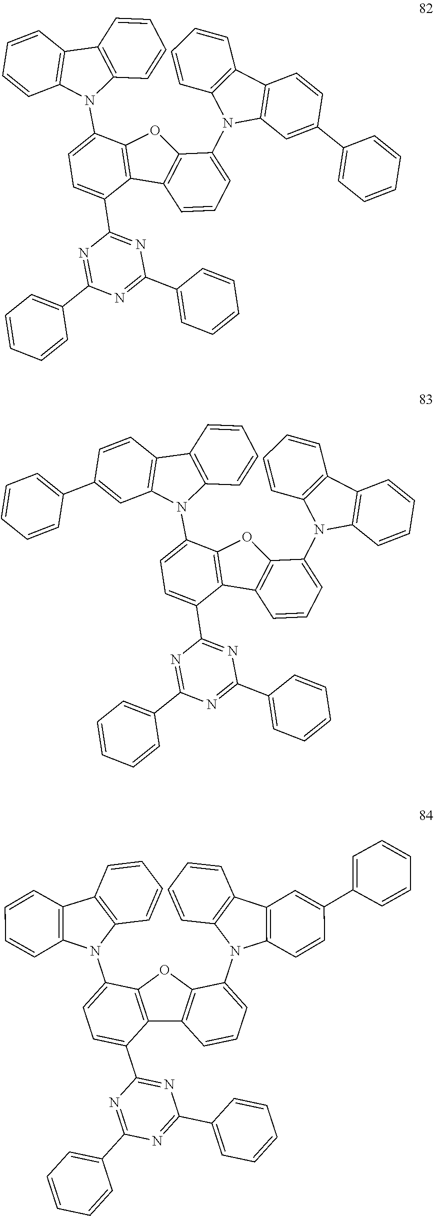 [SMILES: c1ccc(-c2ccc3c4ccccc4n(-c4cccc5c4oc4c(-n6c7ccccc7c7ccccc76)ccc(-c6nc(-c7ccccc7)nc(-c7ccccc7)n6)c45)c3c2)cc1 ; c1ccc(-c2ccc3c4ccccc4n(-c4ccc(-c5nc(-c6ccccc6)nc(-c6ccccc6)n5)c5c4oc4c(-n6c7ccccc7c7ccccc76)cccc45)c3c2)cc1 ; c1ccc(-c2ccc3c(c2)c2ccccc2n3-c2cccc3c2oc2c(-n4c5ccccc5c5ccccc54)ccc(-c4nc(-c5ccccc5)nc(-c5ccccc5)n4)c23)cc1]

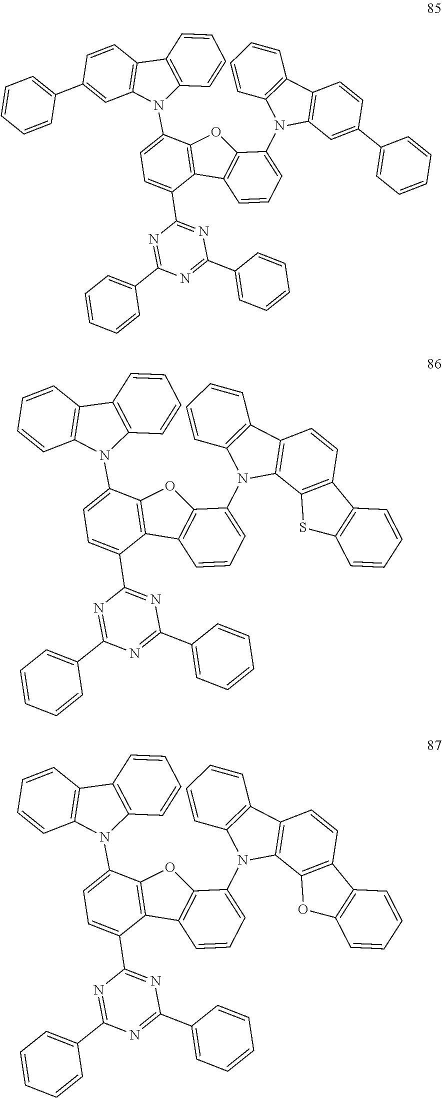

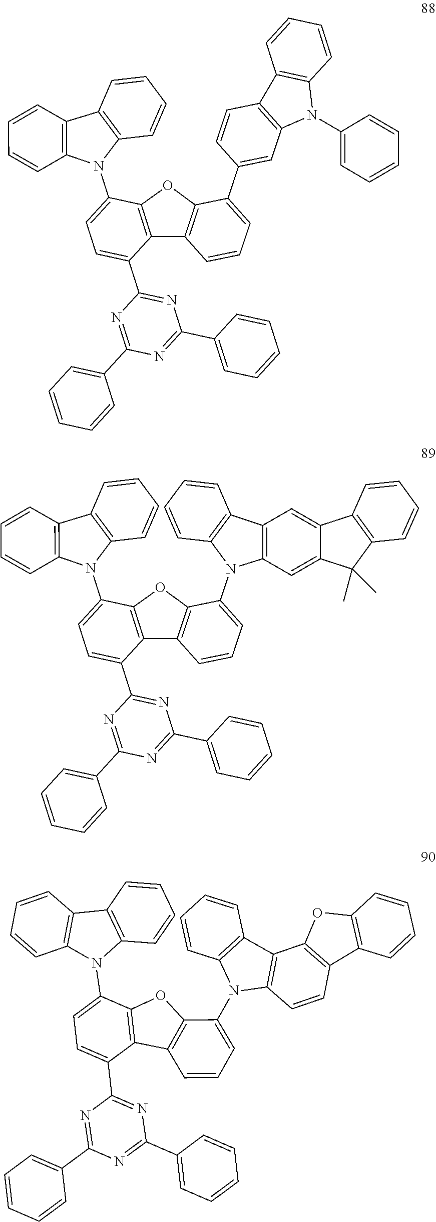 [SMILES: c1ccc(-c2nc(-c3ccccc3)nc(-c3ccc(-n4c5ccccc5c5ccccc54)c4oc5c(-c6ccc7c8ccccc8n(-c8ccccc8)c7c6)cccc5c34)n2)cc1 ; CC1(C)c2ccccc2-c2cc3c4ccccc4n(-c4cccc5c4oc4c(-n6c7ccccc7c7ccccc76)ccc(-c6nc(-c7ccccc7)nc(-c7ccccc7)n6)c45)c3cc21 ; c1ccc(-c2nc(-c3ccccc3)nc(-c3ccc(-n4c5ccccc5c5ccccc54)c4oc5c(-n6c7ccccc7c7c8oc9ccccc9c8ccc76)cccc5c34)n2)cc1]

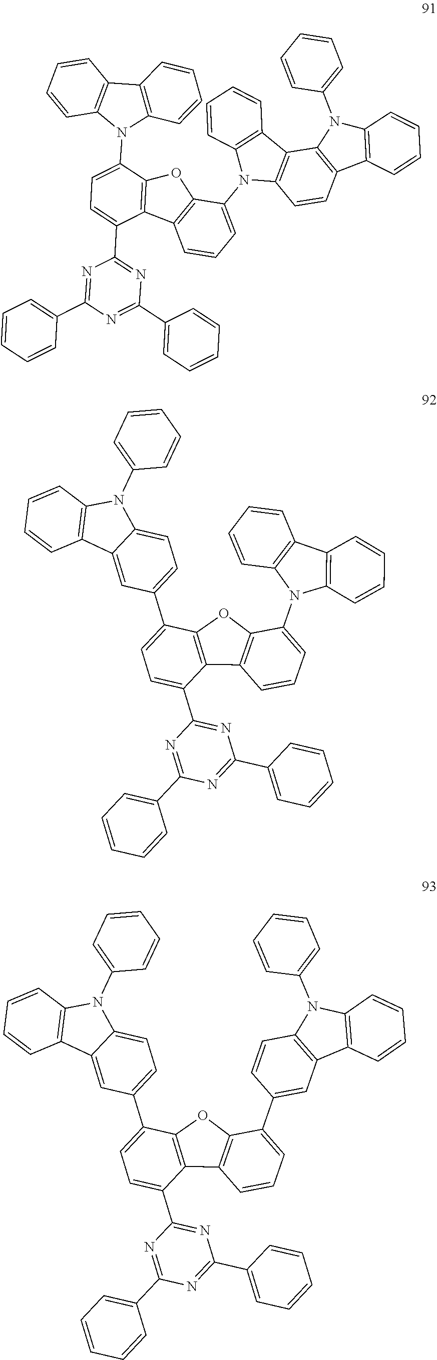 [SMILES: c1ccc(-c2nc(-c3ccccc3)nc(-c3ccc(-n4c5ccccc5c5ccccc54)c4oc5c(-n6c7ccccc7c7c6ccc6c8ccccc8n(-c8ccccc8)c67)cccc5c34)n2)cc1 ; c1ccc(-c2nc(-c3ccccc3)nc(-c3ccc(-c4ccc5c(c4)c4ccccc4n5-c4ccccc4)c4oc5c(-n6c7ccccc7c7ccccc76)cccc5c34)n2)cc1 ; c1ccc(-c2nc(-c3ccccc3)nc(-c3ccc(-c4ccc5c(c4)c4ccccc4n5-c4ccccc4)c4oc5c(-c6ccc7c(c6)c6ccccc6n7-c6ccccc6)cccc5c34)n2)cc1]

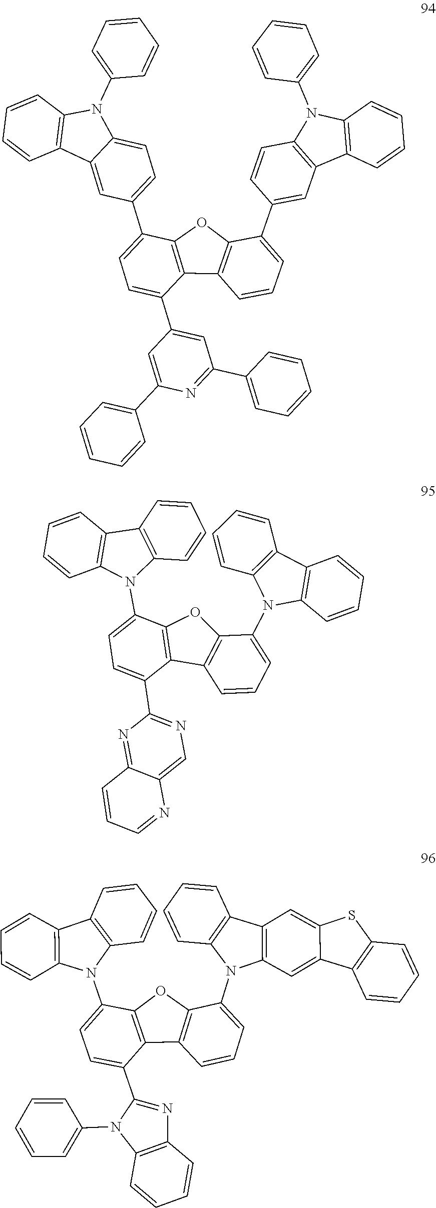 [SMILES: c1ccc(-c2cc(-c3ccc(-c4ccc5c(c4)c4ccccc4n5-c4ccccc4)c4oc5c(-c6ccc7c(c6)c6ccccc6n7-c6ccccc6)cccc5c34)cc(-c3ccccc3)n2)cc1 ; c1cnc2cnc(-c3ccc(-n4c5ccccc5c5ccccc54)c4oc5c(-n6c7ccccc7c7ccccc76)cccc5c34)nc2c1 ; c1ccc(-n2c(-c3ccc(-n4c5ccccc5c5ccccc54)c4oc5c(-n6c7ccccc7c7cc8sc9ccccc9c8cc76)cccc5c34)nc3ccccc32)cc1]

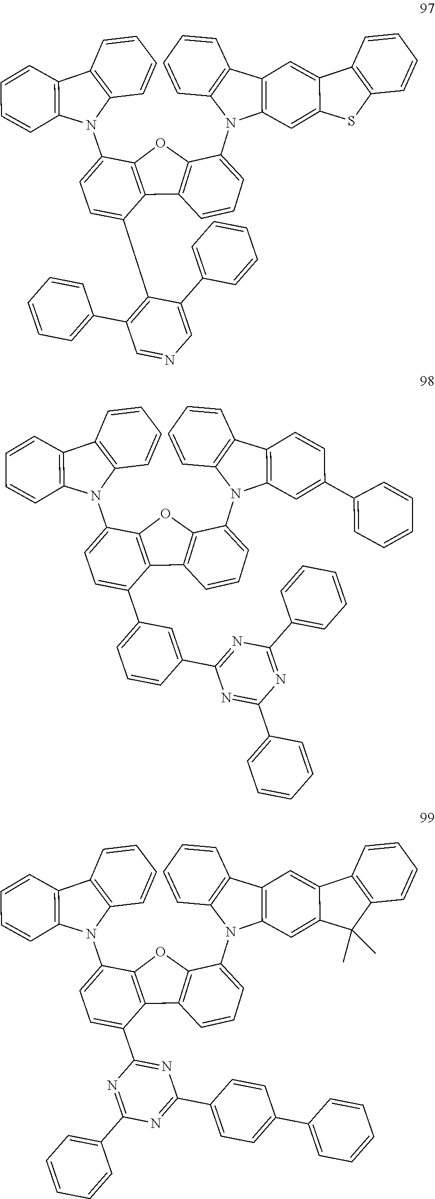 [SMILES: c1ccc(-c2cncc(-c3ccccc3)c2-c2ccc(-n3c4ccccc4c4ccccc43)c3oc4c(-n5c6ccccc6c6cc7c(cc65)sc5ccccc57)cccc4c23)cc1 ; c1ccc(-c2ccc3c4ccccc4n(-c4cccc5c4oc4c(-n6c7ccccc7c7ccccc76)ccc(-c6cccc(-c7nc(-c8ccccc8)nc(-c8ccccc8)n7)c6)c45)c3c2)cc1 ; CC1(C)c2ccccc2-c2cc3c4ccccc4n(-c4cccc5c4oc4c(-n6c7ccccc7c7ccccc76)ccc(-c6nc(-c7ccccc7)nc(-c7ccc(-c8ccccc8)cc7)n6)c45)c3cc21]

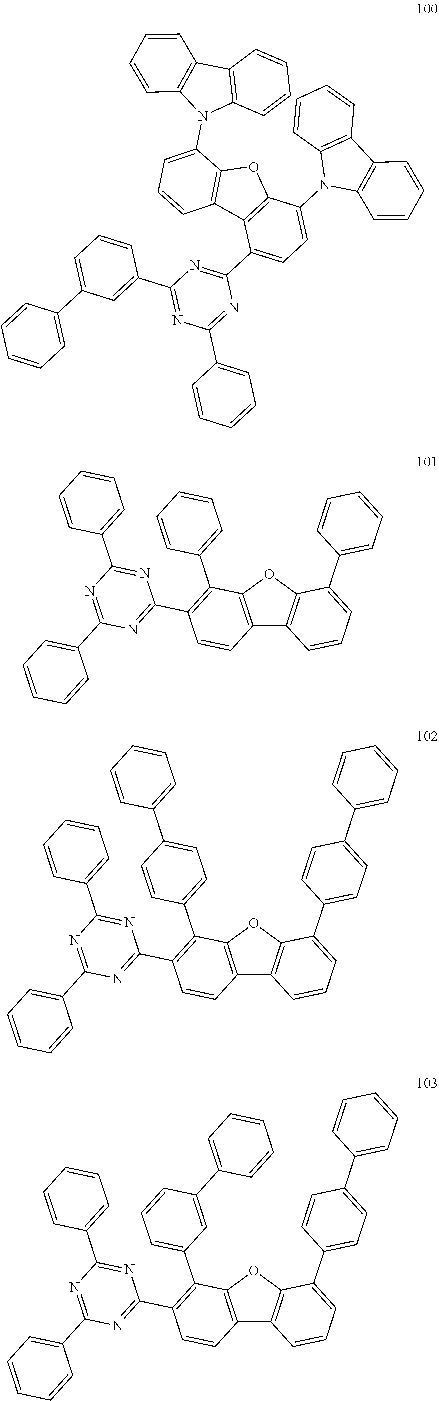 [SMILES: c1ccc(-c2cccc(-c3nc(-c4ccccc4)nc(-c4ccc(-n5c6ccccc6c6ccccc65)c5oc6c(-n7c8ccccc8c8ccccc87)cccc6c45)n3)c2)cc1 ; c1ccc(-c2nc(-c3ccccc3)nc(-c3ccc4c(oc5c(-c6ccccc6)cccc54)c3-c3ccccc3)n2)cc1 ; c1ccc(-c2ccc(-c3cccc4c3oc3c(-c5ccc(-c6ccccc6)cc5)c(-c5nc(-c6ccccc6)nc(-c6ccccc6)n5)ccc34)cc2)cc1 ; c1ccc(-c2ccc(-c3cccc4c3oc3c(-c5cccc(-c6ccccc6)c5)c(-c5nc(-c6ccccc6)nc(-c6ccccc6)n5)ccc34)cc2)cc1]

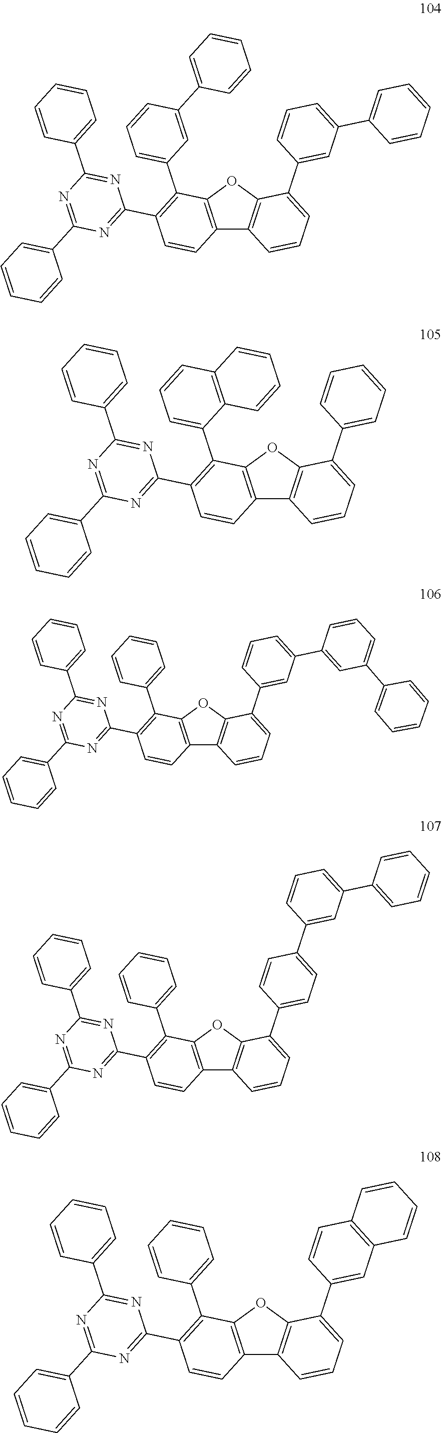 [SMILES: c1ccc(-c2cccc(-c3cccc4c3oc3c(-c5cccc(-c6ccccc6)c5)c(-c5nc(-c6ccccc6)nc(-c6ccccc6)n5)ccc34)c2)cc1 ; c1ccc(-c2nc(-c3ccccc3)nc(-c3ccc4c(oc5c(-c6ccccc6)cccc54)c3-c3cccc4ccccc34)n2)cc1 ; c1ccc(-c2cccc(-c3cccc(-c4cccc5c4oc4c(-c6ccccc6)c(-c6nc(-c7ccccc7)nc(-c7ccccc7)n6)ccc45)c3)c2)cc1 ; c1ccc(-c2cccc(-c3ccc(-c4cccc5c4oc4c(-c6ccccc6)c(-c6nc(-c7ccccc7)nc(-c7ccccc7)n6)ccc45)cc3)c2)cc1 ; c1ccc(-c2nc(-c3ccccc3)nc(-c3ccc4c(oc5c(-c6ccc7ccccc7c6)cccc54)c3-c3ccccc3)n2)cc1]

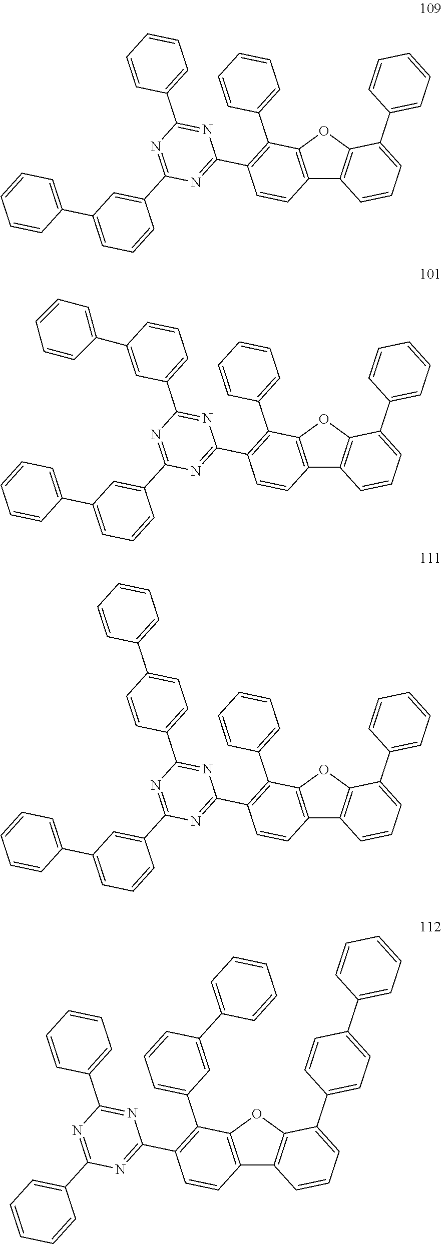 [SMILES: c1ccc(-c2cccc(-c3nc(-c4ccccc4)nc(-c4ccc5c(oc6c(-c7ccccc7)cccc65)c4-c4ccccc4)n3)c2)cc1 ; c1ccc(-c2cccc(-c3nc(-c4cccc(-c5ccccc5)c4)nc(-c4ccc5c(oc6c(-c7ccccc7)cccc65)c4-c4ccccc4)n3)c2)cc1 ; c1ccc(-c2ccc(-c3nc(-c4cccc(-c5ccccc5)c4)nc(-c4ccc5c(oc6c(-c7ccccc7)cccc65)c4-c4ccccc4)n3)cc2)cc1 ; c1ccc(-c2ccc(-c3cccc4c3oc3c(-c5cccc(-c6ccccc6)c5)c(-c5nc(-c6ccccc6)nc(-c6ccccc6)n5)ccc34)cc2)cc1]

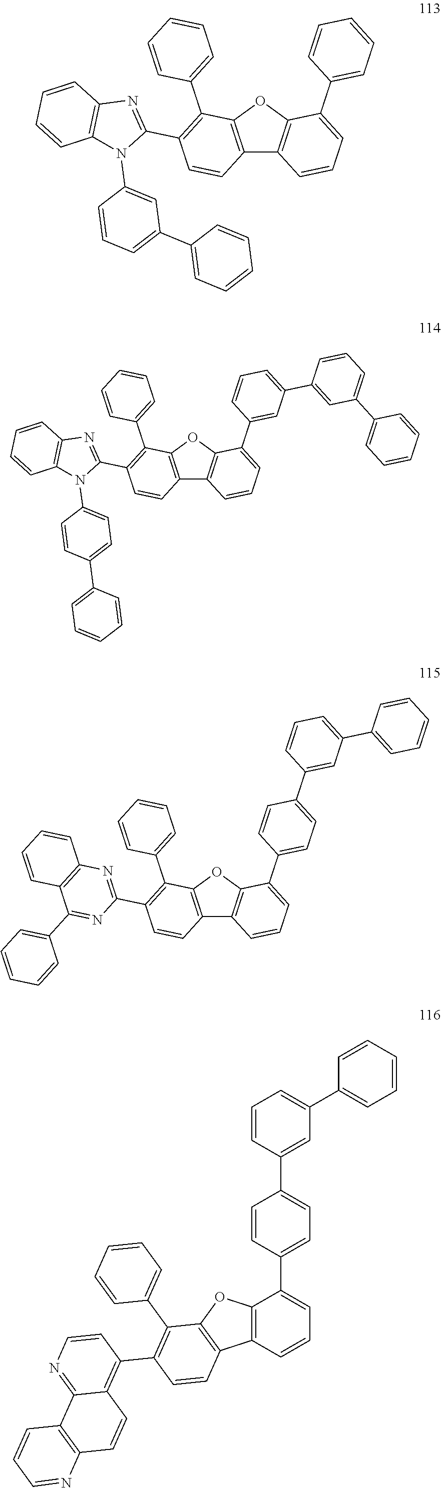 [SMILES: c1ccc(-c2cccc(-n3c(-c4ccc5c(oc6c(-c7ccccc7)cccc65)c4-c4ccccc4)nc4ccccc43)c2)cc1 ; c1ccc(-c2ccc(-n3c(-c4ccc5c(oc6c(-c7cccc(-c8cccc(-c9ccccc9)c8)c7)cccc65)c4-c4ccccc4)nc4ccccc43)cc2)cc1 ; c1ccc(-c2cccc(-c3ccc(-c4cccc5c4oc4c(-c6ccccc6)c(-c6nc(-c7ccccc7)c7ccccc7n6)ccc45)cc3)c2)cc1 ; c1ccc(-c2cccc(-c3ccc(-c4cccc5c4oc4c(-c6ccccc6)c(-c6ccnc7c6ccc6ncccc67)ccc45)cc3)c2)cc1]

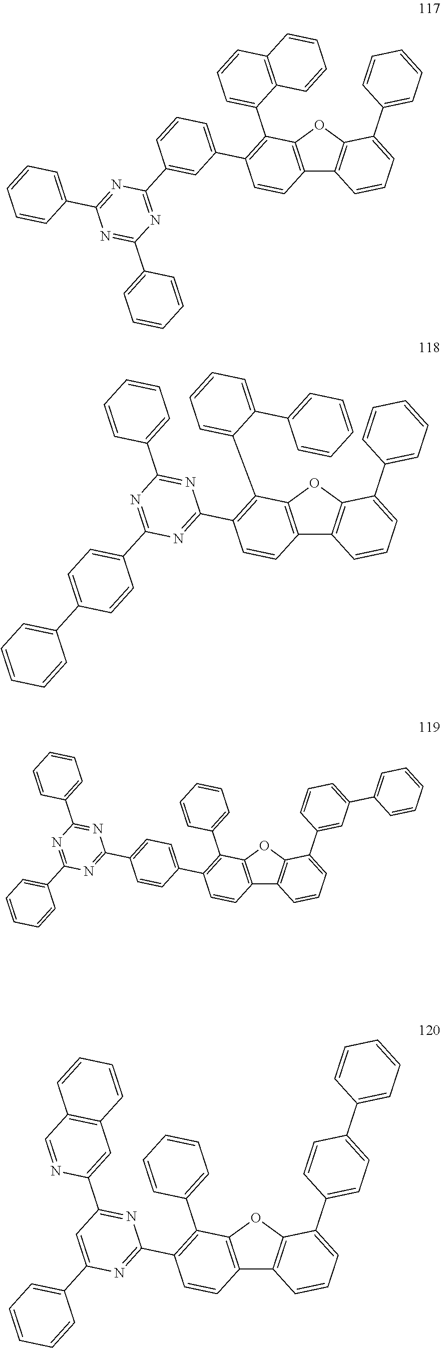 [SMILES: c1ccc(-c2nc(-c3ccccc3)nc(-c3cccc(-c4ccc5c(oc6c(-c7ccccc7)cccc65)c4-c4cccc5ccccc45)c3)n2)cc1 ; c1ccc(-c2ccc(-c3nc(-c4ccccc4)nc(-c4ccc5c(oc6c(-c7ccccc7)cccc65)c4-c4ccccc4-c4ccccc4)n3)cc2)cc1 ; c1ccc(-c2cccc(-c3cccc4c3oc3c(-c5ccccc5)c(-c5ccc(-c6nc(-c7ccccc7)nc(-c7ccccc7)n6)cc5)ccc34)c2)cc1 ; c1ccc(-c2ccc(-c3cccc4c3oc3c(-c5ccccc5)c(-c5nc(-c6ccccc6)cc(-c6cc7ccccc7cn6)n5)ccc34)cc2)cc1]

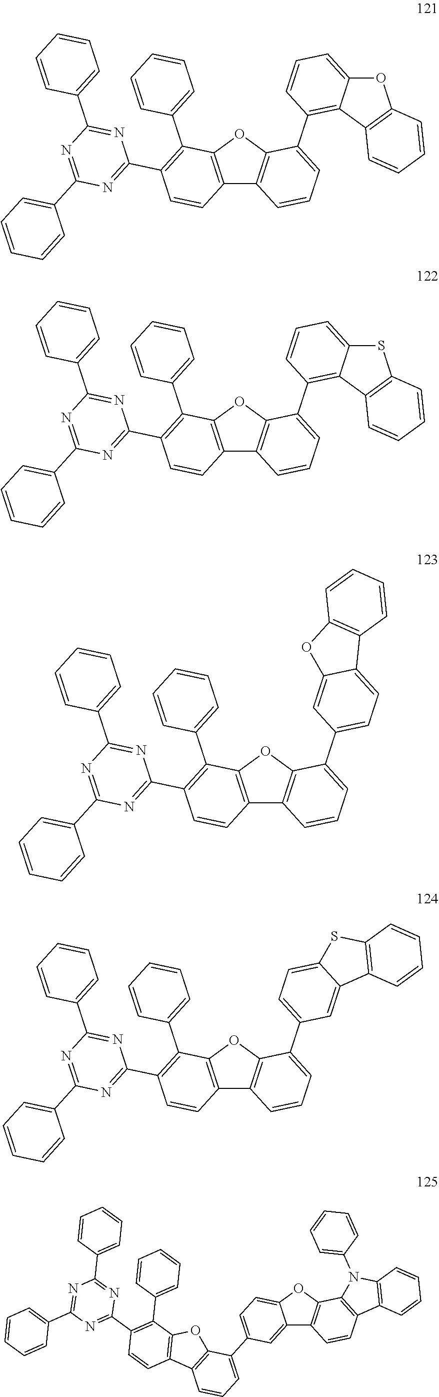

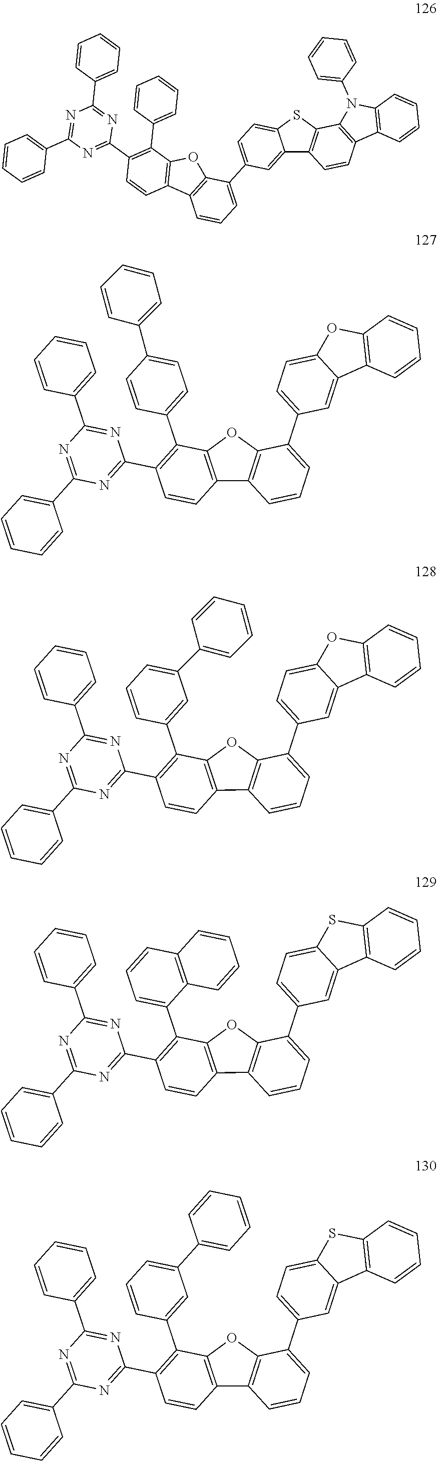

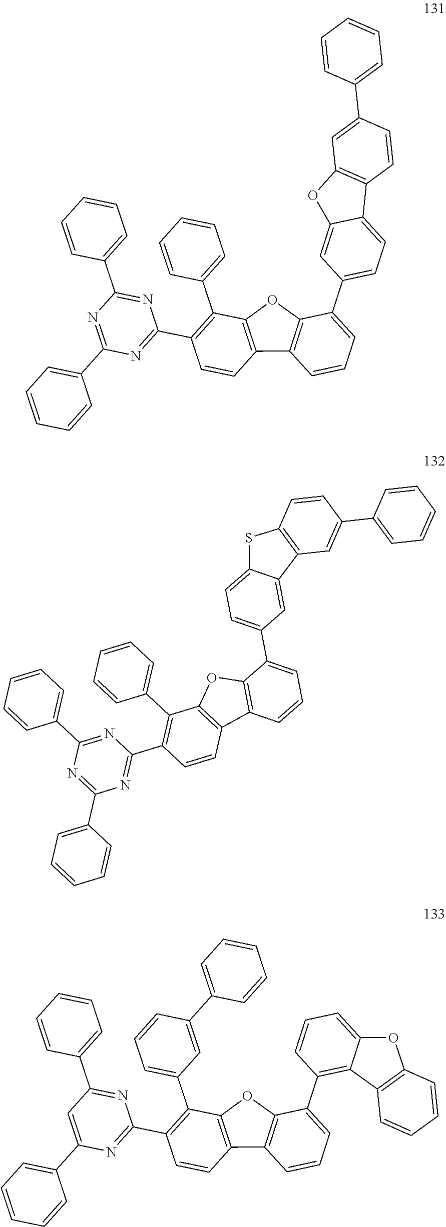 [SMILES: c1ccc(-c2ccc3c(c2)oc2cc(-c4cccc5c4oc4c(-c6ccccc6)c(-c6nc(-c7ccccc7)nc(-c7ccccc7)n6)ccc45)ccc23)cc1 ; c1ccc(-c2ccc3sc4ccc(-c5cccc6c5oc5c(-c7ccccc7)c(-c7nc(-c8ccccc8)nc(-c8ccccc8)n7)ccc56)cc4c3c2)cc1 ; c1ccc(-c2cccc(-c3c(-c4nc(-c5ccccc5)cc(-c5ccccc5)n4)ccc4c3oc3c(-c5cccc6oc7ccccc7c56)cccc34)c2)cc1]

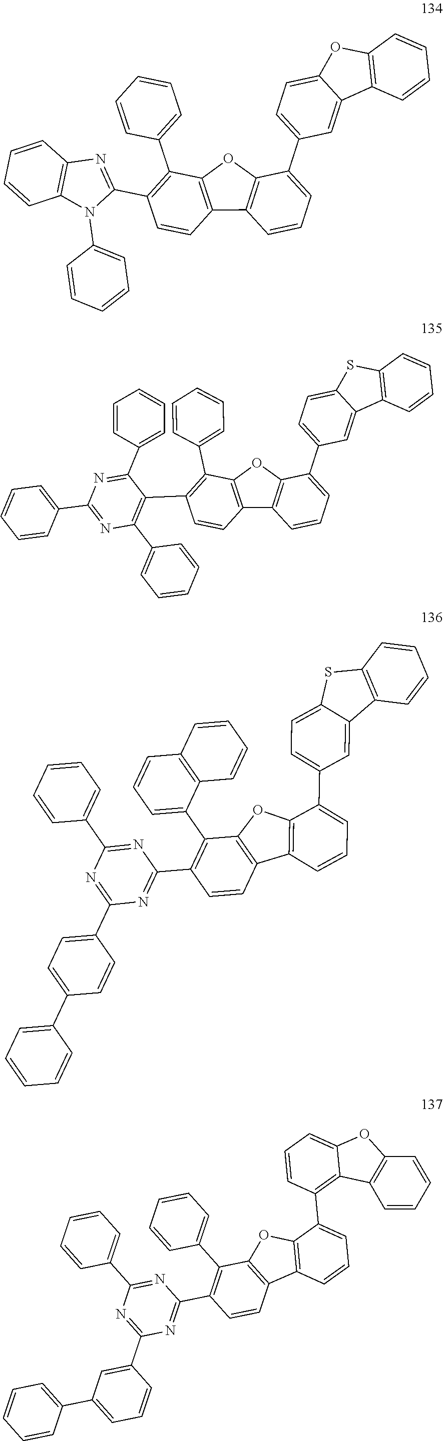 [SMILES: c1ccc(-c2c(-c3nc4ccccc4n3-c3ccccc3)ccc3c2oc2c(-c4ccc5oc6ccccc6c5c4)cccc23)cc1 ; c1ccc(-c2nc(-c3ccccc3)c(-c3ccc4c(oc5c(-c6ccc7sc8ccccc8c7c6)cccc54)c3-c3ccccc3)c(-c3ccccc3)n2)cc1 ; c1ccc(-c2ccc(-c3nc(-c4ccccc4)nc(-c4ccc5c(oc6c(-c7ccc8sc9ccccc9c8c7)cccc65)c4-c4cccc5ccccc45)n3)cc2)cc1 ; c1ccc(-c2cccc(-c3nc(-c4ccccc4)nc(-c4ccc5c(oc6c(-c7cccc8oc9ccccc9c78)cccc65)c4-c4ccccc4)n3)c2)cc1]

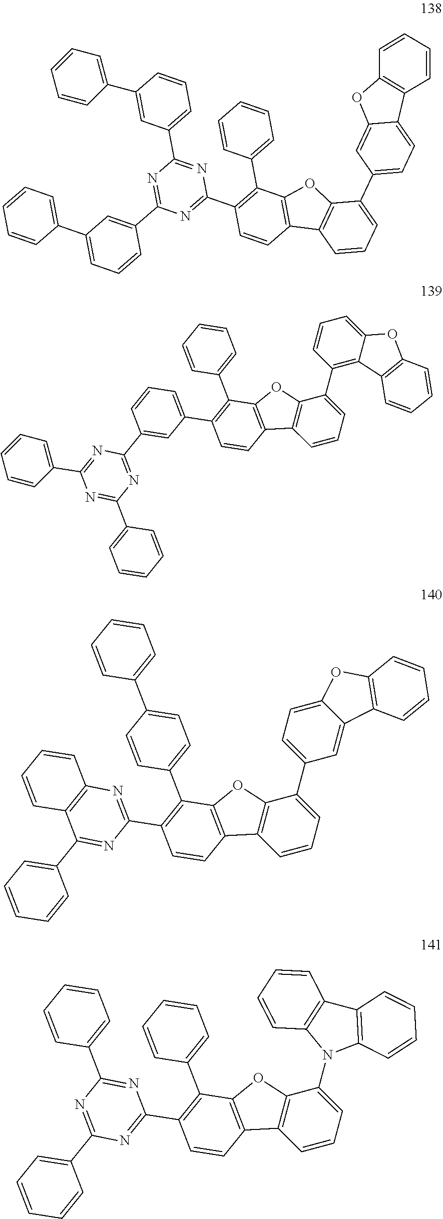 [SMILES: c1ccc(-c2cccc(-c3nc(-c4cccc(-c5ccccc5)c4)nc(-c4ccc5c(oc6c(-c7ccc8c(c7)oc7ccccc78)cccc65)c4-c4ccccc4)n3)c2)cc1 ; c1ccc(-c2nc(-c3ccccc3)nc(-c3cccc(-c4ccc5c(oc6c(-c7cccc8oc9ccccc9c78)cccc65)c4-c4ccccc4)c3)n2)cc1 ; c1ccc(-c2ccc(-c3c(-c4nc(-c5ccccc5)c5ccccc5n4)ccc4c3oc3c(-c5ccc6oc7ccccc7c6c5)cccc34)cc2)cc1 ; c1ccc(-c2nc(-c3ccccc3)nc(-c3ccc4c(oc5c(-n6c7ccccc7c7ccccc76)cccc54)c3-c3ccccc3)n2)cc1]

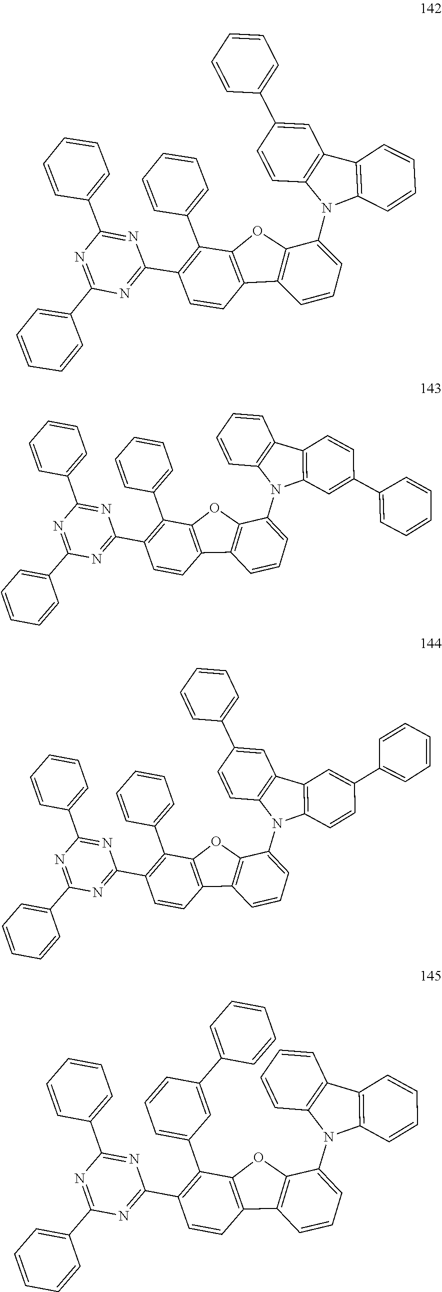 [SMILES: c1ccc(-c2ccc3c(c2)c2ccccc2n3-c2cccc3c2oc2c(-c4ccccc4)c(-c4nc(-c5ccccc5)nc(-c5ccccc5)n4)ccc23)cc1 ; c1ccc(-c2ccc3c4ccccc4n(-c4cccc5c4oc4c(-c6ccccc6)c(-c6nc(-c7ccccc7)nc(-c7ccccc7)n6)ccc45)c3c2)cc1 ; c1ccc(-c2ccc3c(c2)c2cc(-c4ccccc4)ccc2n3-c2cccc3c2oc2c(-c4ccccc4)c(-c4nc(-c5ccccc5)nc(-c5ccccc5)n4)ccc23)cc1 ; c1ccc(-c2cccc(-c3c(-c4nc(-c5ccccc5)nc(-c5ccccc5)n4)ccc4c3oc3c(-n5c6ccccc6c6ccccc65)cccc34)c2)cc1]

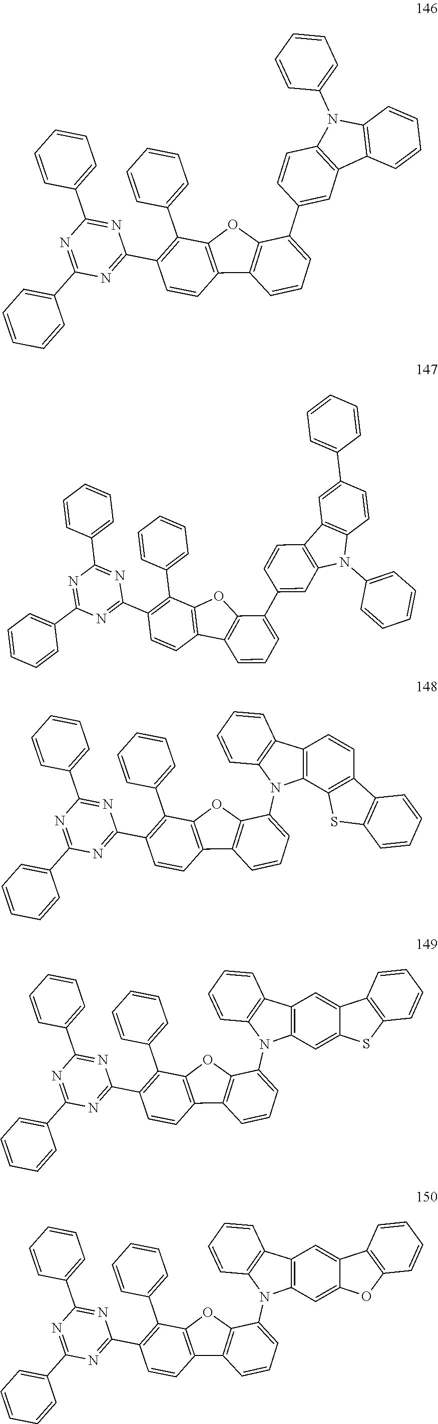

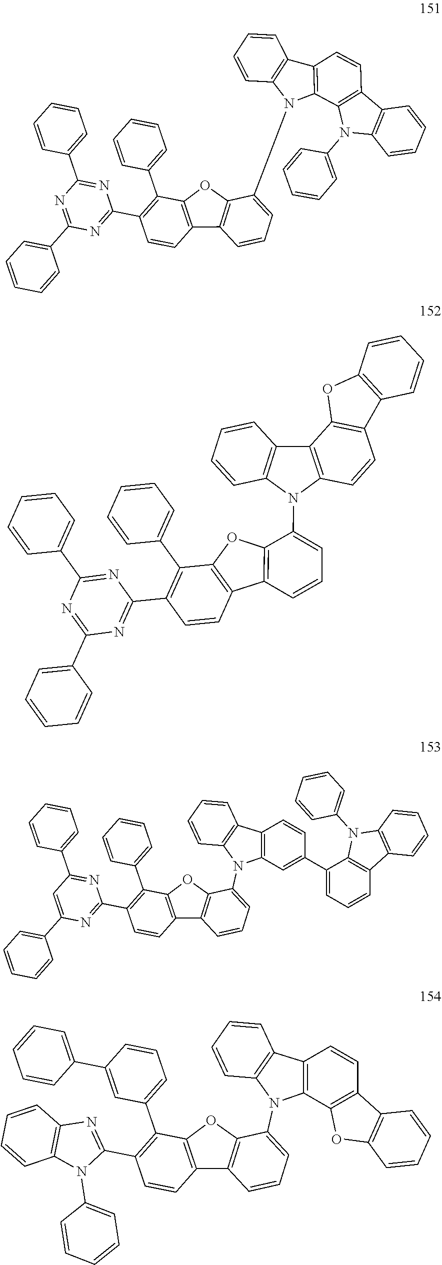 [SMILES: c1ccc(-c2nc(-c3ccccc3)nc(-c3ccc4c(oc5c(-n6c7ccccc7c7ccc8c9ccccc9n(-c9ccccc9)c8c76)cccc54)c3-c3ccccc3)n2)cc1 ; c1ccc(-c2nc(-c3ccccc3)nc(-c3ccc4c(oc5c(-n6c7ccccc7c7c8oc9ccccc9c8ccc76)cccc54)c3-c3ccccc3)n2)cc1 ; c1ccc(-c2cc(-c3ccccc3)nc(-c3ccc4c(oc5c(-n6c7ccccc7c7ccc(-c8cccc9c%10ccccc%10n(-c%10ccccc%10)c89)cc76)cccc54)c3-c3ccccc3)n2)cc1 ; c1ccc(-c2cccc(-c3c(-c4nc5ccccc5n4-c4ccccc4)ccc4c3oc3c(-n5c6ccccc6c6ccc7c8ccccc8oc7c65)cccc34)c2)cc1]

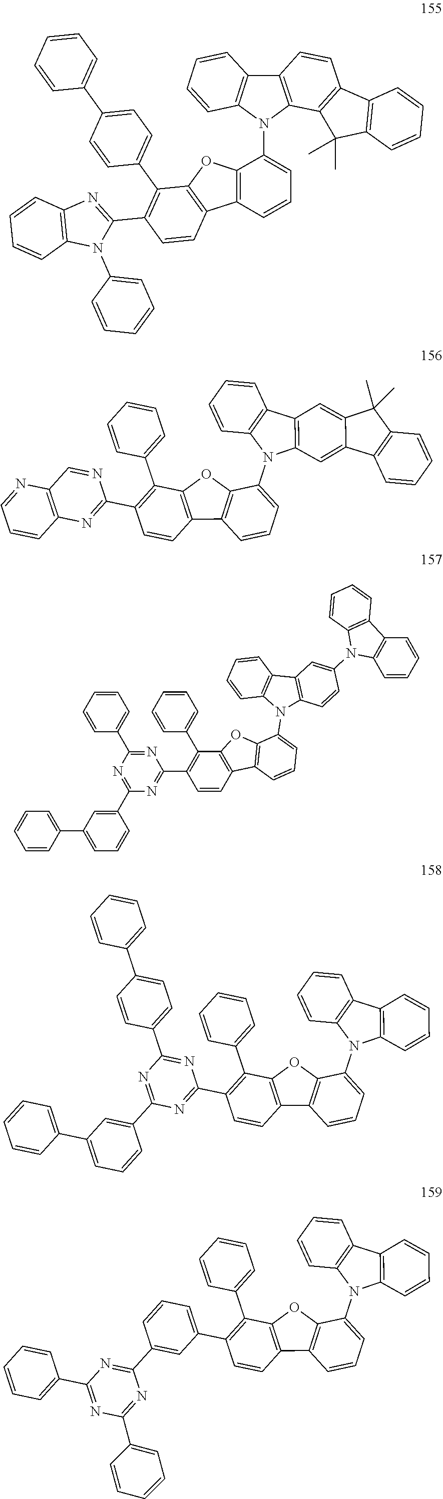

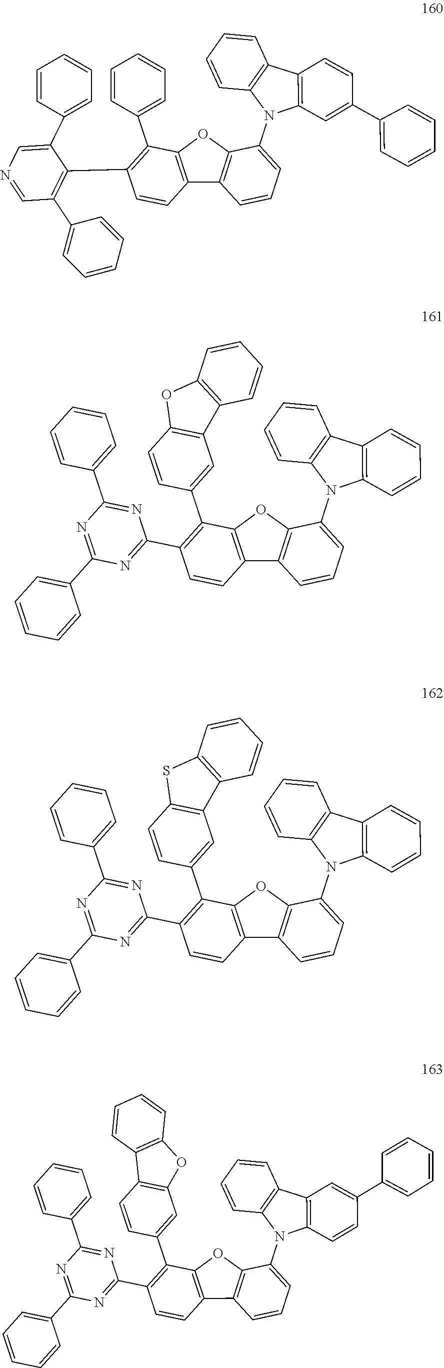 [SMILES: c1ccc(-c2ccc3c4ccccc4n(-c4cccc5c4oc4c(-c6ccccc6)c(-c6c(-c7ccccc7)cncc6-c6ccccc6)ccc45)c3c2)cc1 ; c1ccc(-c2nc(-c3ccccc3)nc(-c3ccc4c(oc5c(-n6c7ccccc7c7ccccc76)cccc54)c3-c3ccc4oc5ccccc5c4c3)n2)cc1 ; c1ccc(-c2nc(-c3ccccc3)nc(-c3ccc4c(oc5c(-n6c7ccccc7c7ccccc76)cccc54)c3-c3ccc4sc5ccccc5c4c3)n2)cc1 ; c1ccc(-c2ccc3c(c2)c2ccccc2n3-c2cccc3c2oc2c(-c4ccc5c(c4)oc4ccccc45)c(-c4nc(-c5ccccc5)nc(-c5ccccc5)n4)ccc23)cc1]

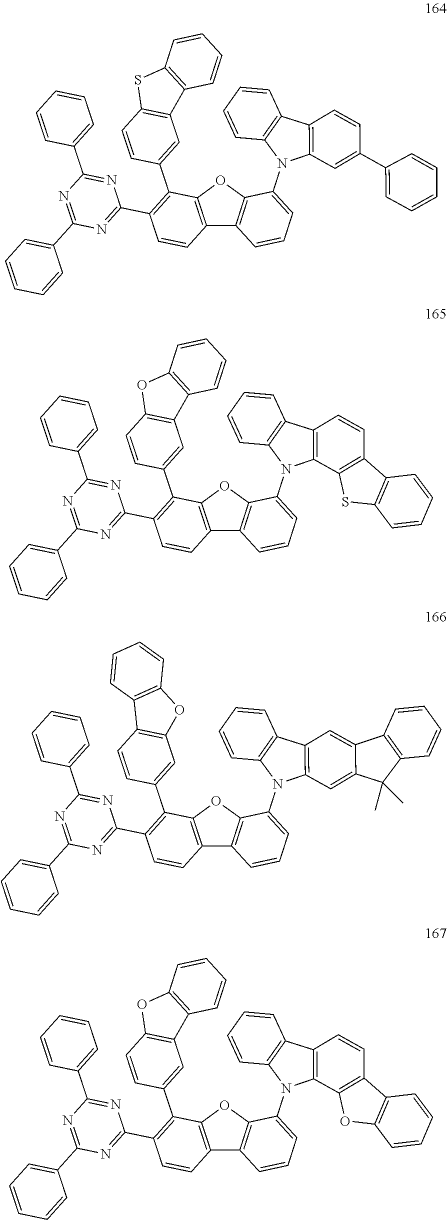 [SMILES: c1ccc(-c2ccc3c4ccccc4n(-c4cccc5c4oc4c(-c6ccc7sc8ccccc8c7c6)c(-c6nc(-c7ccccc7)nc(-c7ccccc7)n6)ccc45)c3c2)cc1 ; c1ccc(-c2nc(-c3ccccc3)nc(-c3ccc4c(oc5c(-n6c7ccccc7c7ccc8c9ccccc9sc8c76)cccc54)c3-c3ccc4oc5ccccc5c4c3)n2)cc1 ; CC1(C)c2ccccc2-c2cc3c4ccccc4n(-c4cccc5c4oc4c(-c6ccc7c(c6)oc6ccccc67)c(-c6nc(-c7ccccc7)nc(-c7ccccc7)n6)ccc45)c3cc21 ; c1ccc(-c2nc(-c3ccccc3)nc(-c3ccc4c(oc5c(-n6c7ccccc7c7ccc8c9ccccc9oc8c76)cccc54)c3-c3ccc4oc5ccccc5c4c3)n2)cc1]

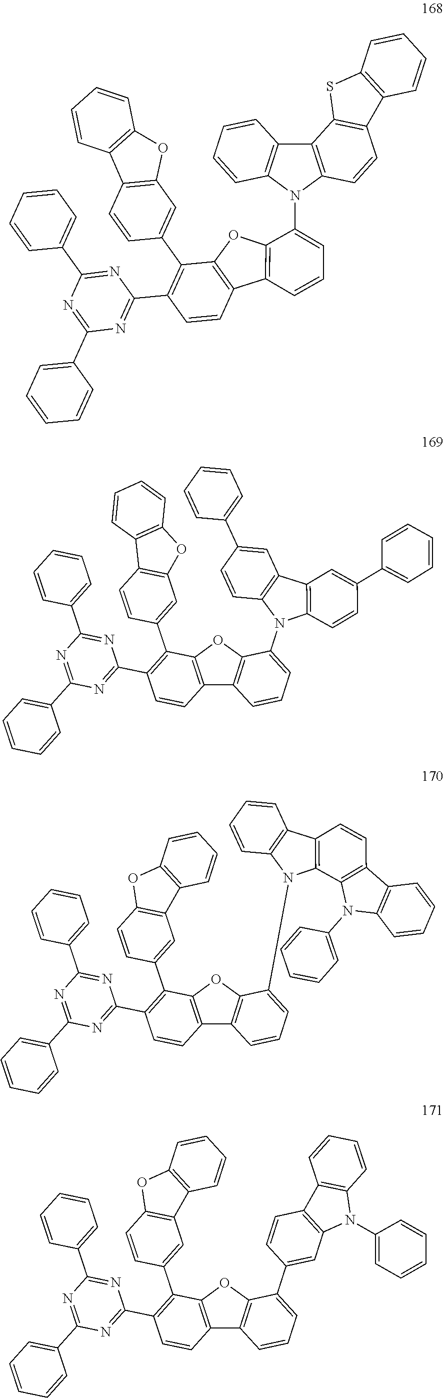 [SMILES: c1ccc(-c2nc(-c3ccccc3)nc(-c3ccc4c(oc5c(-n6c7ccccc7c7c8sc9ccccc9c8ccc76)cccc54)c3-c3ccc4c(c3)oc3ccccc34)n2)cc1 ; c1ccc(-c2ccc3c(c2)c2cc(-c4ccccc4)ccc2n3-c2cccc3c2oc2c(-c4ccc5c(c4)oc4ccccc45)c(-c4nc(-c5ccccc5)nc(-c5ccccc5)n4)ccc23)cc1 ; c1ccc(-c2nc(-c3ccccc3)nc(-c3ccc4c(oc5c(-n6c7ccccc7c7ccc8c9ccccc9n(-c9ccccc9)c8c76)cccc54)c3-c3ccc4oc5ccccc5c4c3)n2)cc1 ; c1ccc(-c2nc(-c3ccccc3)nc(-c3ccc4c(oc5c(-c6ccc7c8ccccc8n(-c8ccccc8)c7c6)cccc54)c3-c3ccc4oc5ccccc5c4c3)n2)cc1]

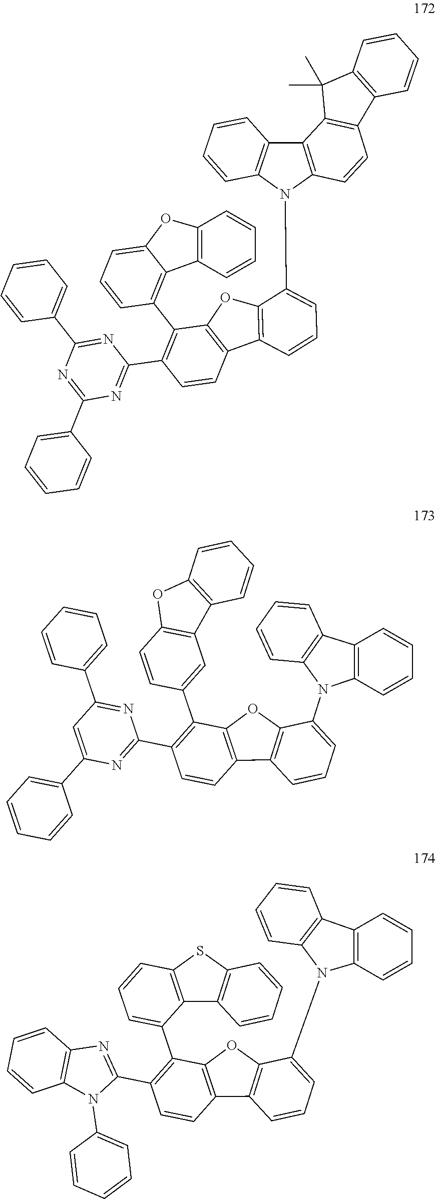 [SMILES: CC1(C)c2ccccc2-c2ccc3c(c21)c1ccccc1n3-c1cccc2c1oc1c(-c3cccc4oc5ccccc5c34)c(-c3nc(-c4ccccc4)nc(-c4ccccc4)n3)ccc12 ; c1ccc(-c2cc(-c3ccccc3)nc(-c3ccc4c(oc5c(-n6c7ccccc7c7ccccc76)cccc54)c3-c3ccc4oc5ccccc5c4c3)n2)cc1 ; c1ccc(-n2c(-c3ccc4c(oc5c(-n6c7ccccc7c7ccccc76)cccc54)c3-c3cccc4sc5ccccc5c34)nc3ccccc32)cc1]

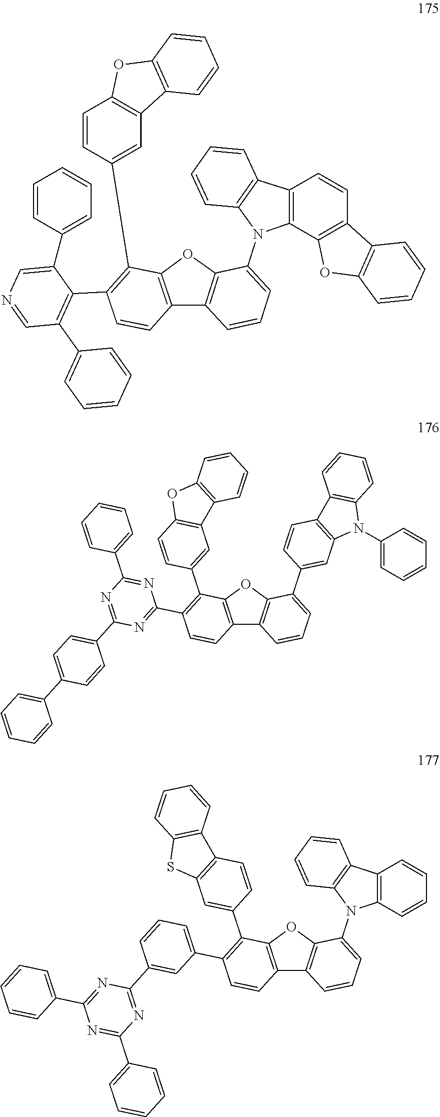 [SMILES: c1ccc(-c2cncc(-c3ccccc3)c2-c2ccc3c(oc4c(-n5c6ccccc6c6ccc7c8ccccc8oc7c65)cccc43)c2-c2ccc3oc4ccccc4c3c2)cc1 ; c1ccc(-c2ccc(-c3nc(-c4ccccc4)nc(-c4ccc5c(oc6c(-c7ccc8c9ccccc9n(-c9ccccc9)c8c7)cccc65)c4-c4ccc5oc6ccccc6c5c4)n3)cc2)cc1 ; c1ccc(-c2nc(-c3ccccc3)nc(-c3cccc(-c4ccc5c(oc6c(-n7c8ccccc8c8ccccc87)cccc65)c4-c4ccc5c(c4)sc4ccccc45)c3)n2)cc1]

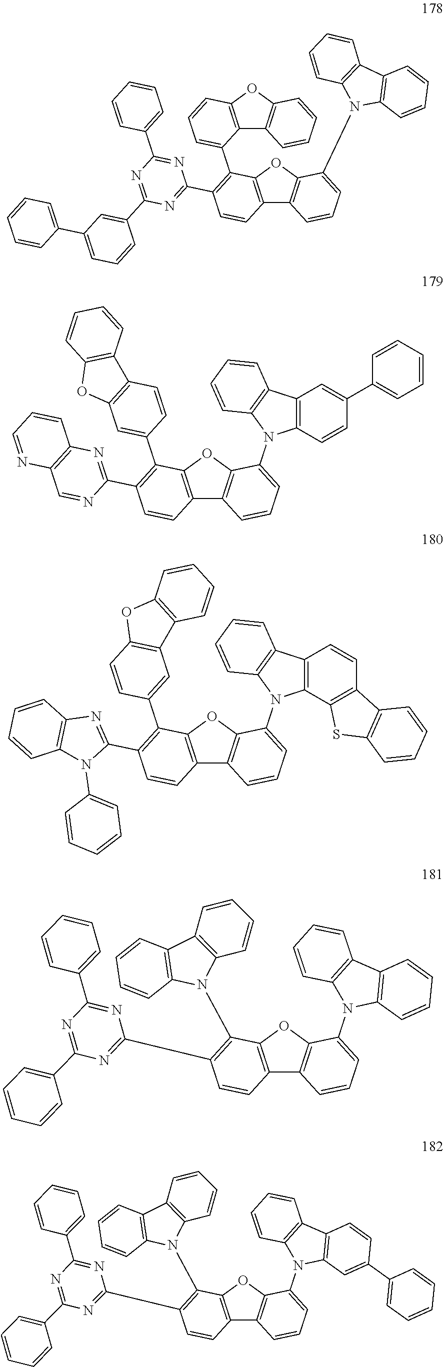

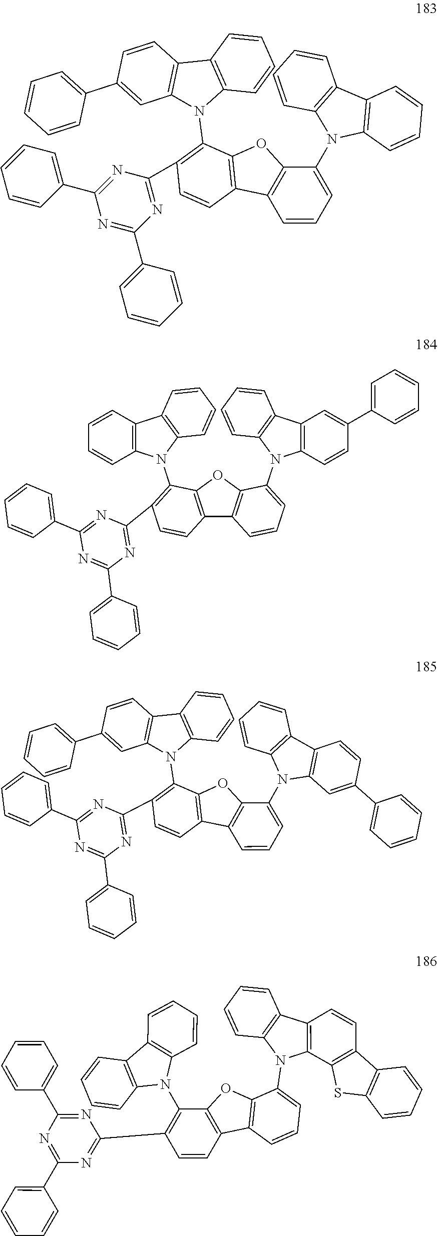 [SMILES: c1ccc(-c2ccc3c4ccccc4n(-c4c(-c5nc(-c6ccccc6)nc(-c6ccccc6)n5)ccc5c4oc4c(-n6c7ccccc7c7ccccc76)cccc45)c3c2)cc1 ; c1ccc(-c2ccc3c(c2)c2ccccc2n3-c2cccc3c2oc2c(-n4c5ccccc5c5ccccc54)c(-c4nc(-c5ccccc5)nc(-c5ccccc5)n4)ccc23)cc1 ; c1ccc(-c2ccc3c4ccccc4n(-c4cccc5c4oc4c(-n6c7ccccc7c7ccc(-c8ccccc8)cc76)c(-c6nc(-c7ccccc7)nc(-c7ccccc7)n6)ccc45)c3c2)cc1 ; c1ccc(-c2nc(-c3ccccc3)nc(-c3ccc4c(oc5c(-n6c7ccccc7c7ccc8c9ccccc9sc8c76)cccc54)c3-n3c4ccccc4c4ccccc43)n2)cc1]

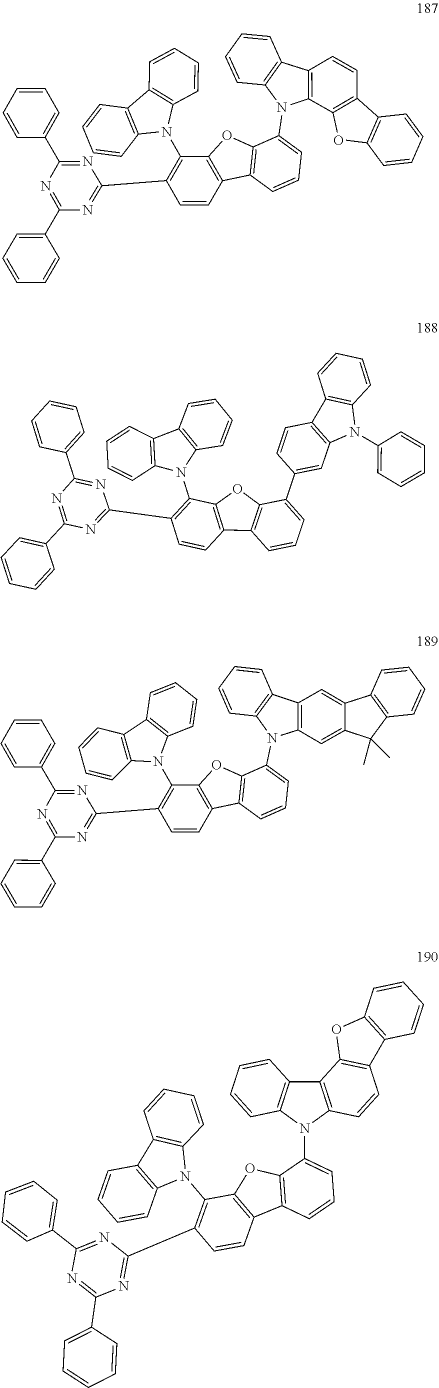 [SMILES: c1ccc(-c2nc(-c3ccccc3)nc(-c3ccc4c(oc5c(-n6c7ccccc7c7ccc8c9ccccc9oc8c76)cccc54)c3-n3c4ccccc4c4ccccc43)n2)cc1 ; c1ccc(-c2nc(-c3ccccc3)nc(-c3ccc4c(oc5c(-c6ccc7c8ccccc8n(-c8ccccc8)c7c6)cccc54)c3-n3c4ccccc4c4ccccc43)n2)cc1 ; CC1(C)c2ccccc2-c2cc3c4ccccc4n(-c4cccc5c4oc4c(-n6c7ccccc7c7ccccc76)c(-c6nc(-c7ccccc7)nc(-c7ccccc7)n6)ccc45)c3cc21 ; c1ccc(-c2nc(-c3ccccc3)nc(-c3ccc4c(oc5c(-n6c7ccccc7c7c8oc9ccccc9c8ccc76)cccc54)c3-n3c4ccccc4c4ccccc43)n2)cc1]

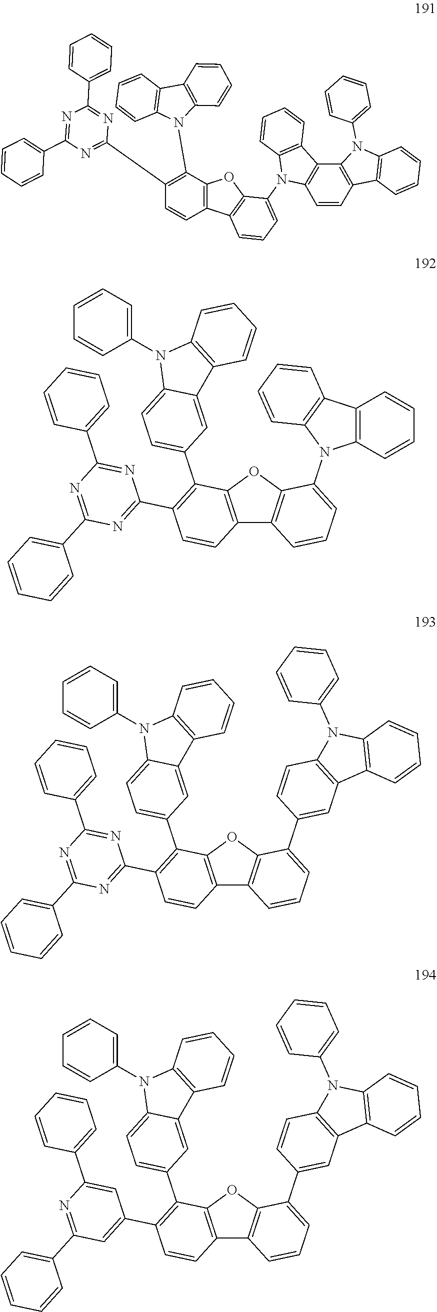 [SMILES: c1ccc(-c2nc(-c3ccccc3)nc(-c3ccc4c(oc5c(-n6c7ccccc7c7c6ccc6c8ccccc8n(-c8ccccc8)c67)cccc54)c3-n3c4ccccc4c4ccccc43)n2)cc1 ; c1ccc(-c2nc(-c3ccccc3)nc(-c3ccc4c(oc5c(-n6c7ccccc7c7ccccc76)cccc54)c3-c3ccc4c(c3)c3ccccc3n4-c3ccccc3)n2)cc1 ; c1ccc(-c2nc(-c3ccccc3)nc(-c3ccc4c(oc5c(-c6ccc7c(c6)c6ccccc6n7-c6ccccc6)cccc54)c3-c3ccc4c(c3)c3ccccc3n4-c3ccccc3)n2)cc1 ; c1ccc(-c2cc(-c3ccc4c(oc5c(-c6ccc7c(c6)c6ccccc6n7-c6ccccc6)cccc54)c3-c3ccc4c(c3)c3ccccc3n4-c3ccccc3)cc(-c3ccccc3)n2)cc1]

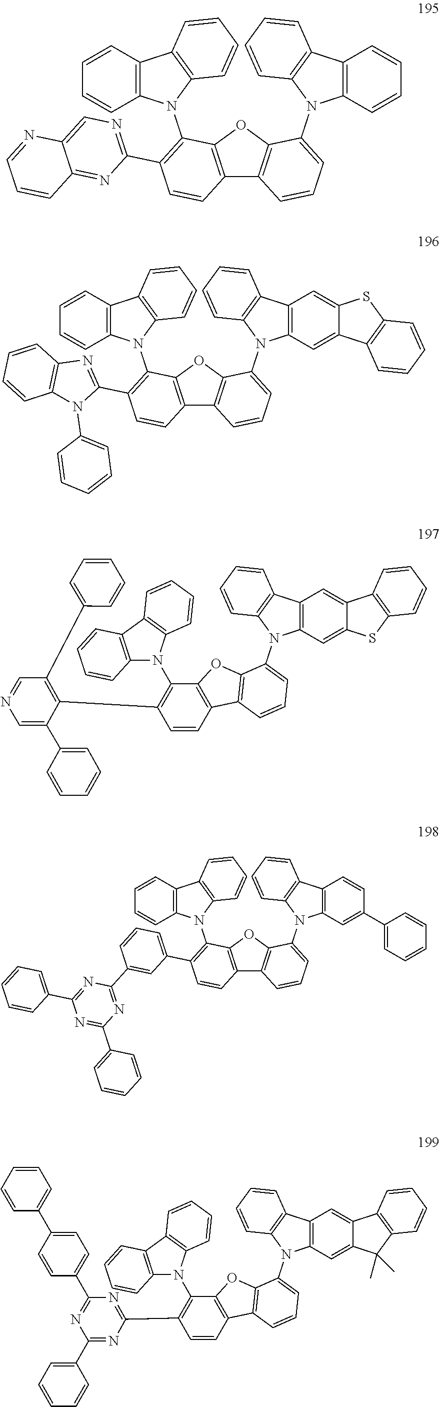

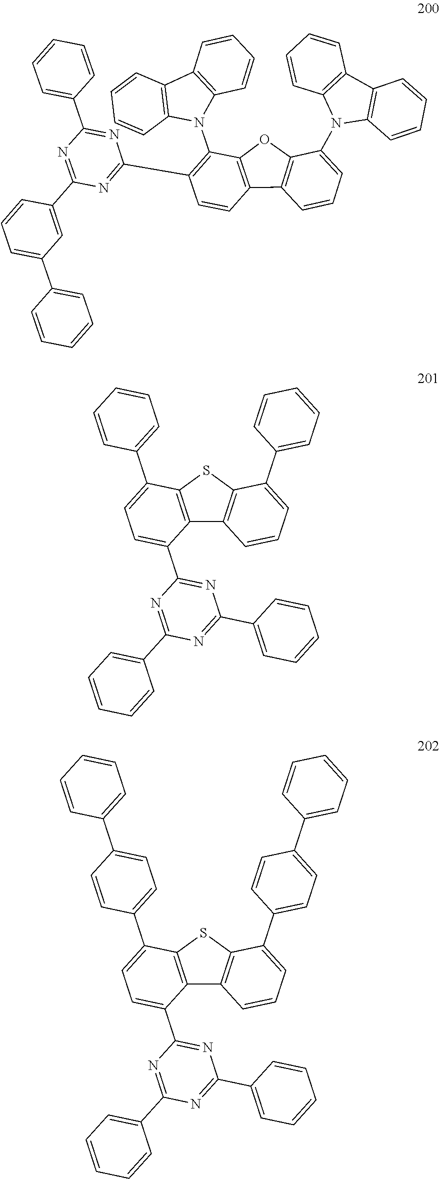 [SMILES: c1ccc(-c2cccc(-c3nc(-c4ccccc4)nc(-c4ccc5c(oc6c(-n7c8ccccc8c8ccccc87)cccc65)c4-n4c5ccccc5c5ccccc54)n3)c2)cc1 ; c1ccc(-c2nc(-c3ccccc3)nc(-c3ccc(-c4ccccc4)c4sc5c(-c6ccccc6)cccc5c34)n2)cc1 ; c1ccc(-c2ccc(-c3cccc4c3sc3c(-c5ccc(-c6ccccc6)cc5)ccc(-c5nc(-c6ccccc6)nc(-c6ccccc6)n5)c34)cc2)cc1]

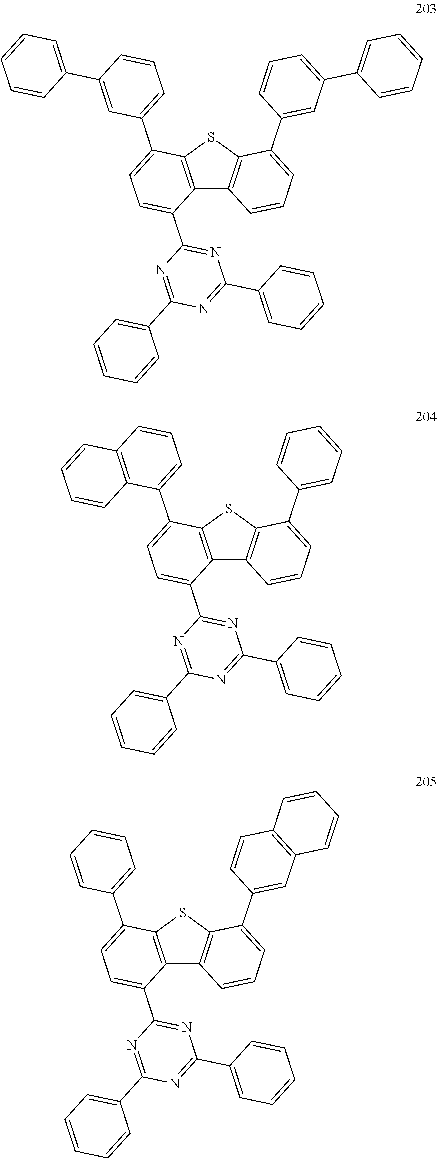

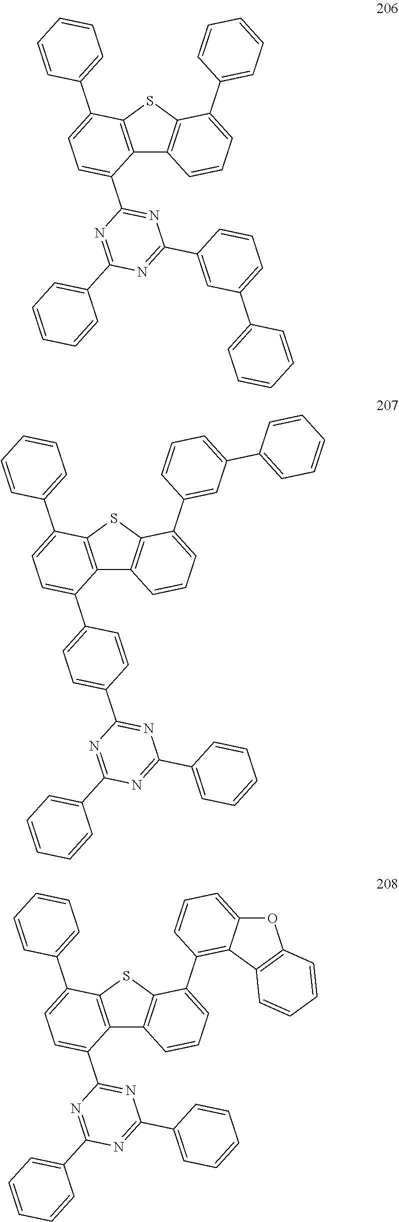

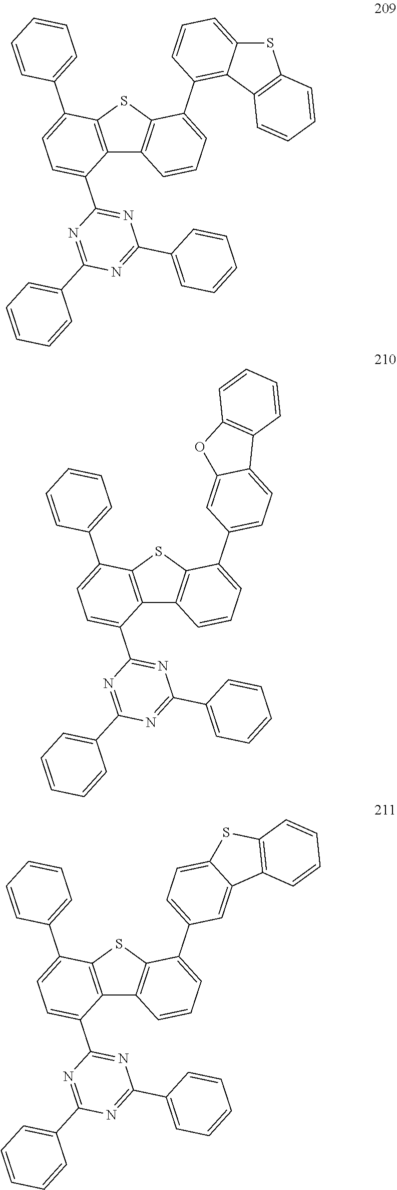 [SMILES: c1ccc(-c2nc(-c3ccccc3)nc(-c3ccc(-c4ccccc4)c4sc5c(-c6cccc7sc8ccccc8c67)cccc5c34)n2)cc1 ; c1ccc(-c2nc(-c3ccccc3)nc(-c3ccc(-c4ccccc4)c4sc5c(-c6ccc7c(c6)oc6ccccc67)cccc5c34)n2)cc1 ; c1ccc(-c2nc(-c3ccccc3)nc(-c3ccc(-c4ccccc4)c4sc5c(-c6ccc7sc8ccccc8c7c6)cccc5c34)n2)cc1]

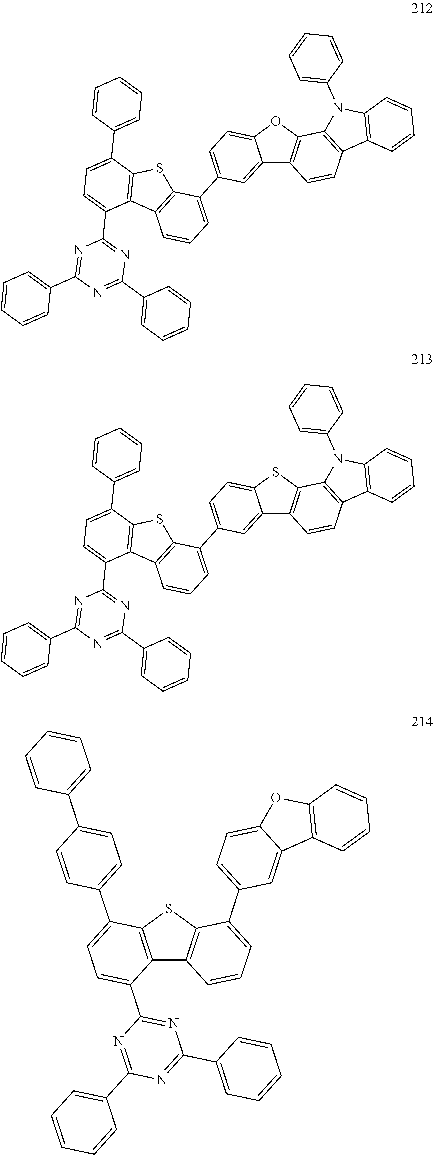 [SMILES: c1ccc(-c2nc(-c3ccccc3)nc(-c3ccc(-c4ccccc4)c4sc5c(-c6ccc7oc8c(ccc9c%10ccccc%10n(-c%10ccccc%10)c98)c7c6)cccc5c34)n2)cc1 ; c1ccc(-c2nc(-c3ccccc3)nc(-c3ccc(-c4ccccc4)c4sc5c(-c6ccc7sc8c(ccc9c%10ccccc%10n(-c%10ccccc%10)c98)c7c6)cccc5c34)n2)cc1 ; c1ccc(-c2ccc(-c3ccc(-c4nc(-c5ccccc5)nc(-c5ccccc5)n4)c4c3sc3c(-c5ccc6oc7ccccc7c6c5)cccc34)cc2)cc1]

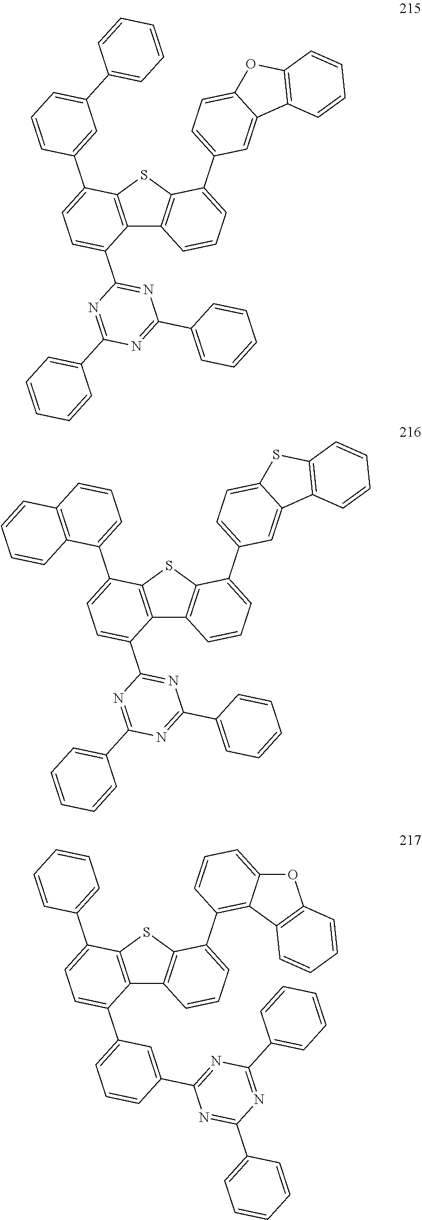

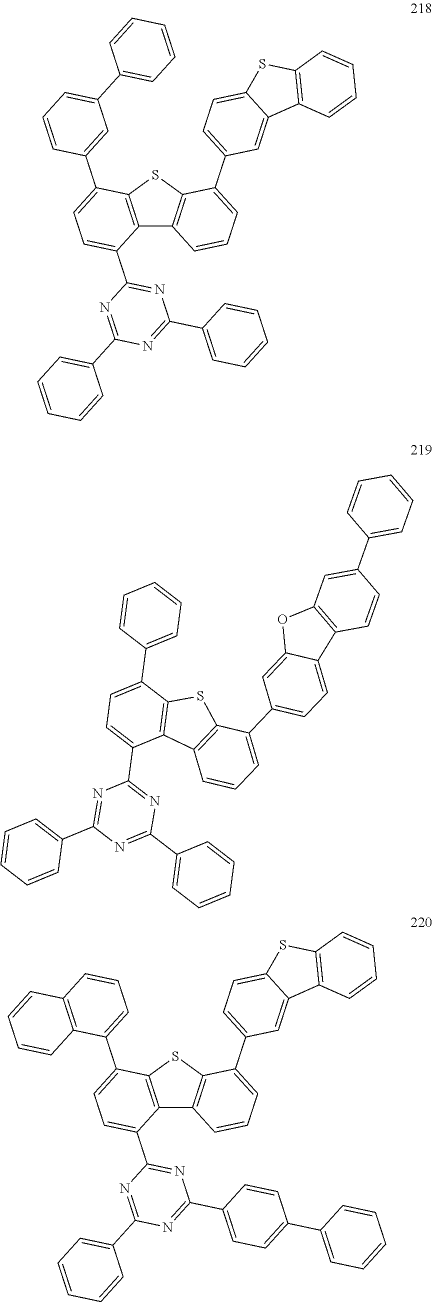 [SMILES: c1ccc(-c2cccc(-c3ccc(-c4nc(-c5ccccc5)nc(-c5ccccc5)n4)c4c3sc3c(-c5ccc6sc7ccccc7c6c5)cccc34)c2)cc1 ; c1ccc(-c2ccc3c(c2)oc2cc(-c4cccc5c4sc4c(-c6ccccc6)ccc(-c6nc(-c7ccccc7)nc(-c7ccccc7)n6)c45)ccc23)cc1 ; c1ccc(-c2ccc(-c3nc(-c4ccccc4)nc(-c4ccc(-c5cccc6ccccc56)c5sc6c(-c7ccc8sc9ccccc9c8c7)cccc6c45)n3)cc2)cc1]

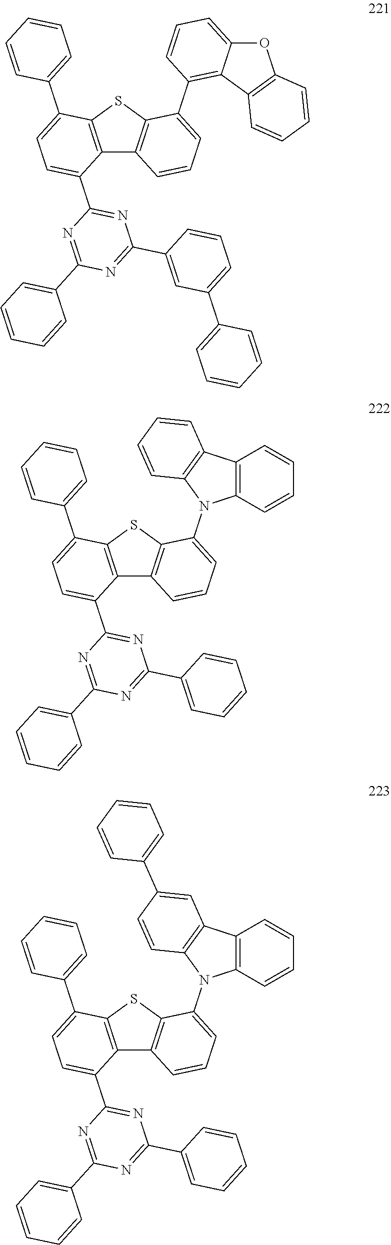

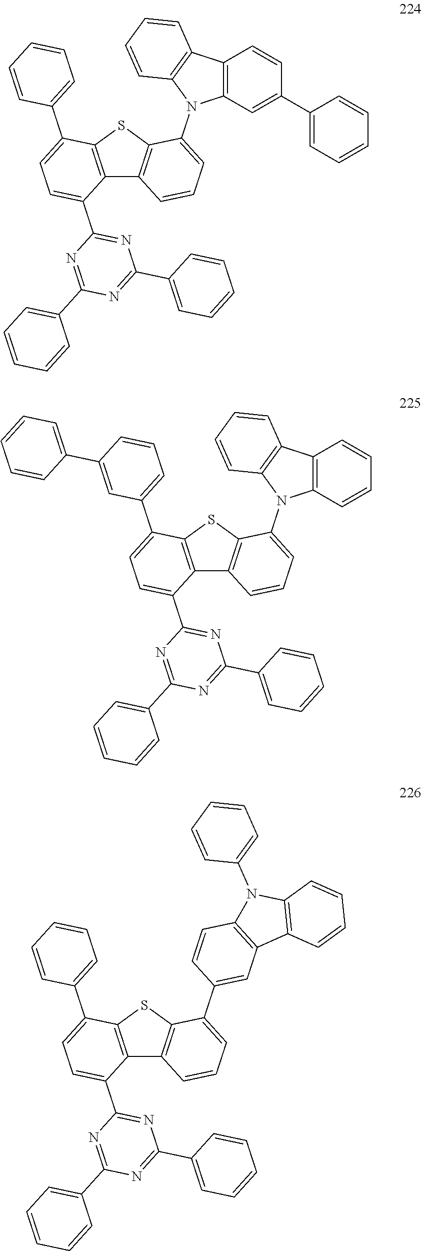 [SMILES: c1ccc(-c2ccc3c4ccccc4n(-c4cccc5c4sc4c(-c6ccccc6)ccc(-c6nc(-c7ccccc7)nc(-c7ccccc7)n6)c45)c3c2)cc1 ; c1ccc(-c2cccc(-c3ccc(-c4nc(-c5ccccc5)nc(-c5ccccc5)n4)c4c3sc3c(-n5c6ccccc6c6ccccc65)cccc34)c2)cc1 ; c1ccc(-c2nc(-c3ccccc3)nc(-c3ccc(-c4ccccc4)c4sc5c(-c6ccc7c(c6)c6ccccc6n7-c6ccccc6)cccc5c34)n2)cc1]

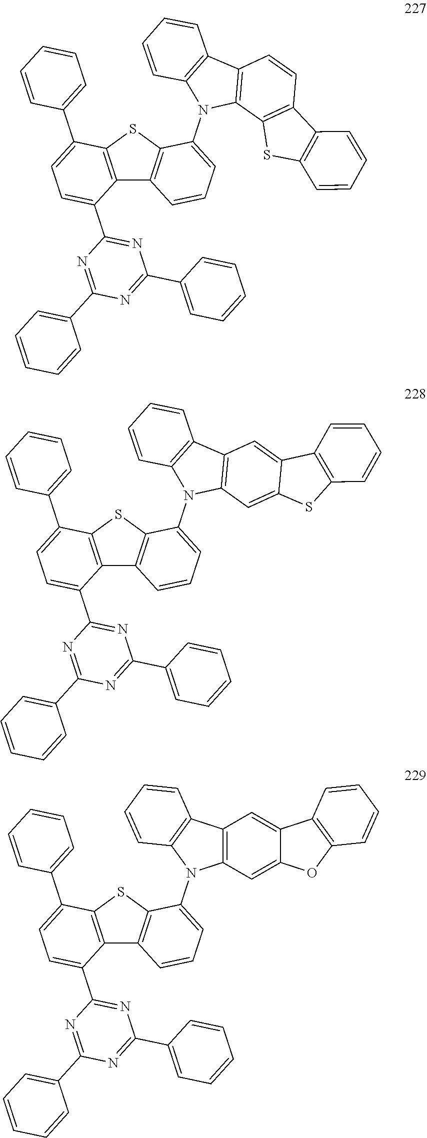 [SMILES: c1ccc(-c2nc(-c3ccccc3)nc(-c3ccc(-c4ccccc4)c4sc5c(-n6c7ccccc7c7ccc8c9ccccc9sc8c76)cccc5c34)n2)cc1 ; c1ccc(-c2nc(-c3ccccc3)nc(-c3ccc(-c4ccccc4)c4sc5c(-n6c7ccccc7c7cc8c(cc76)sc6ccccc68)cccc5c34)n2)cc1 ; c1ccc(-c2nc(-c3ccccc3)nc(-c3ccc(-c4ccccc4)c4sc5c(-n6c7ccccc7c7cc8c(cc76)oc6ccccc68)cccc5c34)n2)cc1]

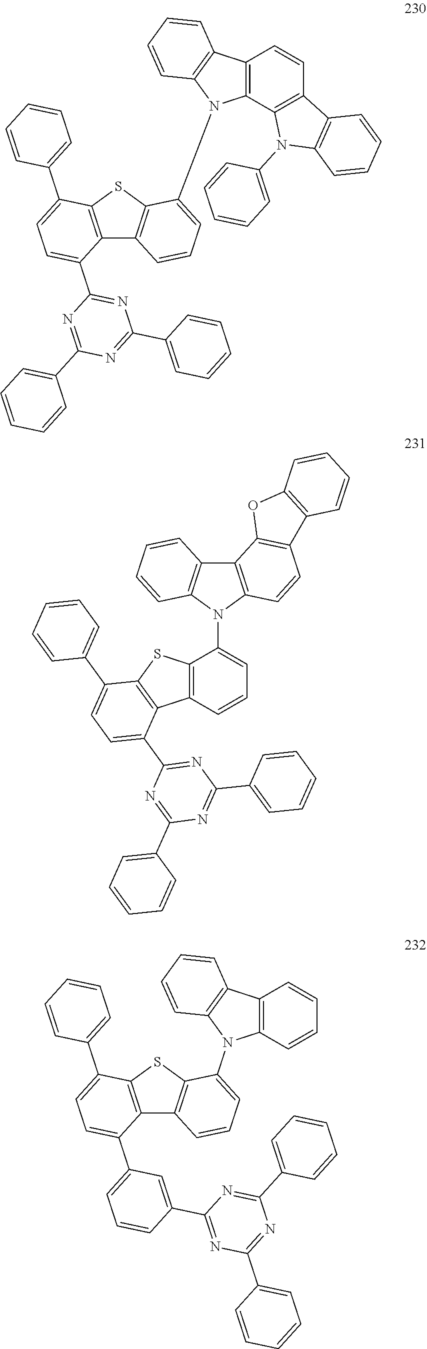 [SMILES: c1ccc(-c2nc(-c3ccccc3)nc(-c3ccc(-c4ccccc4)c4sc5c(-n6c7ccccc7c7ccc8c9ccccc9n(-c9ccccc9)c8c76)cccc5c34)n2)cc1 ; c1ccc(-c2nc(-c3ccccc3)nc(-c3ccc(-c4ccccc4)c4sc5c(-n6c7ccccc7c7c8oc9ccccc9c8ccc76)cccc5c34)n2)cc1 ; c1ccc(-c2nc(-c3ccccc3)nc(-c3cccc(-c4ccc(-c5ccccc5)c5sc6c(-n7c8ccccc8c8ccccc87)cccc6c45)c3)n2)cc1]

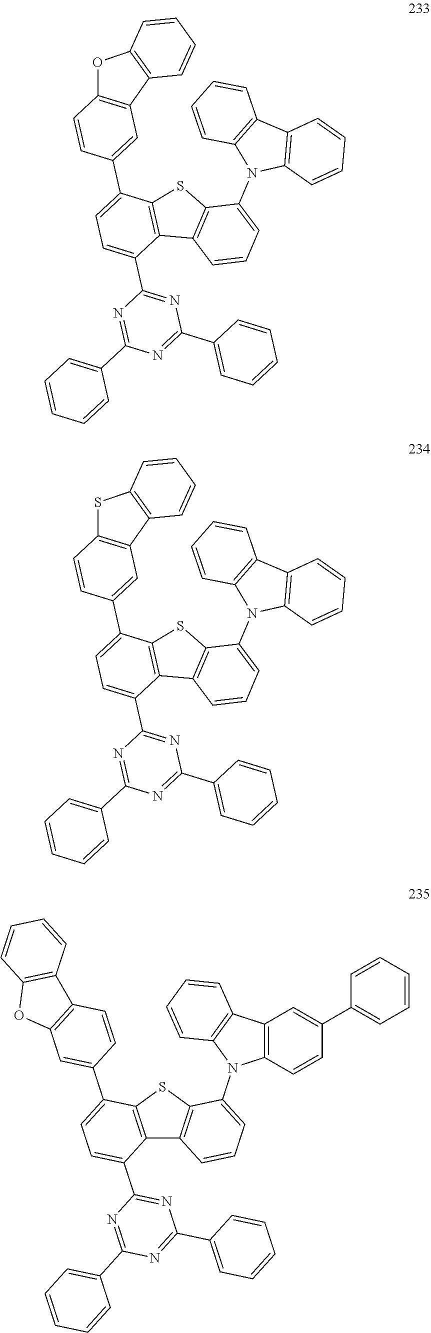 [SMILES: c1ccc(-c2nc(-c3ccccc3)nc(-c3ccc(-c4ccc5oc6ccccc6c5c4)c4sc5c(-n6c7ccccc7c7ccccc76)cccc5c34)n2)cc1 ; c1ccc(-c2nc(-c3ccccc3)nc(-c3ccc(-c4ccc5sc6ccccc6c5c4)c4sc5c(-n6c7ccccc7c7ccccc76)cccc5c34)n2)cc1 ; c1ccc(-c2ccc3c(c2)c2ccccc2n3-c2cccc3c2sc2c(-c4ccc5c(c4)oc4ccccc45)ccc(-c4nc(-c5ccccc5)nc(-c5ccccc5)n4)c23)cc1]

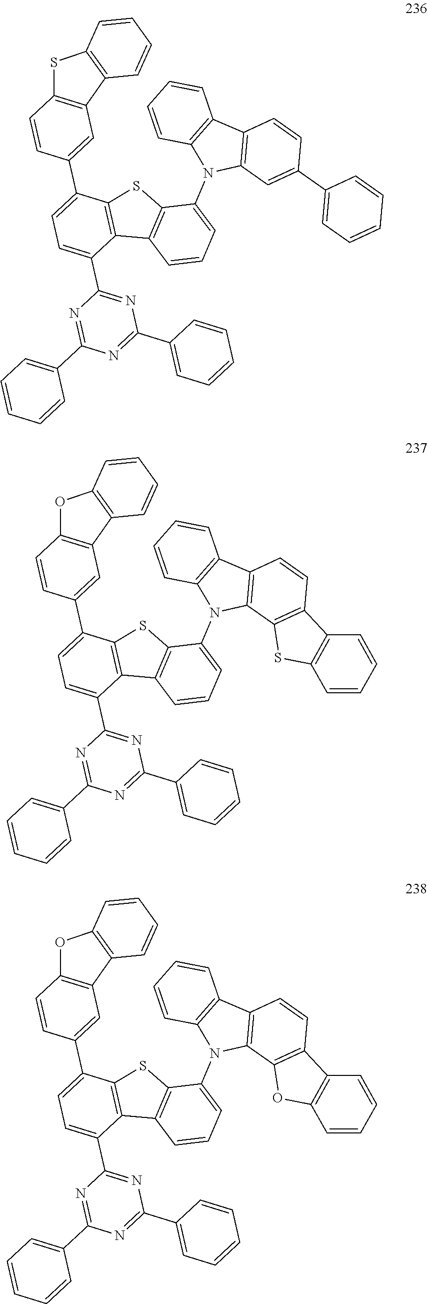 [SMILES: c1ccc(-c2ccc3c4ccccc4n(-c4cccc5c4sc4c(-c6ccc7sc8ccccc8c7c6)ccc(-c6nc(-c7ccccc7)nc(-c7ccccc7)n6)c45)c3c2)cc1 ; c1ccc(-c2nc(-c3ccccc3)nc(-c3ccc(-c4ccc5oc6ccccc6c5c4)c4sc5c(-n6c7ccccc7c7ccc8c9ccccc9sc8c76)cccc5c34)n2)cc1 ; c1ccc(-c2nc(-c3ccccc3)nc(-c3ccc(-c4ccc5oc6ccccc6c5c4)c4sc5c(-n6c7ccccc7c7ccc8c9ccccc9oc8c76)cccc5c34)n2)cc1]

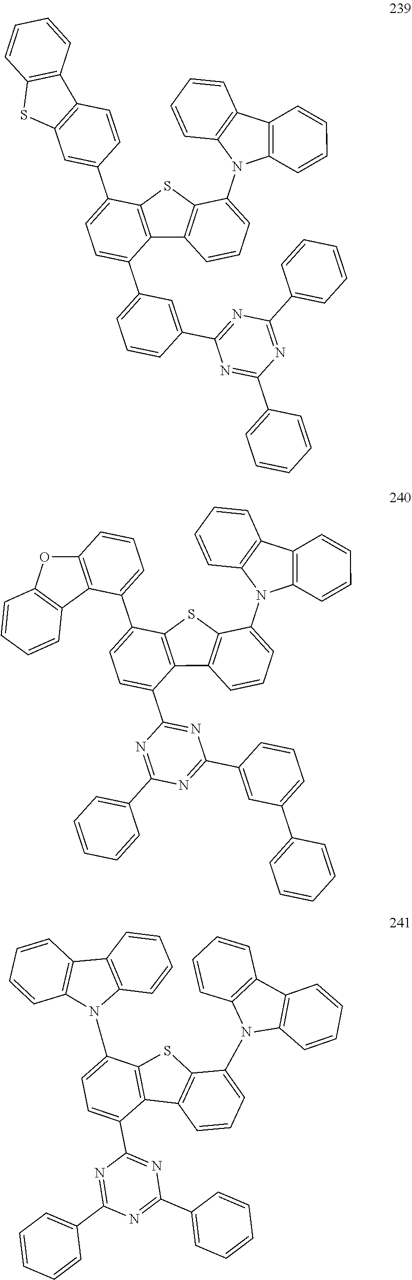

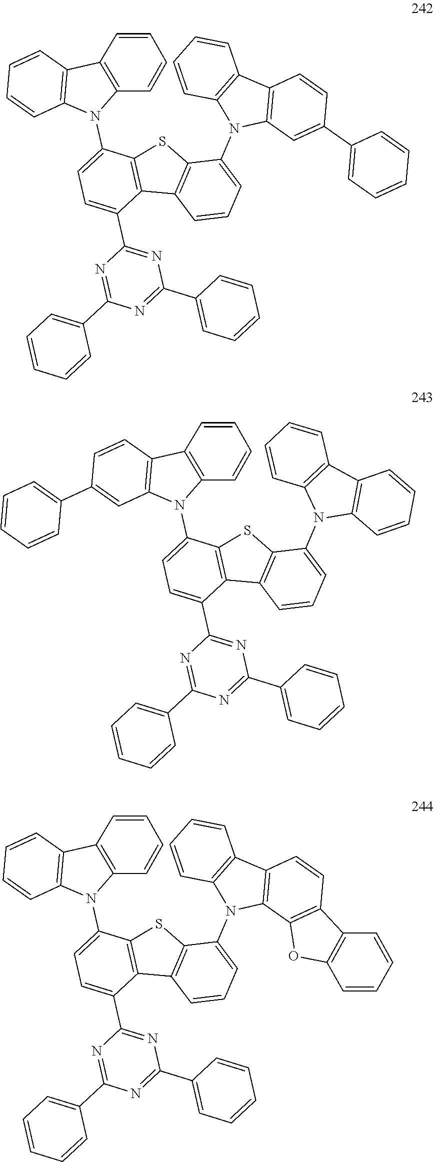 [SMILES: c1ccc(-c2ccc3c4ccccc4n(-c4cccc5c4sc4c(-n6c7ccccc7c7ccccc76)ccc(-c6nc(-c7ccccc7)nc(-c7ccccc7)n6)c45)c3c2)cc1 ; c1ccc(-c2ccc3c4ccccc4n(-c4ccc(-c5nc(-c6ccccc6)nc(-c6ccccc6)n5)c5c4sc4c(-n6c7ccccc7c7ccccc76)cccc45)c3c2)cc1 ; c1ccc(-c2nc(-c3ccccc3)nc(-c3ccc(-n4c5ccccc5c5ccccc54)c4sc5c(-n6c7ccccc7c7ccc8c9ccccc9oc8c76)cccc5c34)n2)cc1]

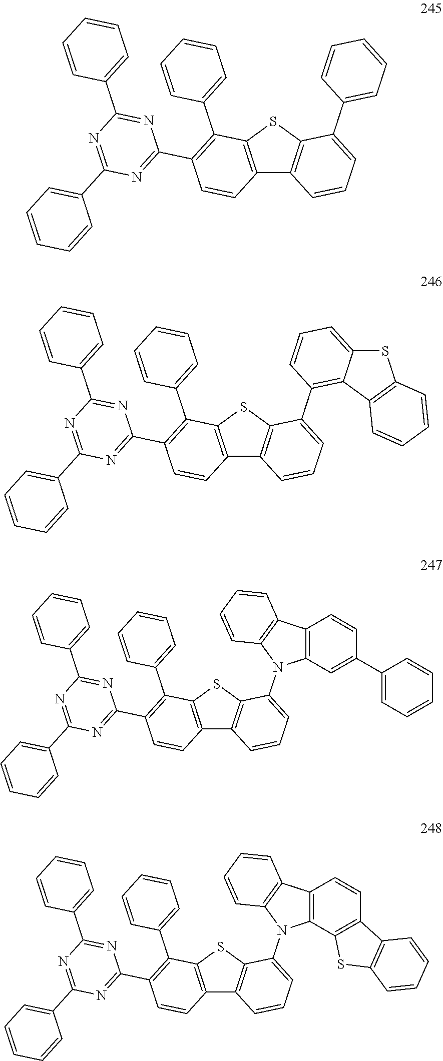 [SMILES: c1ccc(-c2nc(-c3ccccc3)nc(-c3ccc4c(sc5c(-c6ccccc6)cccc54)c3-c3ccccc3)n2)cc1 ; c1ccc(-c2nc(-c3ccccc3)nc(-c3ccc4c(sc5c(-c6cccc7sc8ccccc8c67)cccc54)c3-c3ccccc3)n2)cc1 ; c1ccc(-c2ccc3c4ccccc4n(-c4cccc5c4sc4c(-c6ccccc6)c(-c6nc(-c7ccccc7)nc(-c7ccccc7)n6)ccc45)c3c2)cc1 ; c1ccc(-c2nc(-c3ccccc3)nc(-c3ccc4c(sc5c(-n6c7ccccc7c7ccc8c9ccccc9sc8c76)cccc54)c3-c3ccccc3)n2)cc1]

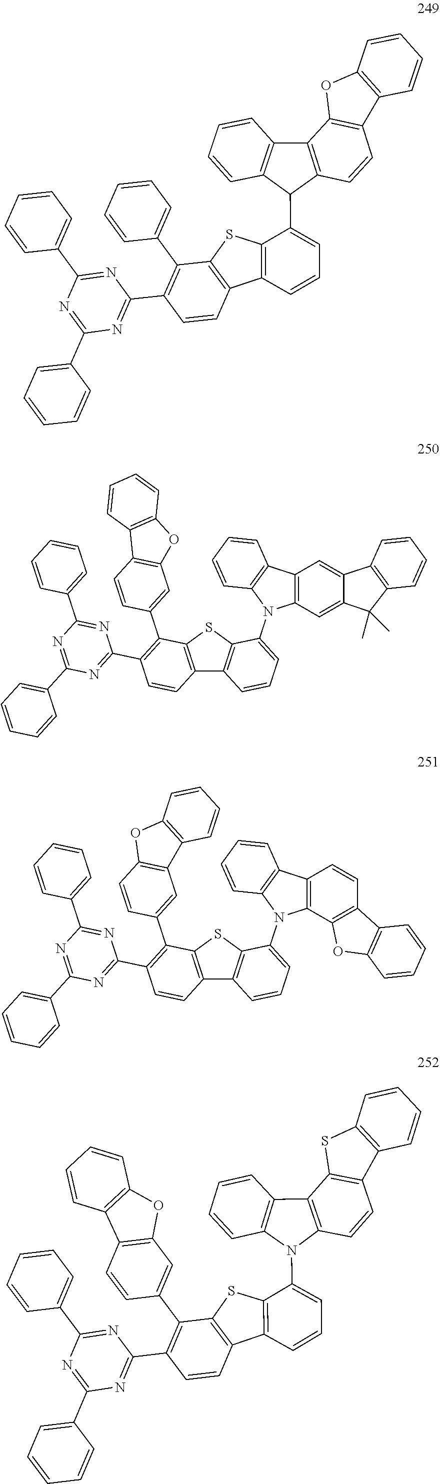 [SMILES: c1ccc(-c2nc(-c3ccccc3)nc(-c3ccc4c(sc5c(C6c7ccccc7-c7c6ccc6c7oc7ccccc76)cccc54)c3-c3ccccc3)n2)cc1 ; CC1(C)c2ccccc2-c2cc3c4ccccc4n(-c4cccc5c4sc4c(-c6ccc7c(c6)oc6ccccc67)c(-c6nc(-c7ccccc7)nc(-c7ccccc7)n6)ccc45)c3cc21 ; c1ccc(-c2nc(-c3ccccc3)nc(-c3ccc4c(sc5c(-n6c7ccccc7c7ccc8c9ccccc9oc8c76)cccc54)c3-c3ccc4oc5ccccc5c4c3)n2)cc1 ; c1ccc(-c2nc(-c3ccccc3)nc(-c3ccc4c(sc5c(-n6c7ccccc7c7c8sc9ccccc9c8ccc76)cccc54)c3-c3ccc4c(c3)oc3ccccc34)n2)cc1]

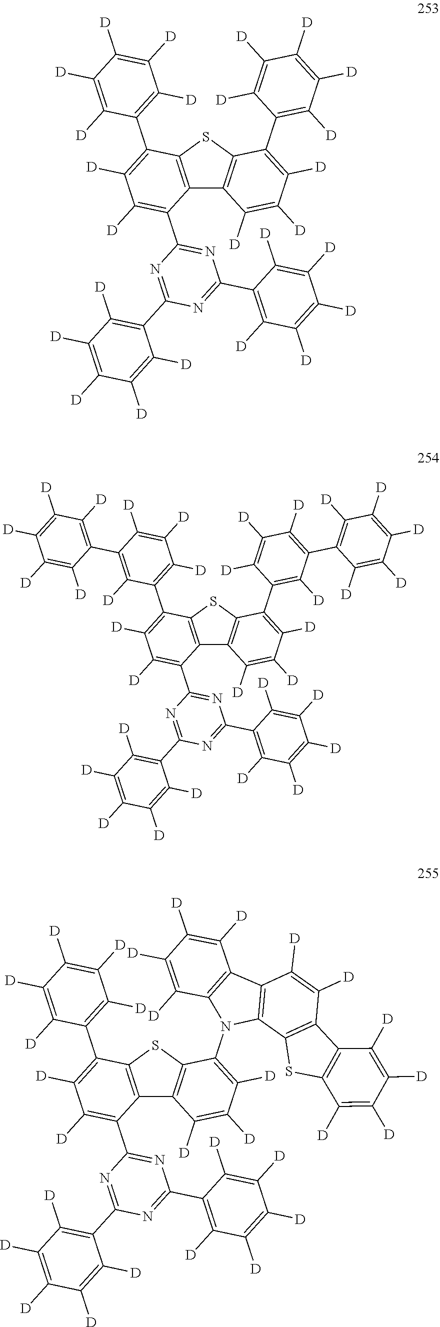

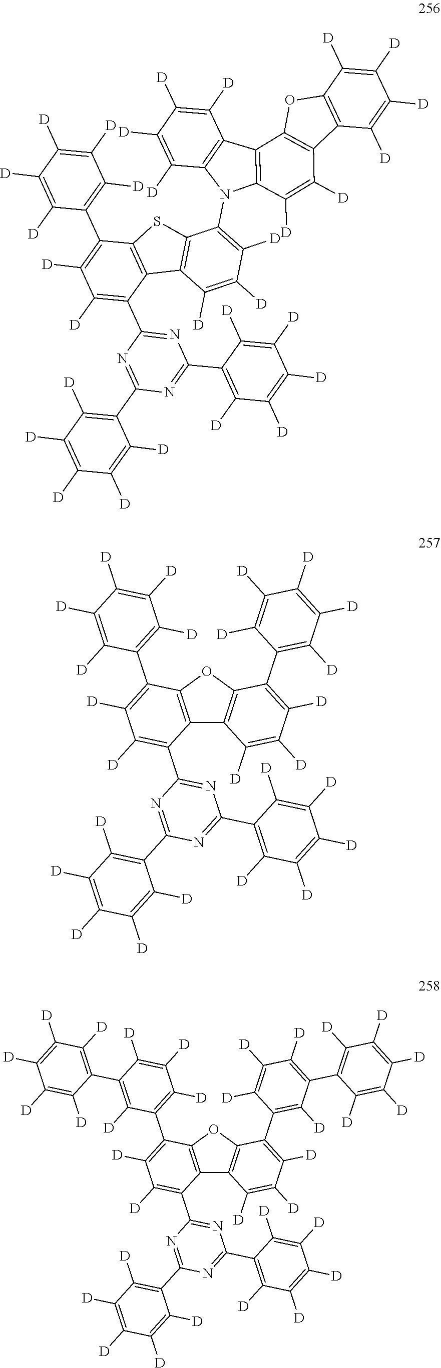

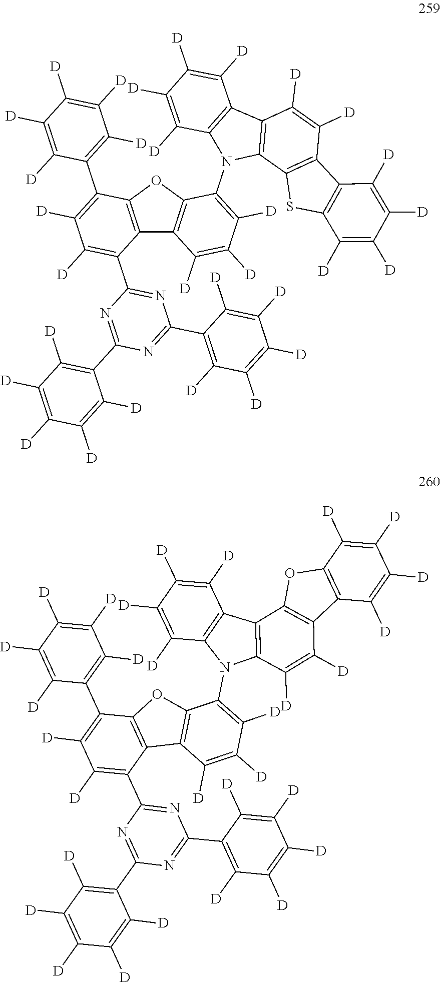

In addition, by introducing various substituents to the structure of Chemical Formula 1, compounds having unique properties of the introduced substituents may be synthesized. For example, by introducing substituents normally used as hole injection layer materials, hole transport layer materials, light emitting layer materials, electron transport layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

Meanwhile, the compound has a high glass transition temperature (Tg), and has excellent thermal stability. Such an increase in the thermal stability becomes an important factor providing driving stability to a device.

In addition, one embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound represented by Chemical Formula 1.

In one embodiment of the present application, the first electrode may be an anode, and the second electrode may be a cathode.

In another embodiment, the first electrode may be a cathode, and the second electrode may be an anode.

In one embodiment of the present application, the organic light emitting device may be a blue organic light emitting device, and the heterocyclic compound according to Chemical Formula 1 may be used as a material of the blue organic light emitting device.

In one embodiment of the present application, the organic light emitting device may be a green organic light emitting device, and the heterocyclic compound represented by Chemical Formula 1 may be used as a material of the green organic light emitting device.

In one embodiment of the present application, the organic light emitting device may be a red organic light emitting device, and the heterocyclic compound represented by Chemical Formula 1 may be used as a material of the red organic light emitting device.

In one embodiment of the present application, the organic light emitting device may be a blue organic light emitting device, and the heterocyclic compound according to Chemical Formula 1 may be used as a light emitting layer material of the blue organic light emitting device.

In one embodiment of the present application, the organic light emitting device may be a green organic light emitting device, and the heterocyclic compound represented by Chemical Formula 1 may be used as a light emitting layer material of the green organic light emitting device.

In one embodiment of the present application, the organic light emitting device may be a red organic light emitting device, and the heterocyclic compound represented by Chemical Formula 1 may be used as a light emitting layer material of the red organic light emitting device.

Specific details on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, or may also be formed in a multilayer structure in which two or more organic material layers are laminated. For example, the organic light emitting device according to one embodiment of the present disclosure may have a structure comprising a hole injection layer, a hole transport layer, a light emitting layer, an electron transport layer, an electron injection layer and the like as the organic material layer. However, the structure of the organic light emitting device is not limited thereto, and may comprise a smaller number of organic material layers.

In the organic light emitting device of the present disclosure, the organic material layer comprises a light emitting layer, and the light emitting layer may comprise the heterocyclic compound of Chemical Formula 1.

In the organic light emitting device of the present disclosure, the organic material layer comprises a light emitting layer, and the light emitting layer may comprise the heterocyclic compound of Chemical Formula 1 as a host of the light emitting layer.

In the organic light emitting device according to one embodiment of the present application, the organic material layer comprising the heterocyclic compound represented by Chemical Formula 1 further comprises a heterocyclic compound represented by the following Chemical Formula A.

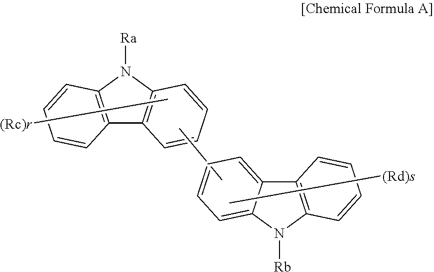

In Chemical Formula A,

Rc and Rd are the same as or different from each other, and each independently selected from the group consisting of hydrogen; deuterium; a halogen group; —CN; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiR201R202R203; —P(═O)R201R202; and —NR201R202, or two or more groups adjacent to each other bond to each other to form a substituted or unsubstituted C6 to C60 aromatic hydrocarbon ring or a substituted or unsubstituted C2 to C60 heteroring,

Ra and Rb are the same as or different from each other, and each independently a substituted or unsubstituted C6 to C60 aryl group; —SiR201R202R203; —P(═O)R201R202; or a substituted or unsubstituted C2 to C60 heteroaryl group,

R201, R202 and R203 are the same as or different from each other, and each independently hydrogen; deuterium; —CN; a substituted or unsubstituted C1 to C60 alkyl group; a substituted or unsubstituted C6 to C60 aryl group; or a substituted or unsubstituted C2 to C60 heteroaryl group,

r and s are an integer of 0 to 7, and

when r and s are 2 or greater, substituents in the parentheses are the same as or different from each other.

Effects of more superior efficiency and lifetime are obtained when comprising the heterocyclic compound of Chemical Formula 1 and the heterocyclic compound of Chemical Formula A in the organic material layer of the organic light emitting device. Such results may lead to a forecast that an exciplex phenomenon occurs when comprising the two compounds at the same time.

The exciplex phenomenon is a phenomenon of releasing energy having sizes of a donor (p-host) HOMO level and an acceptor (n-host) LUMO level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transport ability and an acceptor (n-host) having a favorable electron transport ability are used as a host of a light emitting layer, holes are injected to the p-host and electrons are injected to the n-host, and therefore, a driving voltage may be lowered, which resultantly helps with enhancement in the lifetime.

In one embodiment of the present application, Chemical Formula A may be represented by the following Chemical Formula A-1.

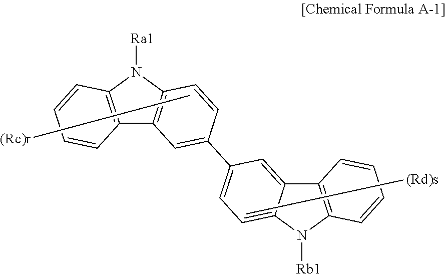

In Chemical Formula A-1,

Rc, Rd, r and s have the same definitions as in Chemical Formula A,

Ra1 and Rb1 are the same as or different from each other, and each independently a substituted or unsubstituted C6 to C60 aryl group; SiR201R202R203; or —P(═O)R201R202, and

R201, R202 and R203 have the same definitions as in Chemical Formula A.

In one embodiment of the present application, Rc and Rd of Chemical Formula A may be hydrogen.

In one embodiment of the present application, Ra1 and Rb1 of Chemical Formula A-1 are the same as or different from each other, and may be each independently a substituted or unsubstituted C6 to C60 aryl group.

In another embodiment, Ra1 and Rb1 of Chemical Formula A-1 are the same as or different from each other, and may be each independently a substituted or unsubstituted C6 to C40 aryl group.

In another embodiment, Ra1 and Rb1 of Chemical Formula A-1 are the same as or different from each other, and may be each independently a C6 to C40 aryl group unsubstituted or substituted with one or more substituents selected from the group consisting of a C1 to C40 alkyl group, a C6 to C40 aryl group, —CN and —SiR201R202R203.

In another embodiment, Ra1 and Rb1 of Chemical Formula A-1 are the same as or different from each other, and may be each independently a phenyl group unsubstituted or substituted with a phenyl group, —CN or —SiR201R202R203; a biphenyl group unsubstituted or substituted with a phenyl group; a naphthyl group; a fluorene group unsubstituted or substituted with a methyl group or a phenyl group; a spirobifluorene group; or a triphenylene group.

In one embodiment of the present application, R201, R202 and R203 of Chemical Formula A-1 may be a phenyl group.

In one embodiment of the present application, R201, R202 and R203 may have the same definitions as R, R′ and R″.

In one embodiment of the present application, the heterocyclic compound of Chemical Formula A may be represented by any one of the following compounds.

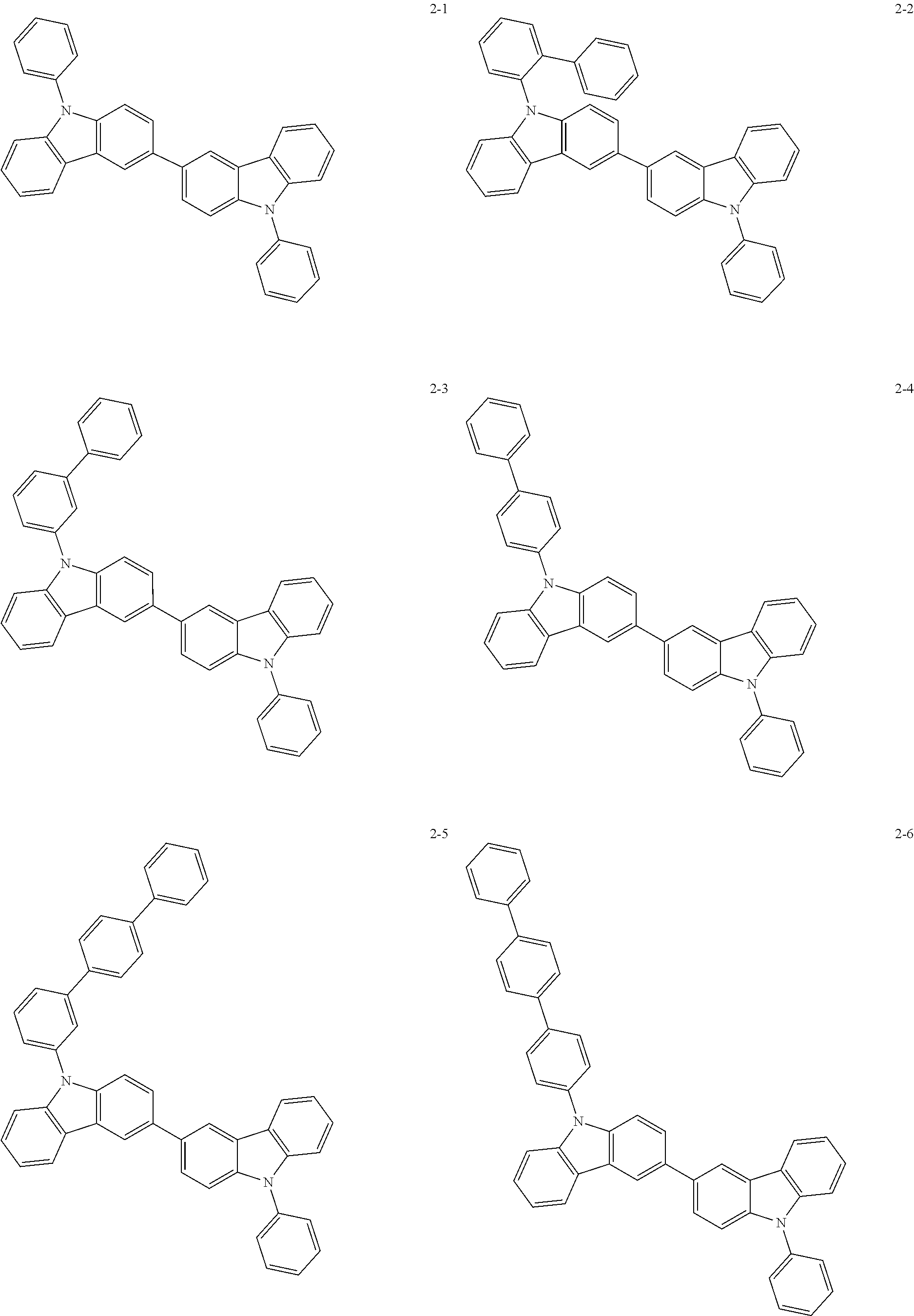 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

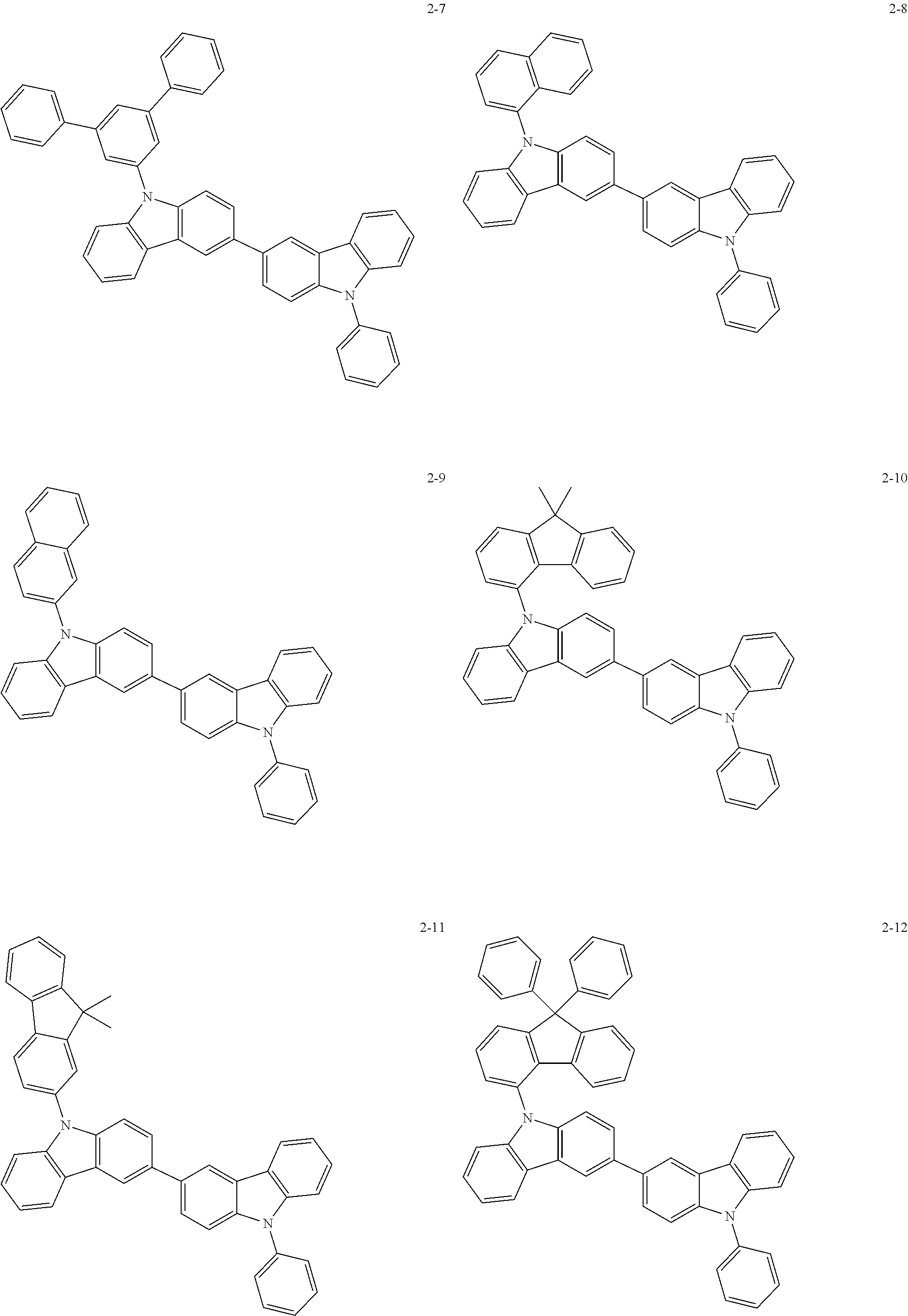 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

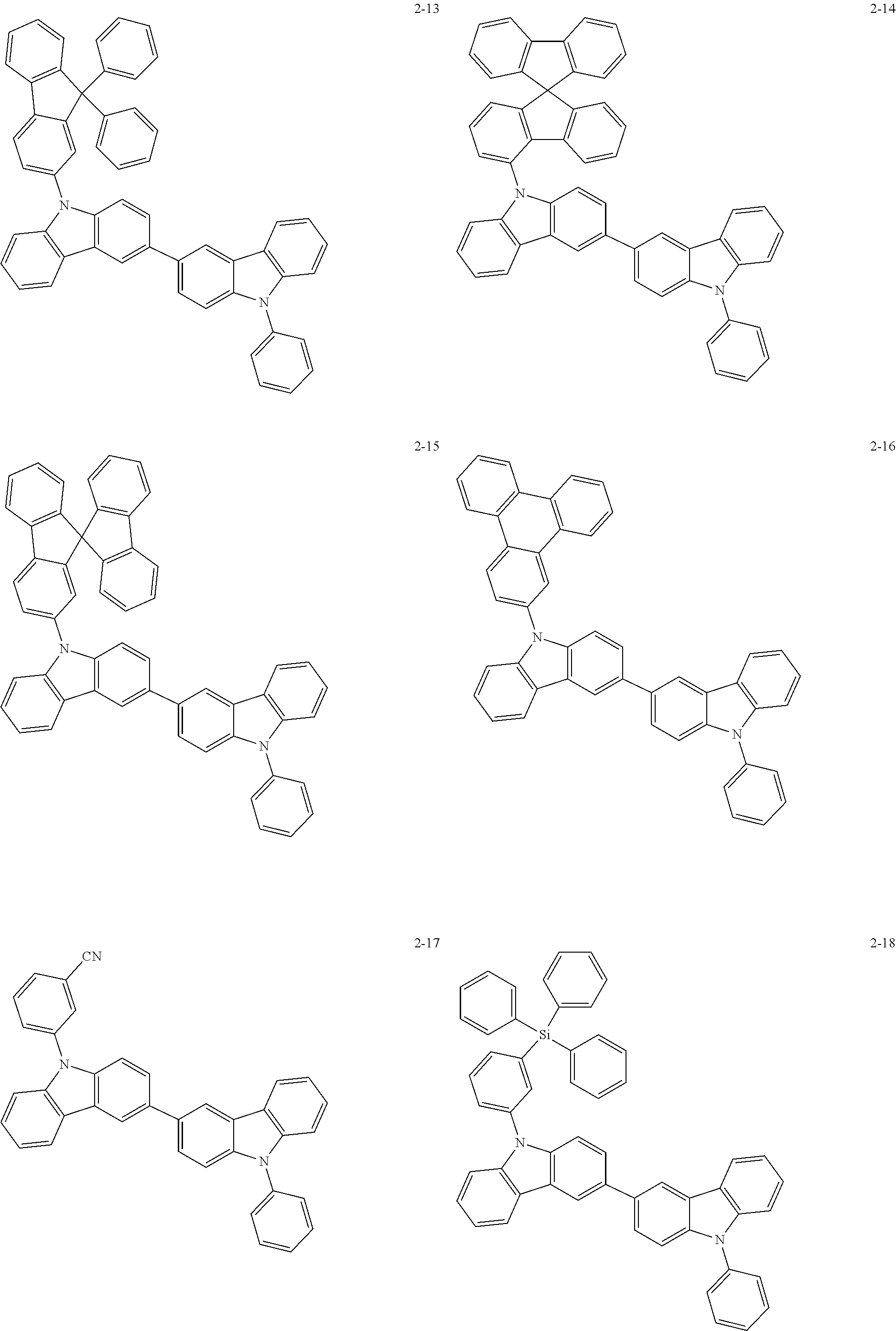

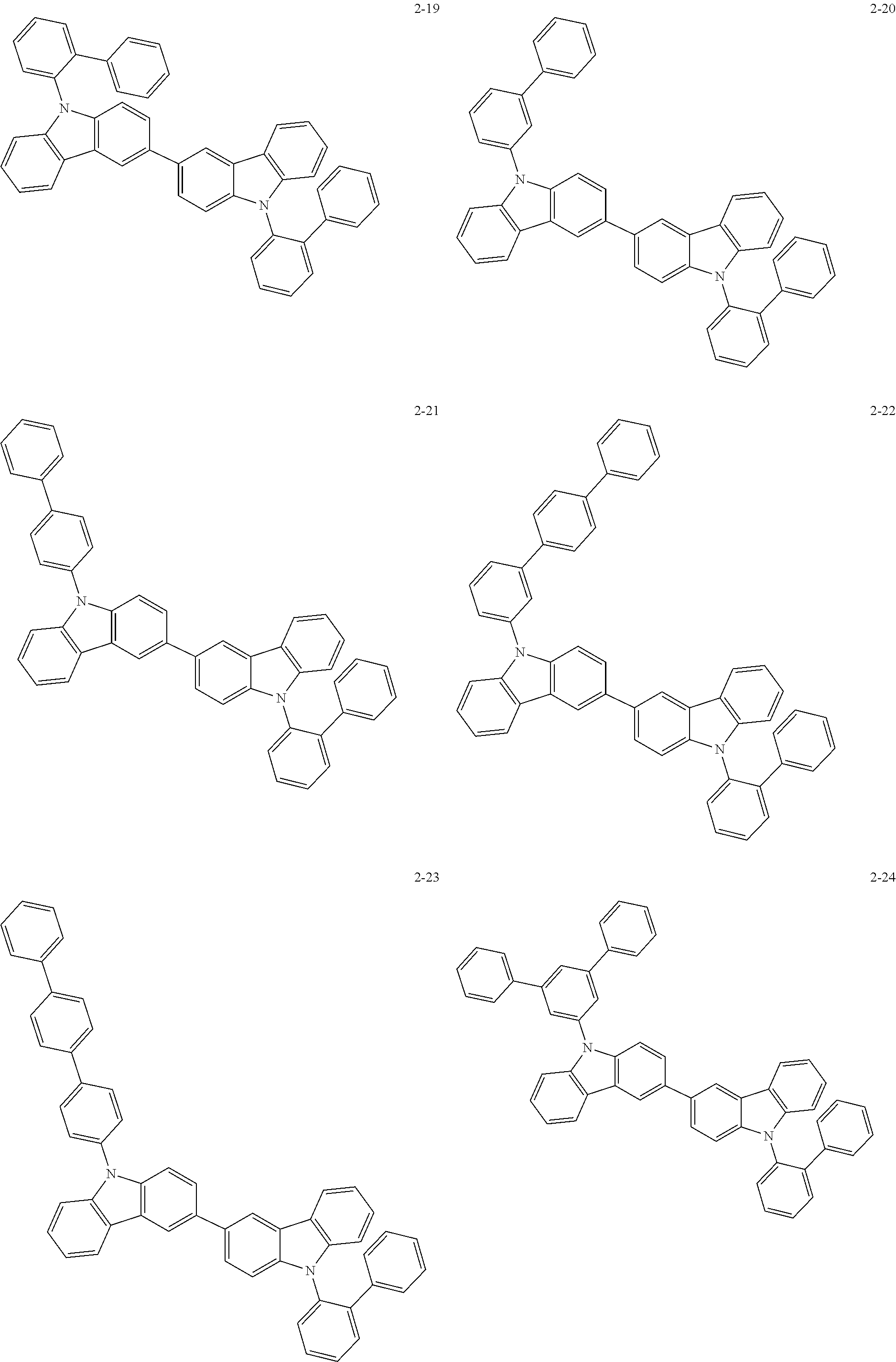

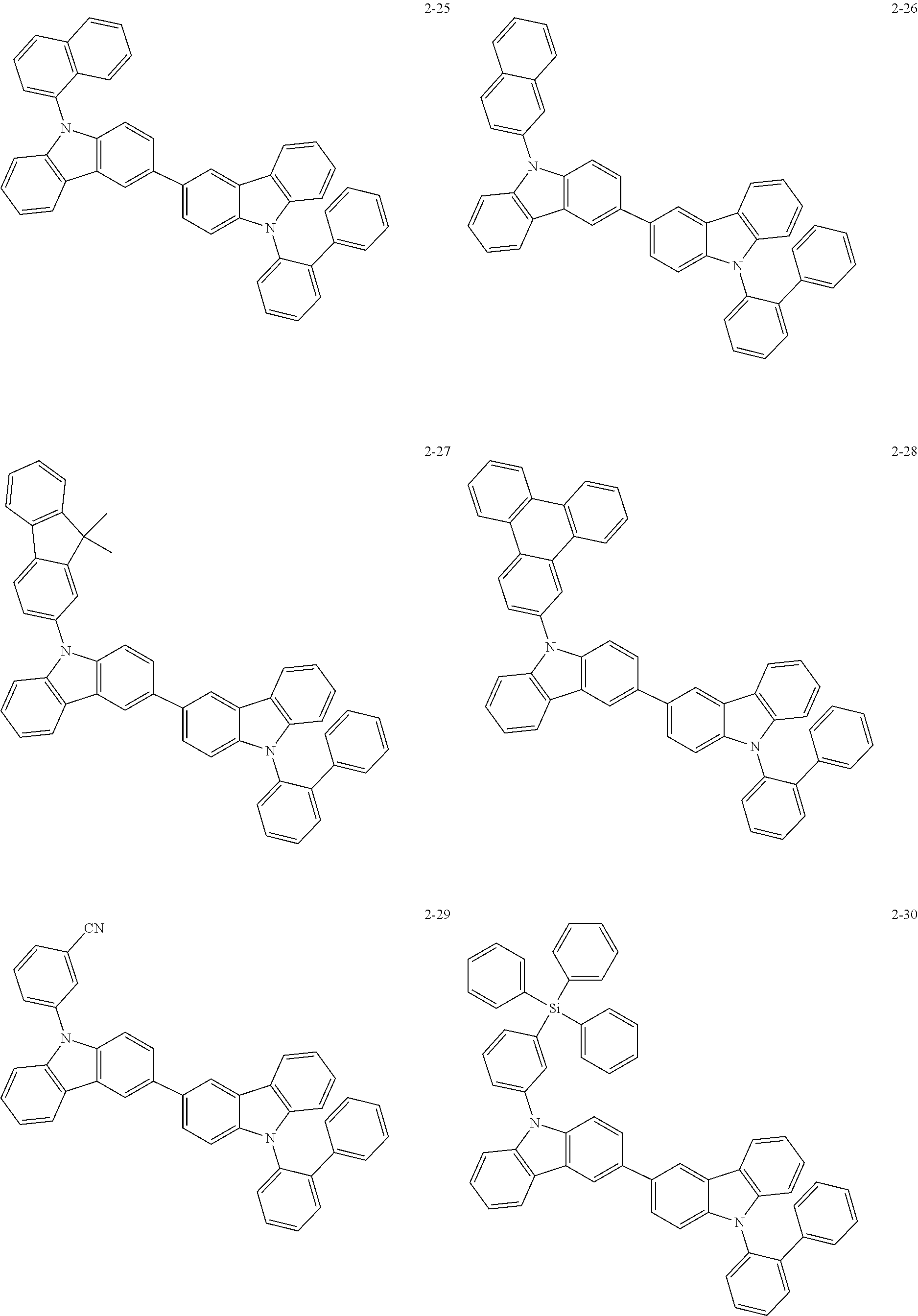

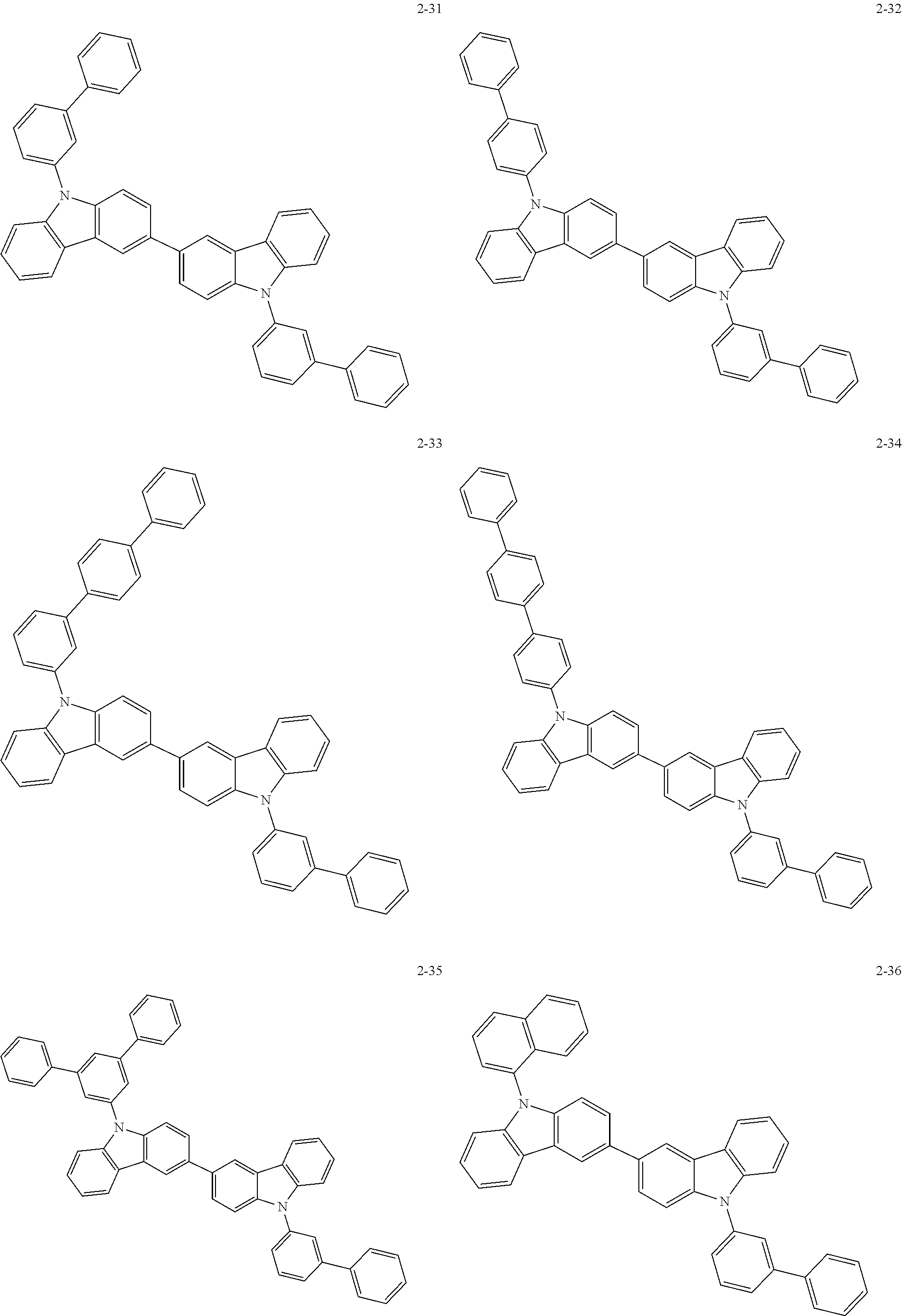

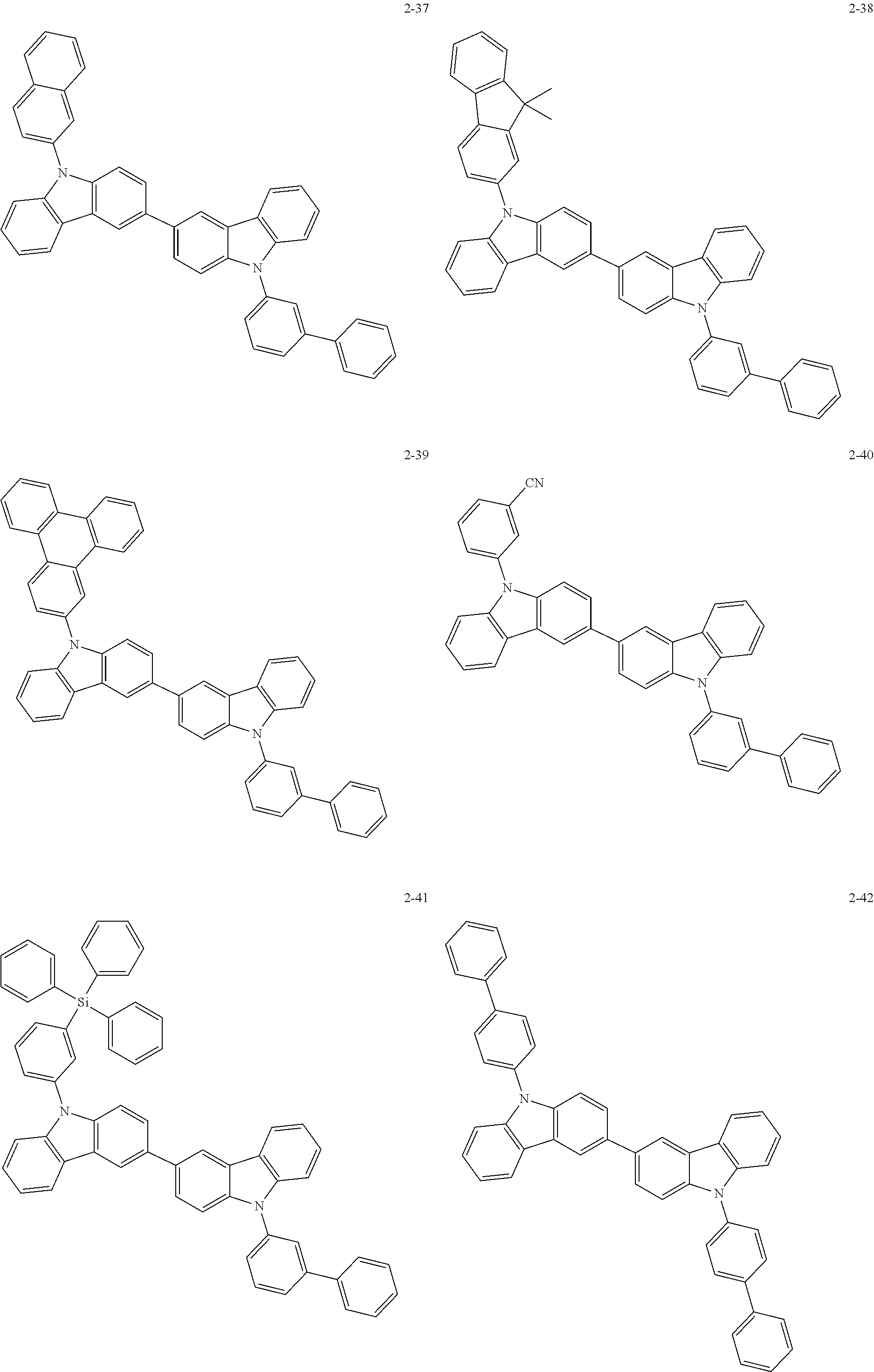

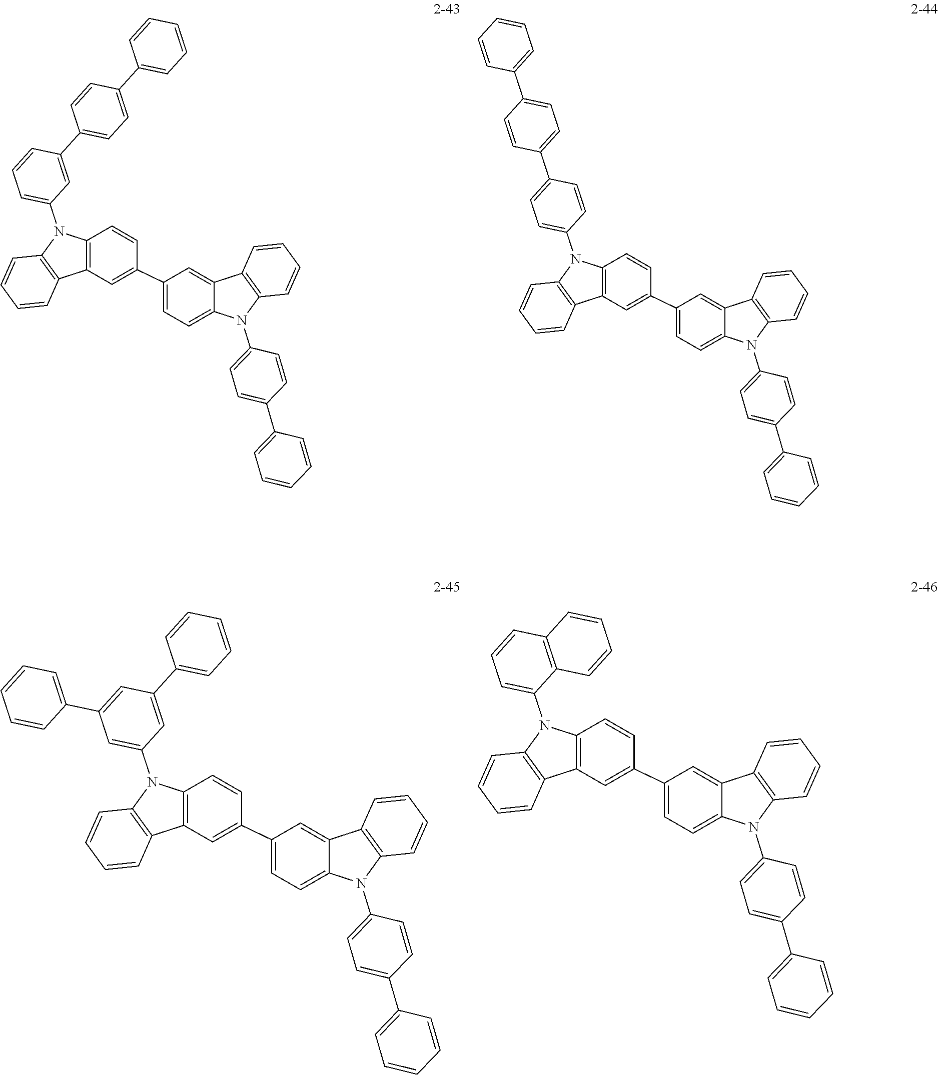 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2ccc(-n3c4ccccc4c4cc(-c5ccc6c(c5)c5ccccc5n6-c5cccc6ccccc56)ccc43)cc2)cc1]

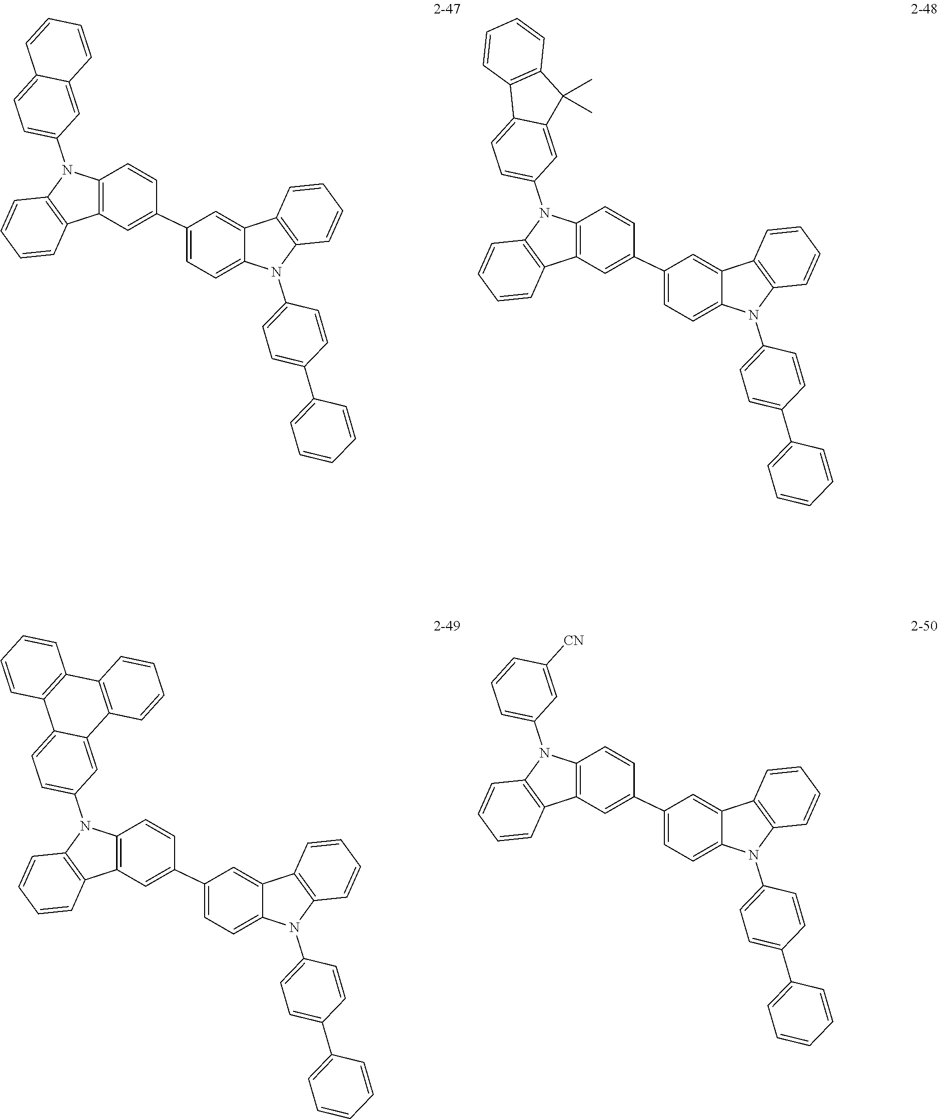 [SMILES: c1ccc(-c2ccc(-n3c4ccccc4c4cc(-c5ccc6c(c5)c5ccccc5n6-c5ccc6ccccc6c5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4ccc(-c5ccccc5)cc4)ccc32)c1]

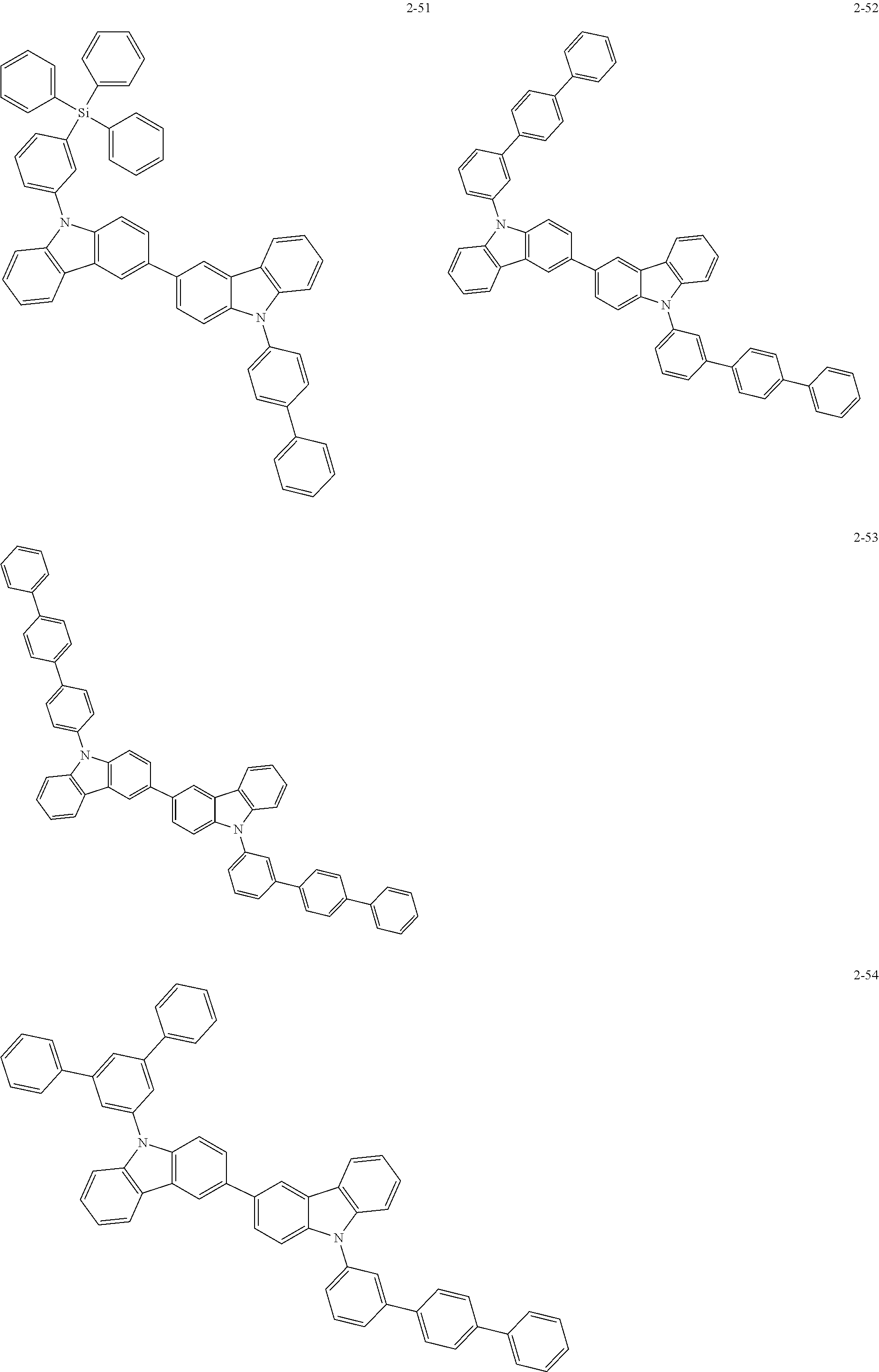 [SMILES: c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1 ; c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1]

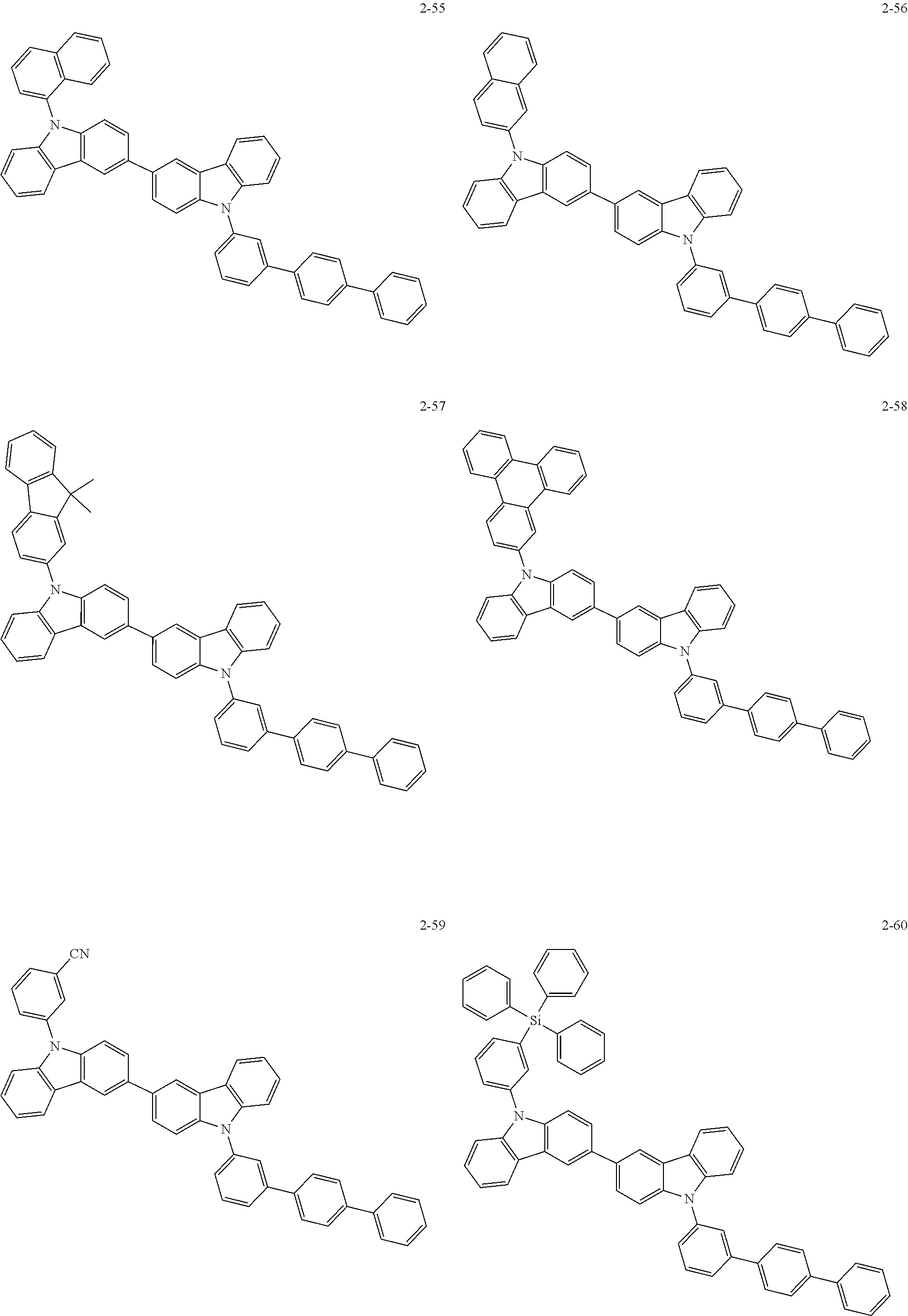

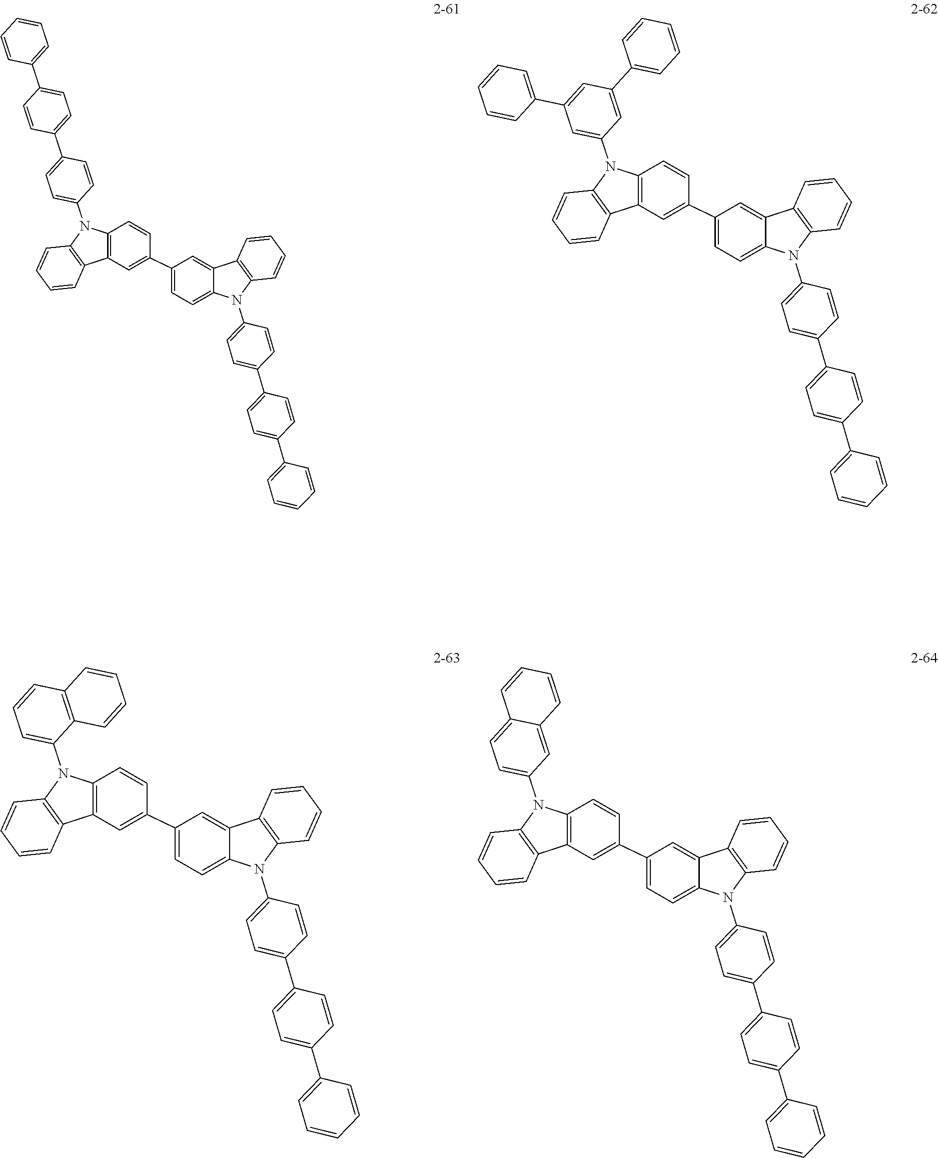 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

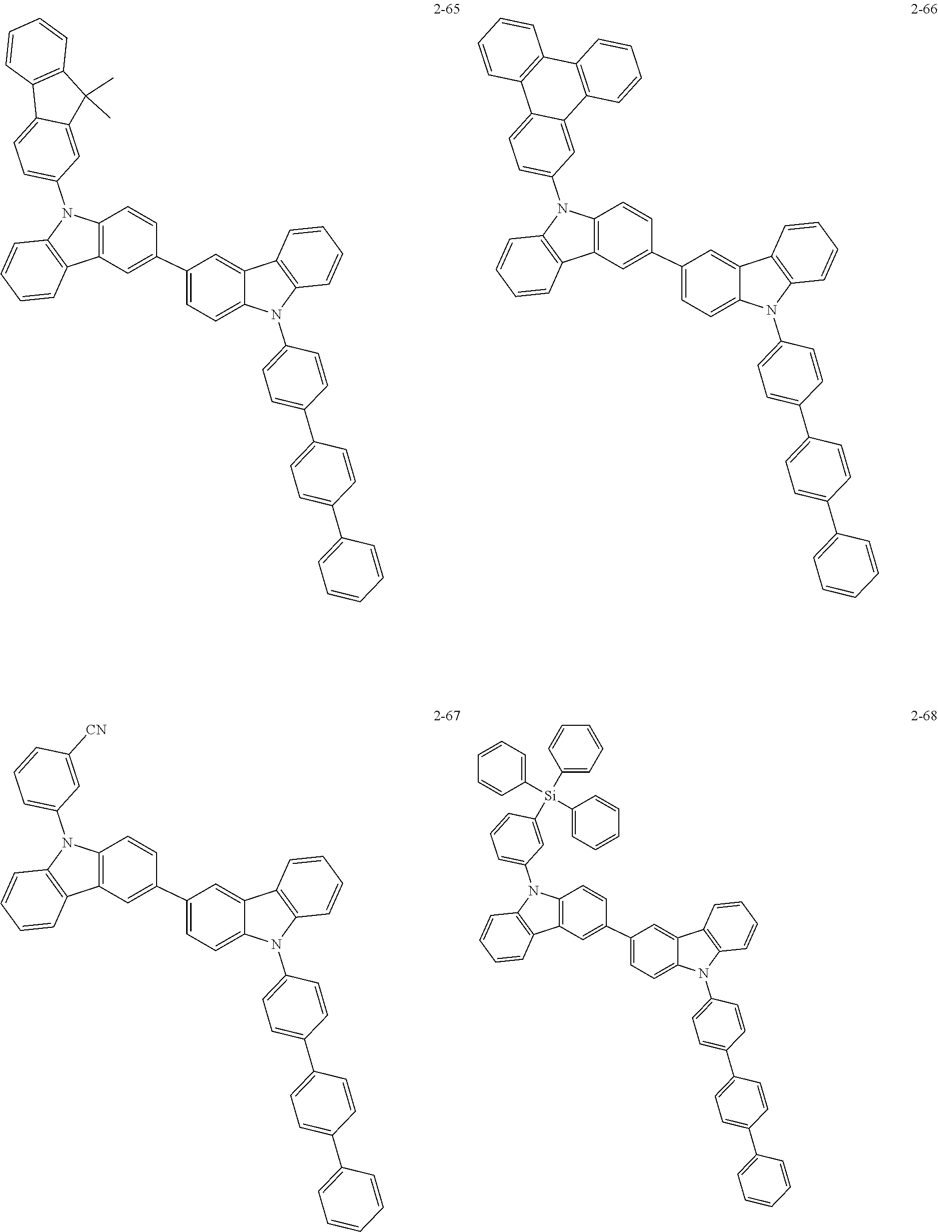 [SMILES: CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1 ; N#Cc1cccc(-n2c3ccccc3c3cc(-c4ccc5c(c4)c4ccccc4n5-c4ccc(-c5ccc(-c6ccccc6)cc5)cc4)ccc32)c1 ; c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1]

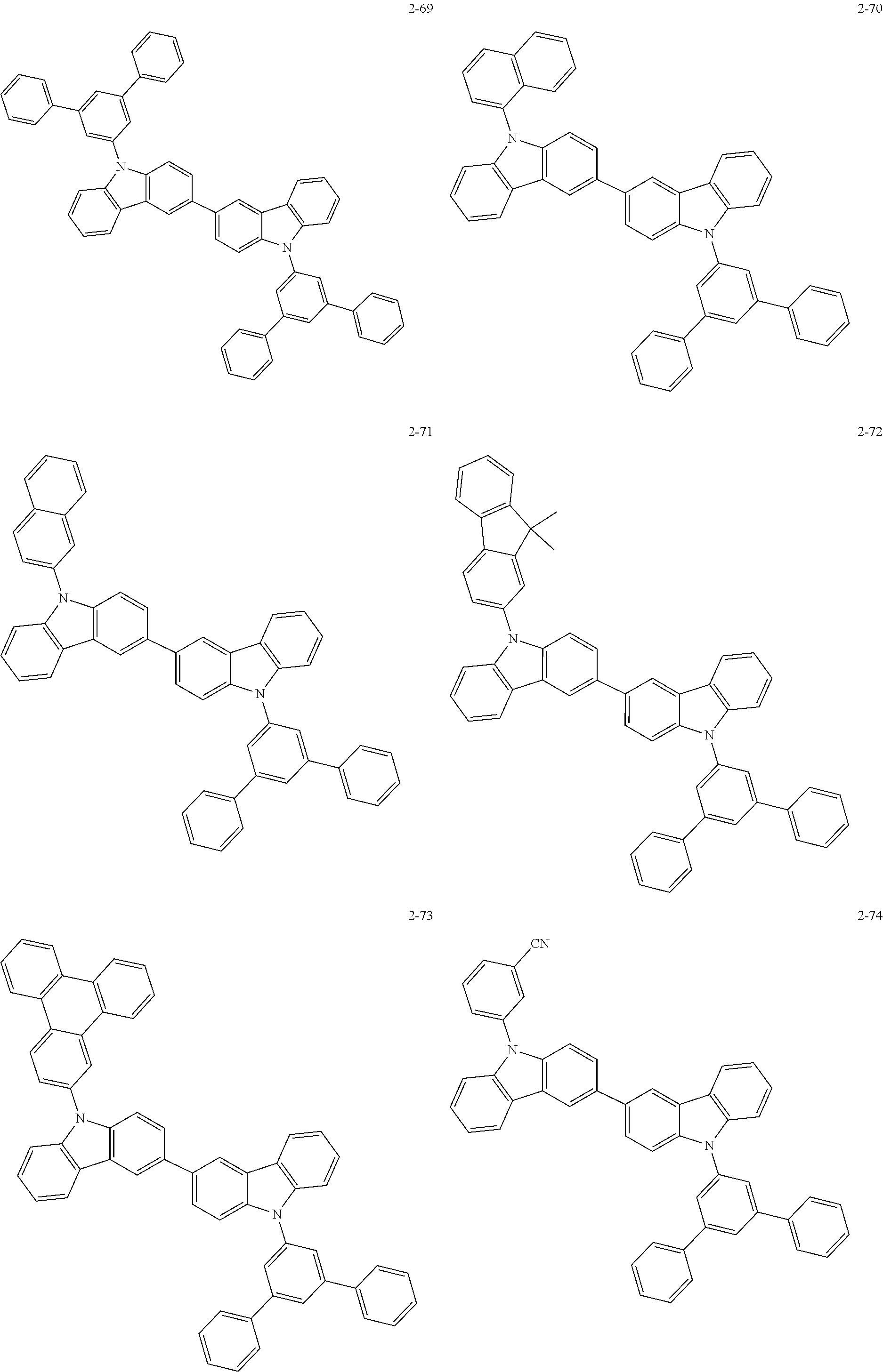

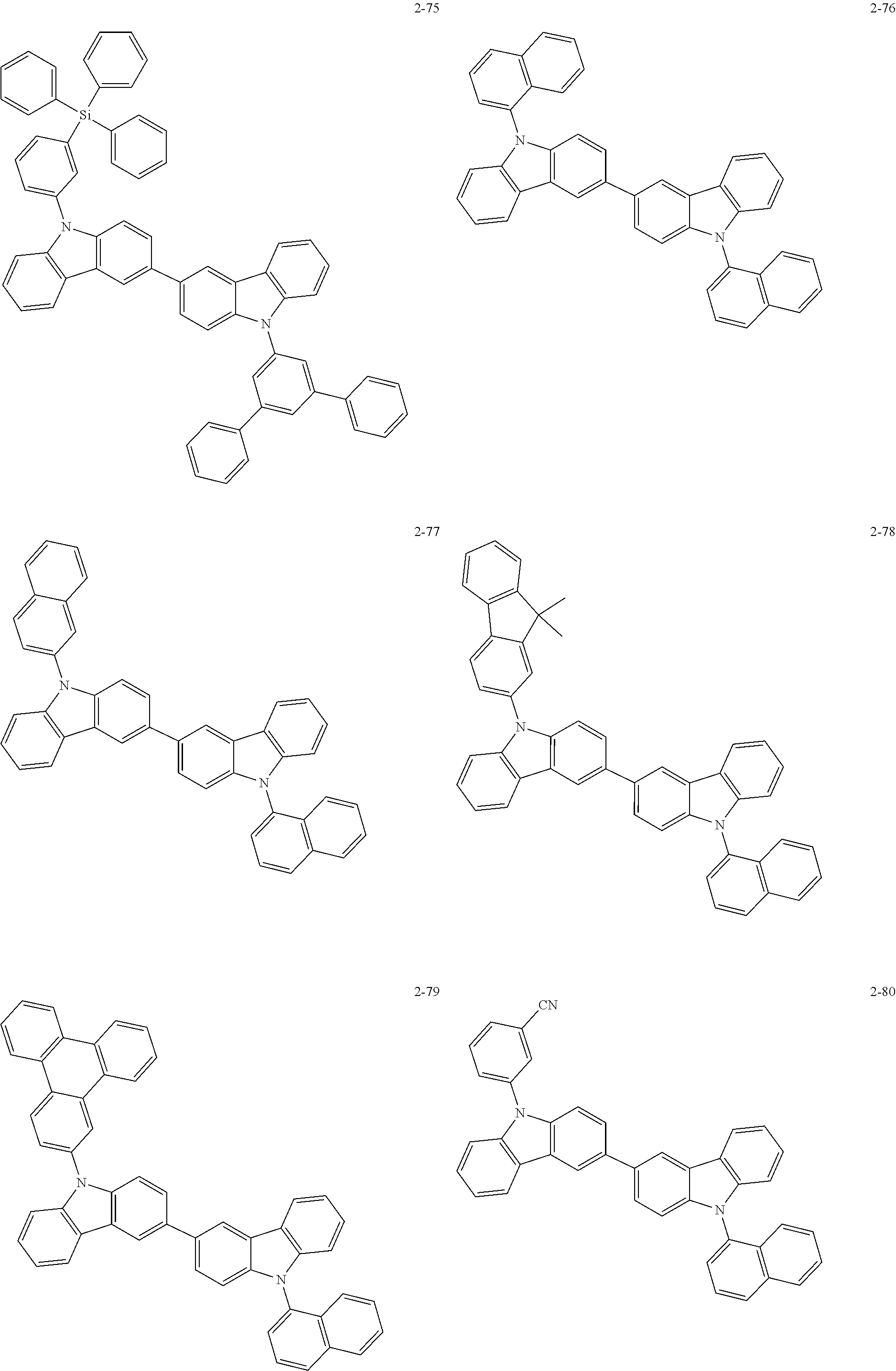

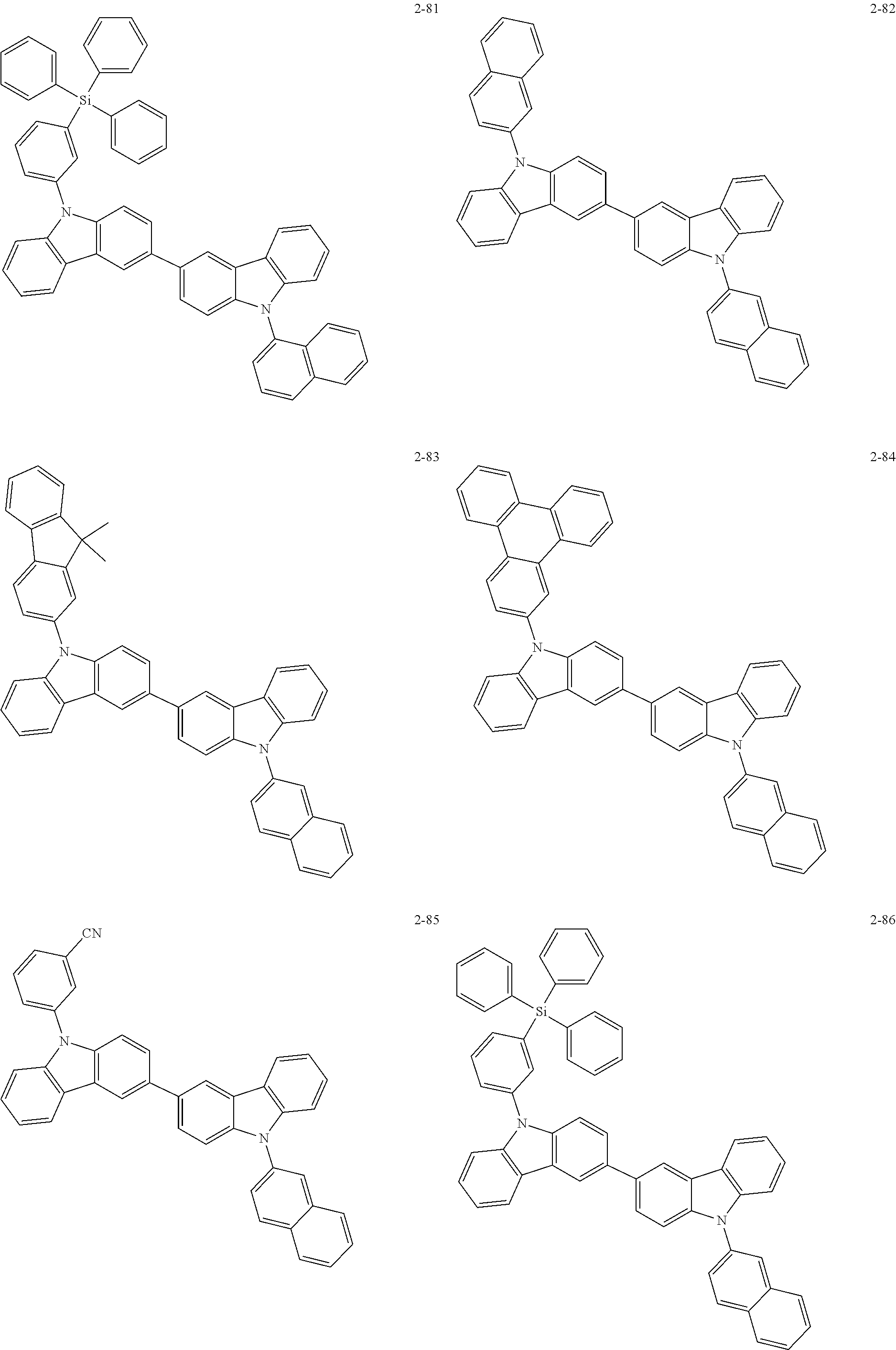

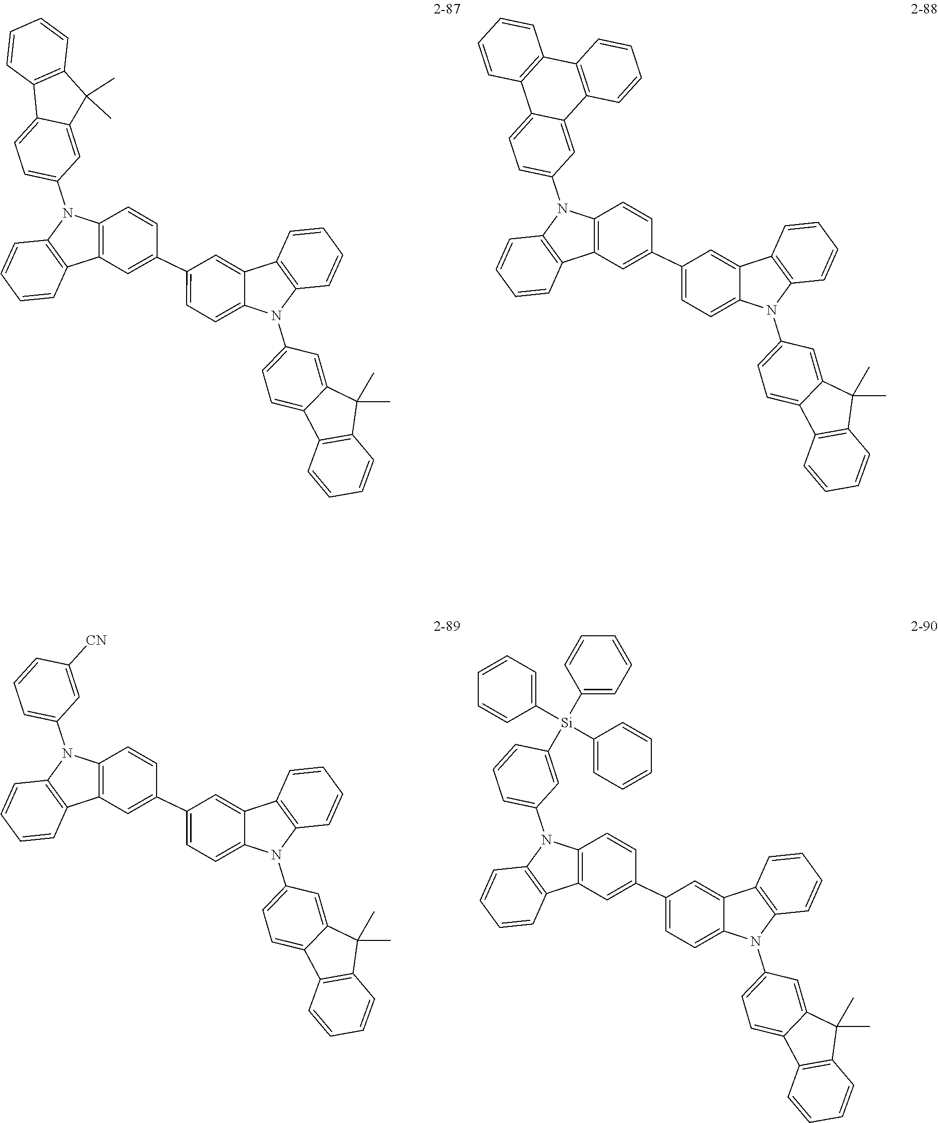 [SMILES: CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5ccccc5n6-c5ccc6c7ccccc7c7ccccc7c6c5)ccc43)cc21 ; CC1(C)c2ccccc2-c2ccc(-n3c4ccccc4c4cc(-c5ccc6c(c5)c5ccccc5n6-c5cccc(C#N)c5)ccc43)cc21 ; CC1(C)c2ccccc2-c2ccc(-n3c4ccccc4c4cc(-c5ccc6c(c5)c5ccccc5n6-c5cccc([Si](c6ccccc6)(c6ccccc6)c6ccccc6)c5)ccc43)cc21]

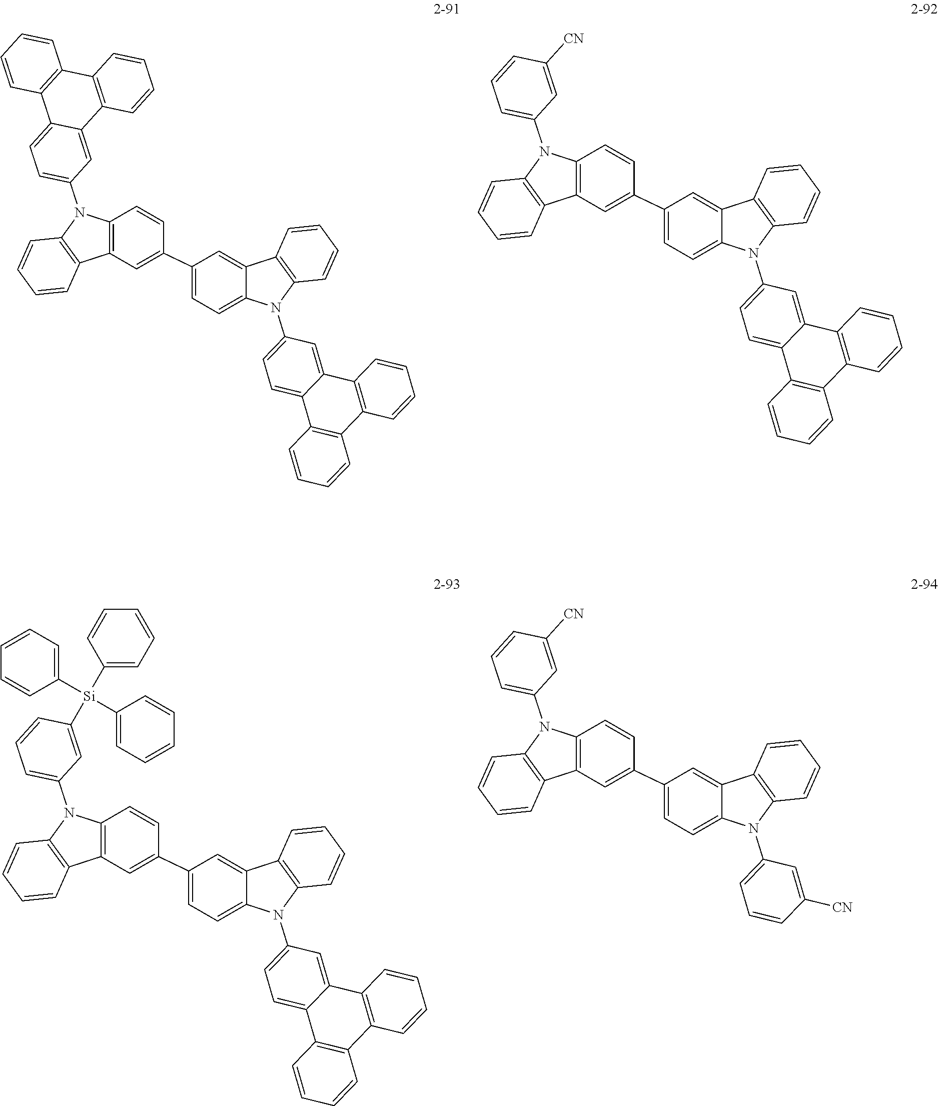 [SMILES: c1ccc2c(c1)c1ccccc1c1cc(-n3c4ccccc4c4cc(-c5ccc6c(c5)c5ccccc5n6-c5ccc6c7ccccc7c7ccccc7c6c5)ccc43)ccc21 ; N#Cc1cccc(-n2c3ccccc3c3cc(-c4ccc5c(c4)c4ccccc4n5-c4ccc5c6ccccc6c6ccccc6c5c4)ccc32)c1 ; c1ccc([Si](c2ccccc2)(c2ccccc2)c2cccc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cccc(C#N)c4)ccc32)c1]

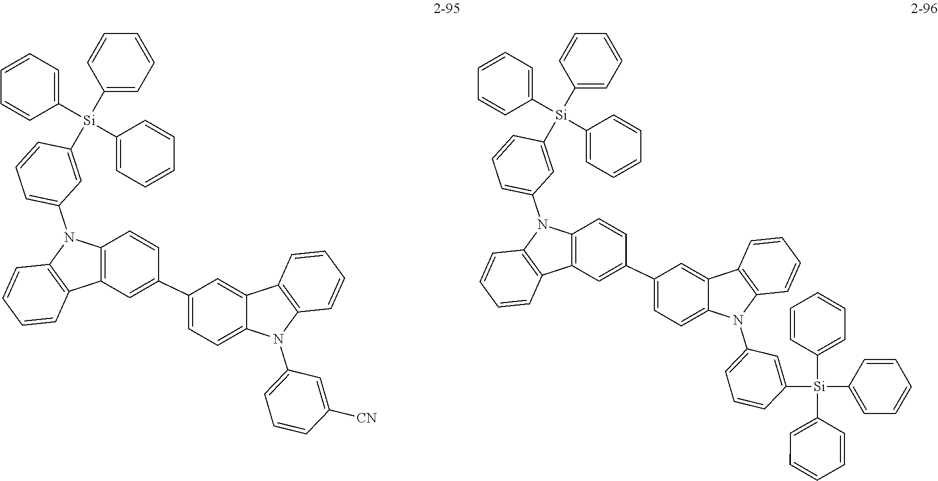 [SMILES: N#Cc1cccc(-n2c3ccccc3c3cc(-c4ccc5c(c4)c4ccccc4n5-c4cccc([Si](c5ccccc5)(c5ccccc5)c5ccccc5)c4)ccc32)c1 ; c1ccc([Si](c2ccccc2)(c2ccccc2)c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1]

In addition, another embodiment of the present application provides a composition for an organic material layer of an organic light emitting device, the composition comprising the heterocyclic compound represented by Chemical Formula 1, and the heterocyclic compound represented by Chemical Formula A.

Specific descriptions on the heterocyclic compound represented by Chemical Formula 1 and the heterocyclic compound represented by Chemical Formula A are the same as the descriptions provided above.

In the composition, the heterocyclic compound represented by Chemical Formula 1: the heterocyclic compound represented by Chemical Formula A may have a weight ratio of 1:10 to 10:1, 1:8 to 8:1, 1:5 to 5:1 or 1:2 to 2:1, however, the weight ratio is not limited thereto.

The composition may be used when forming an organic material of an organic light emitting device, and may be more preferably used when forming a host of a light emitting layer.

In one embodiment of the present application, the organic material layer comprises the heterocyclic compound represented by Chemical Formula 1 and the heterocyclic compound represented by Chemical Formula A, and a phosphorescent dopant may be used therewith.

In one embodiment of the present application, the organic material layer comprises the heterocyclic compound represented by Chemical Formula 1 and the heterocyclic compound represented by Chemical Formula A, and an iridium-based dopant may be used therewith.

As a material of the phosphorescent dopant, those known in the art may be used.

For example, phosphorescent dopant materials represented by LL′MX′, LL′L″M, LMX′X″, L2 mX′ and L3 m may be used, however, the scope of the present disclosure is not limited to these examples.

Herein, L, L′, L″, X′ and X″ are a bidentate ligand different from each other, and M is a metal forming an octahedral complex.

M may comprise iridium, platinum, osmium and the like.

L is an anionic bidentate ligand coordinated to M as the iridium-based dopant by sp2 carbon and heteroatom, and X may function to trap electrons or holes. Nonlimiting examples of L may comprise 2-(1-naphthyl)benzoxazole, (2-phenylbenzoxazole), (2-phenylbenzothiazole), (2-phenylbenzothiazole), (7,8-benzoquinoline), (thiophene group pyrizine), phenylpyridine, benzothiophene group pyrizine, 3-methoxy-2-phenylpyridine, thiophene group pyrizine, tolylpyridine and the like.

Nonlimiting examples of X′ and X″ may comprise acetylacetonate (acac), hexafluoroacetylacetonate, salicylidene, picolinate, 8-hydroxyquinolinate and the like.

More specific examples thereof are described below, however, the phosphorescent dopant is not limited to these examples.

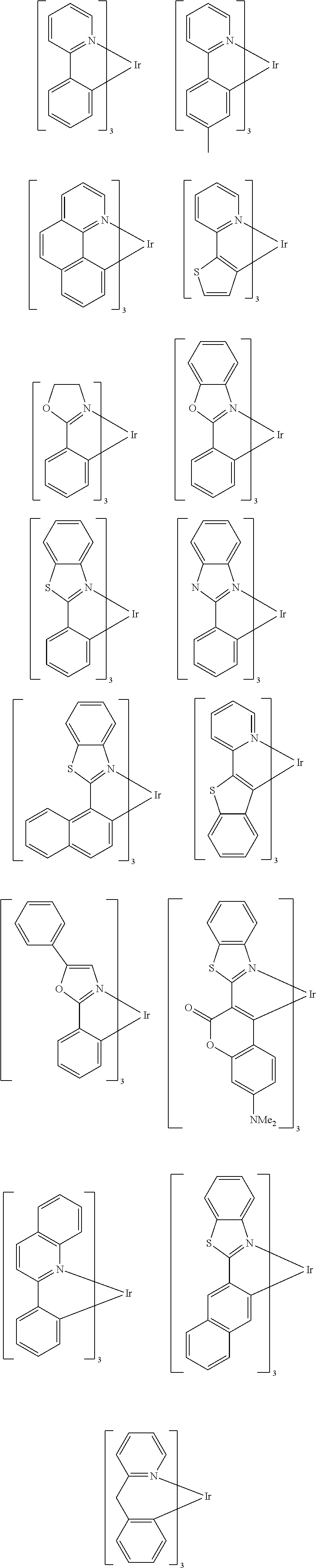

In one embodiment of the present application, as the iridium-based dopant, Ir(ppy)₃ may be used as a green phosphorescent dopant.

In one embodiment of the present application, a content of the dopant may be from 1% to 15%, preferably from 3% to 10% and more preferably from 5% to 10% based on the whole light emitting layer.

The content may mean a weight ratio.

In the organic light emitting device of the present disclosure, the organic material layer comprises an electron injection layer or an electron transport layer, and the electron injection layer or the electron transport layer may comprise the heterocyclic compound.

In another organic light emitting device, the organic material layer comprises an electron blocking layer or a hole blocking layer, and the electron blocking layer or the hole blocking layer may comprise the heterocyclic compound.

In another organic light emitting device, the organic material layer comprises an electron transport layer, a light emitting layer or a hole blocking layer, and the electron transport layer, the light emitting layer or the hole blocking layer may comprise the heterocyclic compound.

The organic light emitting device of the present disclosure may further comprise one, two or more layers selected from the group consisting of a light emitting layer, a hole injection layer, a hole transport layer, an electron injection layer, an electron transport layer, an electron blocking layer and a hole blocking layer.

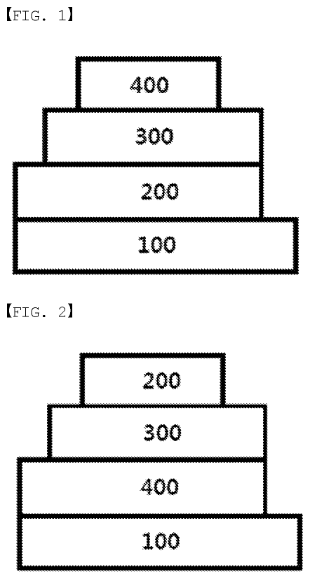

FIG. 1 to FIG. 3 illustrate a lamination order of electrodes and organic material layers of an organic light emitting device according to one embodiment of the present application. However, the scope of the present application is not limited to these diagrams, and structures of organic light emitting devices known in the art may also be used in the present application.

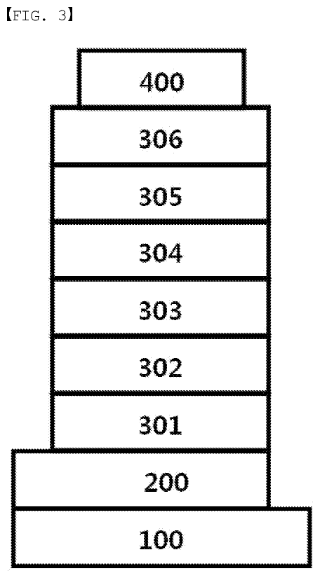

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

FIG. 3 illustrates a case of the organic material layer being a multilayer. The organic light emitting device according to FIG. 3 comprises a hole injection layer (301), a hole transport layer (302), a light emitting layer (303), a hole blocking layer (304), an electron transport layer (305) and an electron injection layer (306). However, the scope of the present application is not limited to such a lamination structure, and as necessary, layers other than the light emitting layer may not be included, and other necessary functional layers may be further added.

One embodiment of the present application provides a method for manufacturing an organic light emitting device, the method comprising preparing a substrate; forming a first electrode on the substrate; forming one or more organic material layers on the first electrode; and forming a second electrode on the organic material layer, wherein the forming of organic material layers comprises forming one or more organic material layers using the composition for an organic material layer according to one embodiment of the present application.

In the method for manufacturing an organic light emitting device provided in one embodiment of the present application, the forming of organic material layers is forming the heterocyclic compound of Chemical Formula 1 and the heterocyclic compound of Chemical Formula A using a thermal vacuum deposition method after pre-mixing.

The pre-mixing means first mixing the heterocyclic compound of Chemical Formula 1 and the heterocyclic compound of Chemical Formula A in one source of supply before depositing on the organic material layer.

The premixed material may be referred to as the composition for an organic material layer according to one embodiment of the present application.

The organic material layer comprising Chemical Formula 1 may further comprise other materials as necessary.

The organic material layer comprising both Chemical Formula 1 and Chemical Formula A may further comprise other materials as necessary.

In the organic light emitting device according to one embodiment of the present application, materials other than the compound of Chemical Formula 1 or Chemical Formula A are illustrated below, however, these are for illustrative purposes only and not for limiting the scope of the present application, and may be replaced by materials known in the art.

As the anode material, materials having relatively large work function may be used, and transparent conductive oxides, metals, conductive polymers or the like may be used. Specific examples of the anode material comprise metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the cathode material, materials having relatively small work function may be used, and metals, metal oxides, conductive polymers or the like may be used. Specific examples of the cathode material comprise metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p.677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrene-sulfonate) that are conductive polymers having solubility, and the like, may be used.

As the hole transport material, pyrazoline derivatives, arylamine-based derivatives, stilbene derivatives, triphenyldiamine derivatives and the like may be used, and low molecular or high molecular materials may also be used.

As the electron transport material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. Herein, two or more light emitting materials may be used by being deposited as individual sources of supply or by being premixed and deposited as one source of supply. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involving in light emission together may also be used.

When mixing light emitting material hosts, same series hosts may be mixed, or different series hosts may be mixed. For example, any two or more types of materials among n-type host materials or p-type host materials may be selected and used as a host material of a light emitting layer.

The organic light emitting device according to one embodiment of the present application may be a top-emission type, a bottom-emission type or a dual-emission type depending on the materials used.

The heterocyclic compound according to one embodiment of the present application may also be used in an organic electronic device comprising an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### <Preparation Example 1> Preparation of Compound 1(D)

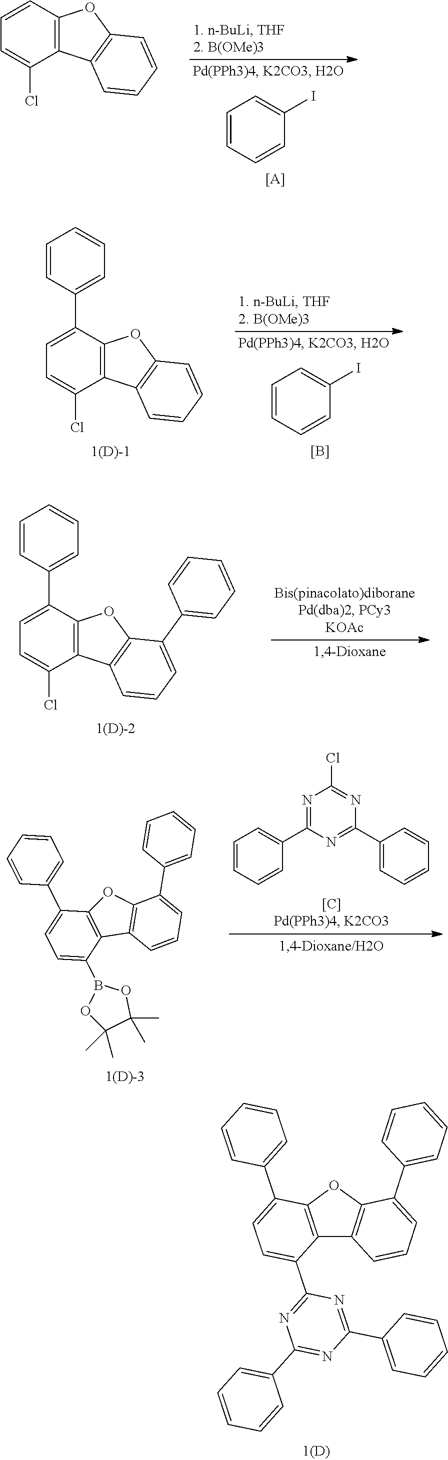

Preparation of Compound 1-1

To a one neck round bottom flask (one neck r.b.f), 1-chlorodibenzo[b,d]furan (15 g, 0.074 mol) and tetrahydrofuran (THF) (150 mL) were introduced, and the flask was nitrogen substituted. After lowering the temperature of the bath to −78° C., n-BuLi (6.16 g, 0.096 mol) was slowly added dropwise thereto.

When the reaction was finished, the temperature of the bath was raised to room temperature, and trimethyl borate (11.53 g, 0.111 mol) was introduced thereto.

When the reaction was finished, a mixture of iodobenzene (16.61 g, 0.081 mol), tetrakis (triphenylphosphine)palladium(0) (4.28 g, 0.004 mol), potassium carbonate (20.46 g, 0.148 mol) and 1,4-dioxane/water (150 mL/30 mL) was refluxed at 120° C.

The result was extracted with dichloromethane, dried with MgSO₄, silica gel filtered, and then concentrated to obtain Compound 1-1 (9.56 g, 46.34%).

Preparation of Compound 1-2

To a one neck round bottom flask (one neck r.b.f), 1-chloro-4-phenyldibenzo[b,d]furan (9.56 g, 0.034 mol) and tetrahydrofuran (THF) (95 mL) were introduced, and the flask was nitrogen substituted. After lowering the temperature of the bath to −78° C., n-BuLi (2.83 g, 0.044 mol) was slowly added dropwise thereto.

When the reaction was finished, the temperature of the bath was raised to room temperature, and trimethyl borate (5.30 g, 0.051 mol) was introduced thereto.

When the reaction was finished, a mixture of iodobenzene (7.63 g, 0.037 mol), tetrakis (triphenylphosphine)palladium(0) (1.96 g, 0.002 mol), potassium carbonate (9.40 g, 0.068 mol) and 1,4-dioxane/water (95 mL/19 mL) was refluxed at 120° C.

The result was extracted with dichloromethane, dried with MgSO₄, silica gel filtered, and then concentrated to obtain Compound 1-2 (10.50 g, 87%).

Preparation of Compound 1-3

In a one neck round bottom flask (one neck r.b.f), a mixture of 1-chloro-4,6-diphenyldibenzo[b,d]furan (10.50 g, 0.030 mol), bis(pinacolato)diboron (15.24 g, 0.060 mol), Pd₂(dba)₃ (2.75 g, 0.003 mol), PCy₃ (1.68 g, 0.006 mol), potassium acetate (8.83 g, 0.090 mol) and 1,4-dioxane (100 mL) was refluxed at 140° C.

The result was extracted with dichloromethane, concentrated, and then silica gel filtered. The result was concentrated, and then treated with dichloromethane/methanol to obtain Compound 1-3 (9.91 g, 74%).

Preparation of Compound 1(D)

In a one neck round bottom flask (one neck r.b.f), a mixture of 2-(4,6-diphenyldibenzo[b,d]furan-1-yl)-4,4,5,5-tetramethyl-1,3,2-dioxaborolane (9.91 g, 0.022 mol), 2-chloro-4,6-diphenyl-1,3,5-triazine (6.48 g, 0.024 mol), tetrakis (triphenylphosphine)palladium(0) (1.27 g, 0.001 mol), potassium carbonate (6.08 g, 0.044 mol) and 1,4-dioxane/water (90 mL/27 mL) was refluxed at 120° C.

The result was extracted with dichloromethane, then dried with MgSO₄, and column purified to obtain Compound 1(D) (8.13 g, 67%).

Each of target Compound D was synthesized in the same manner as in Preparation Example 1 except that Intermediates A, B and C of the following Table 1 were used.

TABLE 1

| Com- | | | | | |
|---|---|---|---|---|---|
| pound | A | B | C | D | Yield |
| 1 | 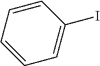 | 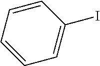 | 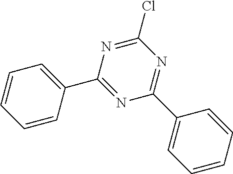 | 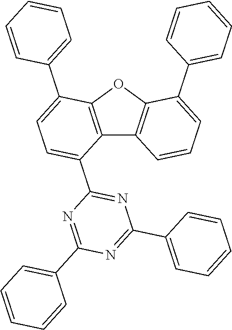 | 67% |
| 2 | 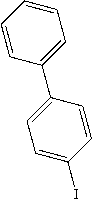 | 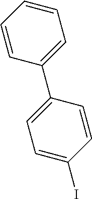 | 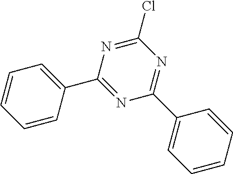 | 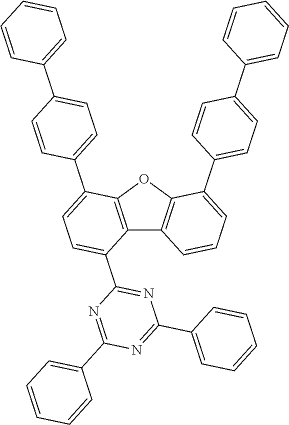 | 71% |
| 3 | 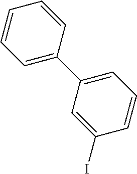 | 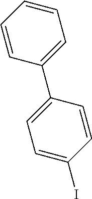 | 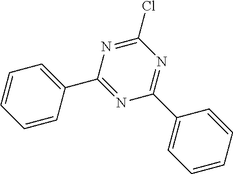 | 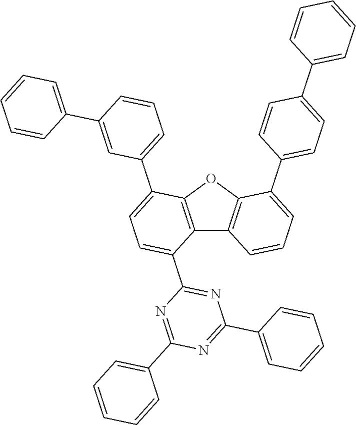 | 53% |
| 4 | 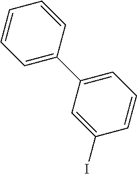 | 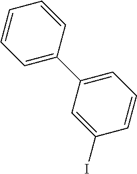 | 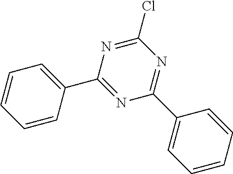 | 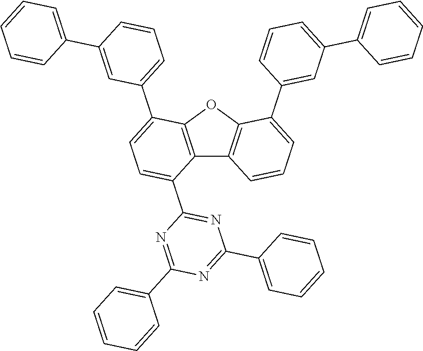 | 81% |
| 6 | 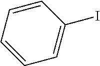 | 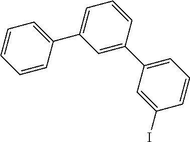 | 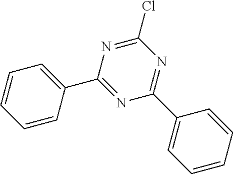 | 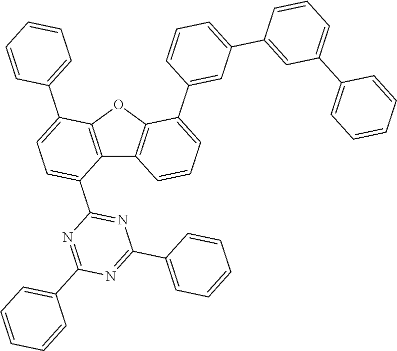 | 83% |
| 9 | 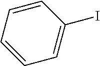 | 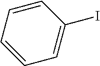 | 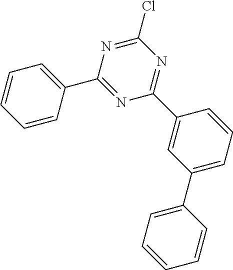 | 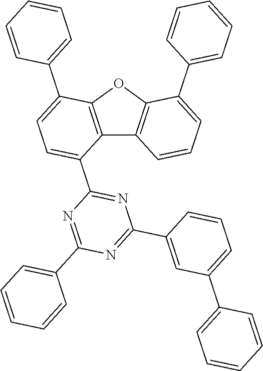 | 84% |
| 11 | 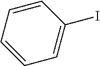 | 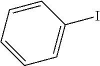 | 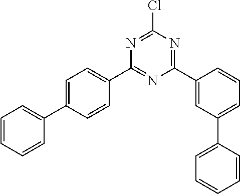 | 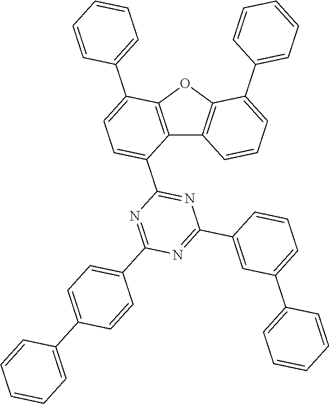 | 60% |
| 13 | 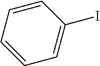 | 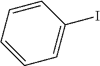 | 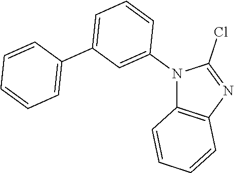 | 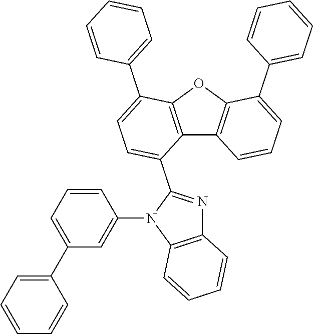 | 77% |
| 16 | 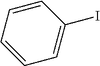 | 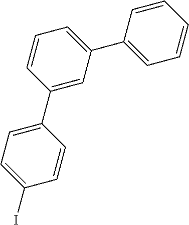 | 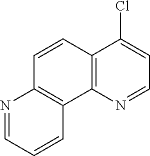 | 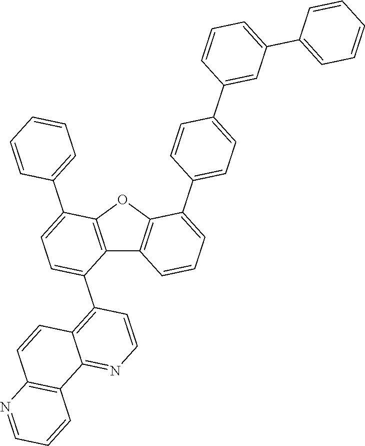 | 65% |
| 20 | 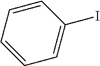 | 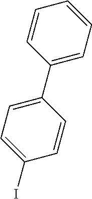 | 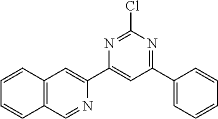 | 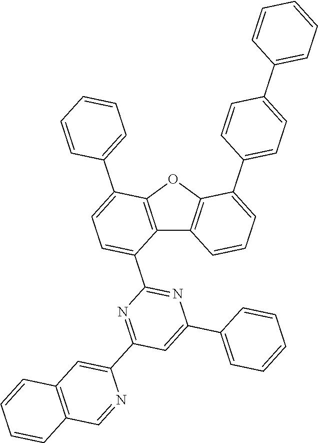 | 79% |
| 21 | 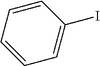 | 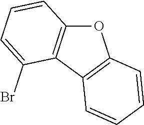 | 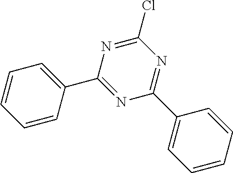 | 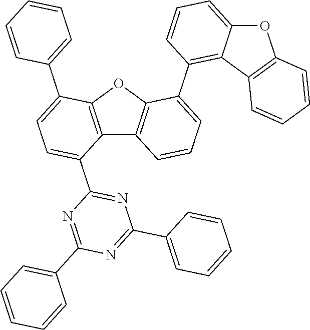 | 84% |
| 23 | 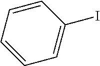 | 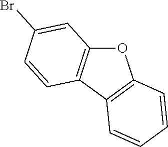 | 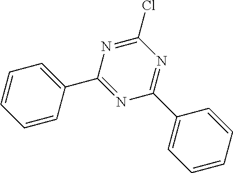 | 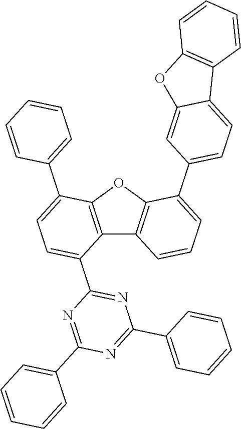 | 76% |
| 24 | 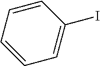 | 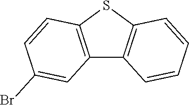 | 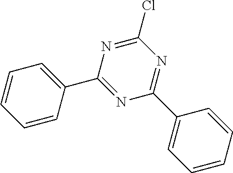 | 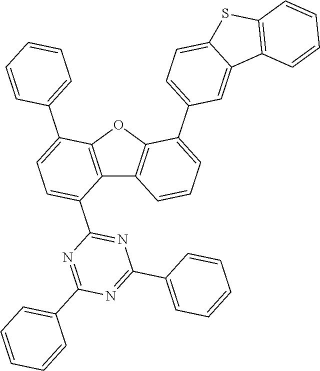 | 61% |
| 26 | 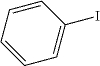 | 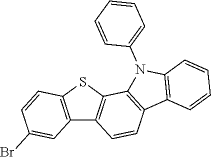 | 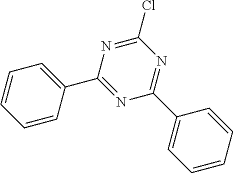 | 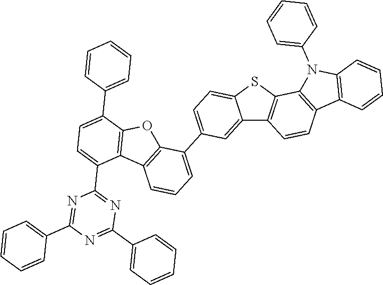 | 59% |
| 28 | 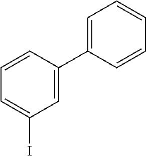 | 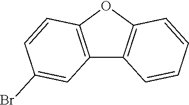 | 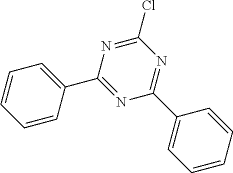 | 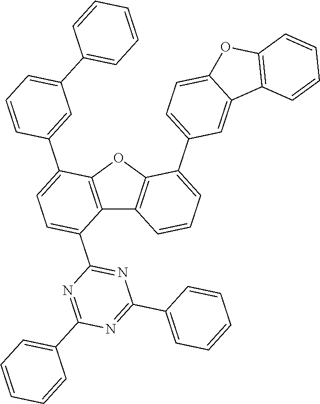 | 92% |
| 30 | 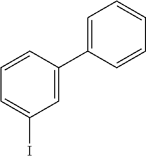 | 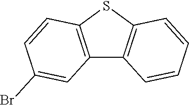 | 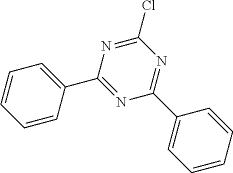 | 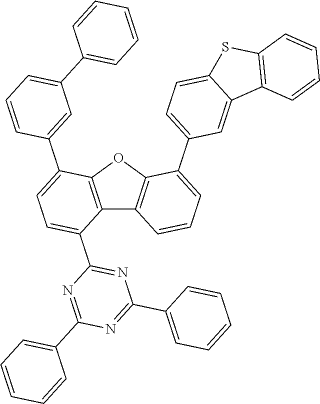 | 68% |
| 37 | 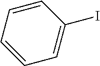 | 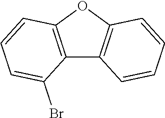 | 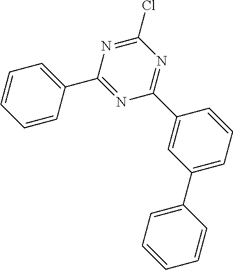 | 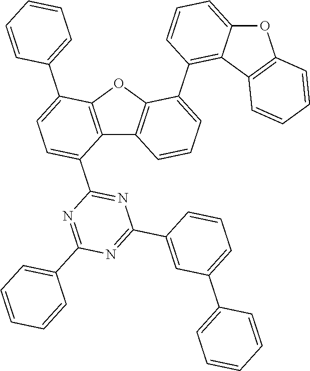 | 96% |
| 39 | 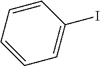 | 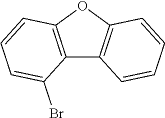 | 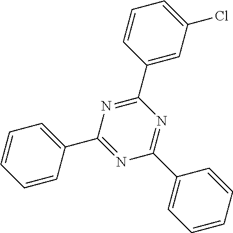 | 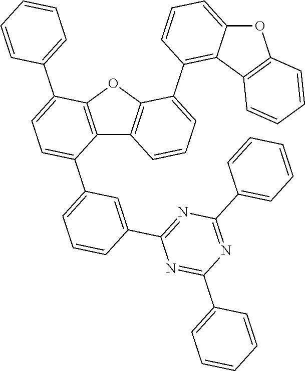 | 49% |

### [Preparation Example 2] Preparation of Compound 41(E)

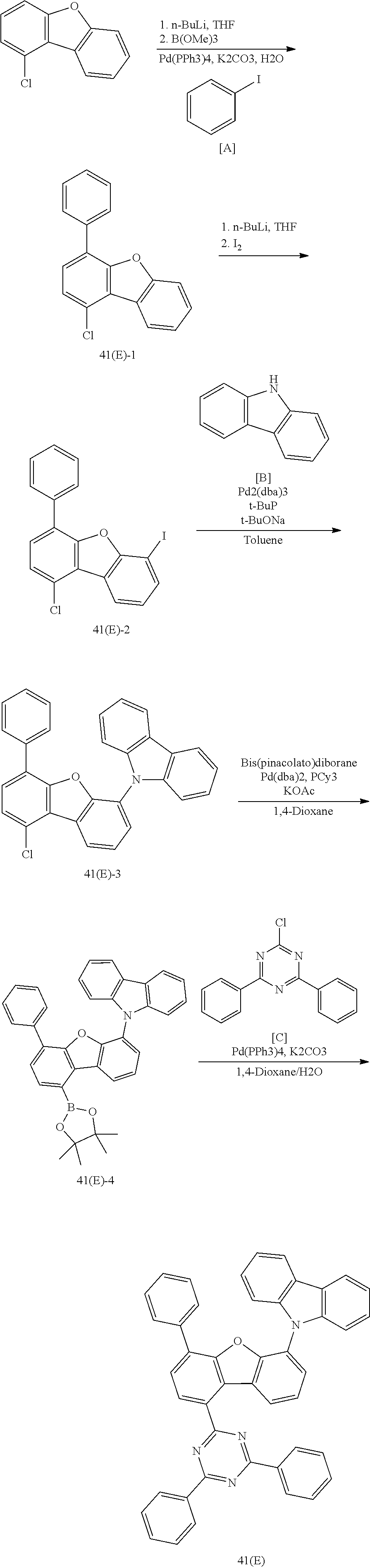

Preparation of Compound 41-1

To a one neck round bottom flask (one neck r.b.f), 1-chlorodibenzo[b,d]furan (15 g, 0.074 mol) and THF (150 mL) were introduced, and the flask was nitrogen substituted. After lowering the temperature of the bath to −78° C., n-BuLi (6.16 g, 0.096 mol) was slowly added dropwise thereto.

When the reaction was finished, the temperature of the bath was raised to room temperature, and trimethyl borate (11.53 g, 0.111 mol) was introduced thereto.

When the reaction was finished, a mixture of iodobenzene (16.61 g, 0.081 mol), tetrakis (triphenylphosphine)palladium(0) (4.28 g, 0.004 mol), potassium carbonate (20.46 g, 0.148 mol) and 1,4-dioxane/water (150 mL/30 mL) was refluxed at 120° C.

The result was extracted with dichloromethane, dried with MgSO₄, silica gel filtered, and then concentrated to obtain Compound 41-1 (9.56 g, 46.34%).

Preparation of Compound 41-2

To a one neck round bottom flask (one neck r.b.f), 1-chloro-4-phenyldibenzo[b,d]furan (9.56 g, 0.034 mol) and THF (95 mL) were introduced, and the flask was nitrogen substituted. After lowering the temperature of the bath to −78° C., n-BuLi (2.83 g, 0.044 mol) was slowly added dropwise thereto.

When the reaction was finished, the temperature of the bath was raised to room temperature, and iodine (12.94 g, 0.051 mol) was introduced thereto.

When the reaction was completed, distilled water was introduced thereto. The result was extracted with dichloromethane, dried with MgSO₄, silica gel filtered, and then concentrated to obtain Compound 41-2 (10.18 g, 74%).

Preparation of Compound 41-3

In a one neck round bottom flask (one neck r.b.f), a mixture of 1-chloro-6-iodo-4-phenyldibenzo[b,d]furan (10.18 g, 0.025 mol), carbazole (4.60 g, 0.028 mol), tris(dibenzylideneacetone)dipalladium(0) (2.29 g, 0.0025 mol), tri-tert-butylphosphine (10 g, 0.049 mol), sodium-tert-butoxide (4.81 g, 0.05 mol) and toluene (100 mL) was refluxed at 130° C.

When the reaction was completed, the result was filtered to remove the solids, and the filtrate was silica gel filtered and then concentrated to obtain Compound 41-3 (9.77 g, 88%).

Preparation of Compound 41-4

In a one neck round bottom flask (one neck r.b.f), a mixture of 9-(9-chloro-6-phenyldibenzo[b,d]furan-4-yl)-9H-carbazole (9.77 g, 0.022 mol), bis(pinacolato)diboron (11.17 g, 0.044 mol), Pd₂(dba)₃ (1.01 g, 0.001 mol), PCy₃ (1.23 g, 0.004 mol), potassium acetate (6.84 g, 0.066 mol) and 1,4-dioxane (90 mL) was refluxed at 140° C.

The result was extracted with dichloromethane, concentrated, and then silica gel filtered. The result was concentrated, and then treated with dichloromethane/methanol to obtain Compound 41-4 (8.36 g, 71%).

Preparation of Compound 41(E)

In a one neck round bottom flask (one neck r.b.f), a mixture of 9-(6-phenyl-9-(4,4,5,5-tetramethyl-1,3,2-dioxaborolan-2-yl)dibenzo[b,d]furan-4-yl)-9H-carbazole (8.36 g, 0.016 mol), 2-chloro-4,6-diphenyl-1,3,5-triazine (4.71 g, 0.018 mol), tetrakis(triphenylphosphine)palladium(0) (0.92 g, 0.001 mol), potassium carbonate (4.42 g, 0.032 mol) and 1,4-dioxane/water (80 mL/24 mL) was refluxed at 120° C.

The result was extracted with dichloromethane, dried with MgSO₄, silica gel filtered, and then concentrated to obtain Compound 41(E) (6.36 g, 62%).

Each of target Compound E was synthesized in the same manner as in Preparation Example 2 except that Intermediates A, B and C of the following Table 2 were used.

TABLE 2

| Com- | | | | | |
|---|---|---|---|---|---|
| pound | A | B | C | E | Yield |
| 41 | 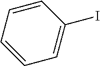 | 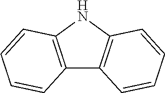 | 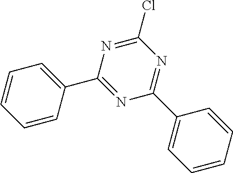 | 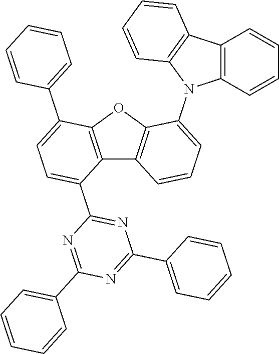 | 62% |
| 42 | 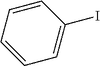 | 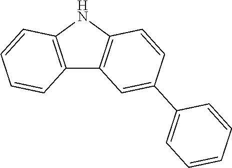 | 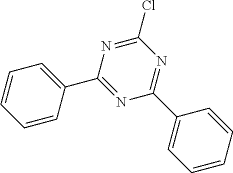 | 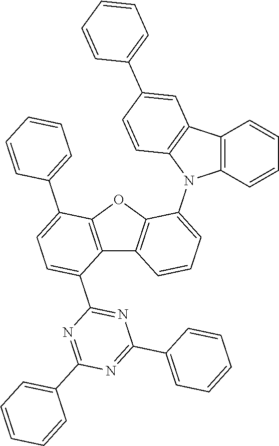 | 75% |
| 43 | 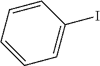 | 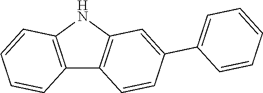 | 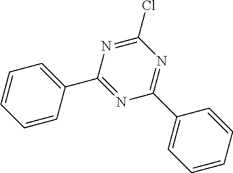 | 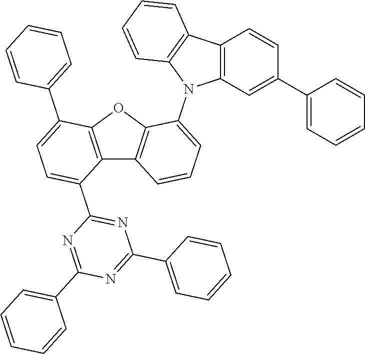 | 74% |
| 45 | 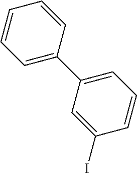 | 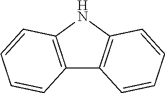 | 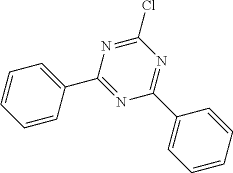 | 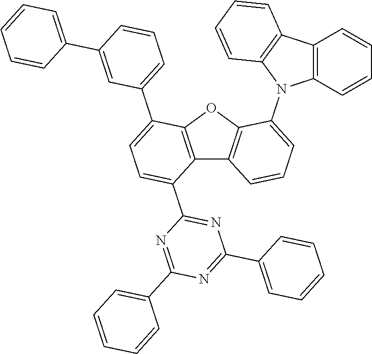 | 65% |
| 48 | 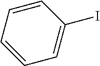 | 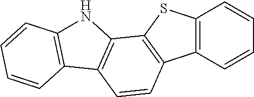 | 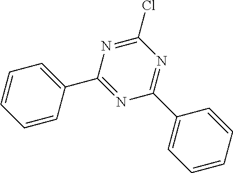 | 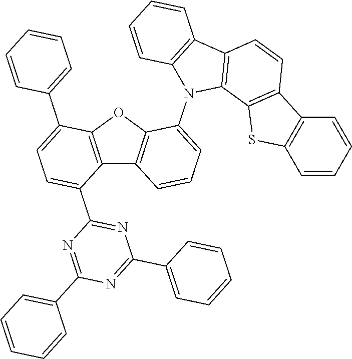 | 53% |
| 49 | 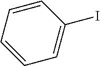 | 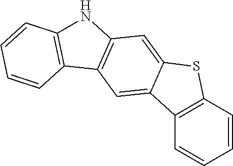 | 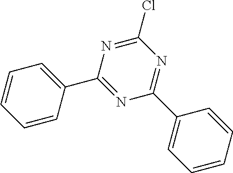 | 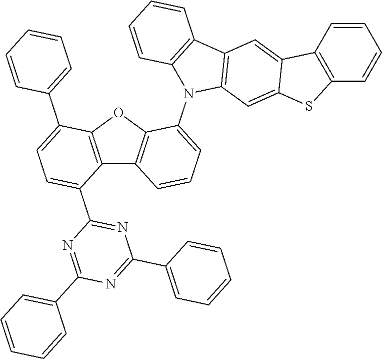 | 90% |
| 51 | 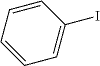 | 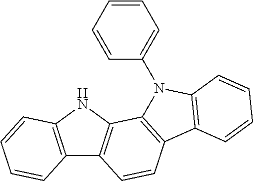 | 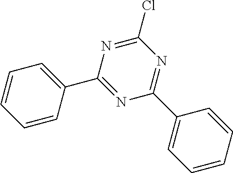 | 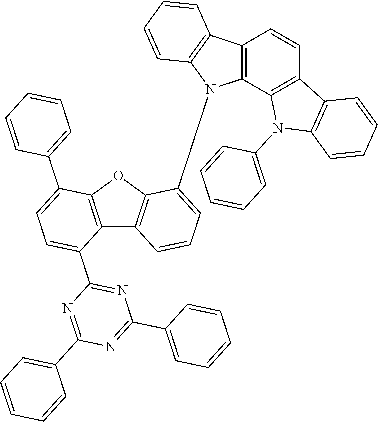 | 57% |
| 54 | 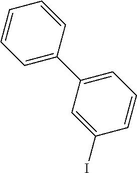 | 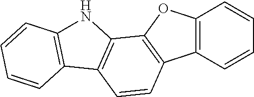 | 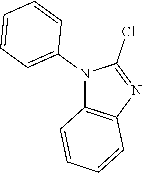 | 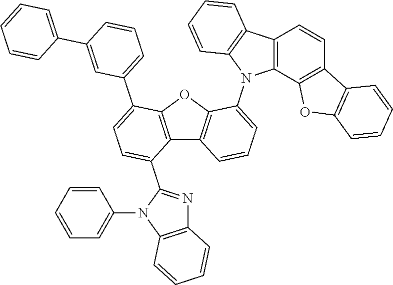 | 65% |
| 59 | 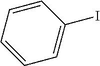 | 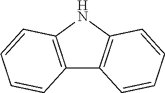 | 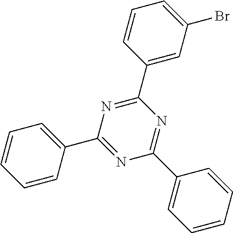 | 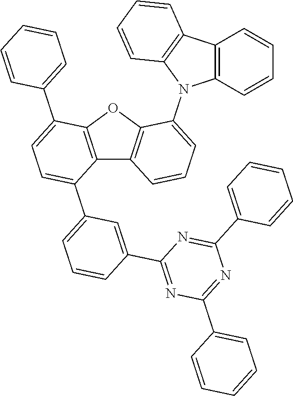 | 69% |
| 61 | 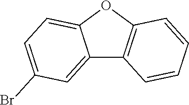 | 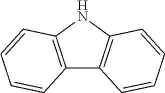 | 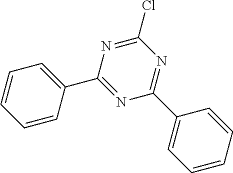 | 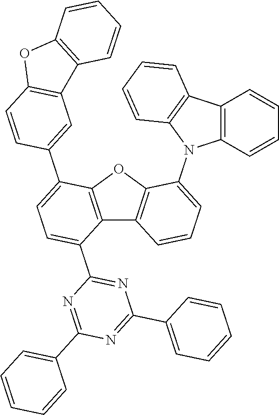 | 79% |
| 62 | 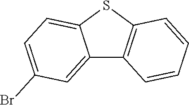 | 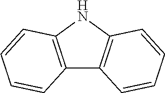 | 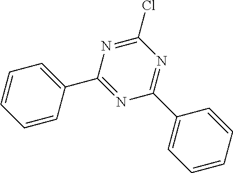 | 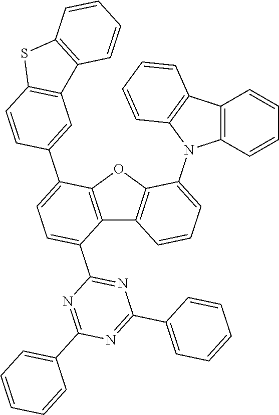 | 80% |
| 63 | 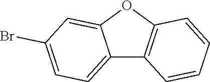 | 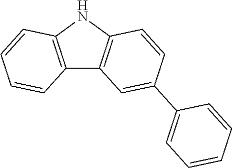 | 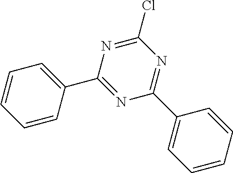 | 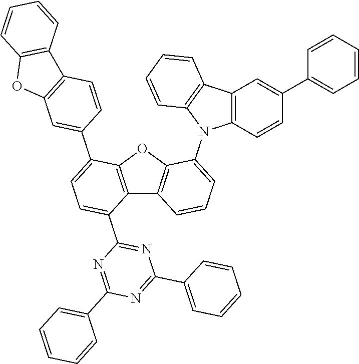 | 71% |
| 65 | 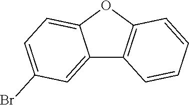 | 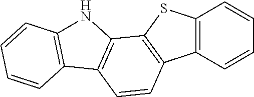 | 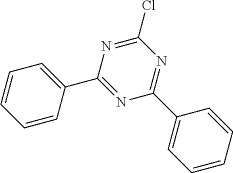 | 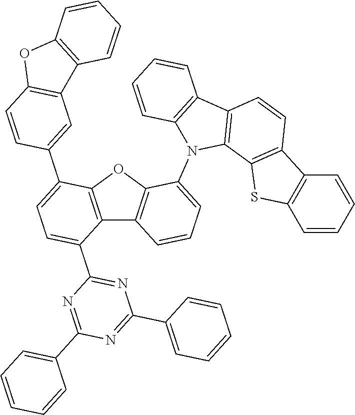 | 47% |
| 67 | 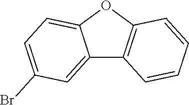 | 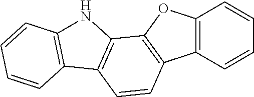 | 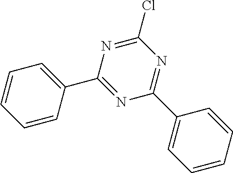 | 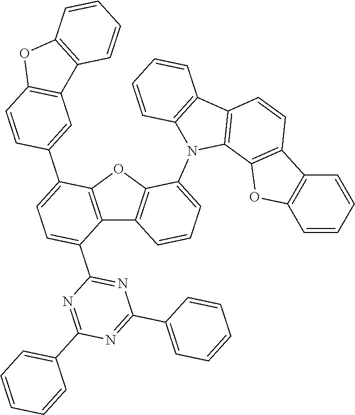 | 49% |
| 68 | 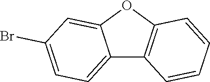 | 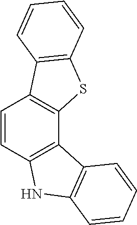 | 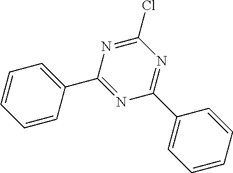 | 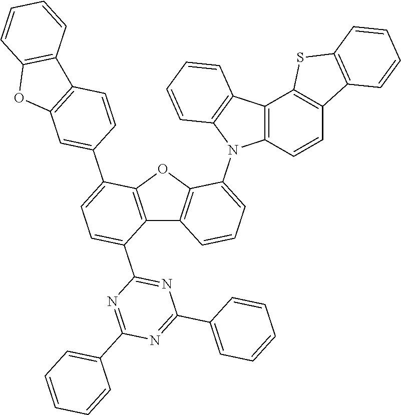 | 53% |
| 70 | 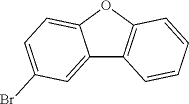 | 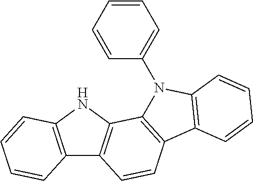 | 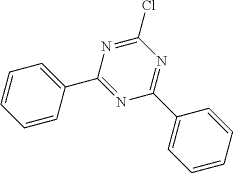 | 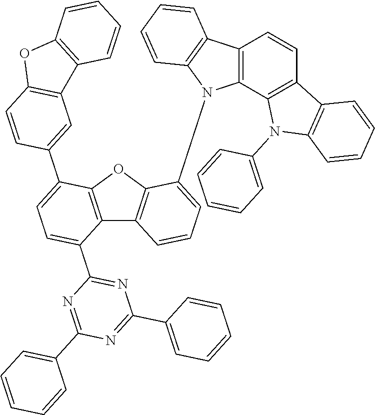 | 39% |
| 71 | 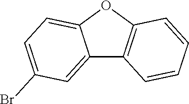 | 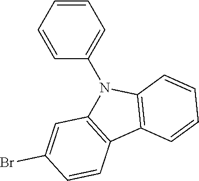 | 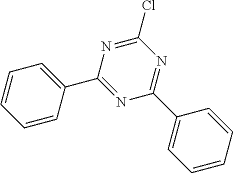 | 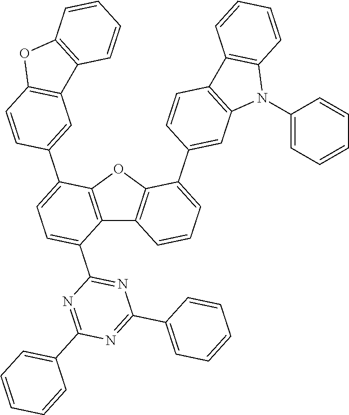 | 85% |
| 74 | 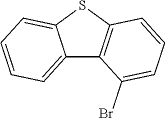 | 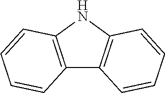 | 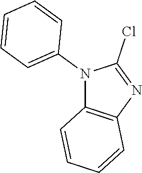 | 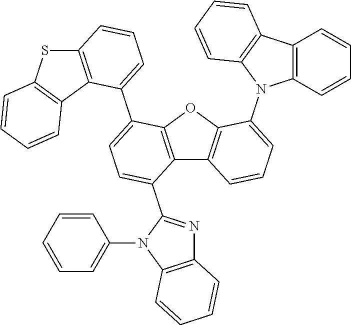 | 77% |
| 78 | 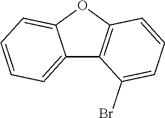 | 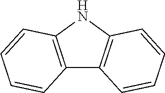 | 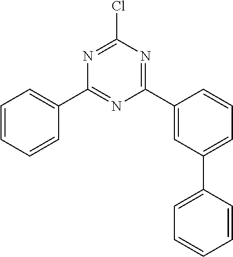 | 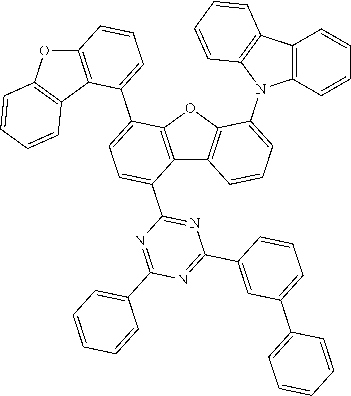 | 60% |
| 80 | 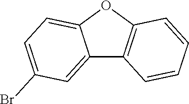 | 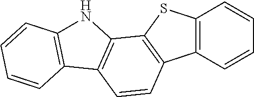 | 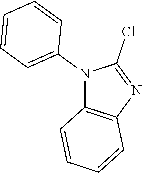 | 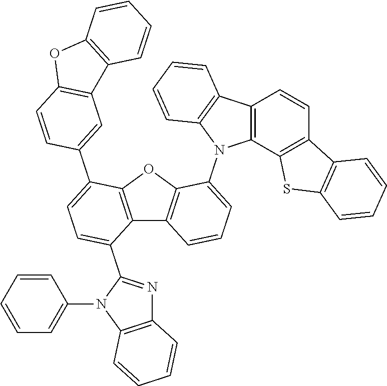 | 42% |
| 227 | 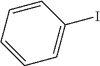 | 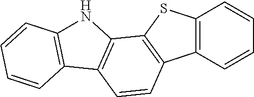 | 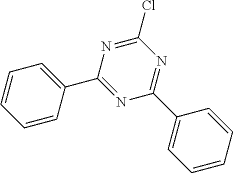 | 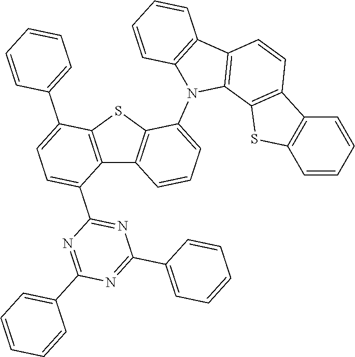 | 53% |
| 231 | 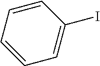 | 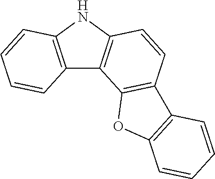 | 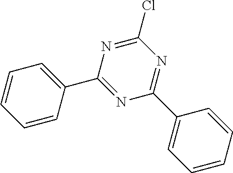 | 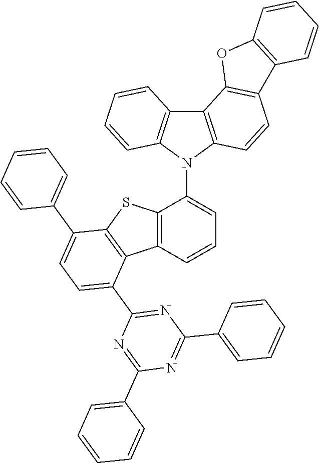 | 67% |

### [Preparation Example 3] Preparation of Compound 81 (F)

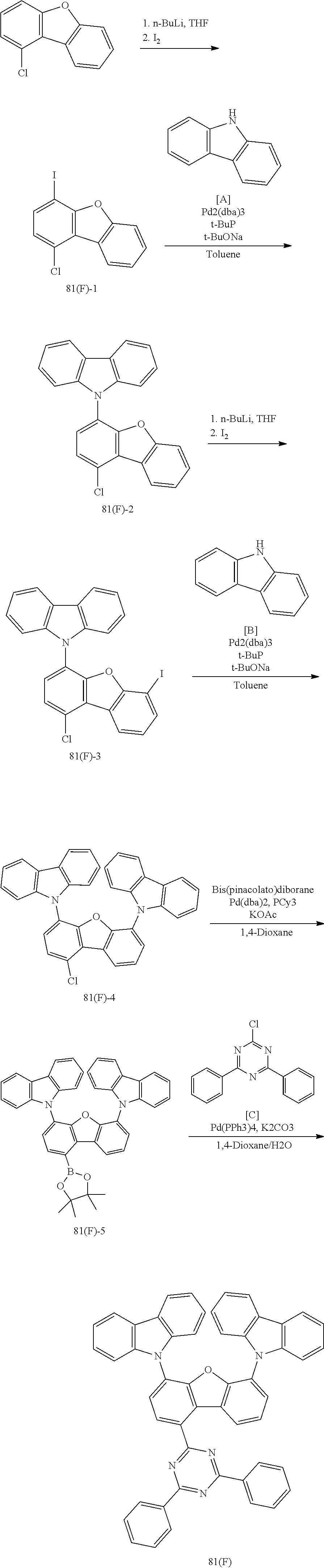

Preparation of Compound 81-1

To a one neck round bottom flask (one neck r.b.f), 1-chlorodibenzo[b,d]furan (15 g, 0.074 mol) and tetrahydrofuran (THF) (150 mL) were introduced, and the flask was nitrogen substituted. After lowering the temperature of the bath to −78° C., n-BuLi (6.16 g, 0.096 mol) was slowly added dropwise thereto.

When the reaction was finished, the temperature of the bath was raised to room temperature, and iodine (28.17 g, 0.11 mol) was introduced thereto.

When the reaction was completed, distilled water was introduced thereto. The result was extracted with dichloromethane, dried with MgSO₄, silica gel filtered, and then concentrated to obtain Compound 81-1 (17.26 g, 71%).

Preparation of Compound 81-2

In a one neck round bottom flask (one neck r.b.f), a mixture of 1-chloro-4-iododibenzo[b,d]furan (17.26 g, 0.053 mol), carbazole (9.75 g, 0.058 mol), tris(dibenzylideneacetone)dipalladium(0) (3.06 g, 0.0027 mol), tri-tert-butylphosphine (17 g, 0.084 mol), sodium-tert-butoxide (10.19 g, 0.12 mol) and toluene (170 mL) was refluxed at 130° C.

When the reaction was completed, the result was filtered to remove the solids, and the filtrate was silica gel filtered and then concentrated to obtain Compound 81-2 (12.48 g, 64%).

Preparation of Compound 81-3

To a one neck round bottom flask (one neck r.b.f), 9-(1-chlorodibenzo[b,d]furan-4-yl)-9H-carbazole (12.48 g, 0.034 mol) and THF (120 mL) were introduced, and the flask was nitrogen substituted. After lowering the temperature of the bath to −78° C., n-BuLi (2.83 g, 0.044 mol) was slowly added dropwise thereto.

When the reaction was finished, the temperature of the bath was raised to room temperature, and iodine (12.94 g, 0.051 mol) was introduced thereto.

When the reaction was completed, distilled water was introduced thereto. The result was extracted with dichloromethane, dried with MgSO₄, silica gel filtered, and then concentrated to obtain Compound 81-3 (11.58 g, 69%).

Preparation of Compound 81-4

In a one neck round bottom flask (one neck r.b.f), a mixture of 9-(1-chloro-6-iododobenzo[b,d]furan-4-yl)-9H-carbazole (11.58 g, 0.023 mol), carbazole (4.23 g, 0.025 mol), tris(dibenzylideneacetone)dipalladium(0) (1.33 g, 0.0012 mol), tri-tert-butylphosphine (11 g, 0.054 mol), sodium-tert-butoxide (4.42 g, 0.046 mol) and toluene (110 mL) was refluxed at 130° C.

When the reaction was completed, the result was filtered to remove the solids, and the filtrate was silica gel filtered and then concentrated to obtain Compound 81-4 (7.72 g, 63%).

Preparation of Compound 81-5

In a one neck round bottom flask (one neck r.b.f), a mixture of 9,9′-(1-chlorodibenzo[b,d]furan-4,6-diyl)bis(9H-carbazole) (7.72 g, 0.014 mol), bis(pinacolato)diboron (7.11 g, 0.028 mol), Pd₂(dba)₃ (0.64 g, 0.0007 mol), PCy₃ (0.79 g, 0.0028 mol), potassium acetate (4.12 g, 0.042 mol) and 1,4-dioxane (70 mL) was refluxed at 140° C.

The result was extracted with dichloromethane, concentrated, and then silica gel filtered. The result was concentrated, and then treated with dichloromethane/methanol to obtain Compound 81-5 (7.69 g, 88%).

Preparation of Compound 81 (F)

In a one neck round bottom flask (one neck r.b.f), a mixture of 9,9′-(1-(4,4,5,5-tetramethyl-1,3,2-dioxaborolan-2-yl)dibenzo[b,d]furan-4,6-diyl)bis(9H-carbazole) (7.69 g, 0.012 mol), 2-chloro-4,6-diphenyl-1,3,5-triazine (3.53 g, 0.013 mol), tetrakis (triphenylphosphine)palladium(0) (0.69 g, 0.0006 mol), potassium acetate and 1,4-dioxane/water (75 mL/23 mL) was refluxed at 120° C.

The result was extracted with dichloromethane, dried with MgSO₄, silica gel filtered, and then concentrated to obtain Compound 81(F) (6.48 g, 74%).

Each of target Compound F was synthesized in the same manner as in Preparation Example 3 except that Intermediates A, B and C of the following Table 3 were used.

TABLE 3

| Com- | | | | | |
|---|---|---|---|---|---|
| pound | A | B | C | F | Yield |
| 81 | 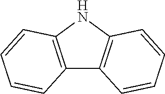 | 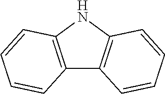 | 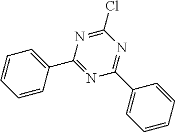 | 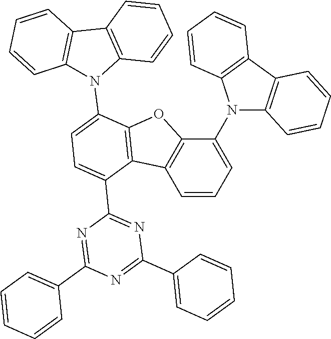 | 74% |
| 82 | 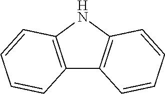 | 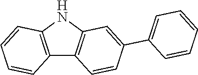 | 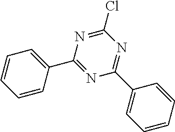 | 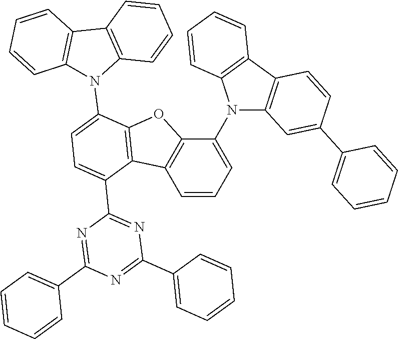 | 66% |
| 85 | 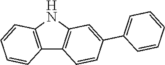 | 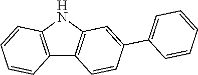 | 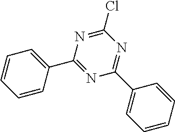 | 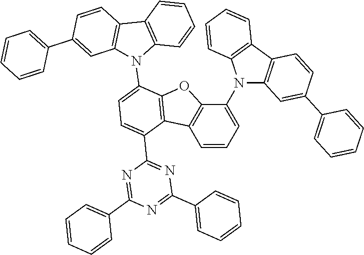 | 42% |
| 86 | 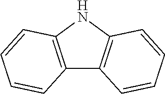 | 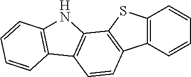 | 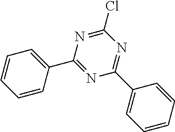 | 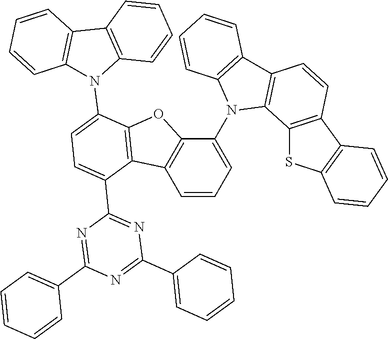 | 41% |
| 88 | 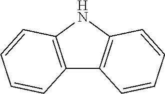 | 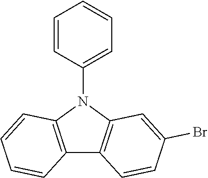 | 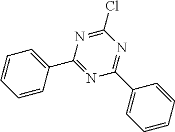 | 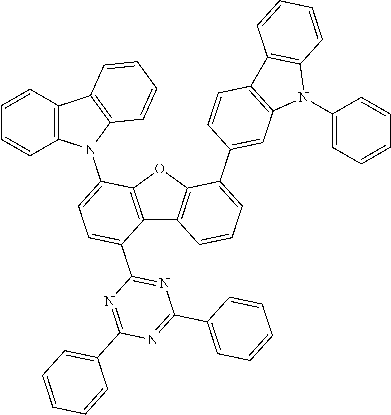 | 89% |
| 91 | 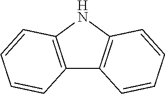 | 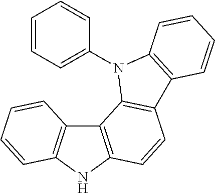 | 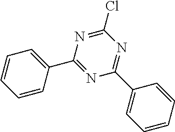 | 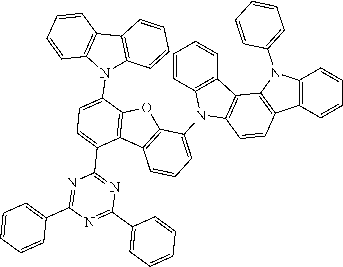 | 56% |
| 92 | 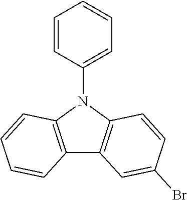 | 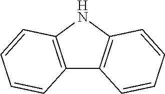 | 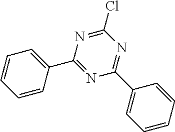 | 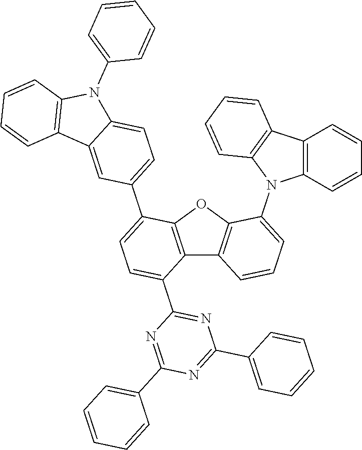 | 75% |
| 93 | 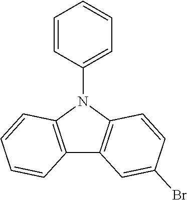 | 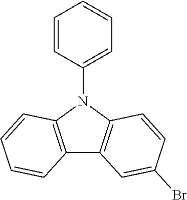 | 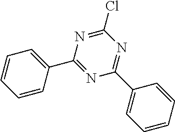 | 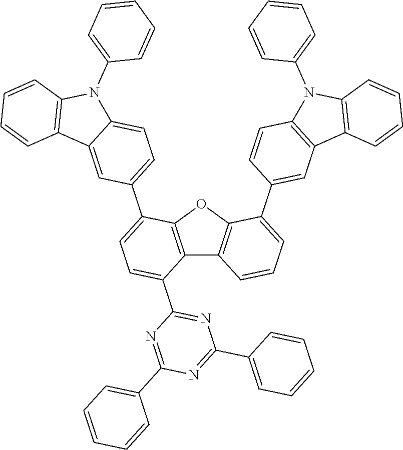 | 65% |
| 96 | 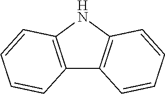 | 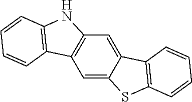 | 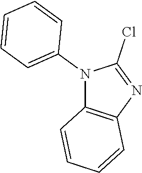 | 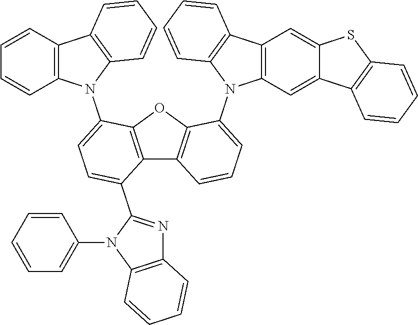 | 63% |
| 98 | 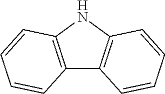 | 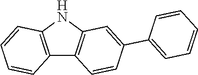 | 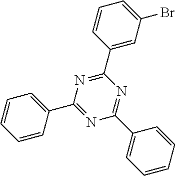 | 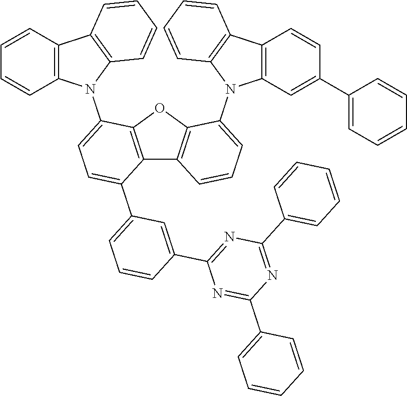 | 74% |
| 100 | 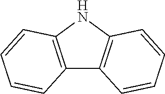 | 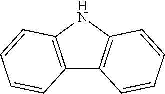 | 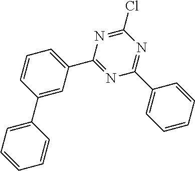 | 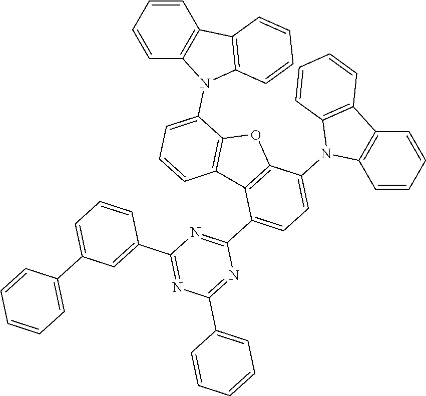 | 91% |

### [Preparation Example 4] Preparation of Compound 101 (G)

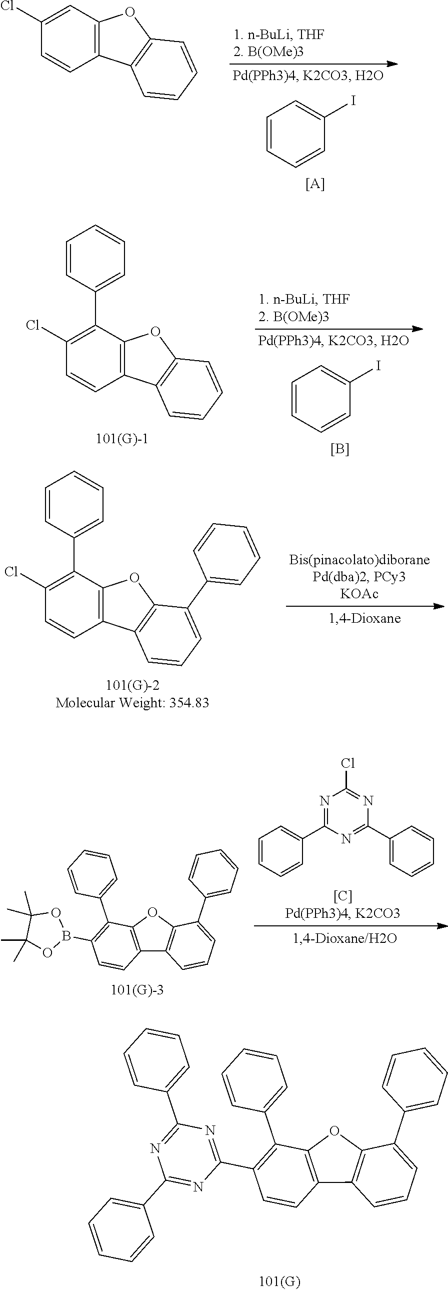

Preparation of Compound 101-1

To a one neck round bottom flask (one neck r.b.f), 3-chlorodibenzo[b,d]furan (15 g, 0.074 mol) and tetrahydrofuran (THF) (150 mL) were introduced, and the flask was nitrogen substituted. After lowering the temperature of the bath to −78° C., n-BuLi (6.16 g, 0.096 mol) was slowly added dropwise thereto.

When the reaction was finished, the temperature of the bath was raised to room temperature, and trimethyl borate (11.53 g, 0.111 mol) was introduced thereto.

When the reaction was finished, a mixture of iodobenzene (16.61 g, 0.081 mol), tetrakis (triphenylphosphine)palladium(0) (4.28 g, 0.004 mol), potassium carbonate (20.46 g, 0.148 mol) and 1,4-dioxane/water (150 mL/30 mL) was refluxed at 120° C.

The result was extracted with dichloromethane, dried with MgSO₄, silica gel filtered, and then concentrated to obtain Compound 101-1 (8.68 g, 42.10%).

Preparation of Compound 101-2

To a one neck round bottom flask (one neck r.b.f), 3-chloro-4-phenyldibenzo[b,d]furan (8.68 g, 0.031 mol) and THF (85 mL) were introduced, and the flask was nitrogen substituted. After lowering the temperature of the bath to −78° C., n-BuLi (2.58 g, 0.040 mol) was slowly added dropwise thereto.

When the reaction was finished, the temperature of the bath was raised to room temperature, and trimethyl borate (4.83 g, 0.047 mol) was introduced thereto.

When the reaction was finished, a mixture of iodobenzene (6.96 g, 0.034 mol), tetrakis (triphenylphosphine)palladium(0) (1.79 g, 0.0016 mol), potassium carbonate (8.57 g, 0.062 mol) and 1,4-dioxane/water (42 mL/8 mL) was refluxed at 120° C.

The result was extracted with dichloromethane, dried with MgSO₄, silica gel filtered, and then concentrated to obtain Compound 101-2 (5.83 g, 53%).

Preparation of Compound 101-3

In a one neck round bottom flask (one neck r.b.f), a mixture of 3-chloro-4,6-diphenyldibenzo[b,d]furan (5.83 g, 0.016 mol), bis(pinacolato)diboron (8.13 g, 0.032 mol), Pd₂(dba)₃ (0.73 g, 0.0008 mol), PCy₃ (0.90 g, 0.0032 mol), potassium acetate (3.14 g, 0.032 mol) and 1,4-dioxane (50 mL) was refluxed at 140° C.

The result was extracted with dichloromethane, concentrated, and then silica gel filtered. The result was concentrated, and then treated with dichloromethane/methanol to obtain Compound 101-3 (9.41 g, 68%).

Preparation of Compound 101 (G)

In a one neck round bottom flask (one neck r.b.f), a mixture of 2-(4,6-diphenyldibenzo[b,d]furan-3-yl)-4,4,5,5-tetramethyl-1,3,2-dioxaborolane (9.41 g, 0.020 mol), 2-chloro-4,6-diphenyl-1,3,5-triazine (5.89 g, 0.022 mol), tetrakis (triphenylphosphine)palladium(0) (1.16 g, 0.001 mol), potassium carbonate (5.53 g, 0.040 mol) and 1,4-dioxane/water (90 mL/27 mL) was refluxed at 120° C.

The result was extracted with dichloromethane, dried with MgSO₄, and column purified to obtain Compound 101(G) (8.50 g, 77%).

Each of target Compound G was synthesized in the same manner as in Preparation Example 4 except that Intermediates A, B and C of the following Table 4 were used.

TABLE 4

| Com- | | | | | |
|---|---|---|---|---|---|
| pound | A | B | C | G | Yield |
| 101 | 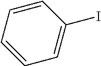 | 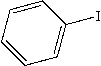 | 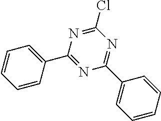 | 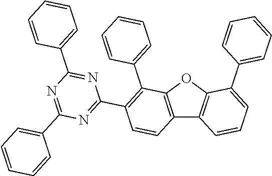 | 77% |
| 102 | 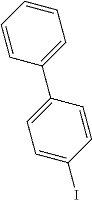 | 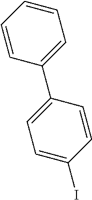 | 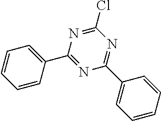 | 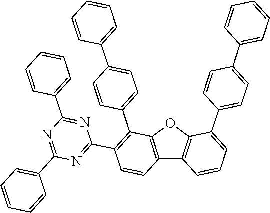 | 65% |
| 103 | 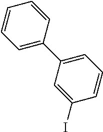 | 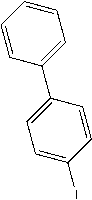 | 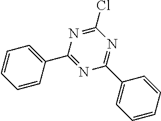 | 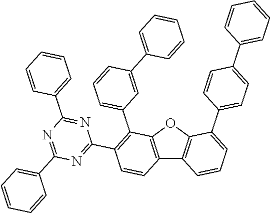 | 53% |
| 104 | 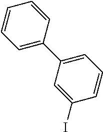 | 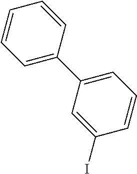 | 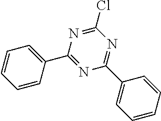 | 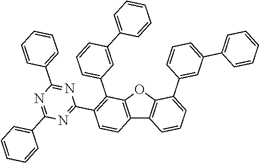 | 41% |
| 107 | 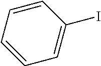 | 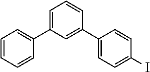 | 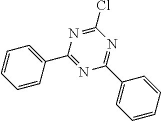 | 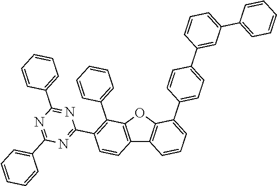 | 82% |
| 109 | 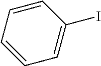 | 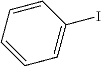 | 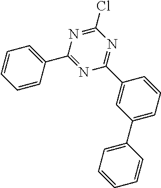 | 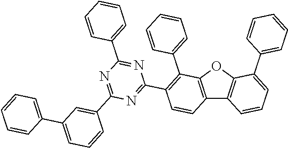 | 73% |
| 111 | 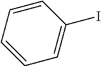 | 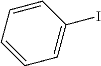 | 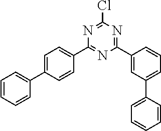 | 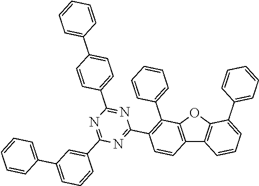 | 63% |
| 113 | 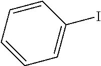 | 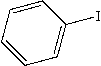 | 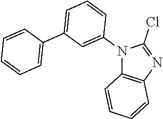 | 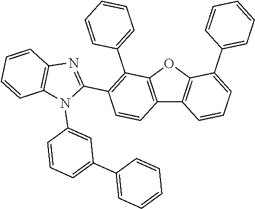 | 71% |
| 118 | 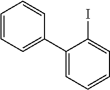 | 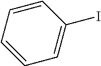 | 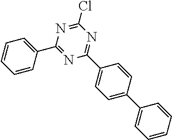 | 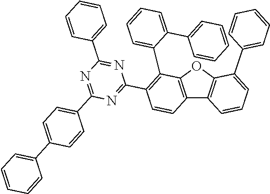 | 65% |
| 119 | 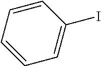 | 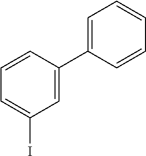 | 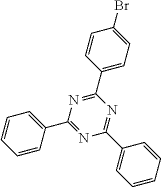 | 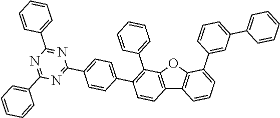 | 78% |
| 121 | 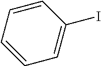 | 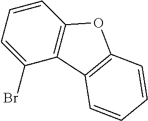 | 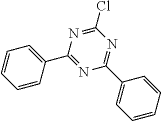 | 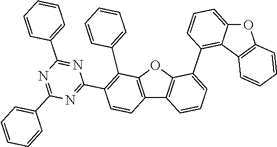 | 63% |
| 122 | 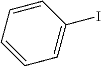 | 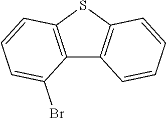 | 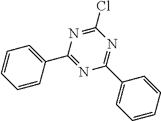 | 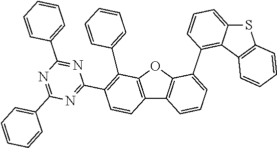 | 71% |
| 123 | 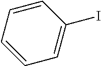 | 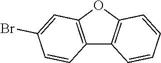 | 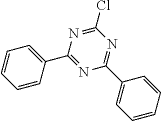 | 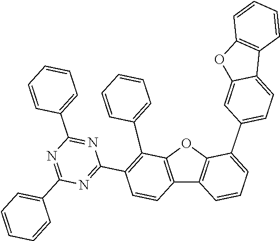 | 59% |
| 124 | 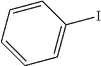 | 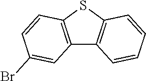 | 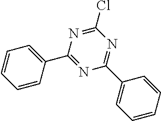 | 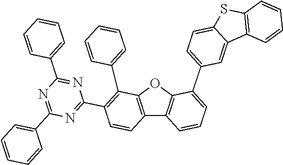 | 66% |
| 125 | 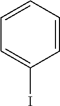 | 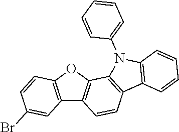 | 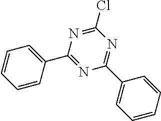 | 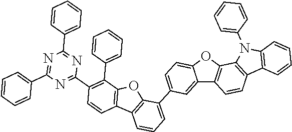 | 91% |
| 126 | 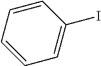 | 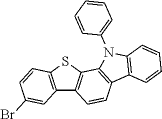 | 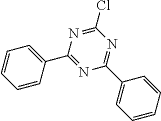 | 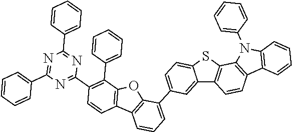 | 54% |
| 130 | 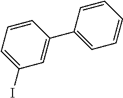 | 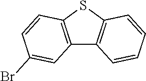 | 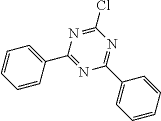 | 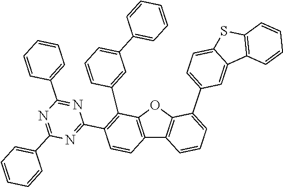 | 69% |
| 136 | 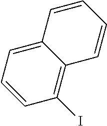 | 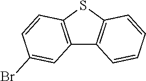 | 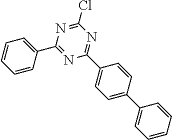 | 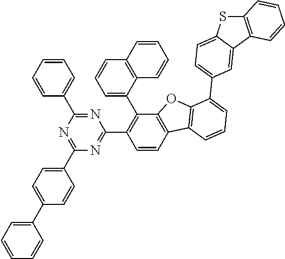 | 72% |
| 137 | 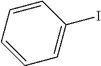 | 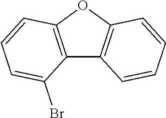 | 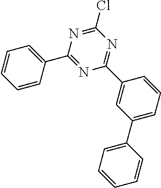 | 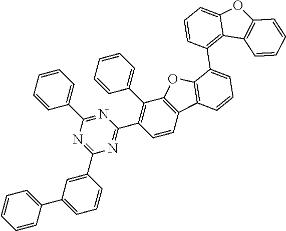 | 58% |
| 139 | 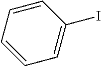 | 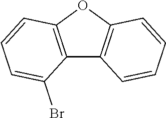 | 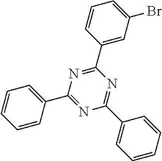 | 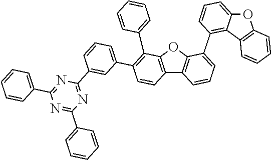 | 84% |

### [Preparation Example 5] Preparation of Compound 141 (H)

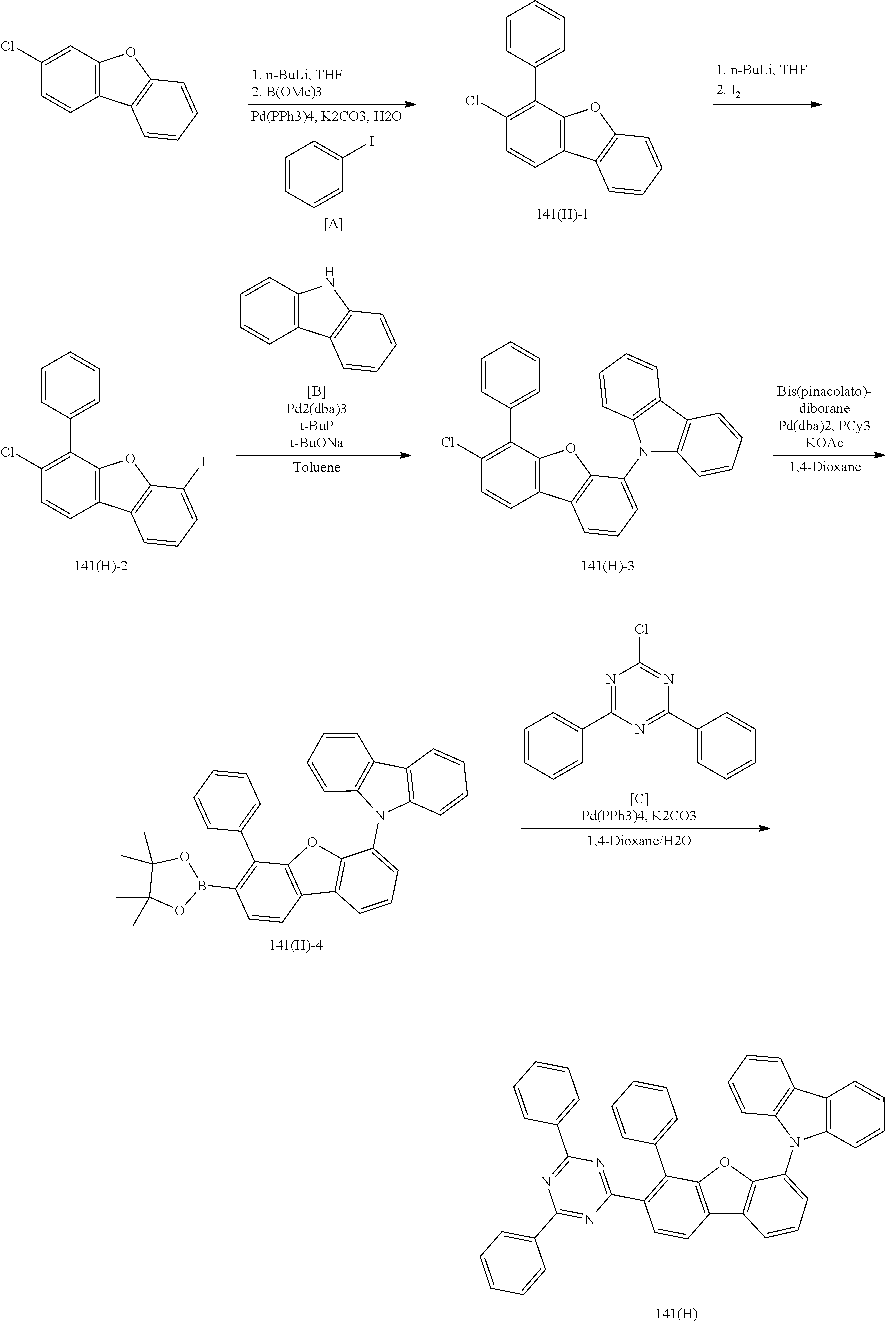

Preparation of Compound 141-1

To a one neck round bottom flask (one neck r.b.f), 3-chlorodibenzo[b,d]furan (15 g, 0.074 mol) and THF (150 mL) were introduced, and the flask was nitrogen substituted. After lowering the temperature of the bath to −78° C., n-BuLi (6.16 g, 0.096 mol) was slowly added dropwise thereto.

When the reaction was finished, the temperature of the bath was raised to room temperature, and trimethyl borate (11.53 g, 0.111 mol) was introduced thereto.

When the reaction was finished, a mixture of iodobenzene (16.61 g, 0.081 mol), tetrakis (triphenylphosphine)palladium(0) (4.28 g, 0.004 mol), potassium carbonate (20.46 g, 0.148 mol) and 1,4-dioxane/water (150 mL/30 mL) was refluxed at 120° C.

The result was extracted with dichloromethane, dried with MgSO₄, silica gel filtered, and then concentrated to obtain Compound 141-1 (9.90 g, 48%).

Preparation of Compound 141-2

To a one neck round bottom flask (one neck r.b.f), 3-chloro-4-phenyldibenzo[b,d]furan (9.90 g, 0.036 mol) and THF (95 mL) were introduced, and the flask was nitrogen substituted. After lowering the temperature of the bath to −78° C., n-BuLi (3.00 g, 0.047 mol) was slowly added dropwise thereto.

When the reaction was finished, the temperature of the bath was raised to room temperature, and iodine (10.05 g, 0.040 mol) was introduced thereto.

When the reaction was completed, distilled water was introduced thereto. The result was extracted with dichloromethane, dried with MgSO₄, silica gel filtered, and then concentrated to obtain Compound 141-2 (12.24 g, 84%).

Preparation of Compound 141-3

In a one neck round bottom flask (one neck r.b.f), a mixture of 3-chloro-6-iodo-4-phenyldibenzo[b,d]furan (12.24 g, 0.030 mol), carbazole (5.52 g, 0.033 mol), tris(dibenzylideneacetone)dipalladium(0) (1.37 g, 0.0015 mol), tri-tert-butylphosphine (6.07 g, 0.030 mol), sodium-tert-butoxide (5.77 g, 0.060 mol) and toluene (120 mL) was refluxed at 130° C.

When the reaction was completed, the result was filtered to remove the solids, and the filtrate was silica gel filtered and then concentrated to obtain Compound 141-3 (8.92 g, 67%).

Preparation of Compound 141-4

In a one neck round bottom flask (one neck r.b.f), a mixture of 9-(7-chloro-6-phenyldibenzo[b,d]furan-4-yl)-9H-carbazole (8.92 g, 0.020 mol), bis(pinacolato)diboron (10.20 g, 0.040 mol), Pd₂(dba)₃ (1.83 g, 0.002 mol), PCy₃ (1.12 g, 0.004 mol), potassium acetate (5.89 g, 0.06 mol) and 1,4-dioxane (90 mL) was refluxed at 140° C.

The result was extracted with dichloromethane, concentrated, and then silica gel filtered. The result was concentrated, and then treated with dichloromethane/methanol to obtain Compound 141-4 (8.35 g, 78%).

Preparation of Compound 141 (H)

In a one neck round bottom flask (one neck r.b.f), a mixture of 9-(6-phenyl-7-(4,4,5,5-tetramethyl-1,3,2-dioxaborolan-2-yl)dibenzo[b,d]furan-4-yl)-9H-carbazole (8.57 g, 0.016 mol), 2-chloro-4,6-diphenyl-1,3,5-triazine (4.71 g, 0.018 mol), tetrakis (triphenylphosphine) palladium (0) (0.92 g, 0.0008 mol), potassium carbonate (4.42 g, 0.032 mol) and 1,4-dioxane/water (85 mL/25 mL) was refluxed at 120° C.

The result was extracted with dichloromethane, dried with MgSO₄, silica gel filtered, and then concentrated to obtain Compound 141(H) (7.89 g, 77%).

Each of target Compound H was synthesized in the same manner as in Preparation Example 5 except that Intermediates A, B and C of the following Table 5 were used.

TABLE 5

| Com- | | | | | |
|---|---|---|---|---|---|
| pound | A | B | C | H | Yield |
| 141 | 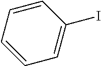 | 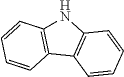 | 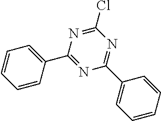 | 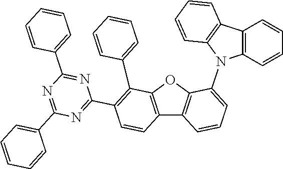 | 77% |
| 142 | 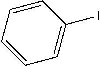 | 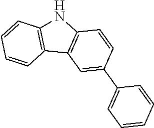 | 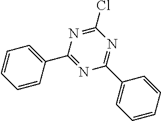 | 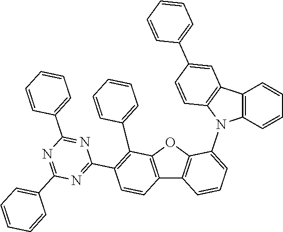 | 63% |
| 143 | 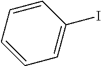 | 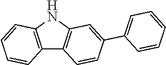 | 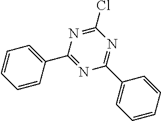 | 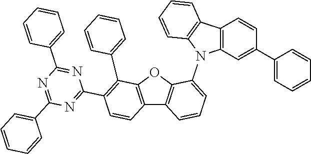 | 55% |
| 145 | 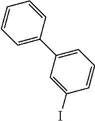 | 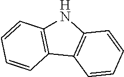 | 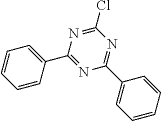 | 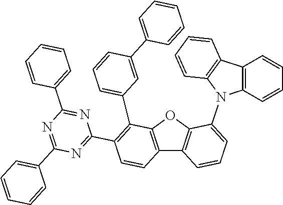 | 57% |
| 148 | 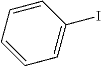 | 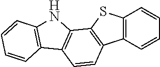 | 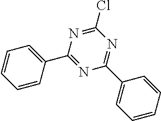 | 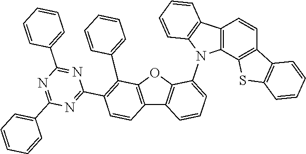 | 69% |
| 149 | 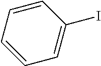 | 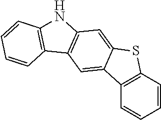 | 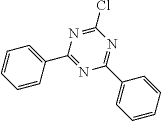 | 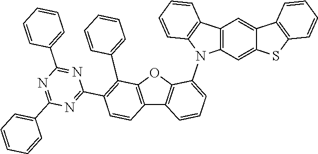 | 87% |
| 151 | 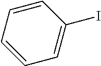 | 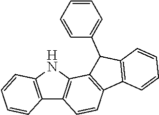 | 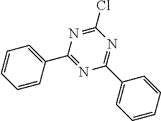 | 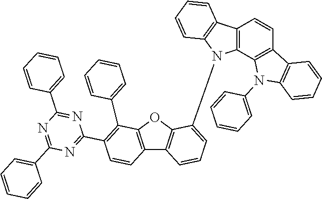 | 61% |
| 152 | 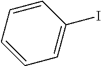 | 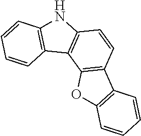 | 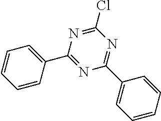 | 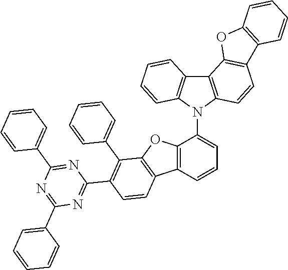 | 57% |
| 154 | 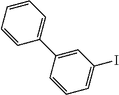 | 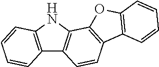 | 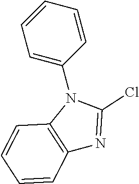 | 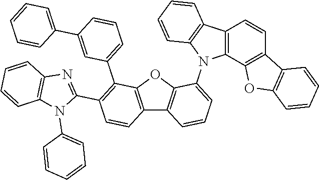 | 63% |
| 157 | 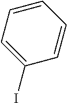 | 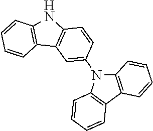 | 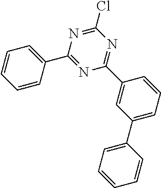 | 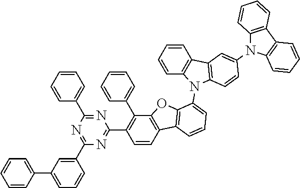 | 72% |
| 159 | 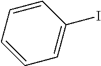 | 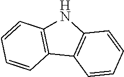 | 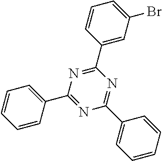 | 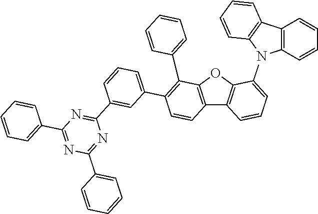 | 57% |
| 161 | 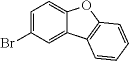 | 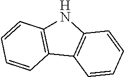 | 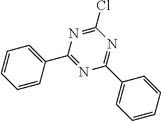 | 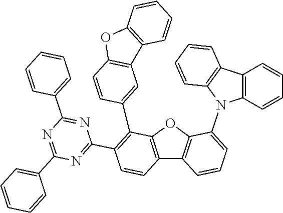 | 59% |
| 162 | 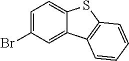 | 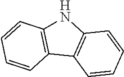 | 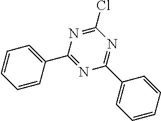 | 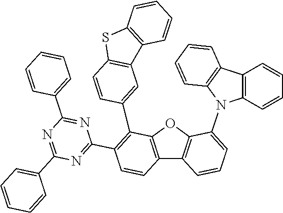 | 73% |
| 164 | 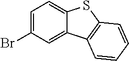 | 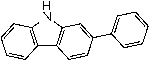 | 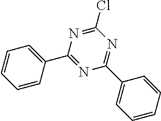 | 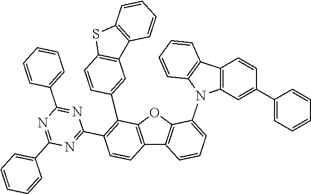 | 47% |
| 165 | 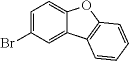 | 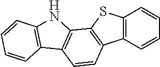 | 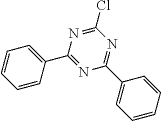 | 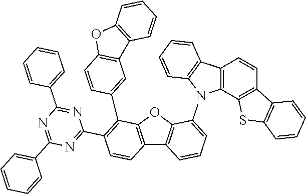 | 89% |
| 167 | 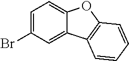 | 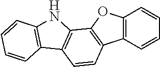 | 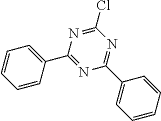 | 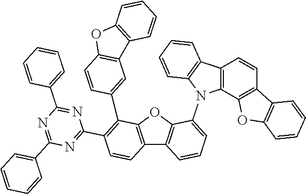 | 68% |
| 168 | 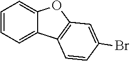 | 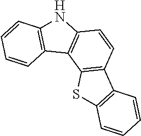 | 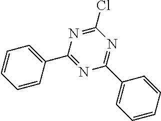 | 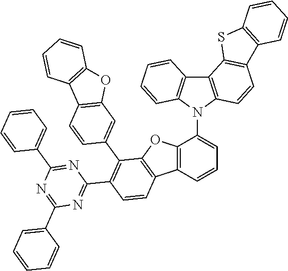 | 53% |
| 169 | 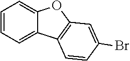 | 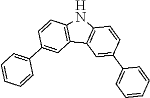 | 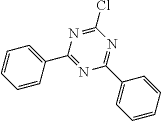 | 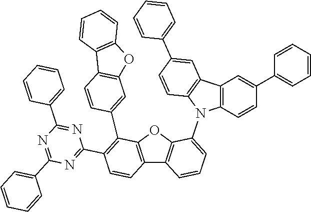 | 75% |
| 170 | 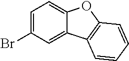 | 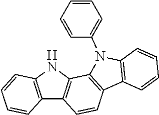 | 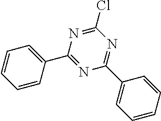 | 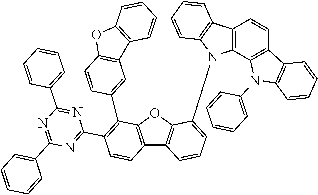 | 62% |
| 171 | 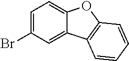 | 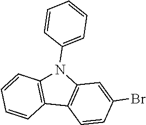 | 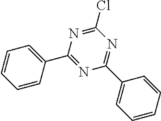 | 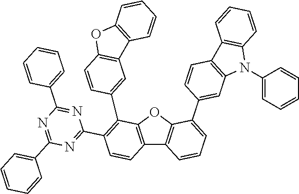 | 47% |
| 174 | 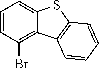 | 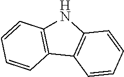 | 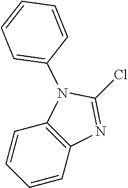 | 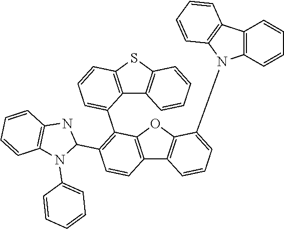 | 83% |
| 175 | 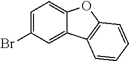 | 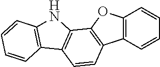 | 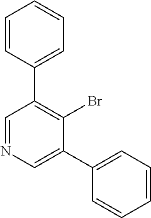 | 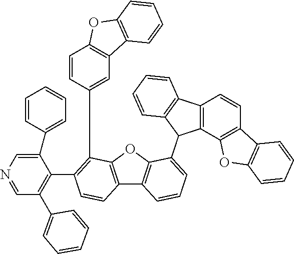 | 49% |
| 176 | 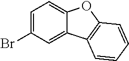 | 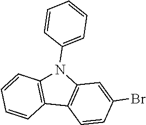 | 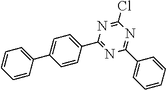 | 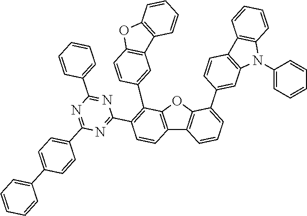 | 51% |
| 177 | 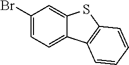 | 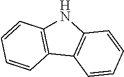 | 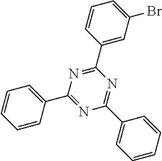 | 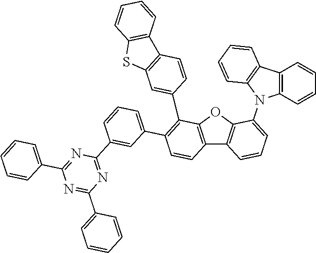 | 91% |
| 179 | 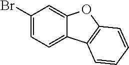 | 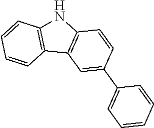 | 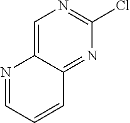 | 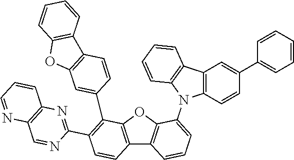 | 74% |
| 180 | 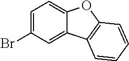 | 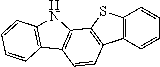 | 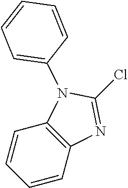 | 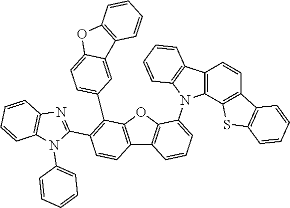 | 82% |

### [Preparation Example 6] Preparation of Compound 181 (I)

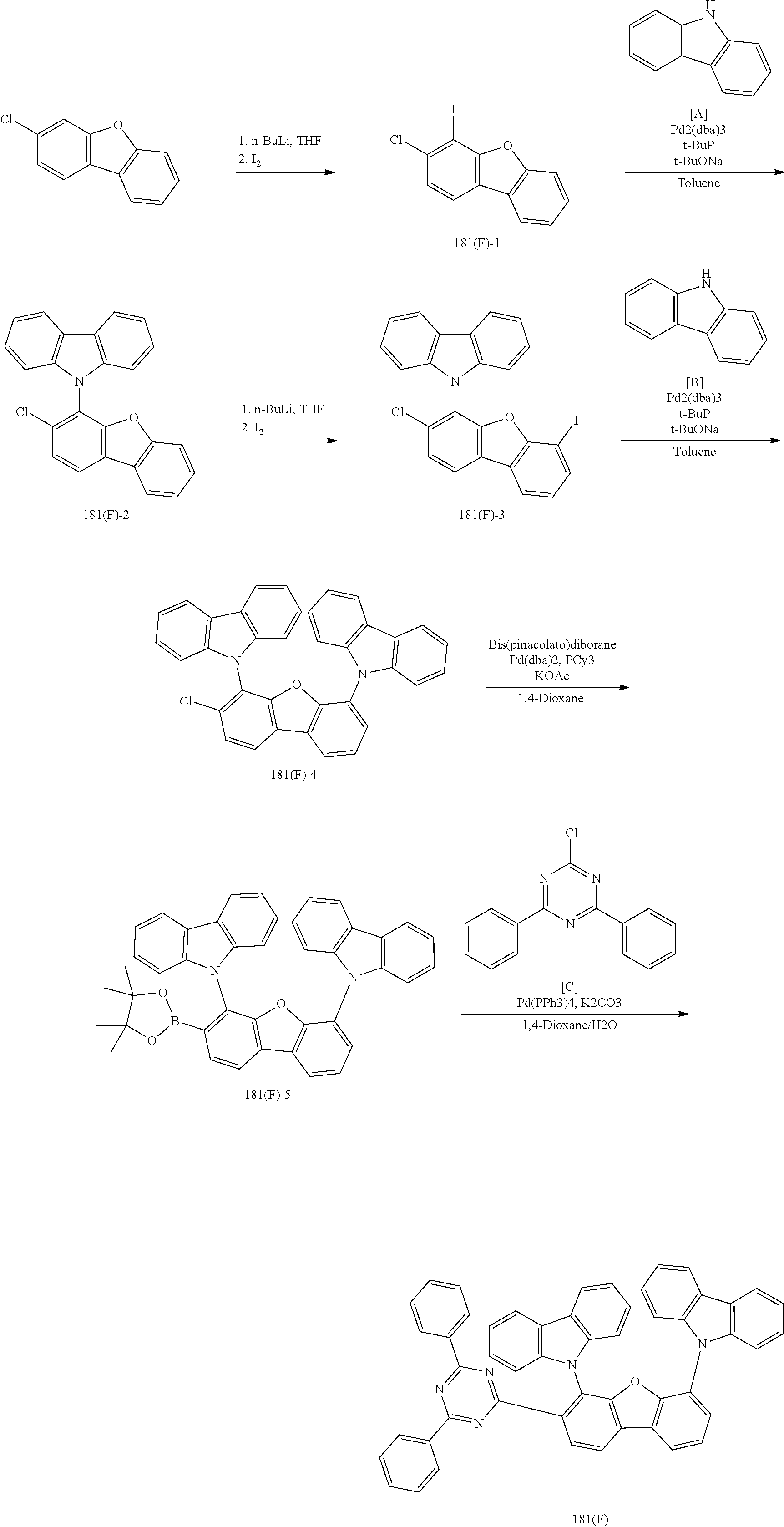

Preparation of Compound 181-1

To a one neck round bottom flask (one neck r.b.f), 3-chlorodibenzo[b,d]furan (15 g, 0.074 mol) and THF (150 mL) were introduced, and the flask was nitrogen substituted. After lowering the temperature of the bath to −78° C., n-BuLi (6.16 g, 0.096 mol) was slowly added dropwise thereto.

When the reaction was finished, the temperature of the bath was raised to room temperature, and iodine (28.17 g, 0.11 mol) was introduced thereto.

When the reaction was completed, distilled water was introduced thereto. The result was extracted with dichloromethane, dried with MgSO₄, silica gel filtered, and then concentrated to obtain Compound 181-1 (16.53 g, 68%).

Preparation of Compound 181-2

In a one neck round bottom flask (one neck r.b.f), a mixture of 3-chloro-4-iododibenzo[b,d]furan (16.53 g, 0.05 mol), carbazole (9.20 g, 0.055 mol), tris(dibenzylideneacetone)dipalladium(0) (2.89 g, 0.0025 mol), tri-tert-butylphosphine (10.12 g, 0.05 mol), sodium-tert-butoxide (9.61 g, 0.10 mol) and toluene (165 mL) was refluxed at 130° C.

When the reaction was completed, the result was filtered to remove the solids, and the filtrate was silica gel filtered and then concentrated to obtain Compound 181-2 (9.75 g, 53%).

Preparation of Compound 181-3

To a one neck round bottom flask (one neck r.b.f), 9-(3-chlorodibenzo[b,d]furan-4-yl)-9H-carbazole (9.75 g, 0.027 mol) and THF (95 mL) were introduced, and the flask was nitrogen substituted. After lowering the temperature of the bath to −78° C., n-BuLi (2.25 g, 0.035 mol) was slowly added dropwise thereto.

When the reaction was finished, the temperature of the bath was raised to room temperature, and iodine (7.54 g, 0.030 mol) was introduced thereto.

When the reaction was completed, distilled water was introduced thereto. The result was extracted with dichloromethane, dried with MgSO₄, silica gel filtered, and then concentrated to obtain Compound 181-3 (7.87 g, 59%).

Preparation of Compound 181-4

In a one neck round bottom flask (one neck r.b.f), a mixture of 9-(3-chloro-6-iododibenzo[b,d]furan-4-yl)-9H-carbazole (7.87 g, 0.016 mol), carbazole (2.94 g, 0.018 mol), tris(dibenzylideneacetone)dipalladium(0) (1.47 g, 0.0016 mol), tri-tert-butylphosphine (3.24 g, 0.016 mol), sodium-tert-butoxide (3.08 g, 0.032 mol) and toluene (75 mL) was refluxed at 130° C.

When the reaction was completed, the result was filtered to remove the solids, and the filtrate was silica gel filtered and then concentrated to obtain Compound 181-4 (6.82 g, 80%).

Preparation of Compound 181-5

In a one neck round bottom flask (one neck r.b.f), a mixture of 9,9′-(3-chlorodibenzo[b,d]furan-4,6-diyl)bis(9H-carbazole) (6.82 g, 0.013 mol), bis(pinacolato)diboron (6.60 g, 0.026 mol), Pd₂(dba)₃ (1.19 g, 0.0013 mol), PCy₃ (0.73 g, 0.0026 mol), potassium acetate (3.59 g, 0.026 mol) and 1,4-dioxane (70 mL) was refluxed at 140° C.

The result was extracted with dichloromethane, concentrated, and then silica gel filtered. The result was concentrated, and then treated with dichloromethane/methanol to obtain Compound 181-5 (5.62 g, 71%).

Preparation of Compound 181 (I)

In a one neck round bottom flask (one neck r.b.f), a mixture of 9,9′-(3-(4,4,5,5-tetramethyl-1,3,2-dioxaborolan-2-yl)dibenzo[b,d]furan-4,6-diyl)bis(9H-carbazole) (5.62 g, 0.009 mol), 2-chloro-4,6-diphenyl-1,3,5-triazine (2.68 g, 0.010 mol), tetrakis (triphenylphosphine)palladium(0) (0.52 g, 0.0005 mol), potassium carbonate (2.49 g, 0.018 mol) and 1,4-dioxane/water (55 mL/16 mL) was refluxed at 120° C.

The result was extracted with dichloromethane, dried with MgSO₄, silica gel filtered, and then concentrated to obtain Compound 181(I) (3.74 g, 57%).

Each of target Compound I was synthesized in the same manner as in Preparation Example 6 except that Intermediates A, B and C of the following Table 6 were used.

TABLE 6

| Com- | | | | | |
|---|---|---|---|---|---|
| pound | A | B | C | I | Yield |
| 181 | 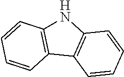 | 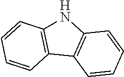 | 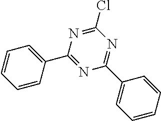 | 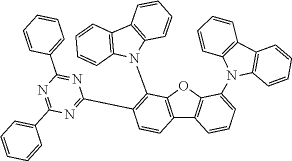 | 57% |
| 182 | 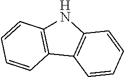 | 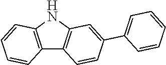 | 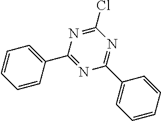 | 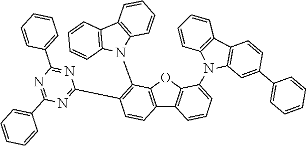 | 61% |
| 183 | 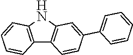 | 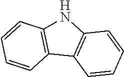 | 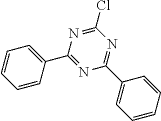 | 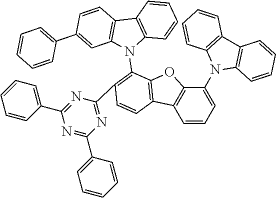 | 45% |
| 184 | 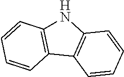 | 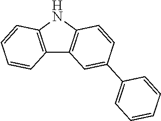 | 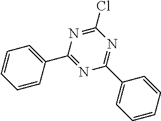 | 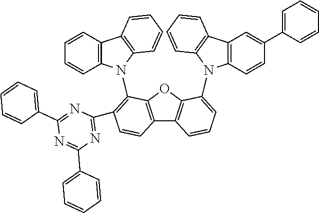 | 87% |
| 186 | 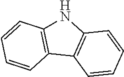 | 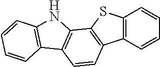 | 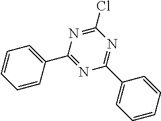 | 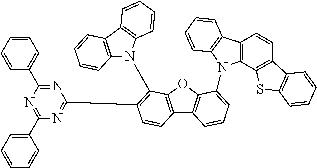 | 59% |
| 187 | 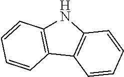 | 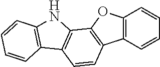 | 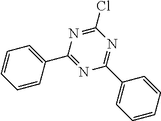 | 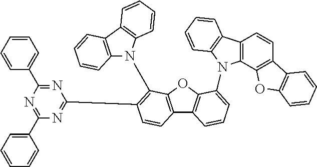 | 75% |
| 188 | 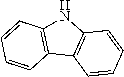 | 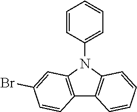 | 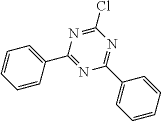 | 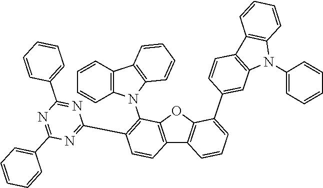 | 69% |
| 189 | 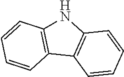 | 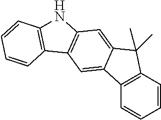 | 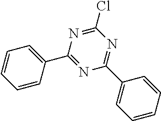 | 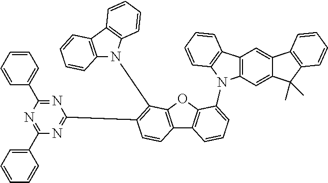 | 48% |
| 191 | 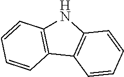 | 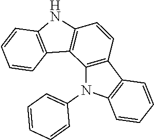 | 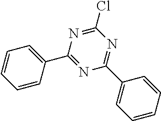 | 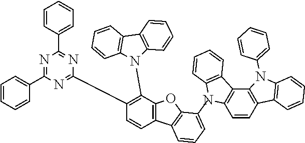 | 72% |
| 192 | 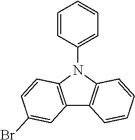 | 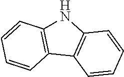 | 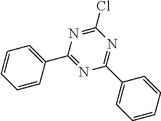 | 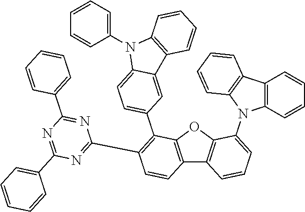 | 49% |
| 193 | 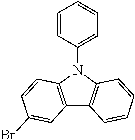 | 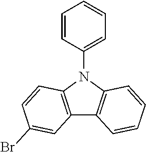 | 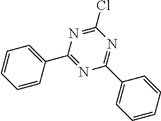 | 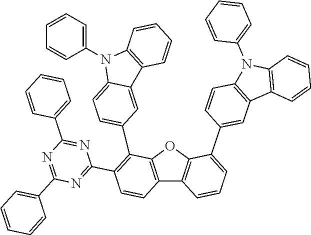 | 89% |
| 196 | 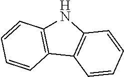 | 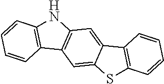 | 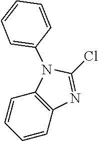 | 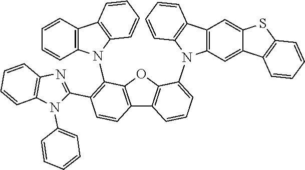 | 56% |
| 198 | 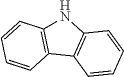 | 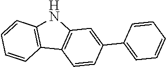 | 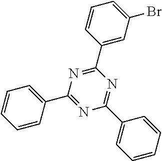 | 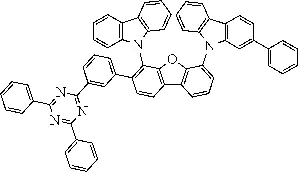 | 74% |
| 199 | 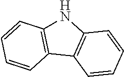 | 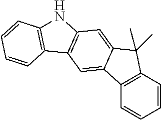 | 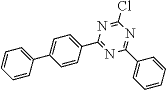 | 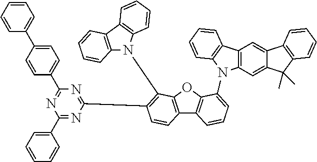 | 76% |
| 200 | 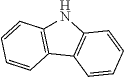 | 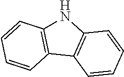 | 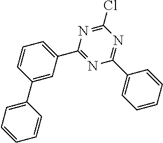 | 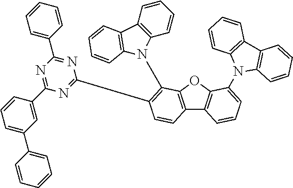 | 59% |

### [Preparation Example 7] Preparation of Compound 2-23(E)

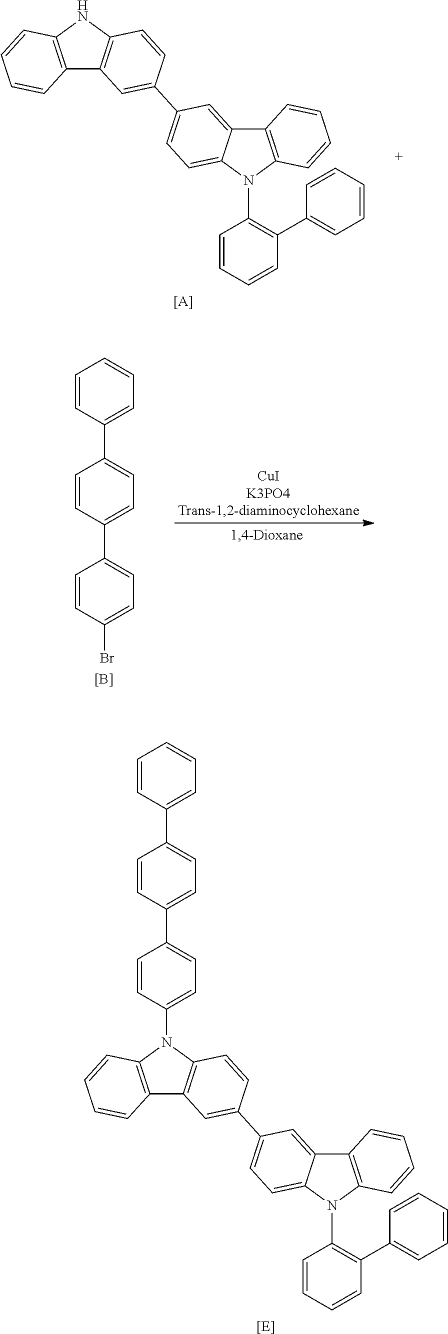

Preparation of Compound 2-23[E]

In a one neck round bottom flask, 9-([1,1′-biphenyl]-2-yl)-9H,9′H-3,3′-bicarbazole (10 g, 0.021 mol), 4-bromo-1,1′: 4′,1″-terphenyl (7.14 g, 0.023 mol), CuI (4.00 g, 0.021 mol), trans-1,4-diaminocyclohexane (2.40 g, 0.021 mol) and K₃PO₄ (8.92 g, 0.042 mol) were dissolved in 1,4-oxane (100 mL), and the mixture was refluxed for 8 hours at 125° C. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain Compound 2-23[E] (13.17 g, 88%).

Compound E corresponding to Chemical Formula A of the following Table 7 was synthesized in the same manner as in Preparation Example 7 except that Intermediates A and B of the following Table 7 were used.

TABLE 7

| Com- | | | | |
|---|---|---|---|---|
| pound | A | B | E | Yield |
| 2-23 | 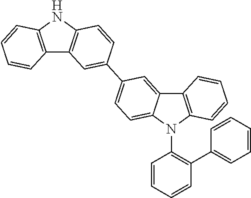 | 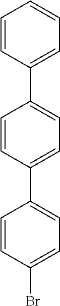 | 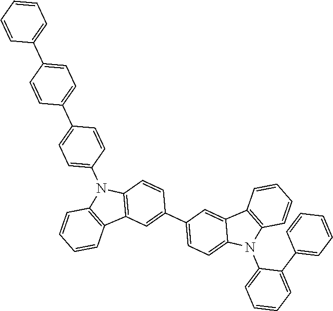 | 88% |
| 2-32 | 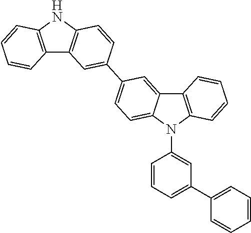 | 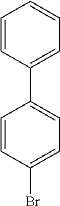 | 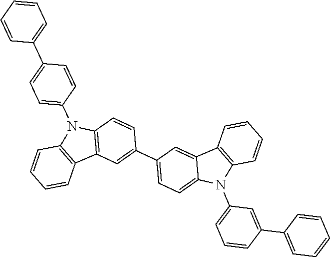 | 92% |
| 2-33 | 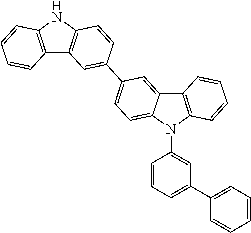 | 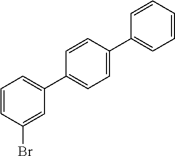 | 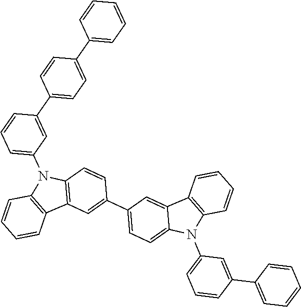 | 74% |
| 2-34 | 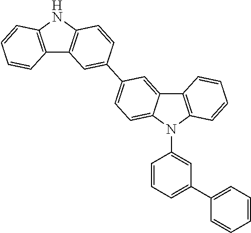 | 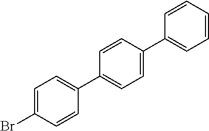 | 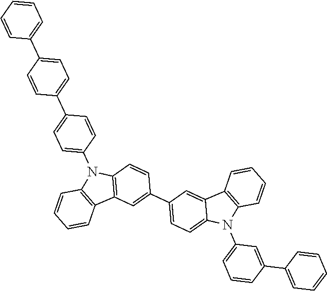 | 65% |
| 2-42 | 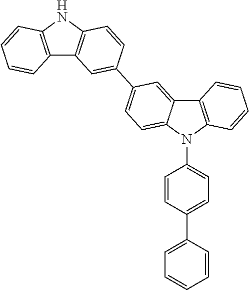 | 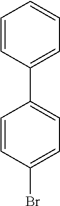 | 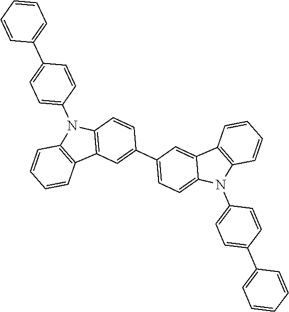 | 69% |
| 2-44 | 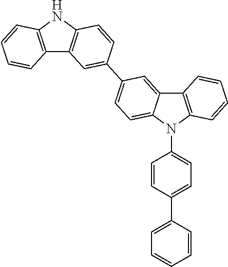 | 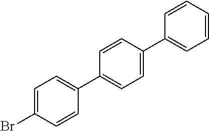 | 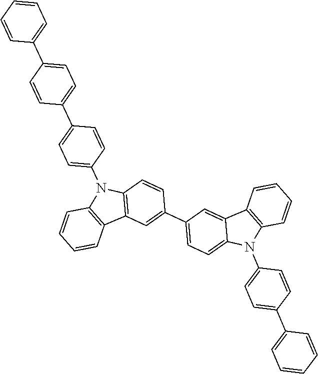 | 85% |

The heterocyclic compound corresponding to Chemical Formula 1 and the heterocyclic compound corresponding to Chemical Formula A other than the compounds described in Preparation Examples 1 to 7 and Table 1 to Table 7 were also prepared in the same manner as in the preparation examples described above.

Synthesis identification data of the compounds prepared above are as described in the following [Table 8] and [Table 9].

TABLE 8

| Compound | FD-Mass | Compound | FD-Mass |
|---|---|---|---|
| 1 | m/z = 551.65 (C39H25N3O = 551.20) | 2 | m/z = 703.85 (C51H33N3O = 703.26) |
| 3 | m/z = 703.85 (C51H33N3O = 703.26) | 4 | m/z = 703.85 (C51H33N3O = 703.26) |
| 6 | m/z = 703.85 (C51H33N3O = 703.26) | 9 | m/z = 627.75 (C45H29N3O = 627.23) |
| 11 | m/z = 703.85 (C51H33N3O = 703.26) | 13 | m/z = 588.71 (C43H28N2O = 588.22) |
| 16 | m/z = 650.78 (C48H30N2O = 650.24) | 20 | m/z = 677.81 (C49H31N3O = 677.25) |
| 21 | m/z = 641.73 (C45H27N3O2 = 641.21) | 23 | m/z = 641.73 (C45H27N3O2 = 641.21) |
| 24 | m/z = 657.79 (C45H27N3OS = 657.19) | 26 | m/z = 822.99 (C57H34N4OS = 822.25) |
| 28 | m/z = 717.83 (C51H31N3O2 = 717.24) | 30 | m/z = 733.89 (C51H31N3OS = 733.22) |
| 37 | m/z = 717.83 (C51H31N3O2 = 717.24) | 39 | m/z = 717.83 (C51H31N3O2 = 717.24) |
| 41 | m/z = 640.75 (C45H28N4O = 640.23) | 42 | m/z = 716.84 (C51H32N4O = 716.26) |
| 43 | m/z = 716.84 (C51H34N4O = 716.26) | 45 | m/z = 716.84 (C51H32N4O = 716.26) |
| 48 | m/z = 746.89 (C51H30N4OS = 746.21) | 49 | m/z = 746.89 (C51H30N4OS = 746.21) |
| 51 | m/z = 805.94 (C57H35N5O = 805.28) | 54 | m/z = 767.89 (C55H33N3O2 = 767.26) |
| 59 | m/z = 716.84 (C51H32N4O = 716.26) | 61 | m/z = 730.83 (C51H30N4O2 = 730.24) |
| 62 | m/z = 746.89 (C51H30N4OS = 746.21) | 63 | m/z = 806.93 (C57H34N4O2 = 806.27) |
| 65 | m/z = 836.97 (C57H32N4O2S = 836.22) | 67 | m/z = 820.91 (C57H32N4O3 = 820.25) |
| 68 | m/z = 836.97 (C57H32N4O2S = 836.22) | 70 | m/z = 896.02 (C63H37N5O2 = 895.29) |
| 71 | m/z = 806.93 (C57H34N4O2 = 806.27) | 74 | m/z = 707.85 (C49H29N3OS = 707.20) |
| 78 | m/z = 806.93 (C57H34N4O2 = 806.27) | 80 | m/z = 7 97.93 |
| | | | (C55H31N3O2S = 797.21) |
| 81 | m/z = 729.84 (C51H31N5O = 729.25) | 82 | m/z = 805.94 (C57H35N5O = 805.28) |
| 85 | m/z = 882.04 (C63H39N5O = 881.32) | 86 | m/z = 835.99 (C57H33N5OS = 835.24) |
| 88 | m/z = 805.94 (C57H35N5O = 805.28) | 91 | m/z = 895.04 (C63H38N6O = 894.31) |
| 92 | m/z = 805.94 (C57H35N4O = 805.28) | 93 | m/z = 882.04 (C63H39N5O = 881.32) |
| 96 | m/z = 796.95 (C55H32N4OS = 796.23) | 98 | m/z = 882.04 (C63H39N5O = 881.32) |
| 100 | m/z = 805.94 (C57H35N5O = 805.28) | 101 | m/z = 551.65 (C39H25N3O = 551.20) |
| 102 | m/z = 703.85 (C51H33N3O = 703.26) | 103 | m/z = 703.85 (C51H33N3O = 703.26) |
| 104 | m/z = 703.85 (C51H33N3O = 703.26) | 107 | m/z = 703.85 (C51H33N3O = 703.26) |
| 109 | m/z = 627.75 (C45H29N3O = 627.23) | 111 | m/z = 703.85 (C51H33N3O = 703.26) |
| 113 | m/z = 588.71 (C43H28N2O = 588.22) | 118 | m/z = 703.85 (C51H33N3O = 703.26) |
| 119 | m/z = 703.85 (C51H33N3O = 703.26) | 121 | m/z = 641.73 (C45H27N3O2 = 641.21) |
| 122 | m/z = 657.79 (C45H27N3OS = 657.19) | 123 | m/z = 641.73 (C45H27N3O2 = 641.21) |
| 124 | m/z = 657.79 (C45H27N3OS = 657.19) | 125 | m/z = 806.93 (C57H34N4O2 = 806.27) |
| 126 | m/z = 822.99 (C57H34N4OS = 822.25) | 130 | m/z = 733.89 (C51H31N3OS = 733.22) |
| 136 | m/z = 783.95 (C55H33N3OS = 783.23) | 137 | m/z = 717.83 (C51H31N3O2 = 717.24) |
| 139 | m/z = 717.83 (C51H31N3O2 = 717.24) | 141 | m/z = 640.75 (C45H28N4O = 640.23) |
| 142 | m/z = 716.84 (C51H32N4O = 716.26) | 143 | m/z = 716.84 (C51H32N4O = 716.26) |
| 145 | m/z = 716.84 (C51H32N4O = 716.26) | 148 | m/z = 746.89 (C51H30N4OS = 746.21) |
| 149 | m/z = 746.89 (C51H30N4OS = 746.21) | 151 | m/z = 805.94 (C57H35N5O = 805.28) |
| 152 | m/z = 730.83 (C51H30N402 = 730.24) | 154 | m/z = 767.89 (C55H33N3O2 = 767.26) |
| 157 | m/z = 882.04 (C63H39N5O = 881.32) | 159 | m/z = 716.84 (C51H32N4O = 716.26) |
| 161 | m/z = 730.83 (C51H30N4O2 = 730.24) | 162 | m/z = 746.89 (C51H30N4OS = 746.21) |
| 164 | m/z = 822.99 (C57H34N4OS = 822.25) | 165 | m/z = 836.97 |
| | | | (C57H32N4O2S = 836.22) |
| 167 | m/z = 820.91 (C57H32N4O3 = 820.25) | 168 | m/z = 836.97 |
| | | | (C57H32N4O2S = 836.22) |
| 169 | m/z = 883.02 (C63H38N4O2 = 882.30) | 170 | m/z = 896.02 (C63H37N5O2 = 895.29) |
| 171 | m/z = 806. 93 (C57H34N4O2 = 806.27) | 174 | m/z = 707.85 (C49H29N3OS = 707.20) |
| 175 | m/z = 818.93 (C59H34N2O3 = 818.26) | 176 | m/z = 883.02 (C63H38N4O2 = 882.30) |
| 177 | m/z = 822.99 (C57H34N4OS = 822.25) | 179 | m/z = 704.79 (C49H28N4O2 = 704.22) |
| 180 | m/z = 797.93 (C55H31N3O2S = 797.21) | 181 | m/z = 729.84 (C51H31N5O = 79.25) |
| 182 | m/z = 805.94 (C57H35N5O = 805.28) | 183 | m/z = 805.94 (C57H35N5O = 805.28) |
| 184 | m/z = 805.94 (C57H35N5O = 805.28) | 186 | m/z = 835.99 (C57H33N5OS = 835.24) |
| 187 | m/z = 819.92 (C57H33N5O2 = 819.26) | 188 | m/z = 805.94 (C57H35N5O = 805.28) |
| 189 | m/z = 846.01 (C60H39N5O = 845.32) | 191 | m/z = 895.04 (C63H38N5O = 894.31) |
| 192 | m/z = 805.94 (C57H35N5O = 805.28) | 193 | m/z = 882.04 (C63H39N5O = 881.32) |
| 196 | m/z = 796.95 (C55H32N4OS = 796.23) | 198 | m/z = 882.04 (C63H39N5O = 881.32) |
| 199 | m/z = 922.10 (C66H43N5O = 921.35) | 200 | m/z = 805.94 (C57H35N5O = 805.28) |
| 227 | m/z = 762.95 (C51H30N4S2 = 762.19) | 231 | m/z = 746.89 (C51H30N4OS = 746.21) |
| 2-23 | m/z = 712.90 (C54H36N2 = 712.29) | 2-32 | m/z = 636.80 (C48H32N2 = 636.26) |
| 2-33 | m/z = 712.90 (C54H36N2 = 712.29) | 2-34 | m/z = 712.90 (C54H36N2 = 712.29) |
| 2-42 | m/z = 636.80 (C48H32N2 = 636.26) | 2-44 | m/z = 712.90 (C54H36N2 = 712.29) |

TABLE 9

| Compound | ¹H NMR (CDCl₃, 200 mz) |
|---|---|
| 1 | δ = 8.36 (4H, m), 8.01~8.08 (4H, m), 7.41~7.51 (17H, m) |
| 2 | δ = 8.36 (4H, m), 8.01~8.08 (4H, m), 7.75 (4H, d), 7.41~7.51 (13H, |
| | m), 7.25 (8H, s) |
| 3 | δ = 8.36 (4H, m), 8.01~8.08 (4H, m), 7.73~7.75 (5H, m), 7.61 (2H, |
| | d), 7.41~7.50 (13H, m), 7.25 (4H, s) |
| 4 | δ = 8.36 (4H, m), 8.01~8.08 (4H, m), 7.94 (2H, s), 7.73~7.75 (6H, |
| | m), 7.61 (4H, d), 7.41~7.51 (13H, m) |
| 6 | δ = 8.36 (4H, m), 8.01~8.07 (4H, m), 7.94 (2H, s), 7.73~7.75 (4H, |
| | m), 7.61 (4H, d), 7.41~7.51 (15H, m) |
| 9 | δ = 8.36 (3H, m), 8.01~8.08 (4H, m), 7.73~7.75 (3H, m), 7.61 (1H, |
| | d), 7.41~7.51 (17H, m) |
| 11 | δ = 8.38 (1H, d), 7.94~8.08 (7H, m), 7.73~7.75 (5H, m), 7.61 (1H, |
| | d), 7.41~7.51 (17H, m), 7.25 (2H, d) |
| 13 | δ = 8.56 (1H, d), 8.01~8.09 (5H, m), 7.75~7.81 (3H, m), 7.41~7.54 |
| | (18H, m), 7.28 (IH, t) |
| 16 | δ = 8.95 (1H, d), 8.87 (1H, d), 8.46 (1H, d), 7.94~8.12 (6H, m), |
| | 7.73~7.75 (3H, m), 7.39~7.63 (13H, m), 7.25 (4H, s), 7.15 (1H, d) |
| 20 | δ = 9.51 (1H, s), 9.19 (1H, s), 7.94~8.08 (7H, m), 7.84 (1H, s), |
| | 7.41~7.75 (17H, m), 7.25 (4H, s) |
| 21 | δ = 8.36 (4H, m), 7.98~8.08 (5H, m), 7.82 (1H, d), 7.69 (1H, d), |
| | 7.31~7.57 (16H, m) |
| 23 | δ = 8.36 (4H, m), 7.98~8.08 (6H, m), 7.82 (1H, d), 7.76 (1H, s), |
| | 7.31~7.54 (15H, m) |
| 24 | δ = 8.45 (1H, d), 8.36 (4H, m), 7.93~8.12 (8H, m), 7.41~7.56 (14H, |
| | m) |
| 26 | δ = 8.55 (1H, d), 8.36 (4H, m), 7.94~8.12 (9H, m), 7.35~7.62 (19H, |
| | m), 7.16 (1H, t) |
| 28 | δ = 8.36 (4H, m), 7.73~8.08 (12H, m), 7.31~7.61 (15H, m) |
| 30 | δ = 8.45 (1H, d), 8.36 (4H, m), 7.93~8.12 (9H, m), 7.73~7.75 (3H, |
| | m), 7.41~7.61 (14H, m) |
| 37 | δ = 8.36~8.38 (3H, m),7.94~8.08 (6H, m),7.69~7.82 (5H, m), |
| | 7.31~7.57 (17H, m) |
| 39 | δ = 8.36~8.38 (5H, m), 7.94~8.08 (6H, m), 7.82 (1H, d), 7.69~7.73 |
| | (2H, m), 7.31~7.57 (17H, m) |
| 41 | δ = 8.55 (1H, d), 8.36 (4H, m), 8.19 (1H, d), 7.94~8.07 (4H, m), |
| | 7.31~7.58 (16H, m), 7.16~7.20 (2H, m) |
| 42 | δ = 8.55 (1H, d), 8.36 (4H, m), 7.89~8.07 (6H, m), 7.75~7.77 (3H, |
| | m), 7.31~7.51 (17H, m), 7.16 (1H, t) |
| 43 | δ = 8.55 (1H, d), 8.31~8.36 (5H, m), 7.91~8.07 (5H, m), 7.74~7.75 |
| | (3H, m), 7.35~7.51 (17H, m), 7.16 (1H, t) |
| 45 | δ = 8.55 (1H, d), 8.36 (4H, m), 8.19 (1H, d), 7.94~8.07 (5H, m), |
| | 7.73~7.75 (3H, m), 7.31~7.61 (16H, m), 7.16~7.20 (2H, m) |
| 48 | δ = 8.55 (1H, d), 8.45 (1H, d), 8.36 (4H, m), 7.93~8.07 (6H, m), |
| | 7.35~7.60 (17H, m), 7.16 (1H, t) |
| 49 | δ = 8.55 (1H, d), 8.45 (1H, d), 8.36 (4H, m), 7.93~8.07 (5H, m), |
| | 7.86 (1H, s), 7.78 (1H, s), 7.31~7.51 (16H, m), 7.16 (1H, t) |
| 51 | δ = 8.55 (2H, d), 8.36 (4H, m), 7.94~8.12 (6H, m), 7.31~7.62 (21H, |
| | m), 7.16 (2H, t) |
| 54 | δ = 8.55~8.56 (2H, m), 7.94~8.07 (6H, m), 7.73~7.84 (5H, m), |
| | 7.28~7.62 (19H, m), 7.16 (1H, t) |
| 59 | δ = 8.55 (1H, d), 8.36~8.38 (5H, m), 8.19 (1H, d), 7.94~8.07 (5H, |
| | m), 7.73 (1H, t), 7.31~7.61 (17H, m), 7.16~7.20 (2H, m) |
| 61 | δ = 8.55 (1H, d), 8.36 (4H, m), 8.19 (1H, d), 7.79~8.07 (8H, m), |
| | 7.31~7.58 (14H, m), 7.16~7.20 (2H, m) |
| 62 | δ = 8.55 (1H, d), 8.45 (1H, d), 8.36 (4H, m), 7.94~8.19 (9H, m), |
| | 7.46~7.58 (11H, m), 7.31~7.35 (2H, m), 7.20~8.45 (2H, m) |
| 63 | δ = 8.55 (1H, d), 8.36 (4H, m), 7.89~8.07 (8H, m), 7.75~7.82 (5H, |
| | m), 7.31~7.54 (15H, m), 7.16 (1H, t) |
| 65 | δ = 8.55 (1H, d), 8.45 (1H, d), 8.36 (4H, m), 7.79~8.07 (10H, m), |
| | 7.31~7.60 (15H, m), 7.16 (1H, t) |
| 67 | δ = 8.55 (1H, d), 8.36 (4H, m), 7.79~8.07 (10H, m), 7.31~7.54 (16H, |
| | m), 7.16 (1H, t) |
| 68 | δ = 8.55 (1H, d), 8.45 (1H, d), 8.36 (4H, m), 7.93~8.07 (8H, m), |
| | 7.76~7.82 (2H, m), 7.31~7.56 (15H, m), 7.16 (1H, t) |
| 70 | δ = 8.55 (2H, d), 8.36 (4H, m), 7.79~8.12 (10H, m), 7.31~7.62 (19H, |
| | m), 7.16 (2H, t) |
| 71 | δ = 8.55 (1H, d), 8.31~8.36 (5H, m), 7.74~8.08 (11H, m), 7.50~7.62 |
| | (13H, m), 7.31~7.39 (3H, m), 7.16 (1H, t) |
| 74 | δ = 8.55 (1H, d), 8.45 (1H, d), 8.19 (1H, d), 7.93~8.07 (7H, m), |
| | 7.81 (1H, d), 7.16 (17H, m) |
| 78 | δ = 8.55 (1H, d), 8.36~8.38 (3H, m), 8.19 (1H, d), 7.94~8.07 (6H, |
| | m), 7.69~7.82 (5H, m), 7.31~7.61 (16H, m), 7.16~7.20 (2H, m) |
| 80 | δ = 8.55 (1H, d), 8.45 (1H, d), 7.79~8.07 (11H, m), 7.28~7.60 (16H, |
| | m), 7.16 (1H, t) |
| 81 | δ = 8.55 (2H, d), 8.36 (4H, m), 8.19 (2H, d), 7.94~7.96 (4H, m), |
| | 7.46~7.58 (12H, m), 7.31~7.35 (3H, m), 7.16~7.20 (4H, m) |
| 82 | δ = 8.55 (2H, d), 8.31~8.36 (5H, m), 8.19 (1H, d), 7.91~7.96 (5H, |
| | m), 7.74~7.75 (3H, m), 7.31~7.58 (16H, m), 7.16~7.20 (3H, m) |
| 85 | δ = 8.55 (2H, d), 8.31~8.36 (6H, m), 7.91~7.96 (6H, m), 7.74~7.75 |
| | (6H, m), 7.31~7.50 (17H, m), 7.16 (2H, t) |
| 86 | δ = 8.55 (2H, d), 8.45 (1H, d), 8.36 (4H, m), 8.19 (1H, d), 8.05 |
| | (1H, d), 7.93~7.96 (5H, m), 7.46~7.60 (13H, m), 7.31~7.35 (3H, m), |
| | 7.16~7.20 (3H, m) |
| 88 | δ = 8.55 (2H, d), 8.31~8.36 (5H, m), 8.19 (1H, d), 7.91~8.08 (6H, |
| | m), 7.74 (1H, s), 7.50~7.62 (15H, m), 7.35 (2H, t), 7.16~7.20 (3H, |
| | m) |
| 91 | δ = 8.55 (3H, d), 8.36 (4H, m), 8.19 (1H, d), 8.12 (1H, d), |
| | 7.94~7.96 (5H, m),7.46~7.62 (15H, m),7.31~7.35 (4H, m), |
| | 7.16~7.20 (5H, m) |
| 92 | δ = 8.55 (1H, d), 8.30~8.36 (5H, m), 7.89~8.19 (8H, m), 7.46~7.62 |
| | (16H, m), 7.31~7.35 (2H, m), 7.16~7.20 (3H, m) |
| 93 | δ = 8.55 (1H, d), 8.30~8.36 (5H, m), 7.89~8.19 (10H, m), 7.77 (1H, |
| | d), 7.50~7.62 (19H, m), 7.35 (1H, t), 7.16~7.20 (2H, m) |
| 96 | δ = 8.55~8.56 (3H, m), 8.45 (1H, d), 8.19 (1H, d), 7.78~7.96 (8H, |
| | m), 7.16~7.58 (19H, m), |
| 98 | δ = 8.55 (2H, d), 8.31~8.38 (6H, m), 8.19 (1H, d), 7.91~7.96 (6H, |
| | m), 7.73~7.75 (4H, m), 7.31~7.61 (17H, m), 7.16~7.20 (3H, m) |
| 100 | δ = 8.55 (2H, d), 8.36~8.38 (3H, m), 8.19 (2H, d), 7.94~7.96 (5H, |
| | m), 7.73~7.75 (3H, m), 7.31~7.61 (16H, m), 7.16~7.20 (4H, m) |
| 101 | δ = 8.36 (4H, m), 8.01~8.13 (4H, m), 7.41~7.51 (17H, m) |
| 102 | δ = 8.36 (4H, m), 8.01~8.13 (4H, m), 7.75 (4H, d), 7.41~7.51 (13H, |
| | m), 7.25 (8H, s) |
| 103 | δ = 8.36 (4H, m), 8.01~8.13 (4H, m), 7.94 (1H, s), 7.73~7.75 (5H, |
| | m), 7.61 (2H, d), 7.41~7.50 (13H, m), 7.25 (4H, s) |
| 104 | δ = 8.36 (4H, m), 8.01~8.13 (4H, m), 7.94 (2H, s), |
| | m), 7.61 (4H, d), 7.41~7.51 (13H, m) |
| 107 | δ = 8.36 (4H, m), 8.01~8.13 (4H, m), 7.94 (1H, s), 7.73~7.75 (3H, |
| | m), 7.61 (2H, d), 7.41~7.51 (15H, m), 7.25 (4H, s) |
| 109 | δ = 8.36~8.38 (3H, m), 8.01~8.13 (4H, m), 7.94 (1H, s), 7.73~7.75 |
| | (3H, m), 7.61 (1H, d), 7.41~7.51 (17H, m) |
| 111 | δ = 8.38 (1H, d), 7.94~8.13 (7H, m), 7.73~7.75 (5H, m), 7.61 (1H, |
| | d), 7.41~7.51 (17H, m), 7.25 (2H, d) |
| 113 | δ = 8.56 (1H, d), 8.01~8.13 (5H, m), 7.75~7.81 (3H, m), 7.41~7.54 |
| | (17H, m), 7.28 (1H, t) |
| 118 | δ = 8.36 (2H, m), 7.96~8.13 (8H, m), 7.75~7.79 (4H, m), 7.41~7.60 |
| | (17H, m), 7.25 (2H, d) |
| 119 | δ = 8.36 (4H, m), 7.94~8.13 (7H, m), 7.73~7.75 (3H, m), 7.61 (2H, |
| | d), 7.41~7.51 (15H, m), 7.25 (2H, d) |
| 121 | δ = 8.36 (4H, m), 7.98~8.13 (5H, m), 7.82 (1H, d), 7.69 (1H, d), |
| | 7.31~7.57 (16H, m) |
| 122 | δ = 8.45 (1H, d), 8.36 (4H, m), 7.93~8.13 (7H, m), 7.68 (1H, t), |
| | 7.41~7.56 (16H, m) |
| 123 | δ = 8.36 (4H, m), 7.98~8.13 (6H, m), 7.82 (1H, d), 7.76 (1H, s), |
| | 7.31~7.54 (15H, m) |
| 124 | δ = 8.45 (1H, d), 8.36 (4H, m), 7.93~8.13 (8H, m), 7.41~7.56 (14H, |
| | m) |
| 125 | δ = 8.55 (1H, d), 8.36 (4H, m), 8.01~8.13 (4H, m), 7.79~7.94 (5H, |
| | m), 7.35~7.62 (19H, m), 7.16 (1H, t) |
| 126 | δ = 8.55 (1H, d), 8.36 (4H, m), 7.94~8.13 (9H, m), 7.35~7.62 (19H, |
| | m), 7.16 (1H, t) |
| 130 | δ = 8.45 (1H, d), 8.36 (4H, m), 7.93~8.13 (9H, m), 7.73~7.75 (3H, |
| | m), 7.41~7.61 (14H, m) |
| 136 | δ = 8.95 (1H, d), 8.45~8.50 (2H, m), 8.36 (2H, m), 7.93~8.20 (12H, |
| | m), 7.75~7.77 (3H, m), 7.39~7.56 (11H, m), 7.25 (2H, d) |
| 137 | δ = 8.36~8.38 (3H, m), 7.94~8.13 (7H, m),7.69~7.82 (8H, m), |
| | 7.41~7.61 (16H, m) |
| 139 | δ = 8.36 (5H, m), 7.94~8.13 (6H, m), 7.82 (1H, d), 7.69~7.73 (2H, |
| | m), 7.31~7.61 (17H, m) |
| 141 | δ = 8.55 (1H, d), 8.36 (4H, m), 8.13~8.19 (2H, m), 7.94~8.01 (3H, |
| | m), 7.31~7.58 (16H, m), 7.16~7.20 (2H, m) |
| 142 | δ = 8.55 (1H, d), 8.36 (4H, m), 8.13 (1H, d), 7.89~8.01 (4H, m), |
| | 7.75~7.77 (3H, m), 7.31~7.51 (17H, m), 7.16 (1H, t) |
| 143 | δ = 8.55 (1H, d), 8.31~8.36 (5H, m), 8.13 (1H, d), 7.91~8.01 (4H, |
| | m), 7.74~7.75 (3H, m), 7.35~7.51 (17H, m), 7.16 (1H, t) |
| 145 | δ = 8.55 (1H, d), 8.36 (4H, m), 8.13~8.19 (2H, m), 7.94~8.01 (4H, |
| | m), 7.73~7.75 (3H, m), 7.31~7.61 (16H, m), 7.16~7.20 (2H, m) |
| 148 | δ = 8.55 (1H, d), 8.45 (1H, d), 8.36 (4H, m), 8.13 (1H, d), |
| | 7.93~8.05 (5H, m), 7.31~7.60 (17H, m), 7.16 (1H, t) |
| 149 | δ = 8.55 (1H, d), 8.45 (1H, d), 8.36 (4H, m), 8.13 (1H, d), |
| | 7.93~8.01 (4H, m), 7.86 (1H, s), 7.78 (1H, s), 7.31~7.56 (16H, m), |
| | 7.16 (1H, t) |
| 151 | δ = 8.55 (2H, d), 8.36 (4H, m), 8.12~8.13 (2H, m), 7.94~8.01 (4H, |
| | m), 7.31~7.62 (21H, m), 7.16 (2H, t) |
| 152 | δ = 8.55 (1H, d), 8.36 (4H, m), 8.13 (1H, d), 7.94~8.01 (4H, m), |
| | 7.84 (1H, d), 7.31~7.51 (17H, m), 7.13~7.16 (2H, m) |
| 154 | δ = 8.55~8.56 (2H, m), 8.13 (1H, d), 7.94~8.01 (5H, m), 7.73~7.81 |
| | (5H, m), 7.28~7.62 (19H, m), 7.16 (1H, t) |
| 157 | δ = 8.55 (2H, d), 8.36~8.38 (3H, m), 8.13~8.19 (2H, m), 7.94~8.01 |
| | (5H, m), 7.31~7.73 (24H, m), 7.16~7.20 (3H, m) |
| 159 | δ = 8.55 (1H, d), 8.36~8.38 (5H, m), 8.13~8.19 (2H, m), 7.94~8.01 |
| | (4H, m), 7.73 (1H, t), 7.31~7.61 (17H, m), 7.16~7.20 (2H, m) |
| 161 | δ = 8.55 (1H, d), 8.36 (4H, m), 8.13~8.19 (2H, m), 7.79~7.98 (7H, |
| | m), 7.31~7.54 (14H, m), 7.16~7.20 (2H, m) |
| 162 | δ = 8.55 (1H, d), 8.45 (1H, d), 8.36 (4H, m), 8.12~8.19 (4H, m), |
| | 7.93~8.01 (5H, m), 7.46~7.58 (11H, m), 7.31~7.35 (2H, m), |
| | 7.16~7.20 (2H, m) |
| 164 | δ = 8.55 (1H, d), 8.45 (1H, d), 8.31~8.36 (5H, m), 8.12~8.13 (3H, |
| | m), 7.91~8.01 (6H, m), 7.74~7.75 (3H, m), 7.31~7.56 (14H, m), 7.16 |
| | (1H, t) |
| 165 | δ = 8.55 (1H, d), 8.45 (1H, d), 8.36 (4H, m), 8.13 (1H, d), |
| | 7.79~8.01 (9H, m), 7.31~7.60 (15H, m), 7.16 (1H, t) |
| 167 | δ = 8.55 (1H, d), 8.36 (4H, m), 8.13 (1H, d), 7.79~8.01 (9H, m), |
| | 7.79~8.01 (9H, m), 7.31~7.54 (16H, m), 7.16 (1H, t) |
| 168 | δ = 8.55 (1H, d), 8.45 (1H, d), 8.36 (4H, m), 8.13 (1H, d), |
| | 7.93~8.03 (7H, m), 7.76~7.82 (2H, m), 7.31~7.56 (15H, m), 7.16 |
| | (1H, t) |
| 169 | δ = 8.30~8.36 (5H, m), 8.13 (2H, d), 7.89~8.03 (7H, m), 7.75~7.82 |
| | (7H, m), 7.31~7.50 (17H, m) |
| 170 | δ = 8.55 (2H, d), 8.36 (4H, m), 8.12~8.13 (2H, m), 7.79~8.01 (8H, |
| | m), 7.31~7.62 (19H, m), 7.16 (2H, t) |
| 171 | δ = 8.55 (1H, d), 8.31~8.36 (5H, m), 7.79~8.13 (UH, m), 7.50~7.62 |
| | (13H, m), 7.31~7.39 (3H, m), 7.16 (1H, t) |
| 174 | δ = 8.55 (IH, d), 8.45 (1H, d), 8.13~8.19 (2H, m), 7.93~8.03 (6H, m), |
| | 7.81 (1H, d), 7.16~7.68 (17H, m) |
| 175 | δ = 9.53 (2H, s), 8.55 (IH, d), 8.13 (1H, d), 7.79~8.01 (9H, m), |
| | 7.31~7.54 (20H, m), 7.16 (IH, t) |
| 176 | δ = 8.55 (IH, d), 8.31~8.36 (3H, m), 7.74~8.13 (15H, m), 7.25~7.62 |
| | (18H, m), 7.16 (IH, t) |
| 177 | δ = 8.55 (IH, d), 8.36~8.45 (6H, m), 8.13~8.24 (5H, m), 7.93~8.01 |
| | (5H, m), 7.73 (IH, t), 7.46~7.61 (12H, m), 7.31~7.35 (2H, m), |
| | 7.16~7.20 (2H, m) |
| 179 | δ = 8.50~8.55 (2H, m), 8.43 (IH, d), 8.13 (IH, d), 7.75~8.03 (13H, |
| | m), 7.16~7.54 (11H, m) |
| 180 | δ = 8.55~8.56 (2H, d), 8.45 (IH, d), 8.13 (IH, d), 7.79~8.05 (9H, |
| | m), 7.28~7.62 (16H, m), 7.16 (IH, t) |
| 181 | δ = 8.55 (2H, d), 8.36 (4H, m), 8.19 (2H, d), 7.94~7.99 (5H, m), |
| | 7.46~7.58 (11H, m), 7.31~7.35 (3H, m), 7.16~7.20 (4H, m) |
| 182 | δ = 8.55 (2H, d), . 8.31~8.36 (5H, m), 8.19 (IH, d), 7.91~7.99 (6H, |
| | m), 7.74~7.75 (3H, m), 7.31~7.58 (15H, m), 7.16~7.20 (3H, m) |
| 183 | δ = 8.55 (2H, d), 8.31~8.36 (5H, m), 8.19 (IH, d), 7.91~7.99 (6H, |
| | m), 74~7.75 (3H, m), 7.31~7.58 (15H, m), 7.16~7.20 (3H, m) |
| 184 | δ = 8.55 (2H, d), 8.36 (4H, m), 8.19 (IH, d), 7.89~7.99 (7H, m), |
| | 7.75~7.77 (3H, m), 7.31~7.58 (15H, m), 7.16~7.20 (3H, m) |
| 186 | δ = 8.55 (2H, d), 8.45 (IH, d), 8.36 (4H, m), 8.19 (IH, d), |
| | 7.93~8.05 (7H, m),7.46~7.60 (12H, m),7.31~7.35 (3H, m), |
| | 7.16~7.20 (3H, m) |
| 187 | δ = 8.55 (2H, d), 8.36 (4H, m), 8.19 (IH, d), 7.94~7.99 (6H, m), |
| | 7.84 (IH, d), 7.31~7.54 (16H, m), 7.16~7.20 (3H, m) |
| 188 | δ = 8.55 (2H, d), 8.31~8.36 (5H, m), 8.19 (IH, d), 7.91~8.08 (7H, |
| | m), 7.74 (IH, s), 7.50~7.62 (14H, m), 7.35 (2H, t), 7.16~7.20 (3H, |
| | m) |
| 189 | δ = 8.55 (2H, d), 8.36 (4H, m), 8.19~8.24 (2H, m), 7.94~7.99 (6H, |
| | m), 7.74 (IH, d), 7.31~7.58 (15H, m), 7.16~7.20 (3H, m) |
| 191 | δ = 8.55 (IH, d), 8.36 (4H, m), 8.19 (IH, d), 8.12 (IH, d), |
| | 7.94~7.99 (6H, m), 7.46~7.62 (14H, m), 7.31~7.35 (4H, m), |
| | 7.16~7.20 (5H, m) |
| 192 | δ = 8.55 (IH, d), 8.30~8.36 (5H, m), 8.13~8.19 (4H, m), 7.89~8.01 |
| | (4H, m), 7.46~7.62 (16H, m), 7.31~7.35 (2H, m), 7.16~7.20 (3H, m) |
| 193 | δ = 8.55 (IH, d), 8.30~8.36 (5H, m), 7.89~8.19 (10H, m), 7.77 (IH, |
| | d), 7.50~7.62 (19H, m), 7.35 (IH, t), 7.16~7.20 (2H, m) |
| 196 | δ = 8.55~8.56 (3H, m), 8.45 (IH, d), 8.19 (IH, d), 7.78~7.99 (9H, |
| | m), 7.16~7.62 (18H, m) |
| 198 | δ = 8.55 (2H, d), 8.31~8.38 (6H, m), 8.19 (1H, d), 7.91~7.99 (7H, |
| | m), 7.73~7.75 (4H, m), 7.35~7.61 (16H, m), 7.16~7.20 (3H, m) |
| 199 | δ = 8.55 (2H, d), 8.36 (2H, m), 8.19~8.24 (2H, m), 7.88~7.99 (8H, |
| | m), 7.74~7.75 (3H, m), 7.16~7.58 (22H, m), 1.69 (6H, s) |
| 200 | δ = 8.55 (2H, d), 8.36~8.38 (3H, m), 8.19 (2H, d), 7.94~7.99 (6H, |
| | m), 7.73~7.75 (3H, m), 7.31~7.61 (15H, m), 7.16~7.20 (4H, m) |
| 227 | δ = 8.55 (1H, d), 8.36~8.45 (6H, m), 8.05~8.13 (3H, m), 7.93~7.94 |
| | (2H, d), 7.35~7.60 (17H, m), 7.16 (1H, t) |
| 231 | δ = 8.55 (1H, d), 8.36~8.41 (5H, m), 8.11~8.13 (2H, m), 7.94~7.98 |
| | (2H, m), 7.84 (1H, d), 7.65 (1H, t), 7.35~7.54 (16H, m), 7.13~7.16 |
| | (2H, m) |
| 2-23 | δ = 8.55 (1H, d), 8.30 (1H, d), 8.13~8.19 (2H, m), 7.91~7.99 (10H, |
| | m), 7.75~7.80 (4H, m), 7.35~7.58 (8H, m), 7.16~7.25 (10H, m) |
| 2-32 | δ = 8.55 (1H, d), 8.30 (1H, d), 8.13~8.21 (3H, m), 7.89~7.99 (8H, |
| | m), 7.35~7.77 (17H, m), 7.16~7.20 (2H, m) |
| 2-33 | δ = 8.55 (1H, d), 8.30 (1H, d), 8.13~8.21 (3H, m), 7.89~7.94 (4H, |
| | m), 7.35~7.77 (20H, m), 7.16~7.25 (6H, m) |
| 2-34 | δ = 8.55 (IH, d), 8.30 (1H, d), 8.13~8.21 (3H, m), 7.89~7.99 (8H, |
| | m), 7.35~7.77 (17H, m), 7.16~7.25 (6H, m) |
| 2-42 | δ = 8.55 (IH, d), 8.30 (1H, d), 8.13~8.19 (2H, m), 7.91~7.99 (12H, |
| | m), 7.75~7.77 (5H, m), 7.58 (IH, d), 7.35~7.50 (8H, m), 7.16~7.20 |
| | (2H, m) |
| 2-44 | δ = 8.55 (IH, d), 8.30 (IH, d), 8.13~8.19 (2H, m), 7.89~7.99 (12H, |
| | m), 7.75~7.77 (5H, m), 7.41~7.50 (8H, m), 7.16~7.25 (6H, m) |

### <Experimental Example 1>—Manufacture of Organic Light Emitting Device

1) Manufacture of Organic Light Emitting Device

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine) and a hole transport layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. The light emitting layer was deposited to 400 Å using a compound described in the following Table 10 as a host and Ir(ppy)₃ (tris(2-phenylpyridine)iridium) as a green phosphorescent dopant by doping the Ir(ppy)₃ to the host in a weight ratio of 7%. After that, BCP was deposited to 60 Å as a hole blocking layer, and Alq₃ was deposited to 200 Å thereon as an electron transport layer. Lastly, an electron injection layer was formed on the electron transport layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁸ torr to 10⁻⁶ torr for each material to be used in the OLED manufacture.

2) Driving Voltage and Light Emission Efficiency of Organic Electroluminescent Device

For each of the organic electroluminescent devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Properties of the organic electroluminescent devices of the present disclosure are as shown in Table 10.

TABLE 10

| | | Driving | Efficiency | Color Coordinate | Lifetime |
|---|---|---|---|---|---|
| | Compound | Voltage (V) | (cd/A) | (x, y) | (T₉₀) |
| Comparative | A | 5.39 | 53.2 | (0.265, 0.672) | 61 |
| Example 1 | | | | | |
| Comparative | B | 5.53 | 49.5 | (0.274, 0.684) | 50 |
| Example 2 | | | | | |
| Comparative | C | 5.62 | 47.2 | (0.276, 0.681) | 53 |
| Example 3 | | | | | |
| Comparative | D | 5.65 | 50.1 | (0.281, 0.675) | 56 |
| Example 4 | | | | | |
| Comparative | E | 5.59 | 52.0 | (0.275, 0.670) | 51 |
| Example 5 | | | | | |
| Comparative | F | 5.10 | 57.0 | (0.279, 0.671) | 69 |
| Example 6 | | | | | |
| Comparative | G | 5.30 | 56.5 | (0.280, 0.665) | 65 |
| Example 7 | | | | | |
| Comparative | H | 5.43 | 48.2 | (0.274, 0.677) | 51 |
| Example 8 | | | | | |
| Comparative | I | 6.79 | 34.0 | (0.286, 0.685) | 40 |
| Example 9 | | | | | |
| Comparative | J | 6.63 | 35.7 | (0.273, 0.679) | 43 |
| Example 10 | | | | | |
| Example 1 | 1 | 4.51 | 78.9 | (0.278, 0.680) | 200 |
| Example 2 | 2 | 4.53 | 76.2 | (0.275, 0.683) | 194 |
| Example 3 | 3 | 4.80 | 79.3 | (0.271, 0.684) | 193 |
| Example 4 | 4 | 4.73 | 76.5 | (0.275, 0.683) | 160 |
| Example 5 | 6 | 4.55 | 79.2 | (0.274, 0.664) | 158 |
| Example 6 | 9 | 4.65 | 78.3 | (0.285, 0.674) | 169 |
| Example 7 | 11 | 4.93 | 75.9 | (0.280, 0.677) | 163 |
| Example 8 | 13 | 4.95 | 77.9 | (0.279, 0.675) | 159 |
| Example 9 | 16 | 4.78 | 78.5 | (0.271, 0.676) | 177 |
| Example 10 | 20 | 4.66 | 77.9 | (0.273, 0.671) | 186 |
| Example 11 | 21 | 4.89 | 75.9 | (0.270, 0.675) | 182 |
| Example 12 | 23 | 5.03 | 78.6 | (0.274, 0.679) | 176 |
| Example 13 | 24 | 4.59 | 76.1 | (0.287, 0.672) | 177 |
| Example 14 | 26 | 4.53 | 76.9 | (0.273, 0.675) | 187 |
| Example 15 | 28 | 4.50 | 77.1 | (0.288, 0.680) | 172 |
| Example 16 | 30 | 4.69 | 76.2 | (0.284, 0.681) | 163 |
| Example 17 | 37 | 5.02 | 80.0 | (0.274, 0.675) | 159 |
| Example 18 | 39 | 5.10 | 75.0 | (0.271, 0.677) | 150 |
| Example 19 | 41 | 4.01 | 82.7 | (0.276, 0.679) | 241 |
| Example 20 | 42 | 4.20 | 83.6 | (0.270, 0.664) | 244 |
| Example 21 | 43 | 4.16 | 84.7 | (0.273, 0.677) | 245 |
| Example 22 | 45 | 4.04 | 82.7 | (0.284, 0.680) | 235 |
| Example 23 | 48 | 4.06 | 84.4 | (0.281, 0.682) | 230 |
| Example 24 | 49 | 4.49 | 83.9 | (0.275, 0.680) | 244 |
| Example 25 | 51 | 4.37 | 83.6 | (0.281, 0.660) | 239 |
| Example 26 | 54 | 4.24 | 82.4 | (0.271, 0.675) | 248 |
| Example 27 | 59 | 4.13 | 85.0 | (0.275, 0.680) | 250 |
| Example 28 | 61 | 3.52 | 94.6 | (0.280, 0.682) | 274 |
| Example 29 | 62 | 3.59 | 93.8 | (0.274, 0.682) | 300 |
| Example 30 | 63 | 3.61 | 93.4 | (0.270, 0.675) | 294 |
| Example 31 | 65 | 3.51 | 92.8 | (0.275, 0.670) | 293 |
| Example 32 | 67 | 3.60 | 94.8 | (0.279, 0.674) | 285 |
| Example 33 | 68 | 3.58 | 94.2 | (0.270, 0.677) | 276 |
| Example 34 | 70 | 3.67 | 93.6 | (0.283, 0.675) | 283 |
| Example 35 | 71 | 3.66 | 93.0 | (0.273, 0.671) | 276 |
| Example 36 | 74 | 3.59 | 92.7 | (0.274, 0.665) | 286 |
| Example 37 | 78 | 3.55 | 94.7 | (0.271, 0.660) | 277 |
| Example 38 | 80 | 3.50 | 93.8 | (0.284, 0.681) | 300 |
| Example 39 | 81 | 3.04 | 94.7 | (0.280, 0.669) | 319 |
| Example 40 | 82 | 3.20 | 94.8 | (0.271, 0.672) | 320 |
| Example 41 | 85 | 3.25 | 92.2 | (0.285, 0.680) | 311 |
| Example 42 | 86 | 3.09 | 95.0 | (0.270, 0.679) | 318 |
| Example 43 | 88 | 3.11 | 94.2 | (0.278, 0.683) | 315 |
| Example 44 | 91 | 3.06 | 94.4 | (0.270, 0.683) | 319 |
| Example 45 | 92 | 3.12 | 93.7 | (0.278, 0.681) | 321 |
| Example 46 | 93 | 3.05 | 92.6 | (0.270, 0.683) | 318 |
| Example 47 | 96 | 3.41 | 92.8 | (0.273, 0.660) | 315 |
| Example 48 | 98 | 3.48 | 94.5 | (0.275, 0.664) | 312 |
| Example 49 | 100 | 3.02 | 95.0 | (0.284, 0.660) | 320 |
| Example 50 | 101 | 4.87 | 73.8 | (0.270, 0.677) | 148 |
| Example 51 | 102 | 4.66 | 73.5 | (0.285, 0.685) | 147 |
| Example 52 | 103 | 4.63 | 74.1 | (0.273, 0.679) | 142 |
| Example 53 | 104 | 4.56 | 75.0 | (0.273, 0.682) | 131 |
| Example 54 | 107 | 4.58 | 73.7 | (0.275, 0.683) | 122 |
| Example 55 | 109 | 4.57 | 74.2 | (0.277, 0.685) | 128 |
| Example 56 | 111 | 4.91 | 70.8 | (0.275, 0.683) | 135 |
| Example 57 | 113 | 4.58 | 71.9 | (0.275, 0.675) | 104 |
| Example 58 | 118 | 4.56 | 73.0 | (0.287, 0.681) | 108 |
| Example 59 | 119 | 4.57 | 74.8 | (0.284, 0.683) | 150 |
| Example 60 | 121 | 4.43 | 72.2 | (0.275, 0.675) | 134 |
| Example 61 | 122 | 4.50 | 70.9 | (0.271, 0.670) | 128 |
| Example 62 | 123 | 4.56 | 71.2 | (0.275, 0.664) | 123 |
| Example 63 | 124 | 4.76 | 74.7 | (0.274, 0.670) | 145 |
| Example 64 | 125 | 4.82 | 73.5 | (0.277, 0.675) | 110 |
| Example 65 | 126 | 4.78 | 70.9 | (0.280, 0.683) | 148 |
| Example 66 | 130 | 4.55 | 71.3 | (0.271, 0.670) | 125 |
| Example 67 | 136 | 4.89 | 74.2 | (0.273, 0.682) | 143 |
| Example 68 | 137 | 5.04 | 72.2 | (0.272, 0.679) | 150 |
| Example 69 | 139 | 5.08 | 75.0 | (0.270, 0.662) | 223 |
| Example 70 | 141 | 4.28 | 86.3 | (0.273, 0.677) | 219 |
| Example 71 | 142 | 4.06 | 86.9 | (0.280, 0.682) | 225 |
| Example 72 | 143 | 4.12 | 87.5 | (0.281, 0.685) | 223 |
| Example 73 | 145 | 4.17 | 87.9 | (0.273, 0.680) | 217 |
| Example 74 | 148 | 4.05 | 85.6 | (0.274, 0.682) | 224 |
| Example 75 | 149 | 4.09 | 86.7 | (0.271, 0.680) | 210 |
| Example 76 | 151 | 4.14 | 87.4 | (0.275, 0.685) | 217 |
| Example 77 | 152 | 4.16 | 85.9 | (0.274, 0.683) | 229 |
| Example 78 | 154 | 4.02 | 85.2 | (0.275, 0.670) | 215 |
| Example 79 | 157 | 4.43 | 86.7 | (0.283, 0.680) | 225 |
| Example 80 | 159 | 4.00 | 87.3 | (0.270, 0.672) | 230 |
| Example 81 | 161 | 3.80 | 87.9 | (0.270, 0.683) | 259 |
| Example 82 | 162 | 3.67 | 85.3 | (0.270, 0.685) | 260 |
| Example 83 | 164 | 3.68 | 85.5 | (0.277, 0.680) | 263 |
| Example 84 | 165 | 3.90 | 87.7 | (0.284, 0.681) | 257 |
| Example 85 | 167 | 3.78 | 86.3 | (0.280, 0.660) | 269 |
| Example 86 | 168 | 3.68 | 85.9 | (0.274, 0.672) | 263 |
| Example 87 | 169 | 3.55 | 86.2 | (0.273, 0.679) | 255 |
| Example 88 | 170 | 3.51 | 87.4 | (0.280, 0.675) | 253 |
| Example 89 | 171 | 3.57 | 85.8 | (0.271, 0.670) | 261 |
| Example 90 | 174 | 3.53 | 86.0 | (0.277, 0.670) | 268 |
| Example 91 | 175 | 3.59 | 86.6 | (0.273, 0.673) | 269 |
| Example 92 | 176 | 3.55 | 87.9 | (0.271, 0.685) | 257 |
| Example 93 | 177 | 3.47 | 85.6 | (0.274, 0.680) | 259 |
| Example 94 | 179 | 3.59 | 87.5 | (0.275, 0.673) | 264 |
| Example 95 | 180 | 3.55 | 88.0 | (0.283, 0.687) | 270 |
| Example 96 | 181 | 3.41 | 91.4 | (0.270, 0.670) | 317 |
| Example 97 | 182 | 3.28 | 90.7 | (0.275, 0.683) | 315 |
| Example 98 | 183 | 3.22 | 90.5 | (0.271, 0.675) | 301 |
| Example 99 | 184 | 3.14 | 91.7 | (0.272, 0.680) | 318 |
| Example 100 | 186 | 3.02 | 90.2 | (0.275, 0.672) | 308 |
| Example 101 | 187 | 3.50 | 91.7 | (0.277, 0.664) | 305 |
| Example 102 | 188 | 3.41 | 92.0 | (0.284, 0.663) | 311 |
| Example 103 | 189 | 3.29 | 90.7 | (0.275, 0.677) | 312 |
| Example 104 | 191 | 3.18 | 91.3 | (0.280, 0.685) | 302 |
| Example 105 | 192 | 3.28 | 90.5 | (0.274, 0.664) | 315 |
| Example 106 | 193 | 3.22 | 90.9 | (0.285, 0.670) | 308 |
| Example 107 | 196 | 3.15 | 91.0 | (0.282, 0.675) | 301 |
| Example 108 | 198 | 3.08 | 91.7 | (0.279, 0.668) | 300 |
| Example 109 | 199 | 3.45 | 92.0 | (0.276, 0.679) | 309 |
| Example 110 | 200 | 3.50 | 90.5 | (0.273, 0.661) | 310 |
| Example 111 | 227 | 3.02 | 96.7 | (0.275, 0.668) | 233 |
| Example 112 | 231 | 3.28 | 95.8 | (0.272, 0.665) | 245 |

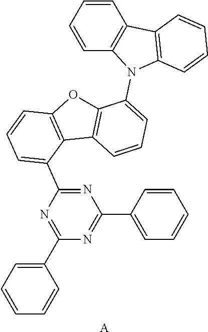

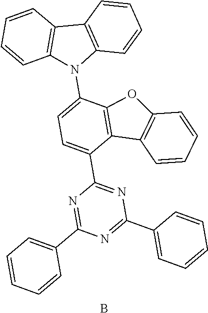

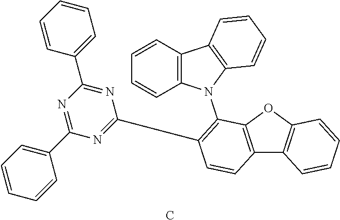

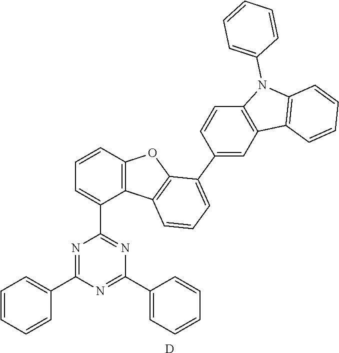

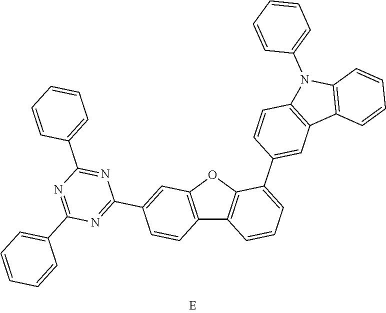 [SMILES: c1ccc(-c2nc(-c3ccccc3)nc(-c3ccc4c(c3)oc3c(-c5ccc6c(c5)c5ccccc5n6-c5ccccc5)cccc34)n2)cc1]

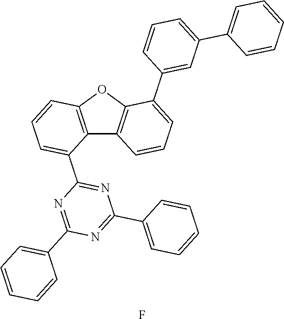

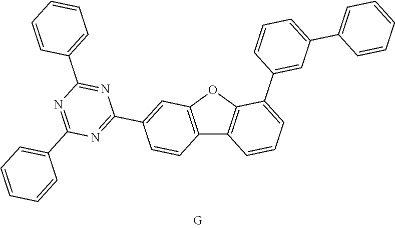 [SMILES: c1ccc(-c2cccc(-c3cccc4c3oc3cc(-c5nc(-c6ccccc6)nc(-c6ccccc6)n5)ccc34)c2)cc1]

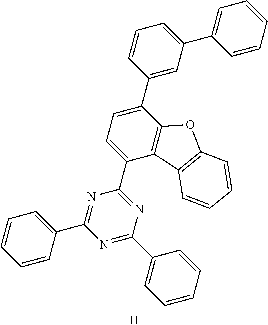

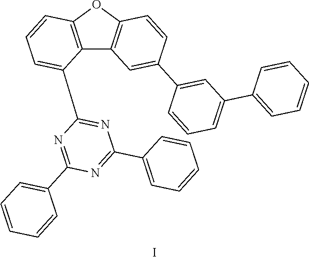

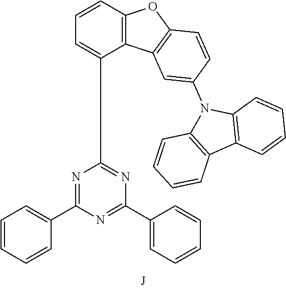 [SMILES: c1ccc(-c2nc(-c3ccccc3)nc(-c3cccc4oc5ccc(-n6c7ccccc7c7ccccc76)cc5c34)n2)cc1]

### <Experimental Example 2>—Manufacture of Organic Light Emitting Device

A glass substrate on which ITO was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine) and a hole transport layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. As the light emitting layer, one type of the compound described in Chemical Formula 1 and one type of the compound described in Chemical Formula A (light emitting layer compound of Table 11) were pre-mixed and deposited to 400 Å in one source of supply as a host, and, as a green phosphorescent dopant, Ir(ppy)₃ was doped and deposited thereto by 7% with respect to the deposited thickness of the light emitting layer. After that, BCP was deposited to 60 Å as a hole blocking layer, and Alq₃ was deposited to 200 Å thereon as an electron transport layer. Lastly, an electron injection layer was formed on the electron transport layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁸ torr to 10⁻⁶ torr for each material to be used in the OLED manufacture.

For each of the organic electroluminescent devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc.

Results of measuring driving voltage, light emission efficiency, color coordinate (CIE) and lifetime of the organic light emitting devices manufactured according to the present disclosure are as shown in the following Table 11.

TABLE 11

| | Light | | | | | |
|---|---|---|---|---|---|---|
| | Emitting | | | | | |
| | Layer | | Driving | Effi- | Color | Life- |
| | Com- | | Voltage | ciency | Coordinate | time |
| | pound | Ratio | (V) | (cd/A) | (x, y) | (T₉₀) |
| Example 113 | 1:2-33 | 1:2 | 3.24 | 85.6 | (0.244, 0.660) | 320 |
| Example 114 | | 1:1 | 3.22 | 86.0 | (0.240, 0.645) | 322 |
| Example 115 | | 2:1 | 3.21 | 86.2 | (0.281, 0.679) | 330 |
| Example 116 | 21:2-34 | 1:2 | 3.76 | 83.7 | (0.257, 0.614) | 293 |
| Example 117 | | 1:1 | 3.75 | 84.5 | (0.248, 0.654) | 295 |
| Example 118 | | 2:1 | 3.73 | 84.9 | (0.253, 0.702) | 299 |
| Example 119 | 41:2-32 | 1:2 | 3.56 | 87.9 | (0.253, 0.724) | 380 |
| Example 120 | | 1:1 | 3.55 | 89.3 | (0.241, 0.623) | 383 |
| Example 121 | | 2:1 | 3.54 | 89.5 | (0.254, 0.620) | 389 |
| Example 123 | 61:2-33 | 1:2 | 3.15 | 106.8 | (0.240, 0.754) | 399 |
| Example 124 | | 1:1 | 3.14 | 109.2 | (0.245, 0.657) | 402 |
| Example 125 | | 2:1 | 3.12 | 110.1 | (0.250, 0.731) | 408 |
| Example 126 | 81:2-33 | 1:2 | 2.62 | 110.2 | (0.243, 0.705) | 421 |
| Example 127 | | 1:1 | 2.59 | 112.5 | (0.230, 0.741) | 426 |
| Example 128 | | 2:1 | 2.58 | 112.8 | (0.235, 0.714) | 430 |
| Example 129 | 227:2-34 | 1:2 | 2.68 | 126.5 | (0.241, 0.702) | 378 |
| Example 130 | | 1:1 | 2.67 | 128.2 | (0.232, 0.625) | 385 |
| Example 131 | | 2:1 | 2.65 | 128.5 | (0.246, 0.710) | 386 |
| Example 132 | 231:2-34 | 1:2 | 3.04 | 128.3 | (0.245, 0.713) | 373 |
| Example 133 | | 1:1 | 3.03 | 129.8 | (0.235, 0.710) | 375 |
| Example 134 | | 2:1 | 3.01 | 130.2 | (0.250, 0.618) | 380 |

As seen from Table 10, the heterocyclic compound according to the present application has each of substituents of Ar1 and Ar2 at a No. 4 position of each benzene ring of dibenzofuran, and it was identified that, by increasing thermal stability through blocking an electronically weak position of the dibenzofuran with the substituent, the organic light emitting device comprising the same had properties of a particularly improved lifetime.

In other words, dibenzofuran and dibenzothiophene have an oxygen atom and a sulfur atom with higher electronegativity than carbon at the center. Within the molecular structure, the oxygen atom and the sulfur atom tend to attract electrons of adjacent carbons. Accordingly, a No. 4 position of each benzene ring of dibenzofuran and dibenzothiophene is relatively weak position-wise due to insufficient electrons compared to other positions. The purpose of the present disclosure is to increase lifetime and efficiency by attaching an aryl group, a heteroaryl group and the like capable of supplying electrons to such an electron-deficient position.

In Table 10, it was identified that, for some of the experimental results, there was a tendency depending on the bonding position of N-Het, an ET unit, in each of the compounds. When N-Het bonds to a No. 3 position of the dibenzofuran core, steric hinderance of the molecule itself increases compared to when N-Het bonds to a No. 1 position of the dibenzofuran core. Accordingly, structural stability is much higher when N-Het bonds to a No. 1 position of the dibenzofuran core. In addition, when a material is deposited to make a device, stability for packing structure of the material increases as well. For such a reason, lifetime and efficiency tend to be more superior when N-Het bonds to a No. 1 position of the dibenzofuran. On the other hand, it was seen that the driving voltage was similar.

Specifically, in Table 10, Compounds 1 to 20 and 101 to 120 of the specific examples in which a No. 4 position of each benzene ring of the dibenzofuran is all attached by an aryl group as a substituent and Compounds 21 to 40 and 121 to 140 of the specific examples in which a No. 4 position of each benzene ring of the dibenzofuran is attached by an aryl group and a heteroaryl group other than carbazole may be compared as follows.

When a No. 4 position of each benzene ring of the dibenzofuran is all attached by an aryl group as a substituent, the molecular structure is unipolar. On the other hand, the molecule is bipolar when one side is attached by an aryl group and the other side is attached by a heteroaryl group, and electrons are better balanced and charge transfer more favorably occurs. However, when a No. 4 position of each benzene ring of the dibenzofuran is attached by an aryl group and a heteroaryl group other than carbazole, the degree of giving electrons to the electron-deficient position of the dibenzofuran is similar, and driving voltage, efficiency and lifetime were identified to be similar.

In addition, when using an aryl group and a carbazole group having a stronger tendency to give electrons than an aryl group as a substituent at a No. 4 position of each benzene ring of the dibenzofuran, electrons become more abundant in the molecule, and a more stable structure is obtained. Accordingly, it was identified that Compounds 41 to 80 and 141 to 180 of the specific examples of Table 9 were far more superior in driving voltage, efficiency and lifetime compared to Compounds 1 to 40 and 101 to 140 of the specific examples. For the same reason, the effect is maximized when a No. 4 position of each benzene ring of the dibenzofuran is all attached by a carbazole group, and it was identified that the driving voltage was lowered, and the lifetime and the efficiency were enhanced.

In addition, from the results of Table 11, it was identified that effects of more superior efficiency and lifetime were obtained when comprising the compound of Chemical Formula 1 and the compound of Chemical Formula A at the same time. Such a result may lead to a forecast that an exciplex phenomenon occurs when comprising the two compounds at the same time.

The exciplex phenomenon is a phenomenon of releasing energy having sizes of a donor (p-host) HOMO level and an acceptor (n-host) LUMO level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transport ability and an acceptor (n-host) having a favorable electron transport ability are used as a host of a light emitting layer, holes are injected to the p-host and electrons are injected to the n-host, and therefore, a driving voltage may be lowered, which resultantly helps with enhancement in the lifetime. In the disclosure of the present application, it was identified that superior device properties were obtained when the compound of Chemical Formula A having a donor role and the compound of Chemical Formula 1 having an acceptor role were used as a host of the light emitting layer.

1. A heterocyclic compound represented by the following Chemical Formula 1: 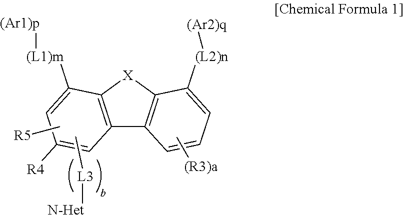
wherein, in Chemical Formula 1,
X is O; or S;
N-Het is a monocyclic or polycyclic heterocyclic group substituted or unsubstituted, and comprising one or more Ns;
Ar1 and Ar2 are the same as or different from each other, and each independently a substituted or unsubstituted C1 to C60 alkyl group; a substituted or unsubstituted C6 to C60 aryl group; a substituted or unsubstituted C2 to C60 heteroaryl group; —P(═O)RR′; or —SiRR′R″;
R3 to R5 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; —SiRR′R″ and —NRR′, or two or more groups adjacent to each other bond to each other to form a substituted or unsubstituted C6 to C60 aliphatic or aromatic hydrocarbon ring or a substituted or unsubstituted C2 to C60 aliphatic or aromatic heteroring;
L1 to L3 are the same as or different from each other, and each independently a direct bond; a substituted or unsubstituted C6 to C60 arylene group; or a substituted or unsubstituted C2 to C60 heteroarylene group;
R, R′ and R″ are the same as or different from each other, and each independently a substituted or unsubstituted C1 to C60 alkyl group; a substituted or unsubstituted C6 to C60 aryl group; or a substituted or unsubstituted C2 to C60 heteroaryl group;
p and q are an integer of 1 to 4;
b, m and n are an integer of 0 to 4;
a is an integer of 0 to 3; and
when p, q, a, b, m and n are 2 or greater, substituents in the parentheses are the same as or different from each other.

2. The heterocyclic compound of claim 1, wherein Chemical Formula 1 is represented by the following Chemical Formula 2 or 3: 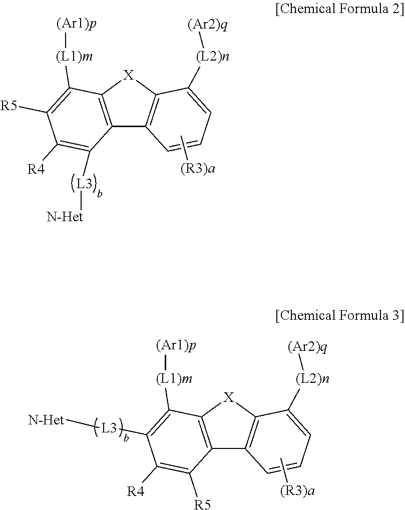
in Chemical Formulae 2 and 3,
X, R3 to R5, N-Het, Ar1, Ar2, L1 to L3, a, b, m, n, p and q have the same definitions as in Chemical Formula 1.

3. The heterocyclic compound of claim 1, wherein Chemical Formula 1 is represented by any one of the following Chemical Formula 4 to Chemical Formula 10: 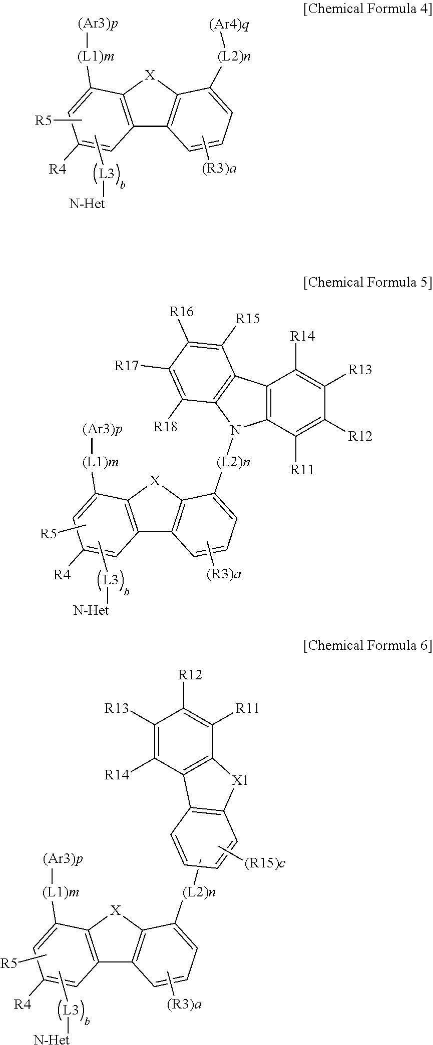 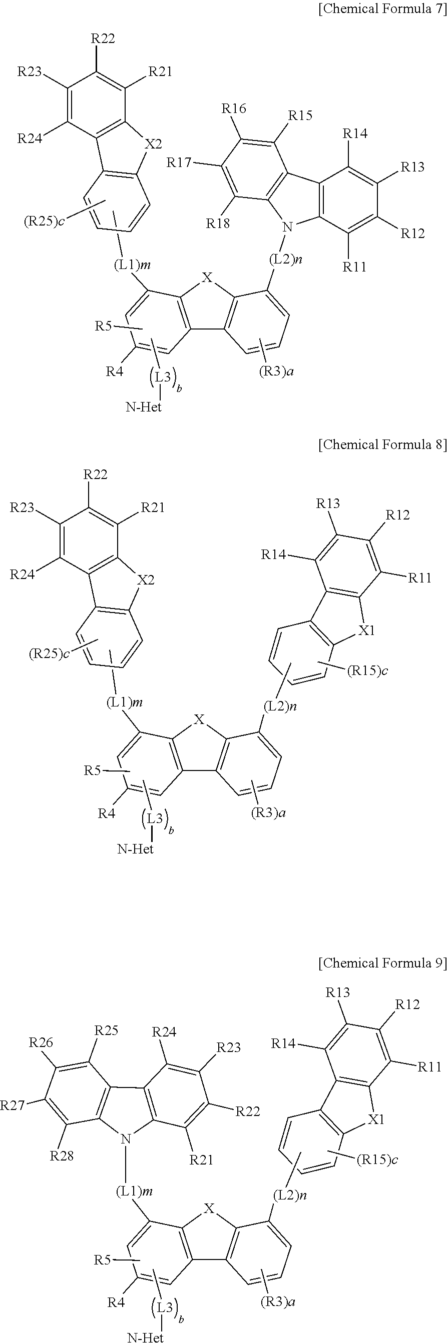 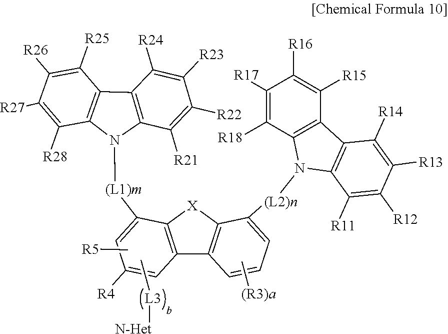
in Chemical Formulae 4 to 10,
X, N-Het, R3 to R5, L1 to L3, m, n, p, q, a and b have the same definitions as in Chemical Formula 1;
X1 and X2 are the same as or different from each other, and each independently O; S; or NR31;
Ar3 and Ar4 are the same as or different from each other, and each independently a substituted or unsubstituted C6 to C60 aryl group;
R11 to R18 and R21 to R28 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; —SiRR′R″ and —NRR′, or two or more groups adjacent to each other bond to each other to form a substituted or unsubstituted C6 to C60 aliphatic or aromatic hydrocarbon ring or a substituted or unsubstituted C2 to C60 aliphatic or aromatic heteroring;
R31, R, R′ and R″ are the same as or different from each other, and each independently a substituted or unsubstituted C1 to C60 alkyl group; a substituted or unsubstituted C6 to C60 aryl group; or a substituted or unsubstituted C2 to C60 heteroaryl group; and
c is an integer of 0 to 3, and when c is 2 or greater, substituents in the parentheses are the same as or different from each other.

4. The heterocyclic compound claim 1, wherein N-Het is a substituted or unsubstituted triazine group; a substituted or unsubstituted pyrimidine group; a substituted or unsubstituted pyridine group; a substituted or unsubstituted quinoline group; a substituted or unsubstituted 1,10-phenanthroline group; a substituted or unsubstituted 1,7-phenanthroline group; a substituted or unsubstituted quinazoline group; substituted or unsubstituted pyrido[3,2-d]pyrimidine; or a substituted or unsubstituted benzimidazole group.

5. The heterocyclic compound of claim 1, wherein R3 to R5 are hydrogen; or deuterium.

6. The heterocyclic compound of claim 1, wherein Chemical Formula 1 is represented by any one of the following compounds:

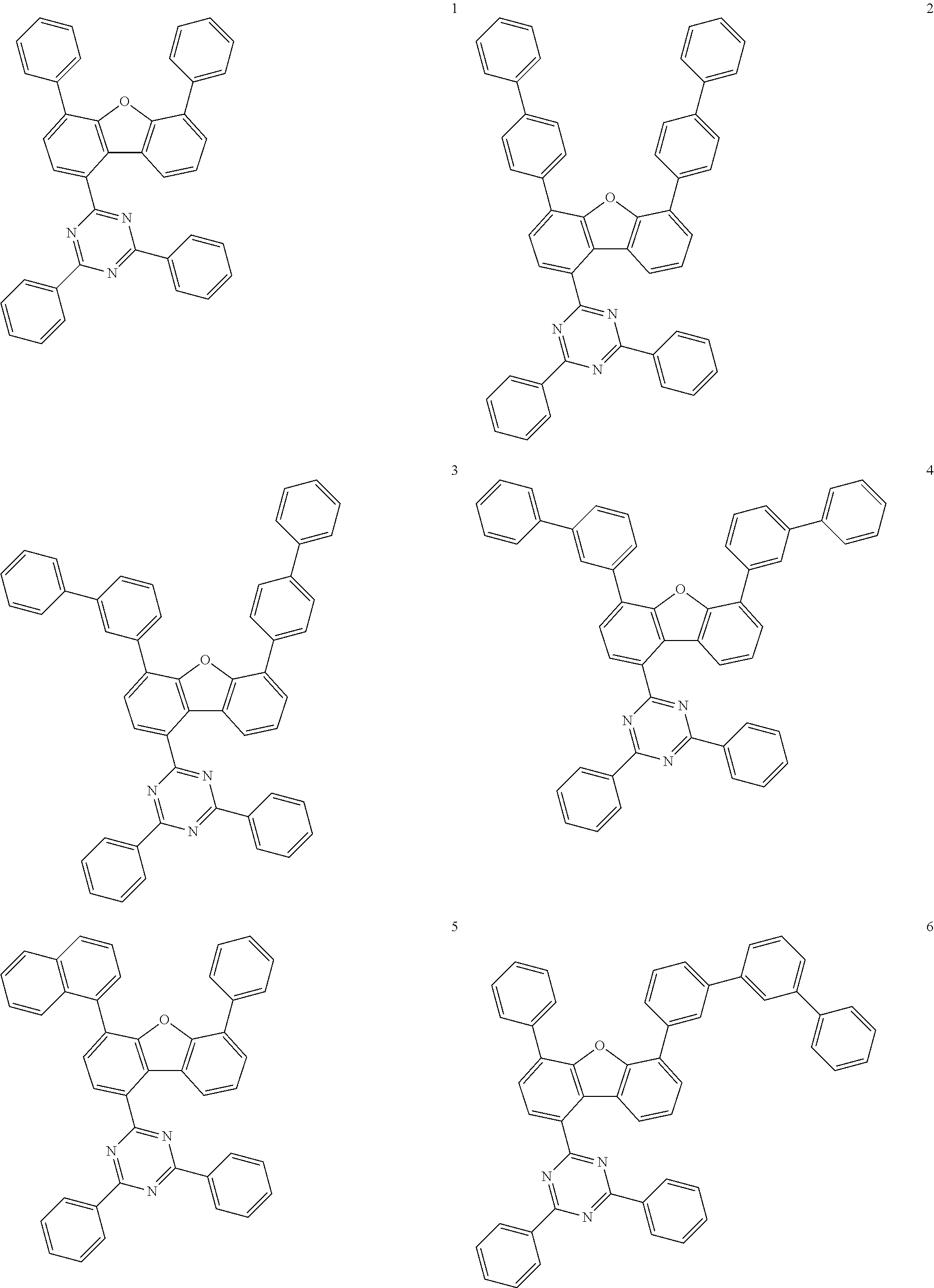

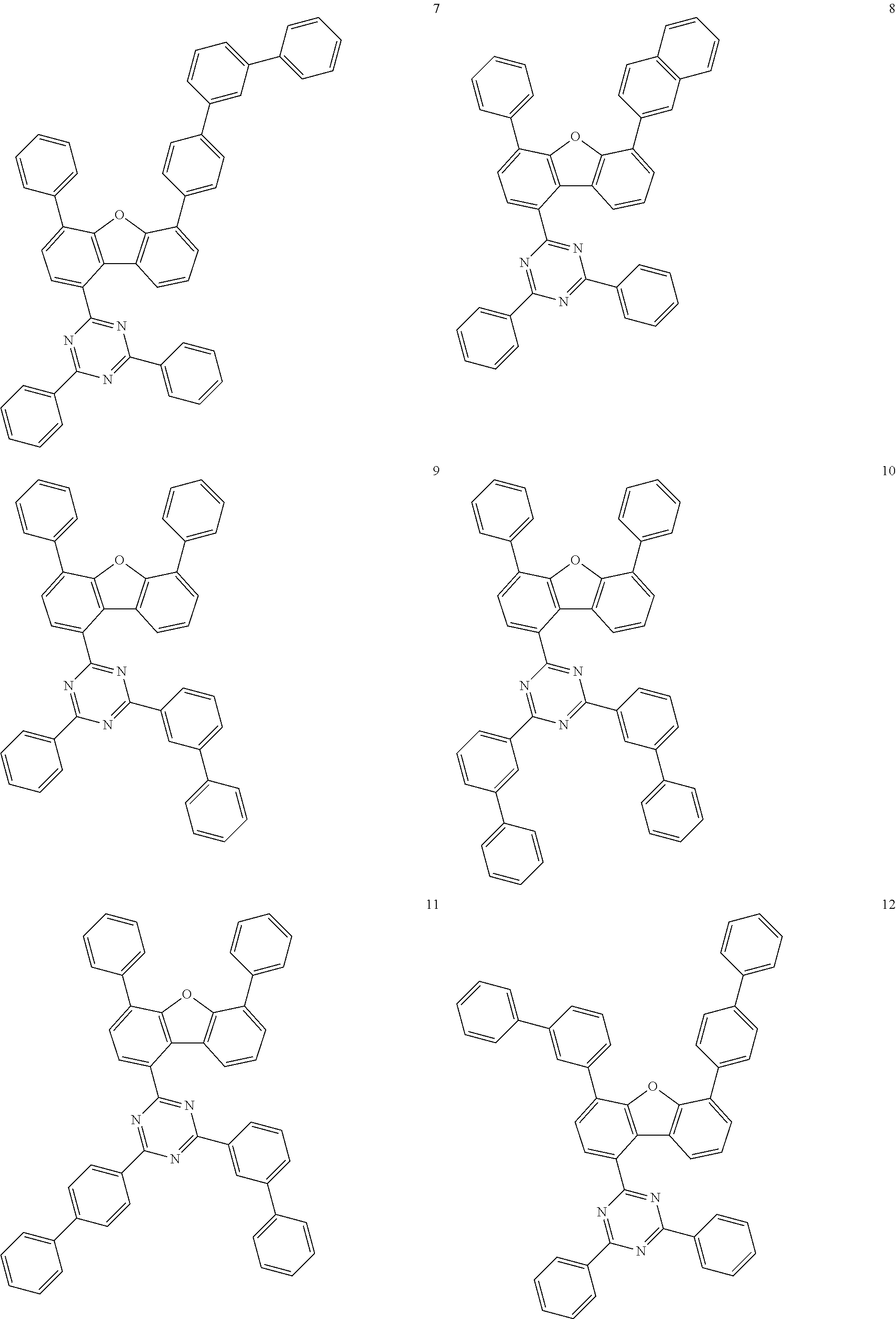

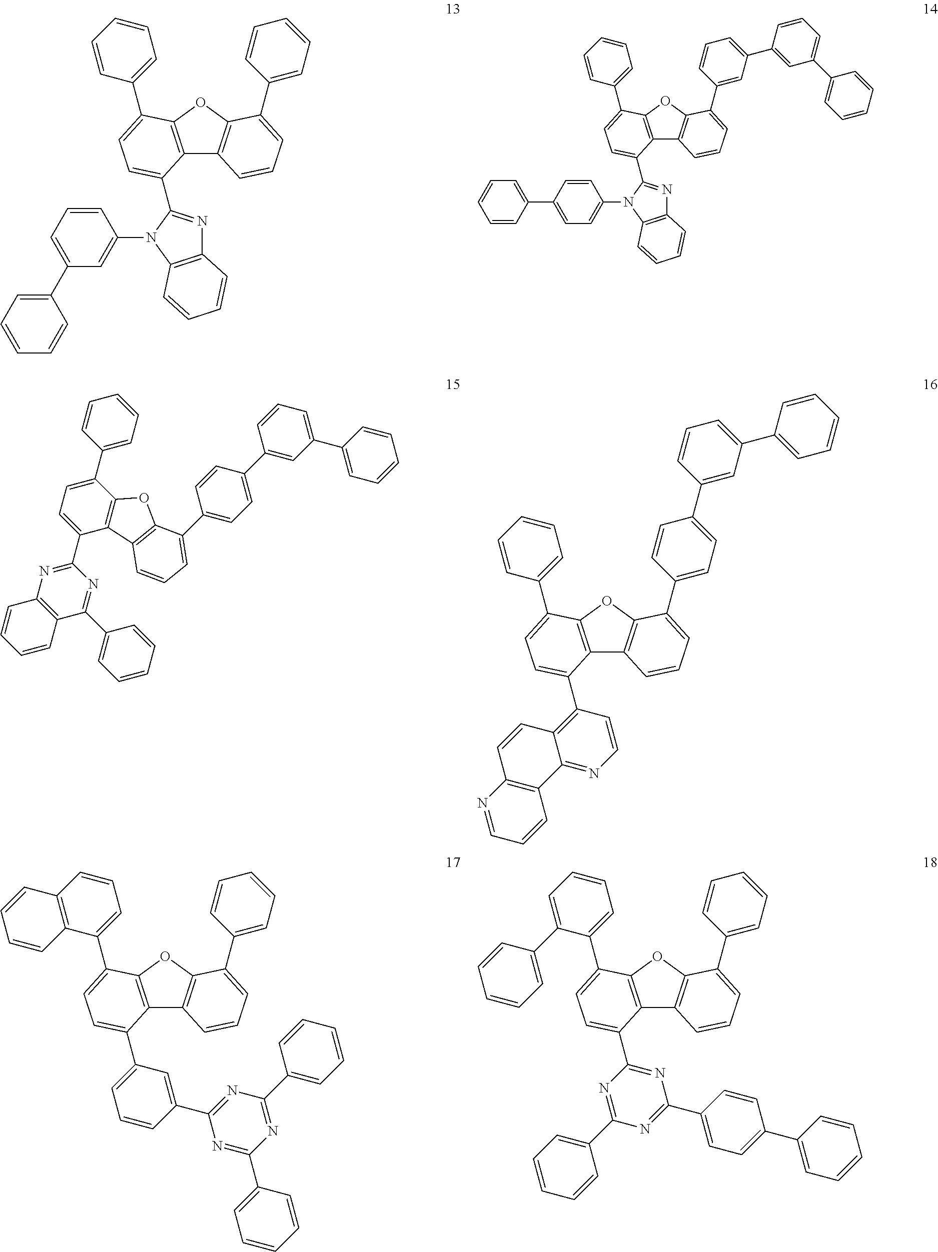

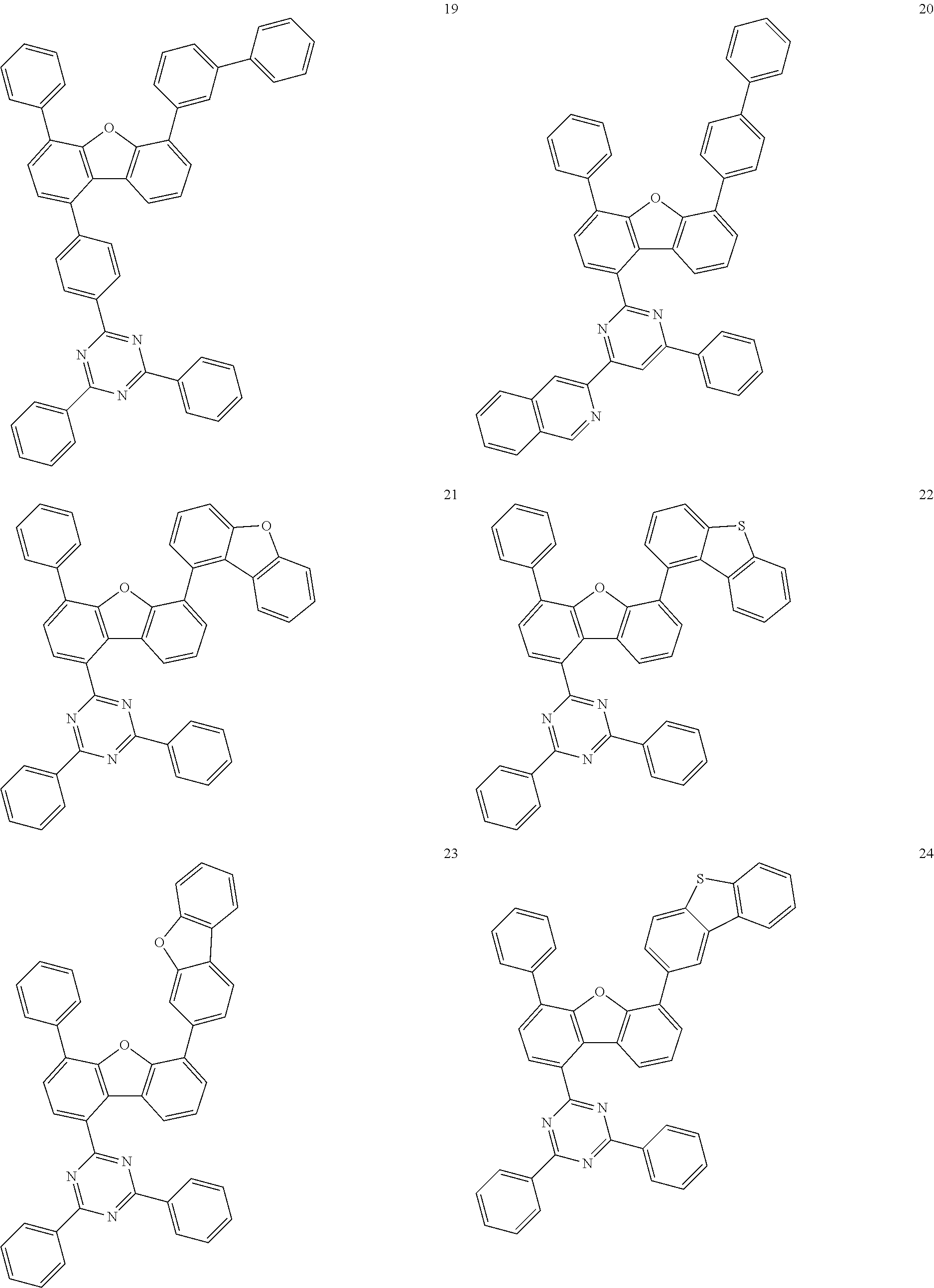

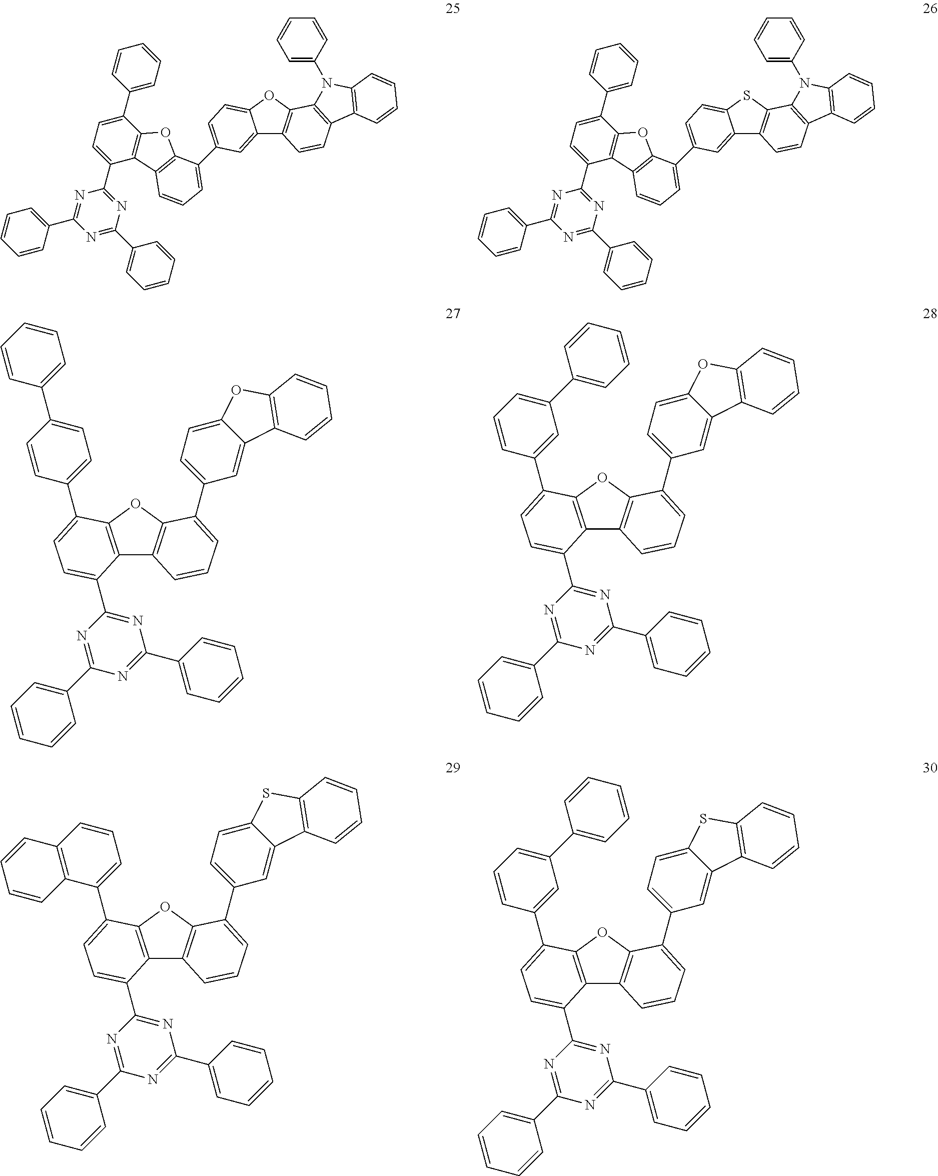

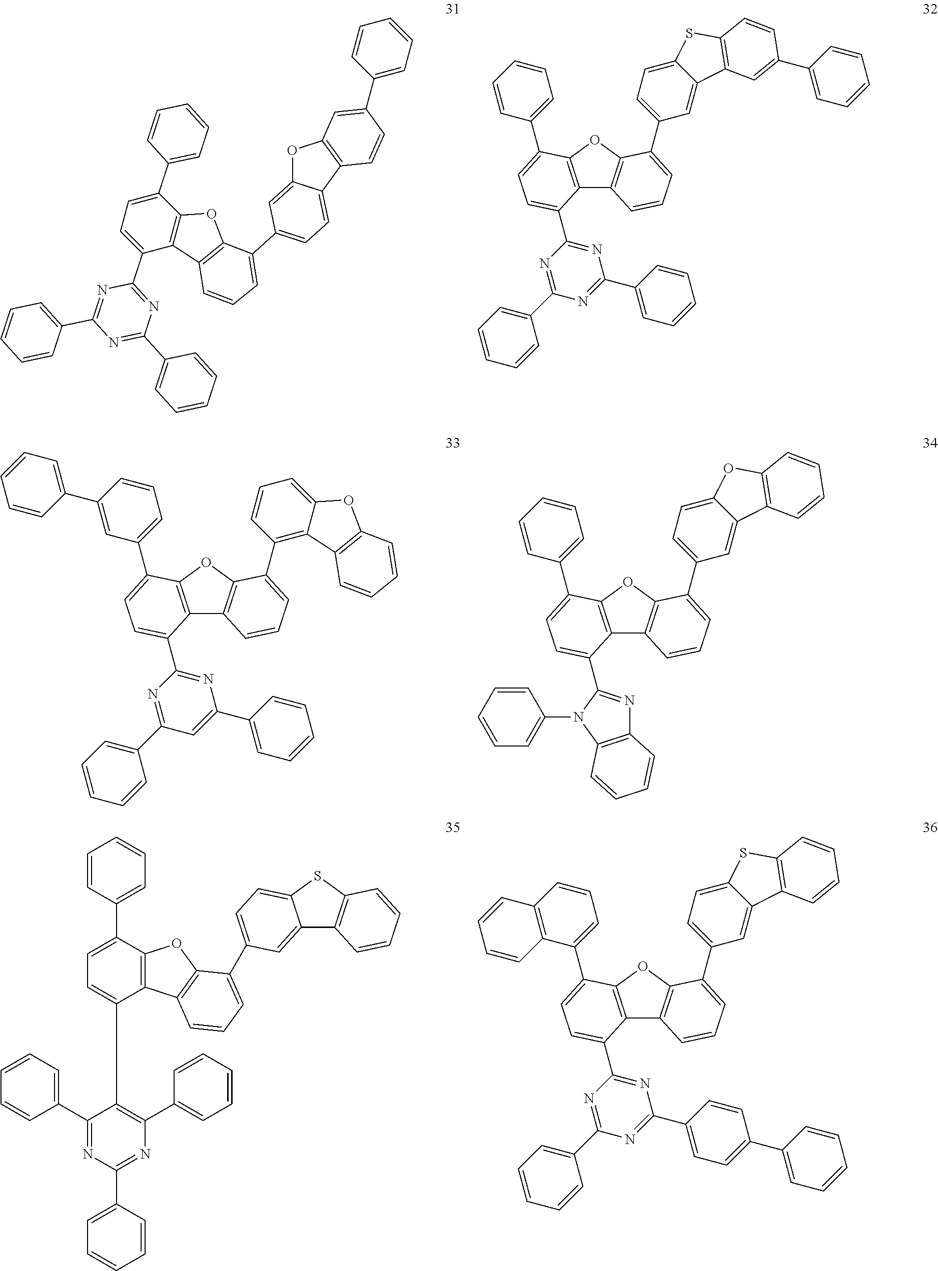

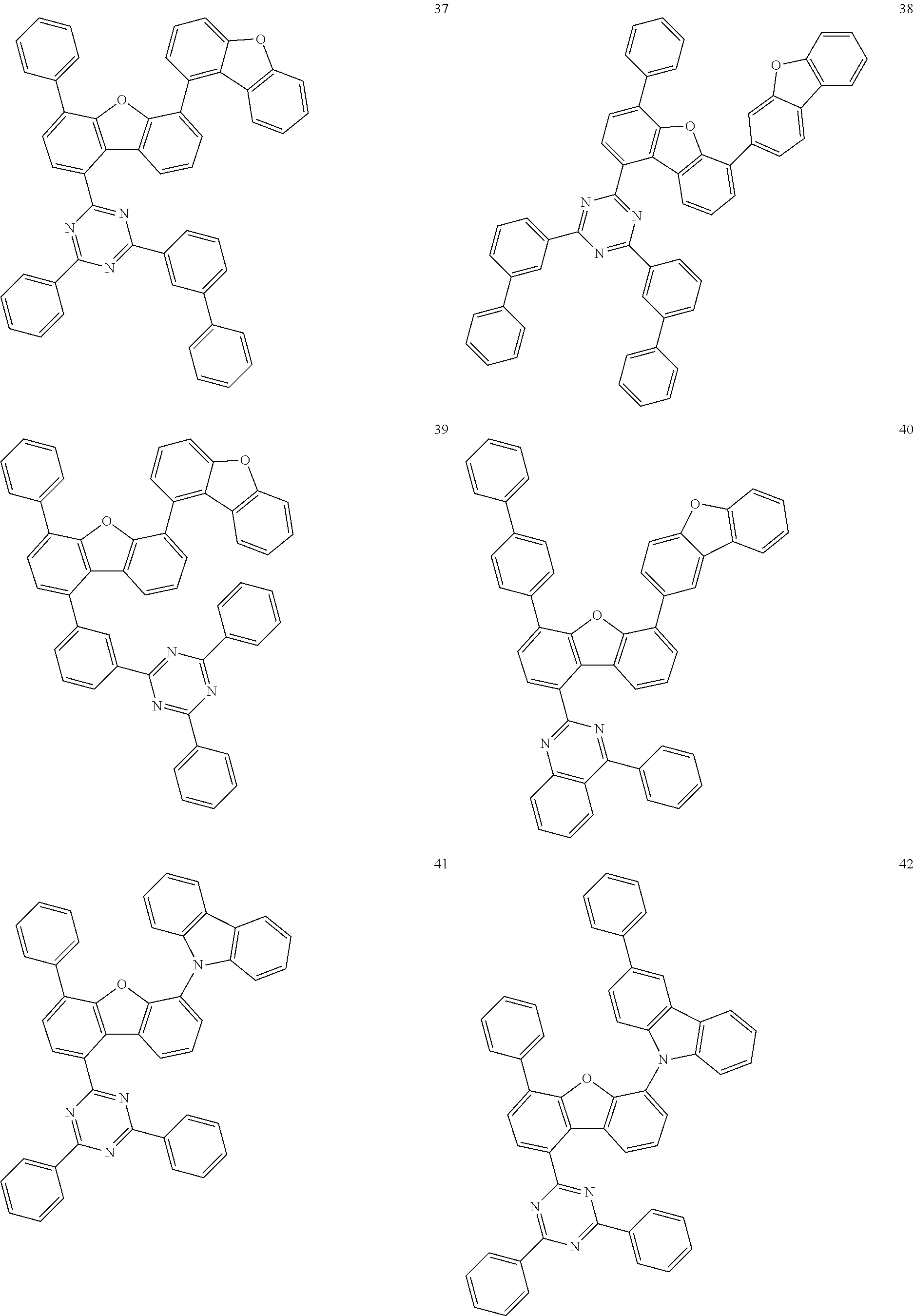

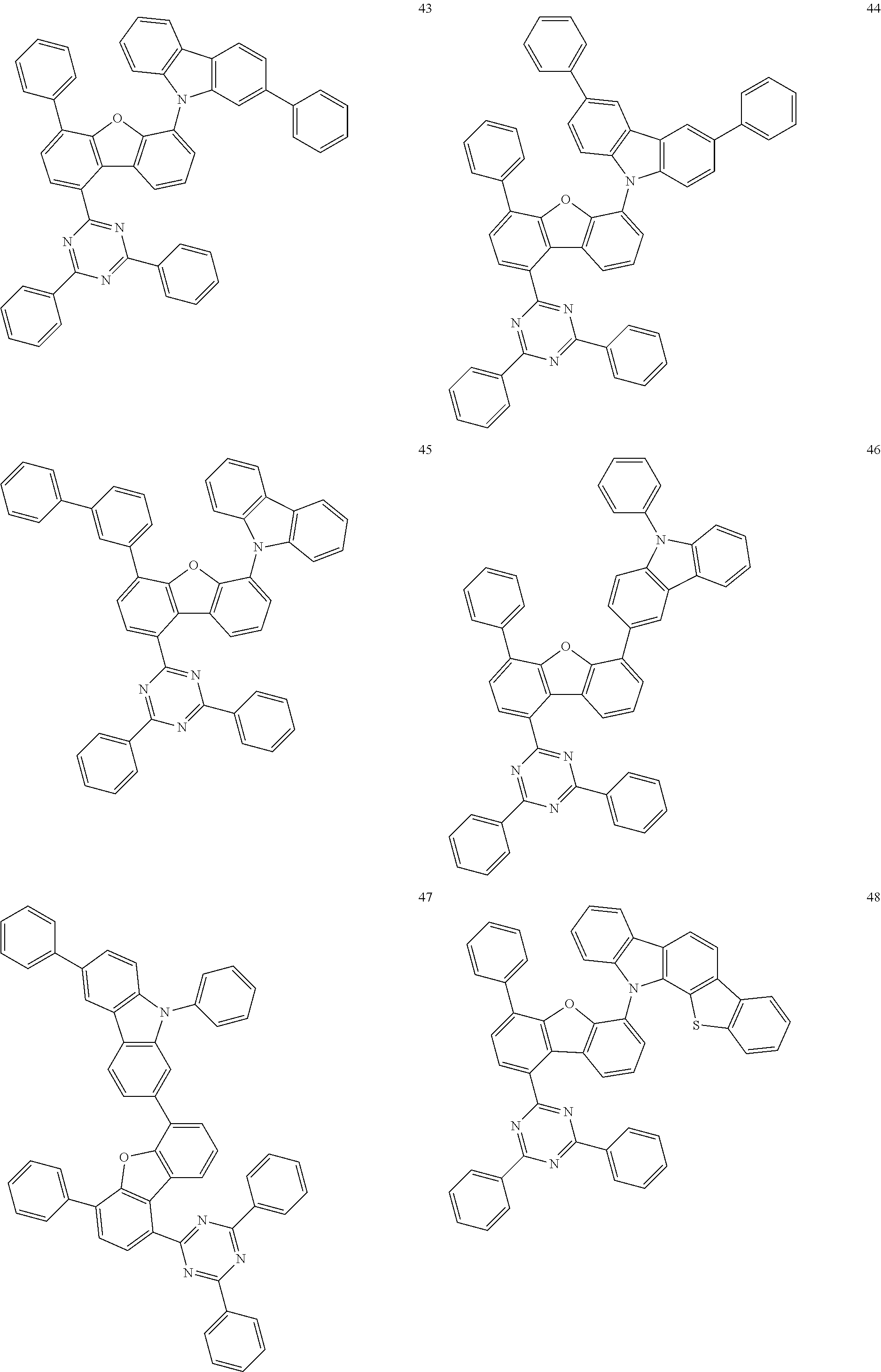

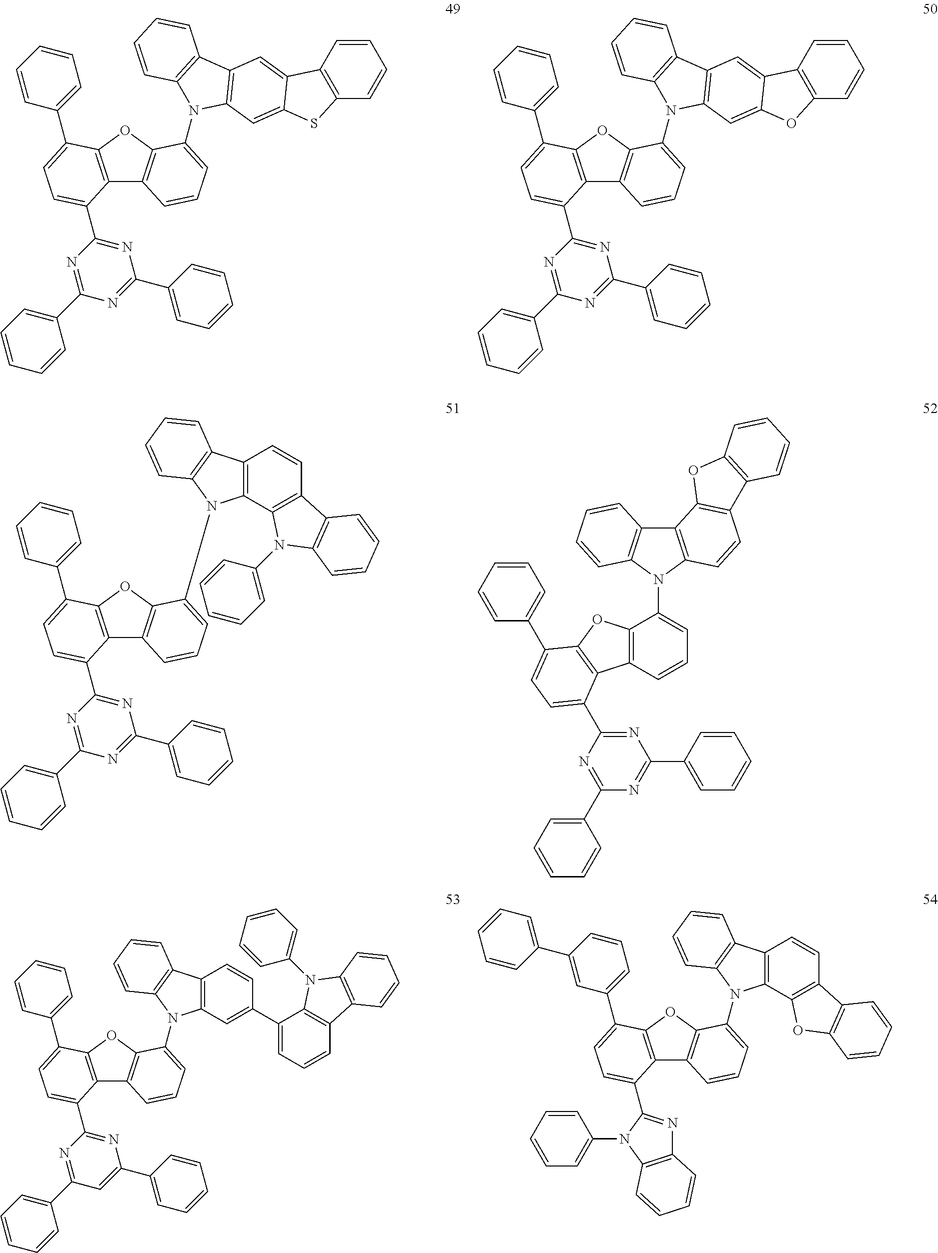

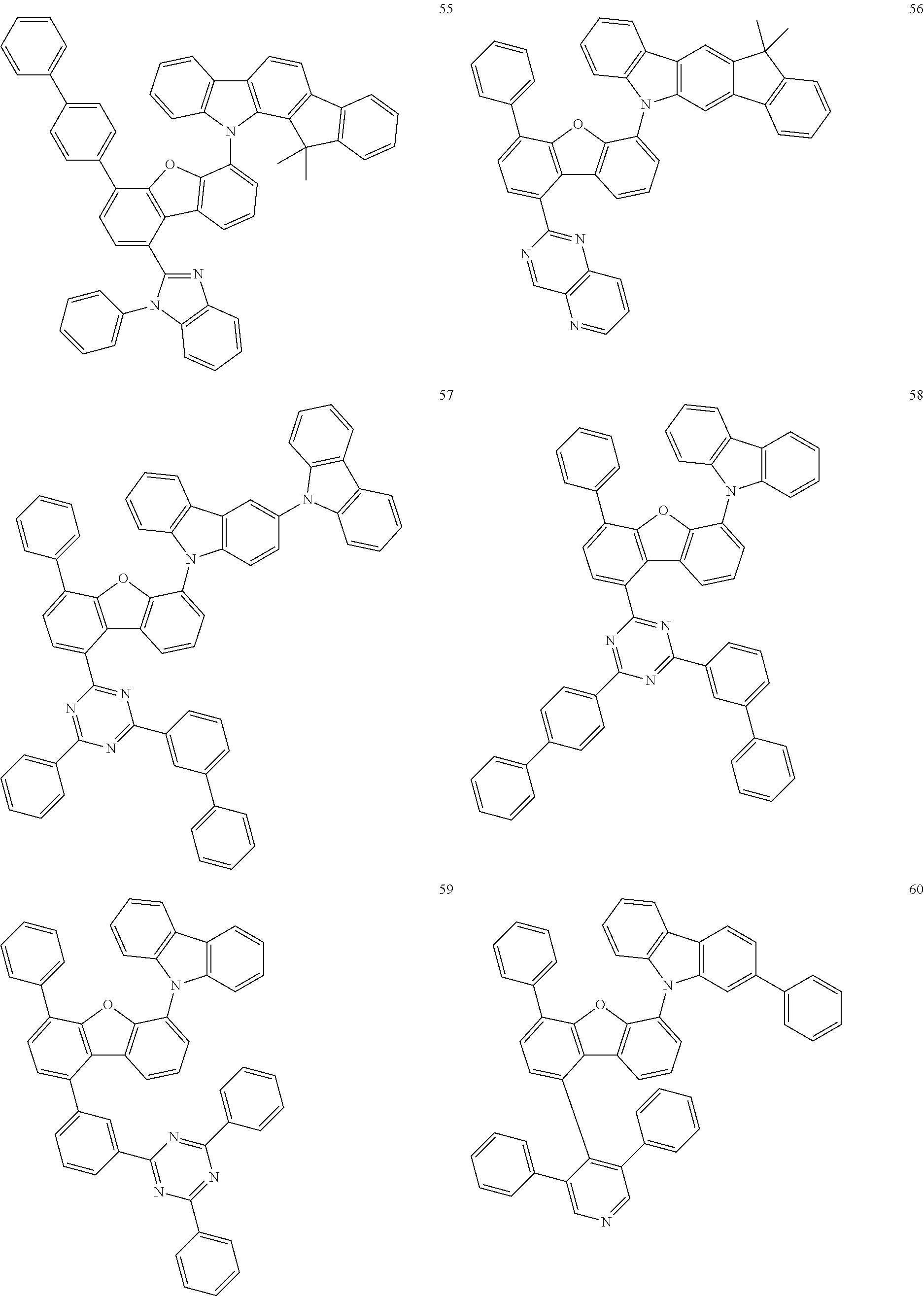

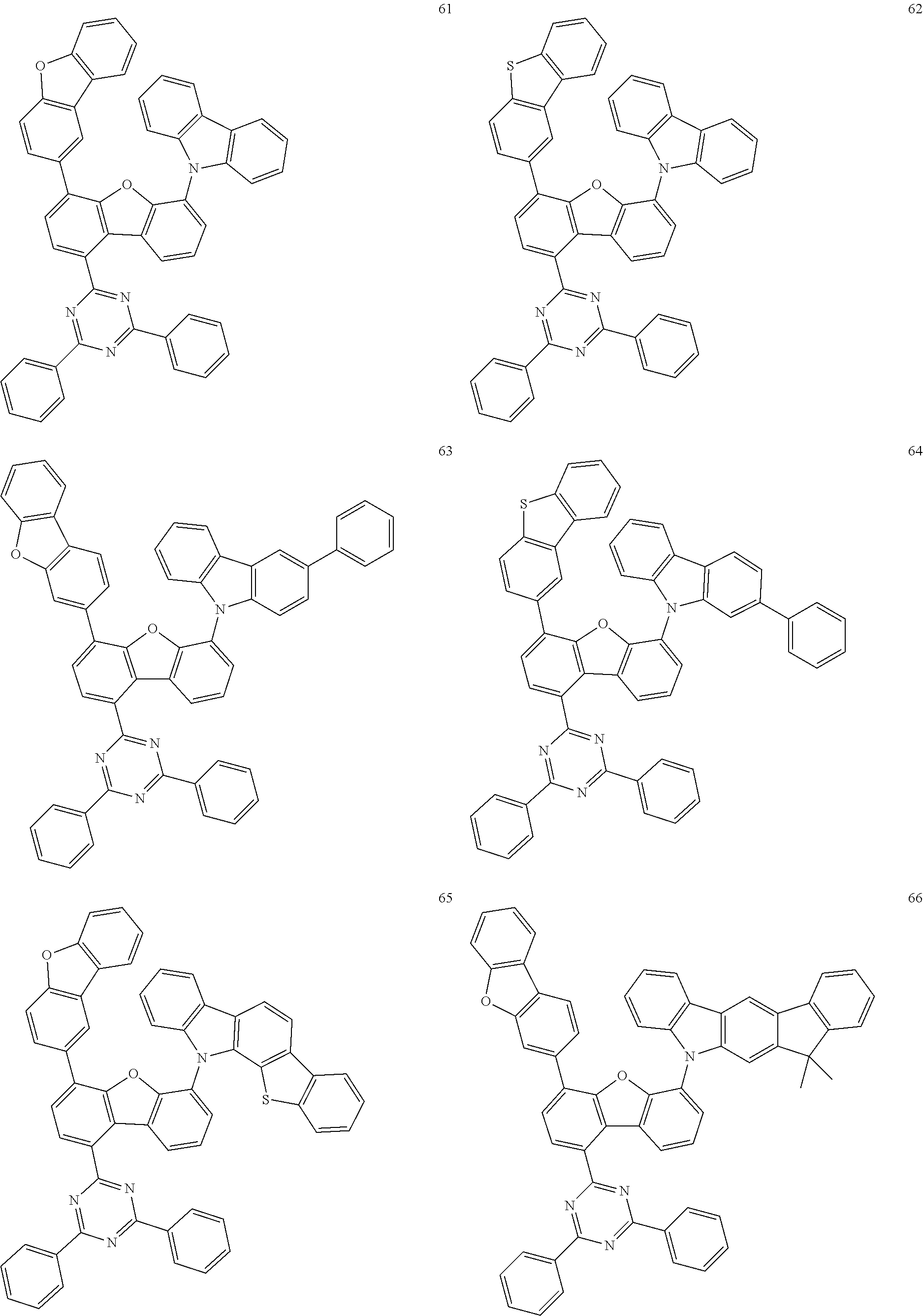

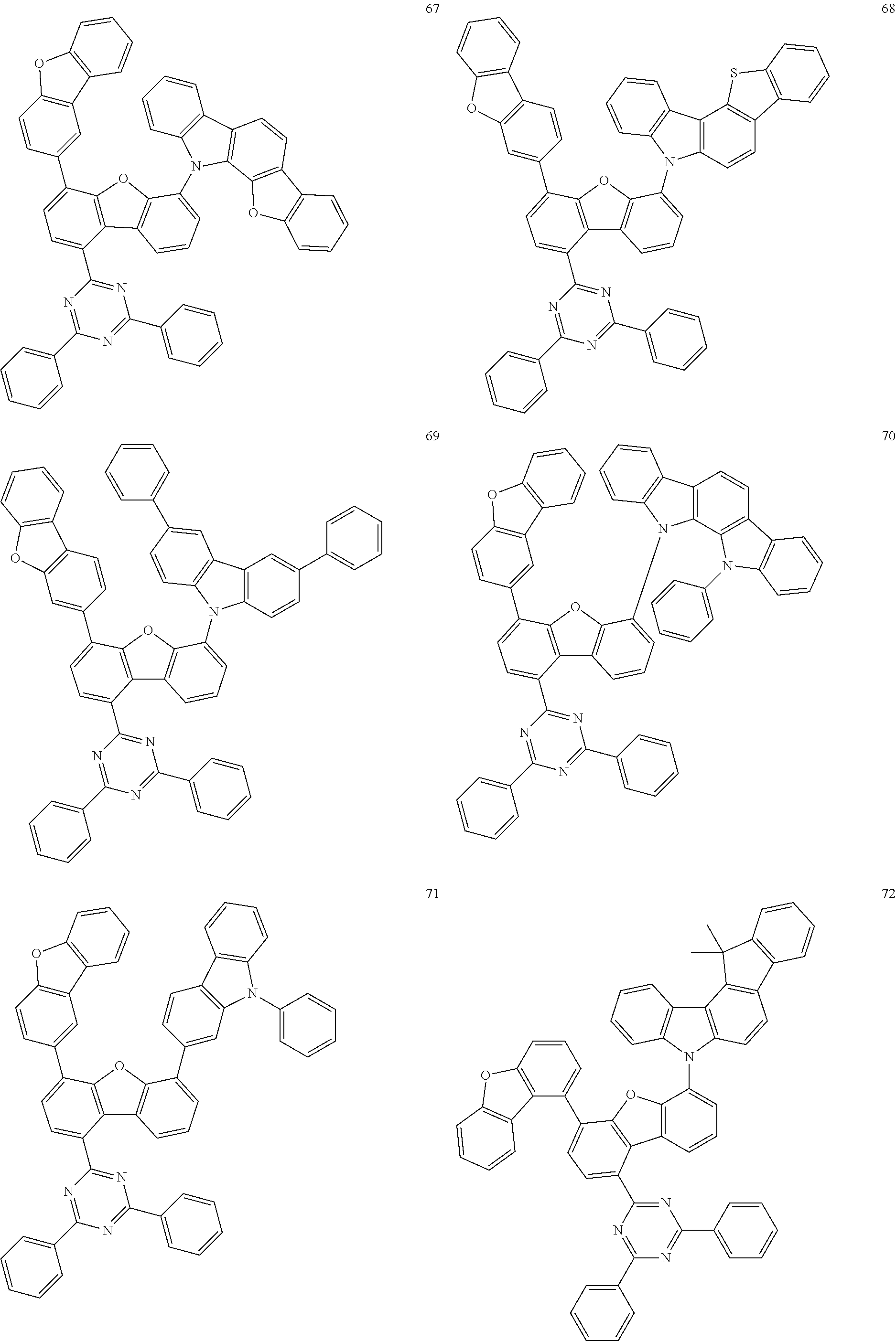

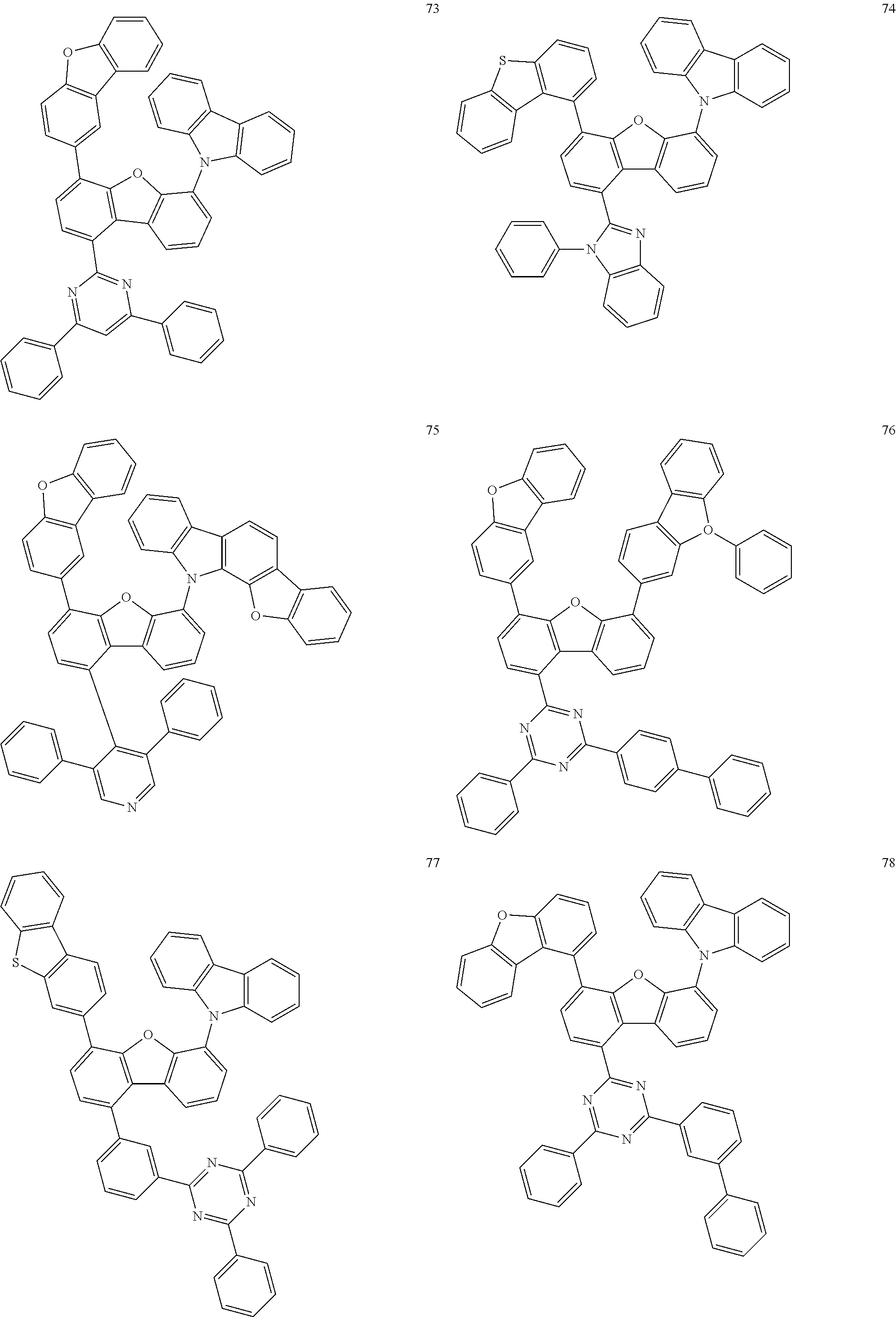

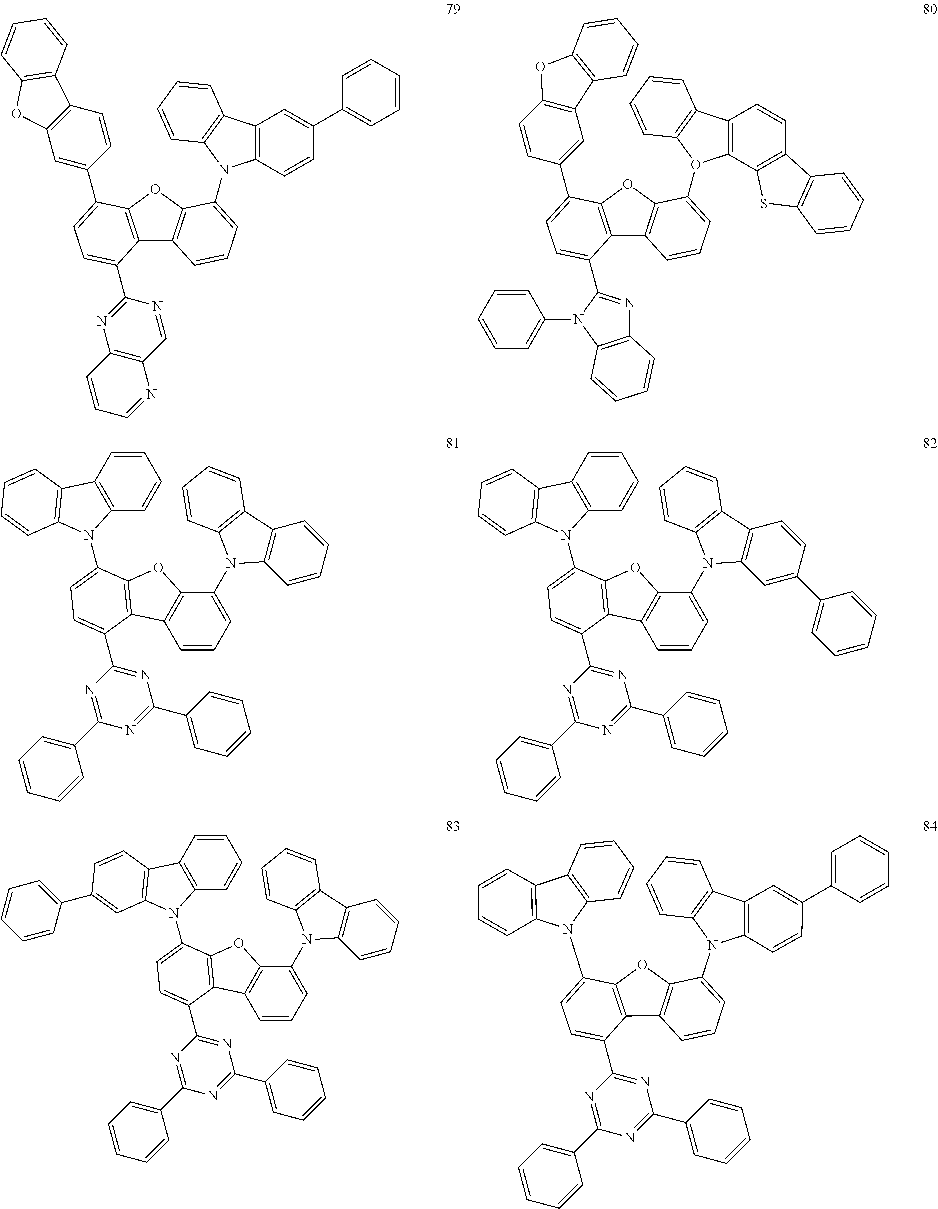

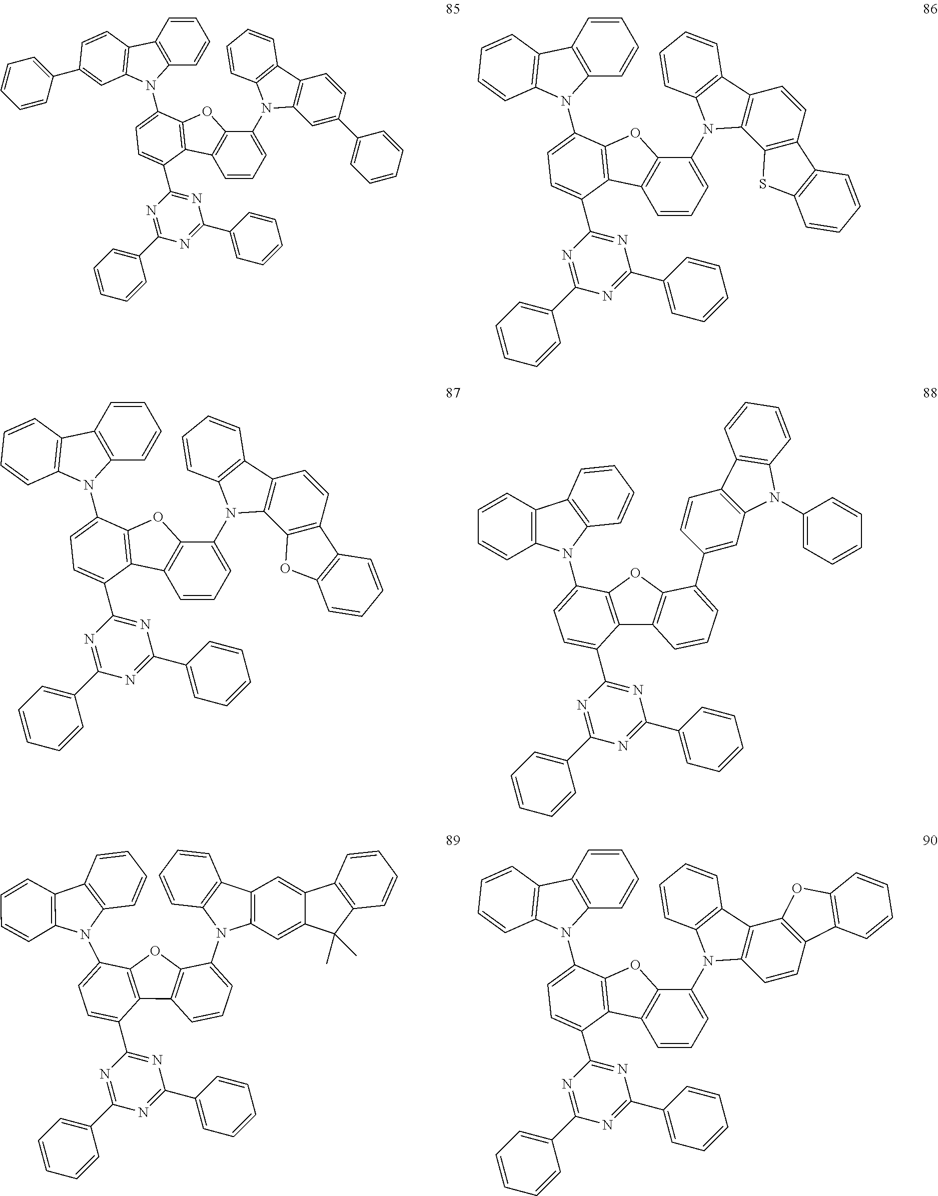

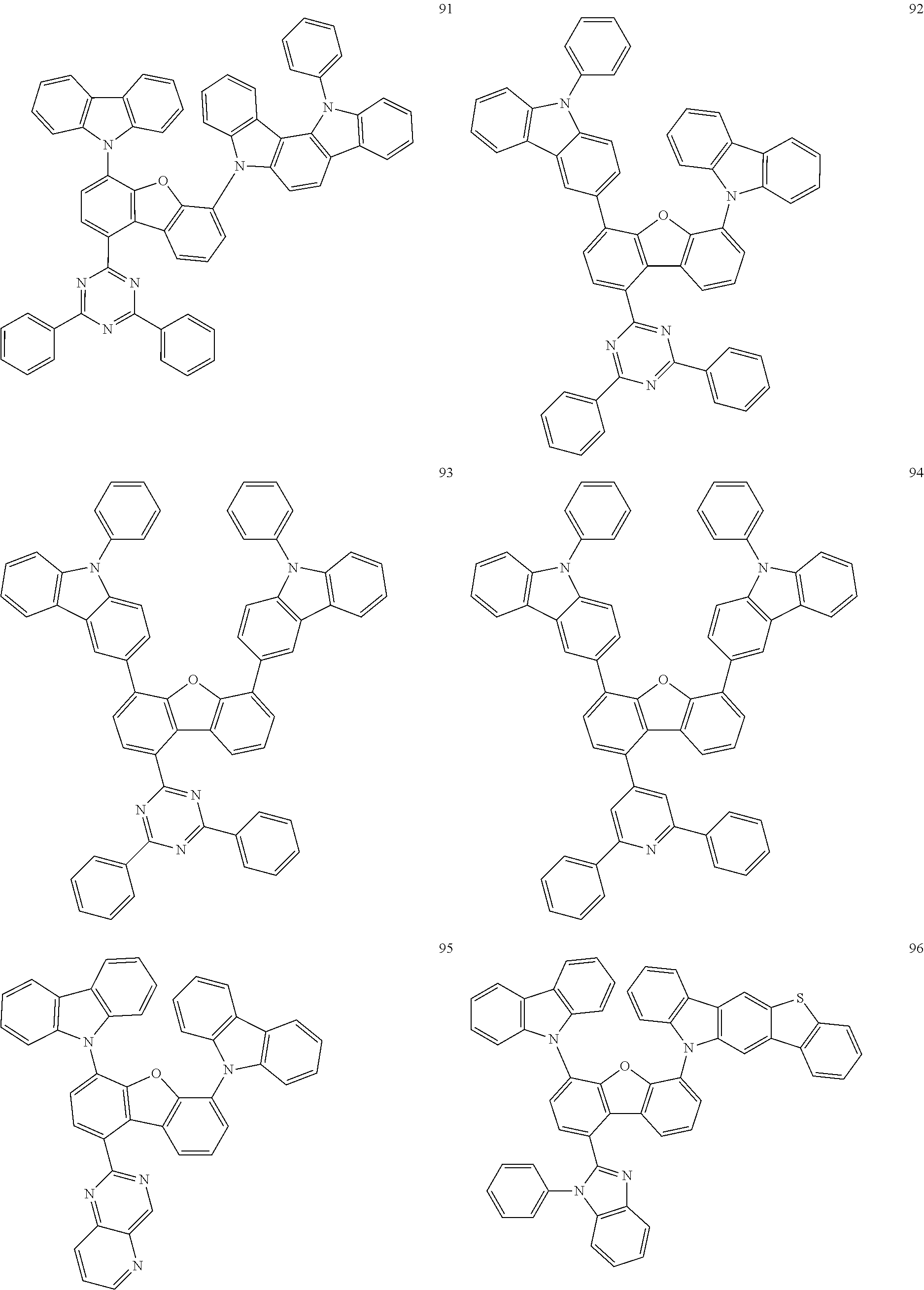

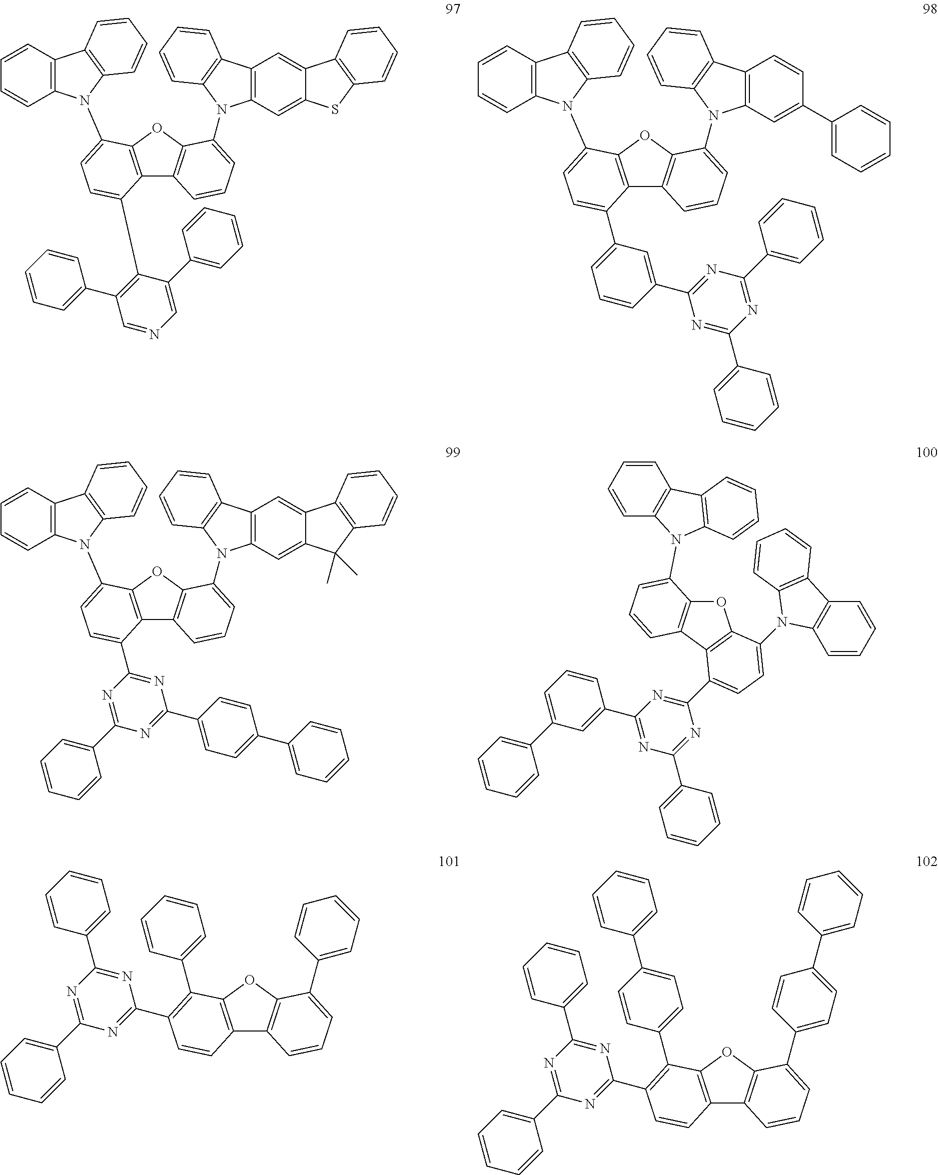

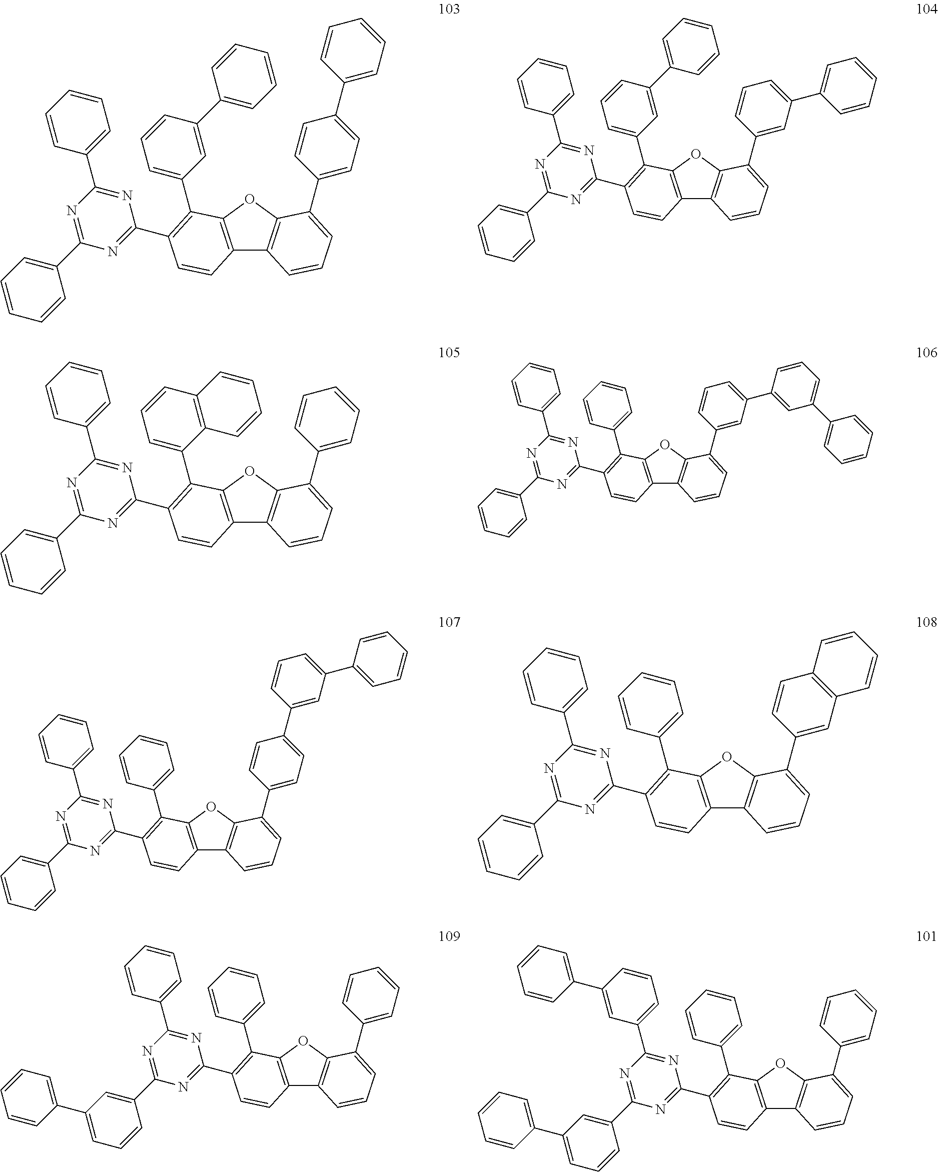

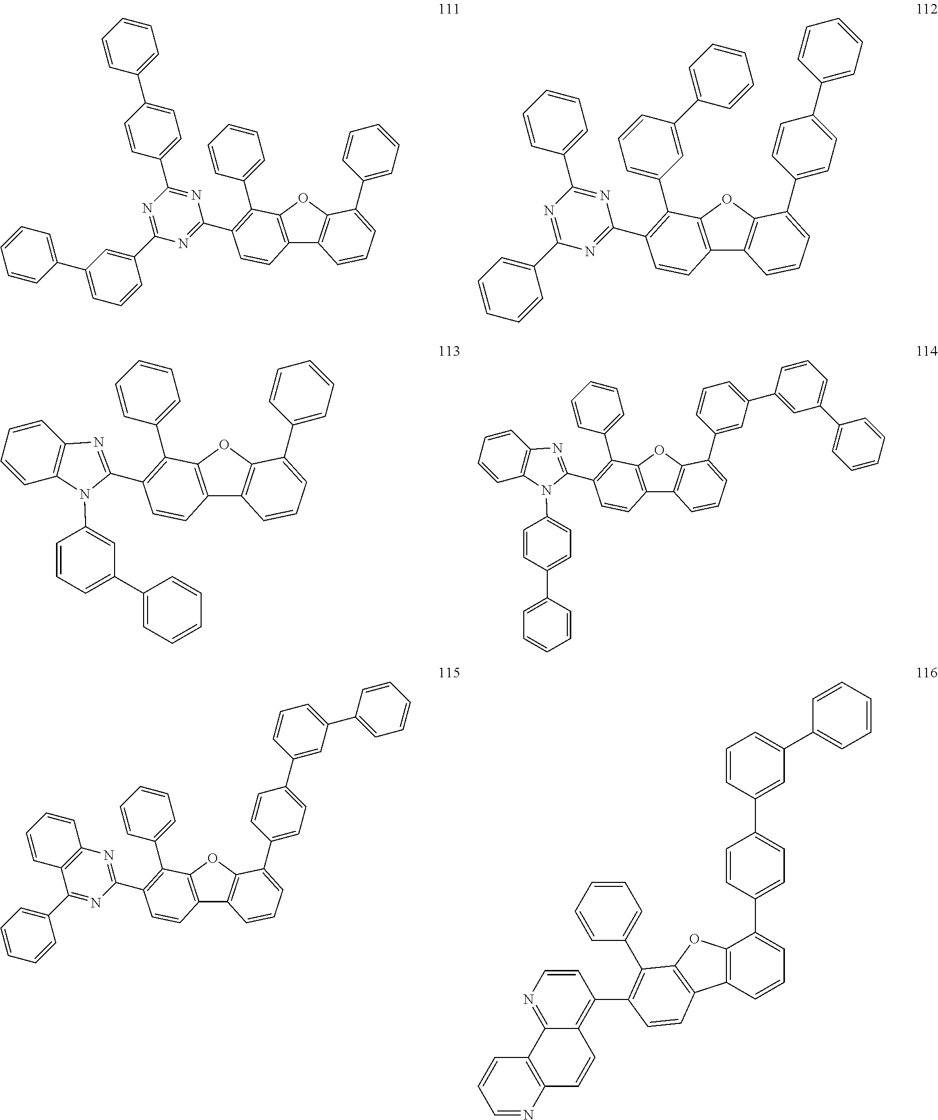

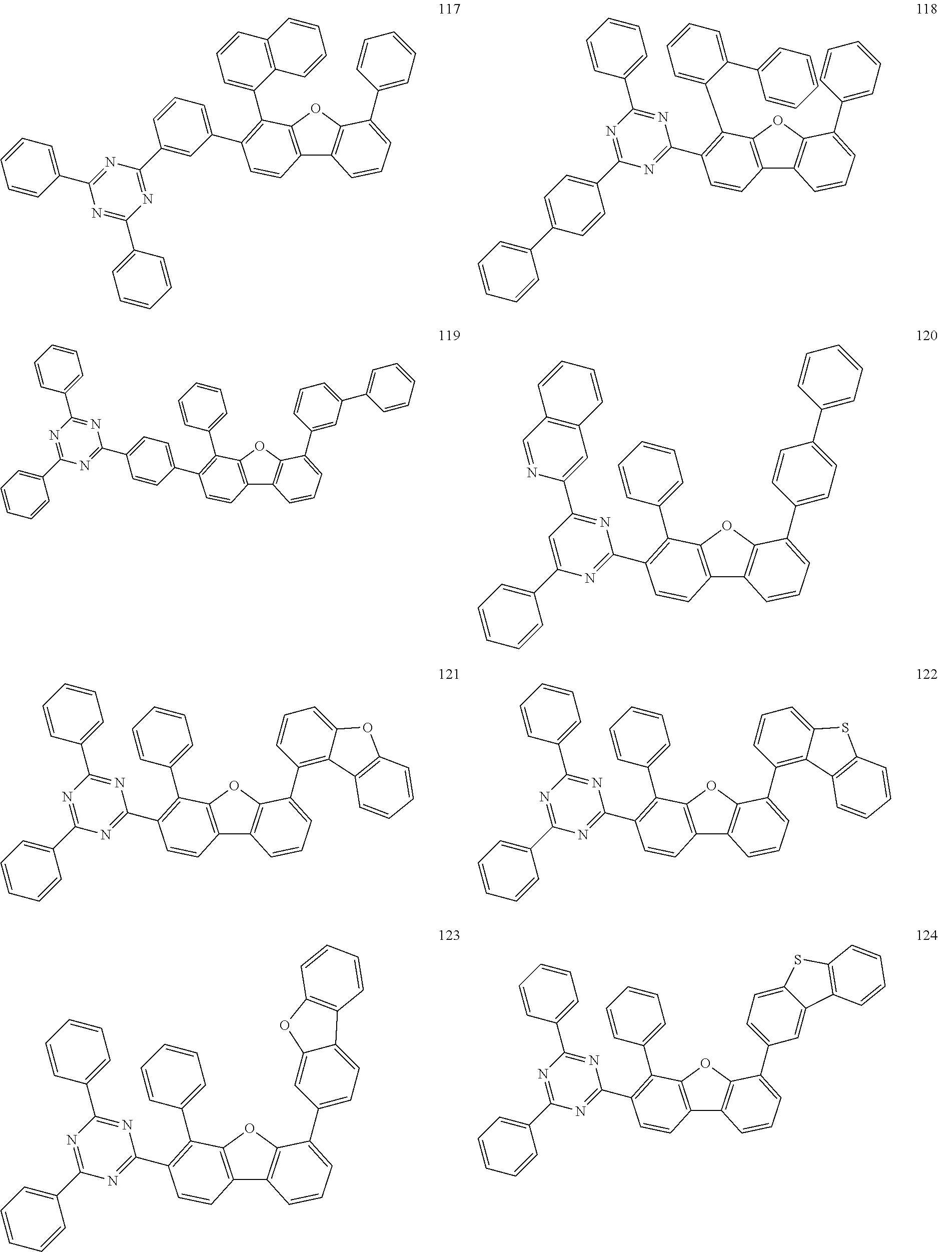

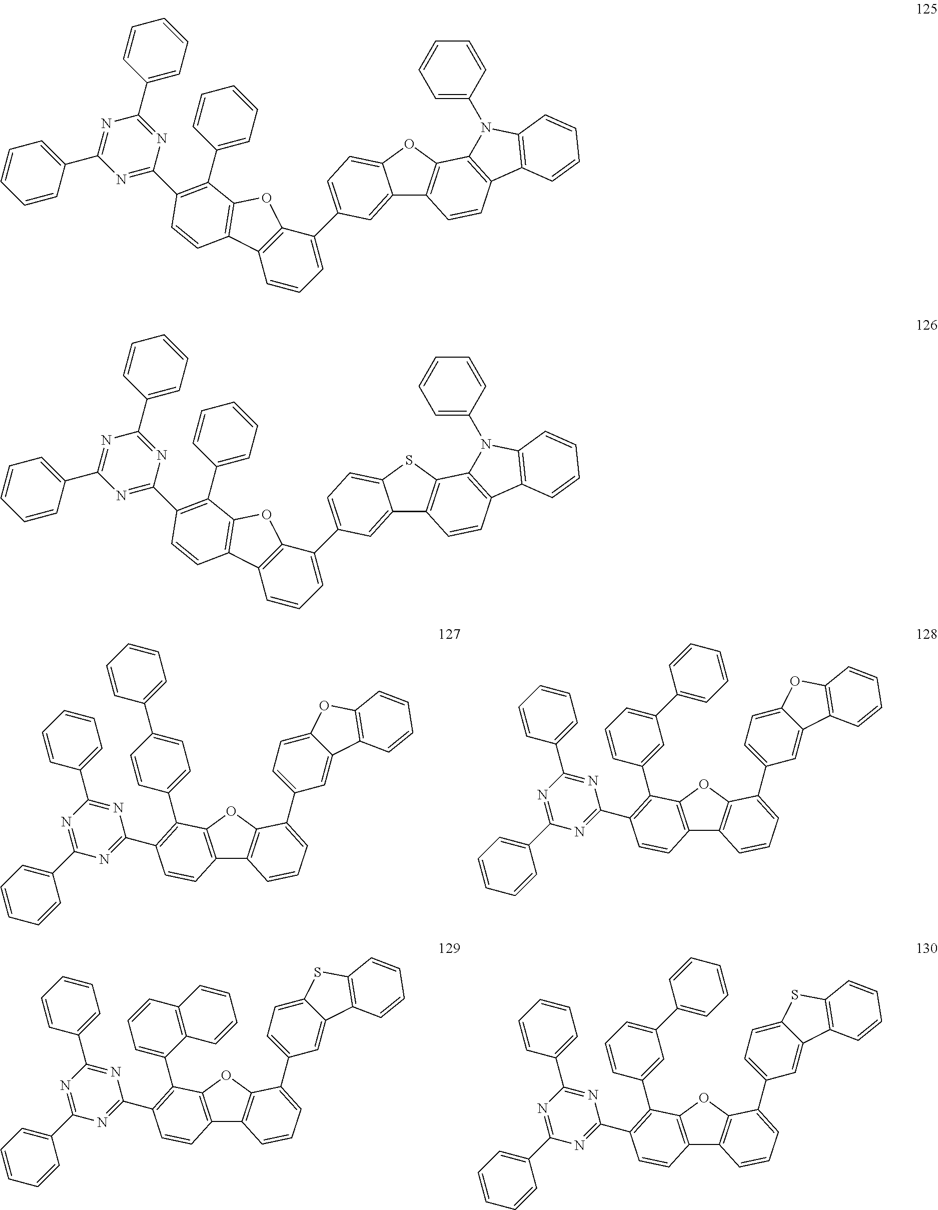

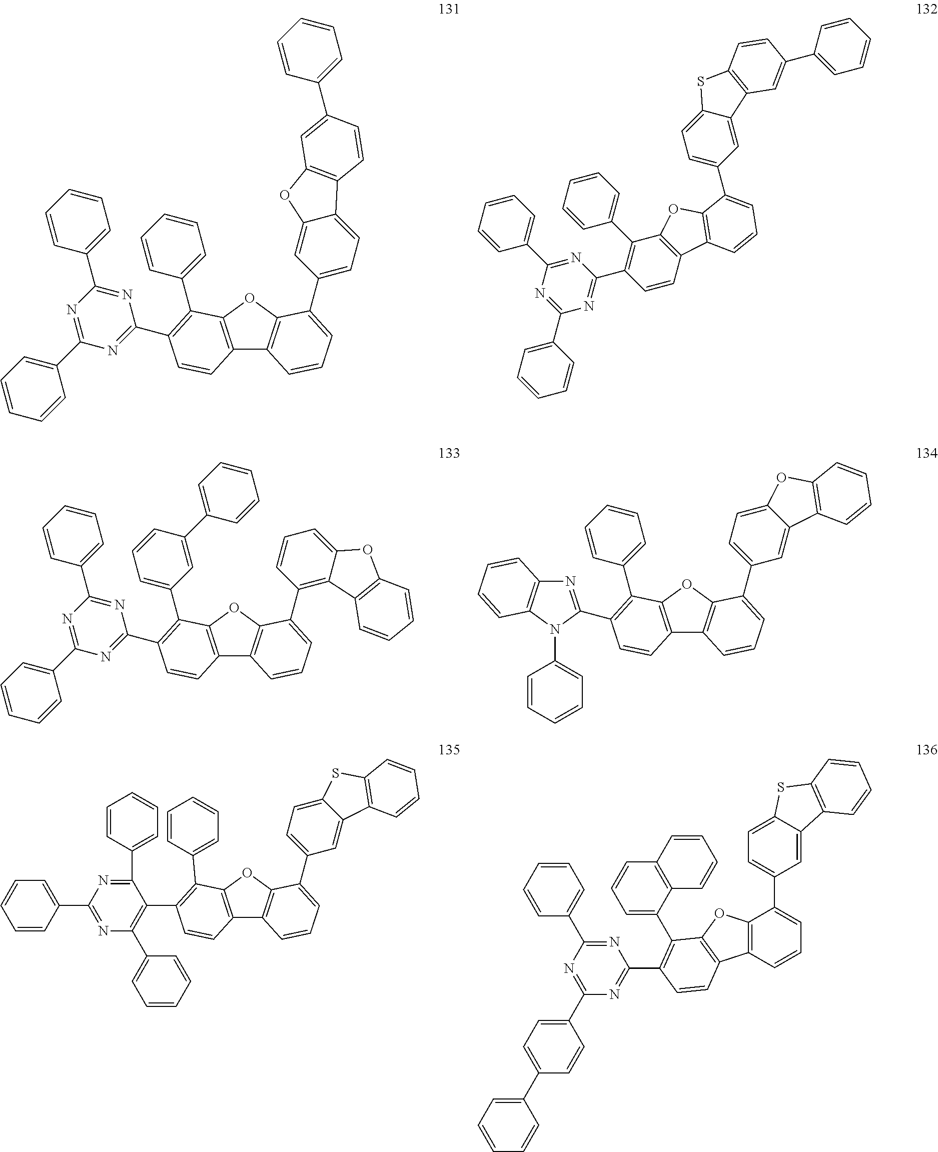

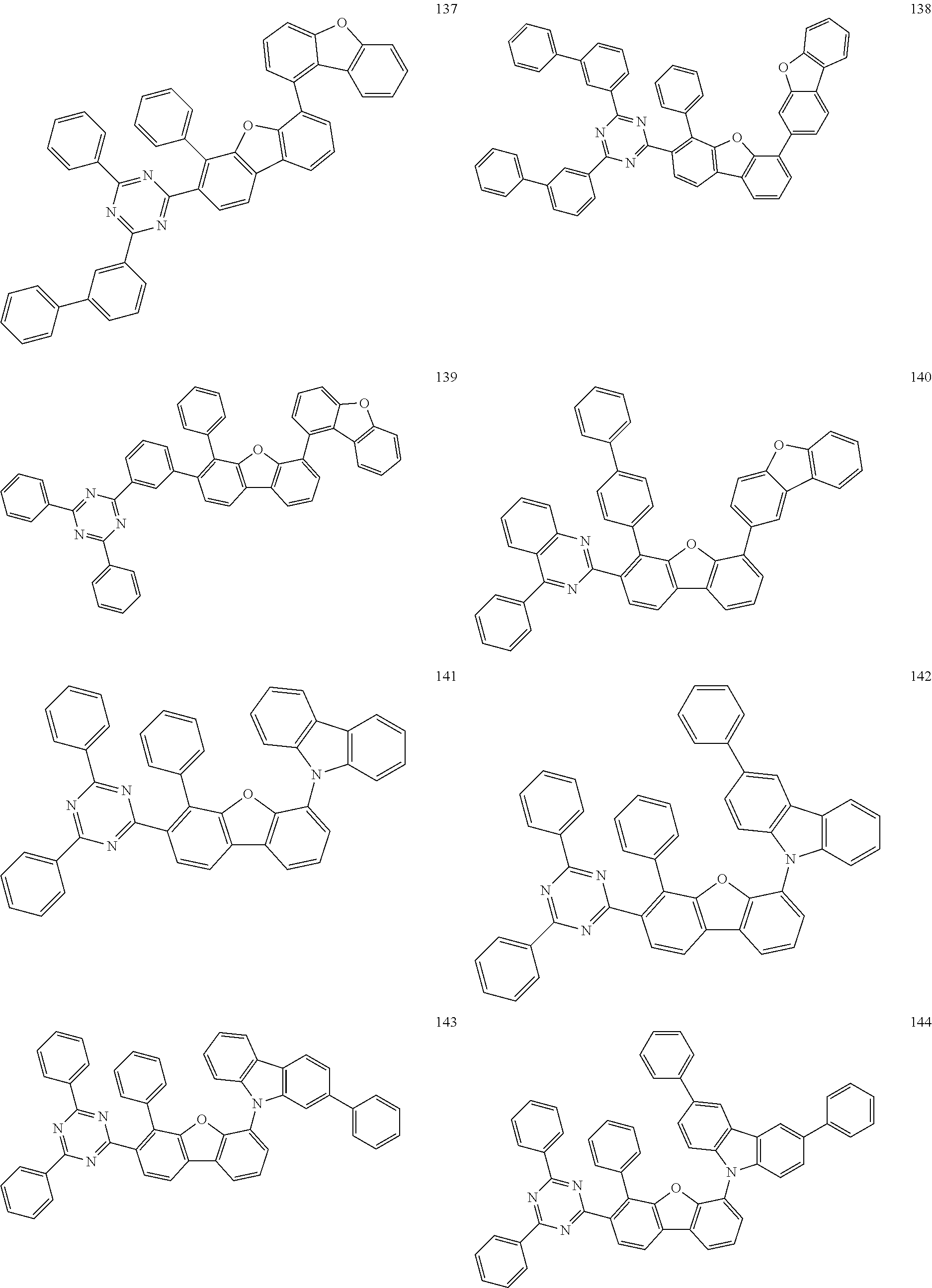

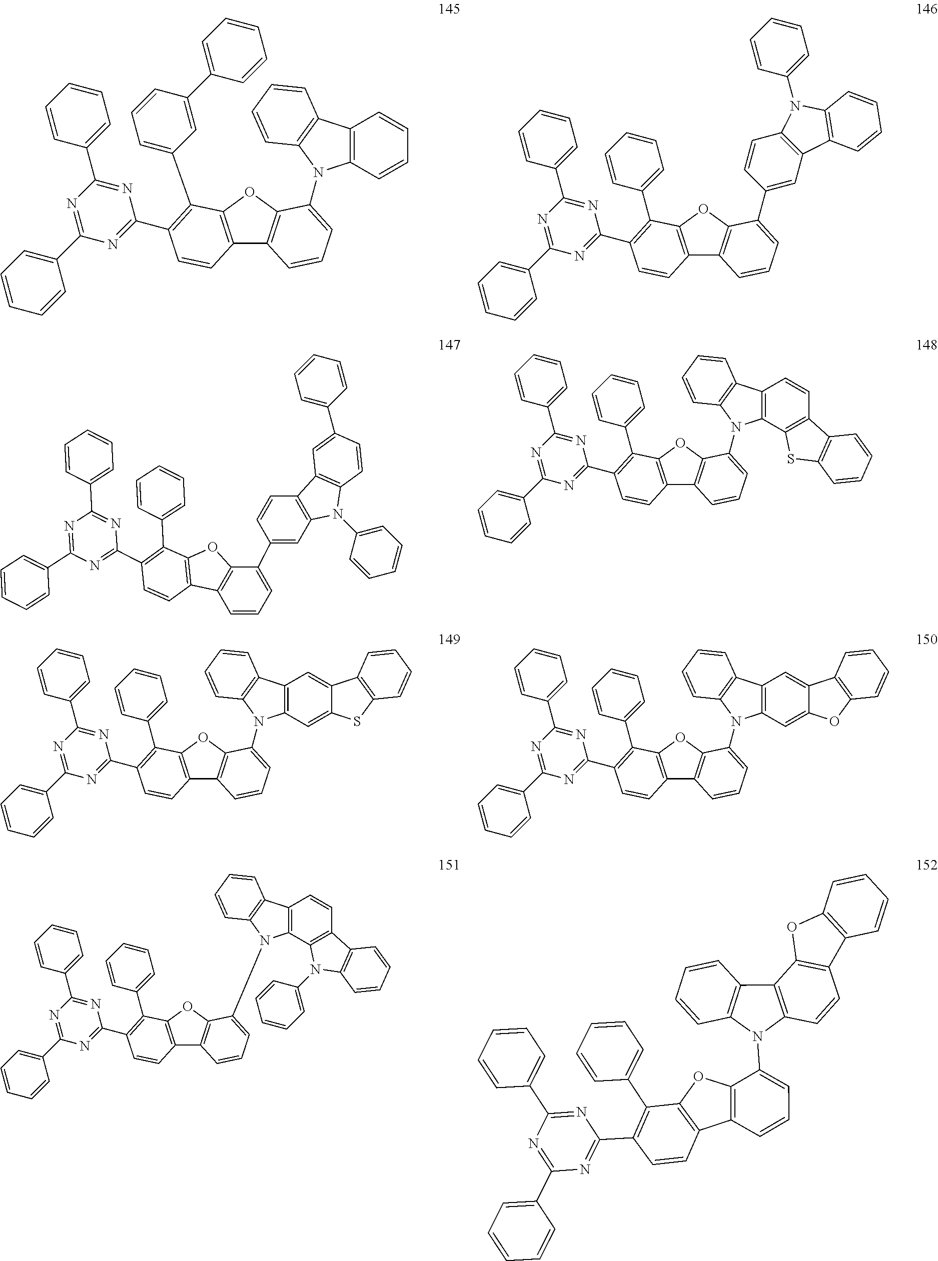

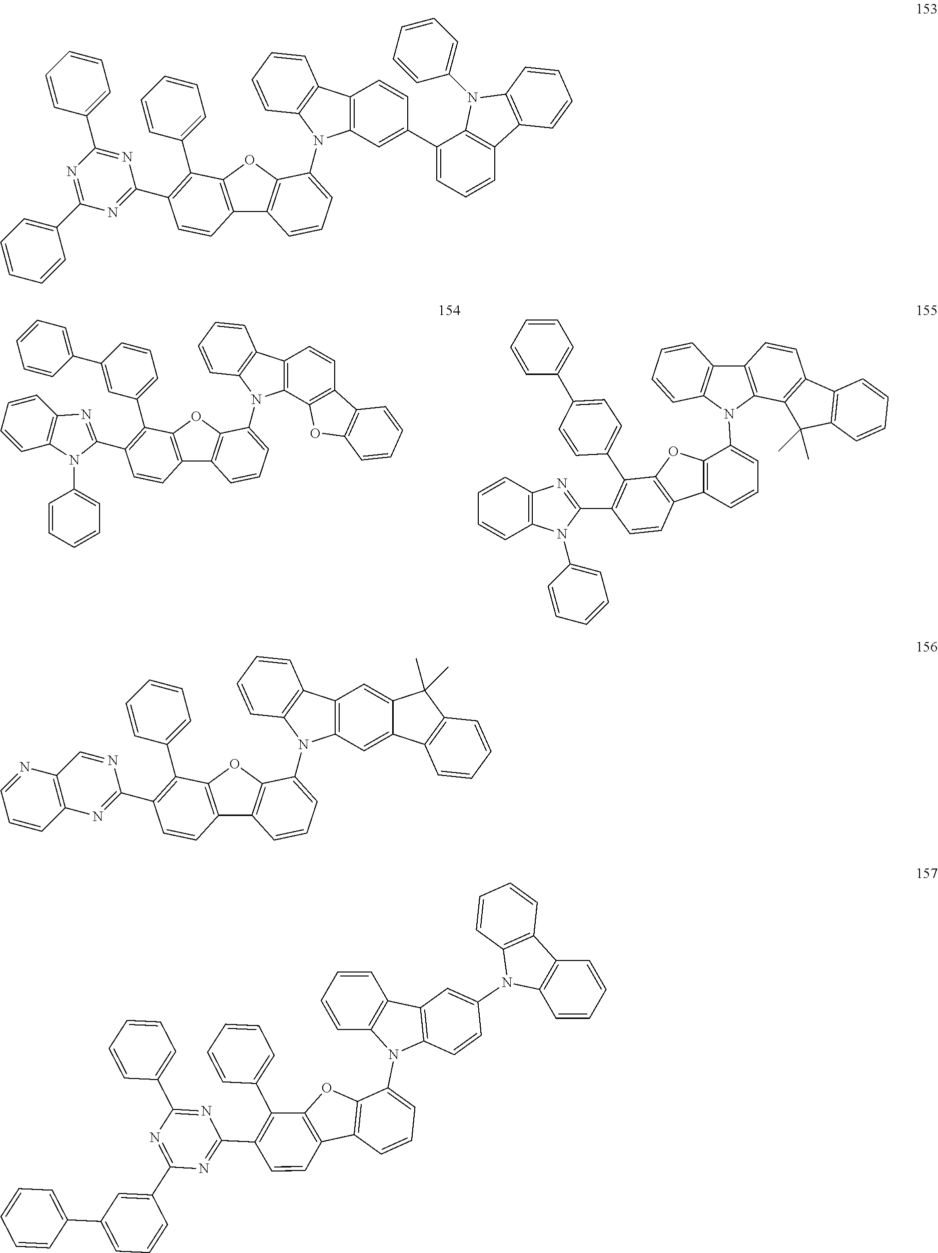

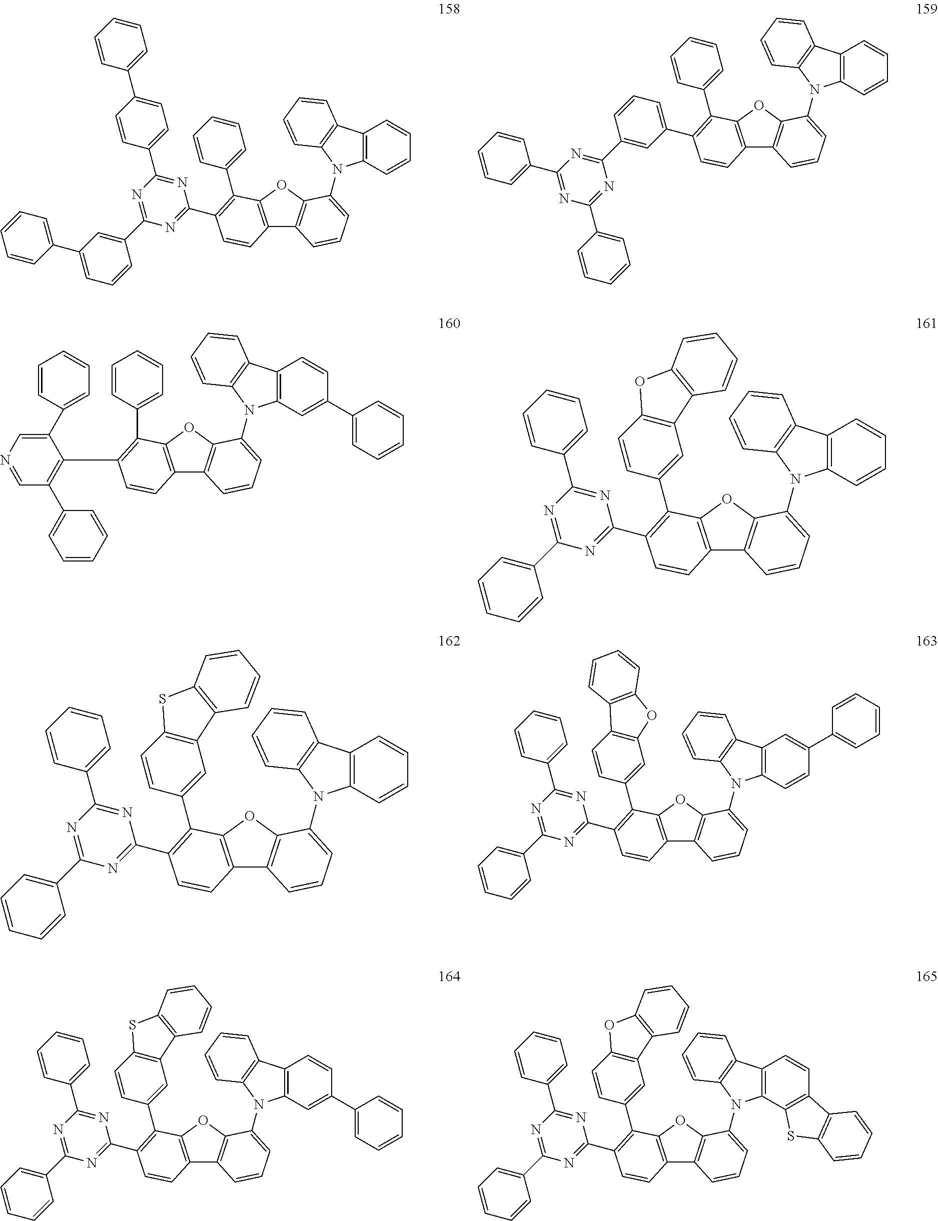

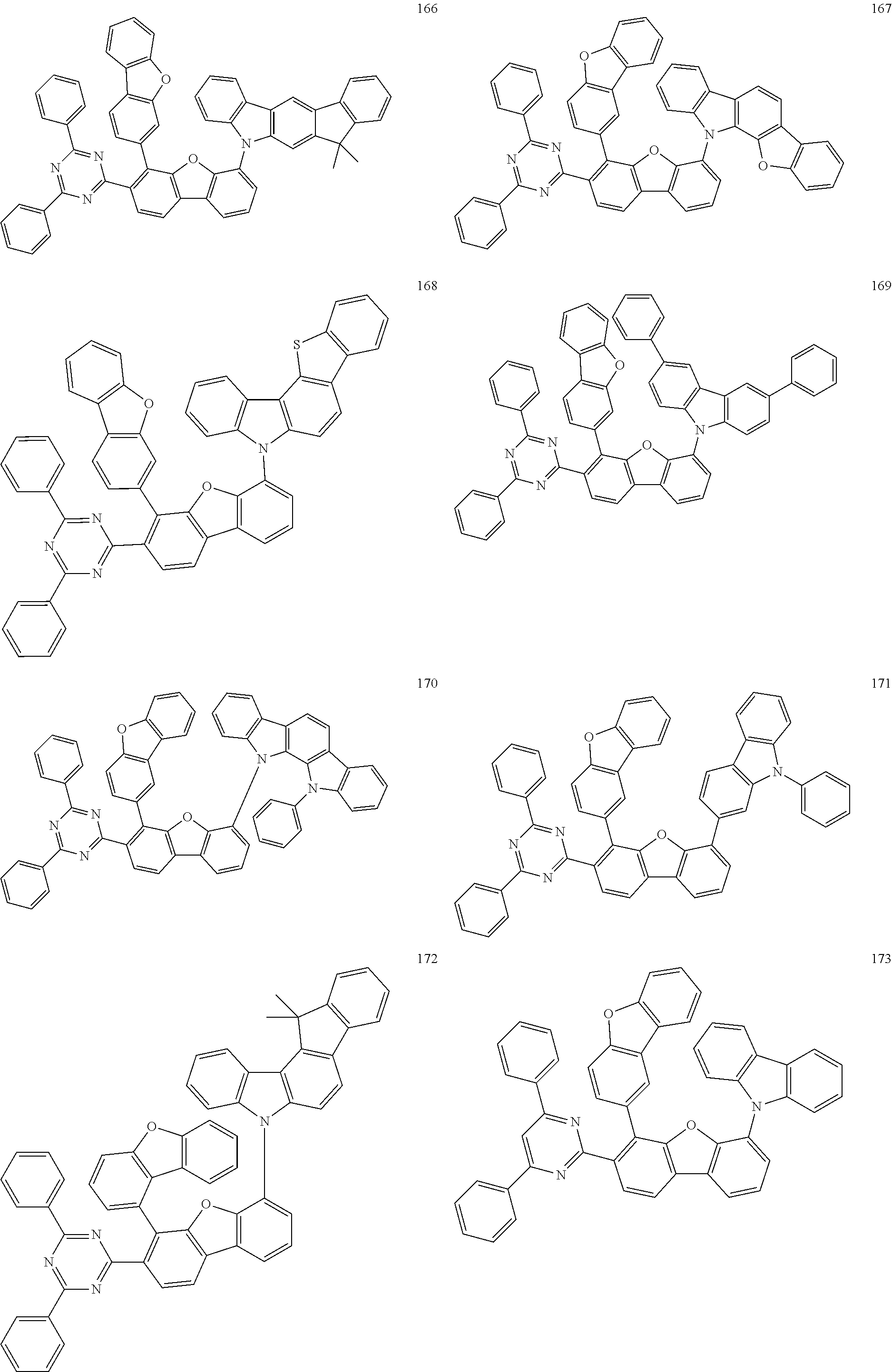

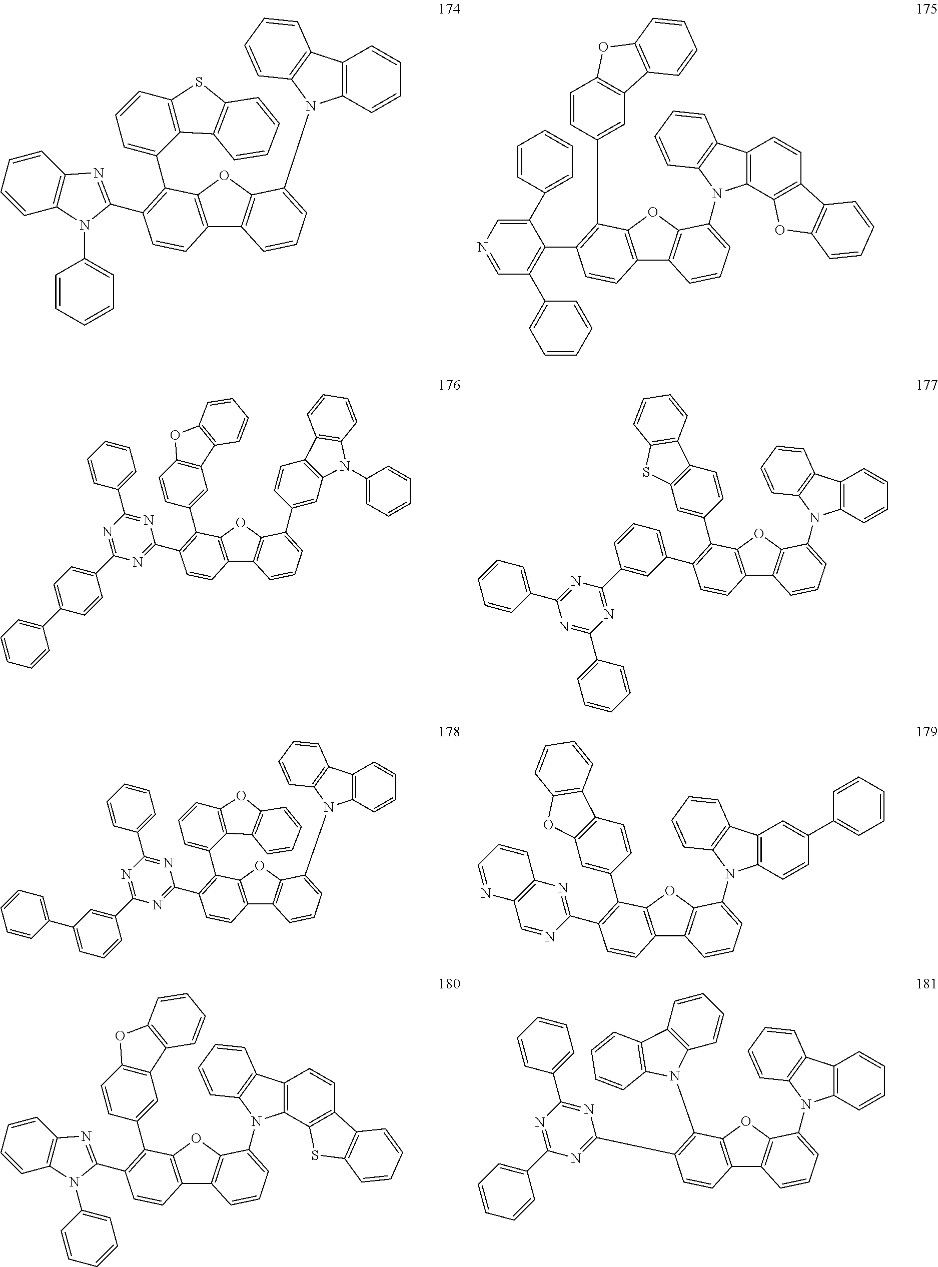

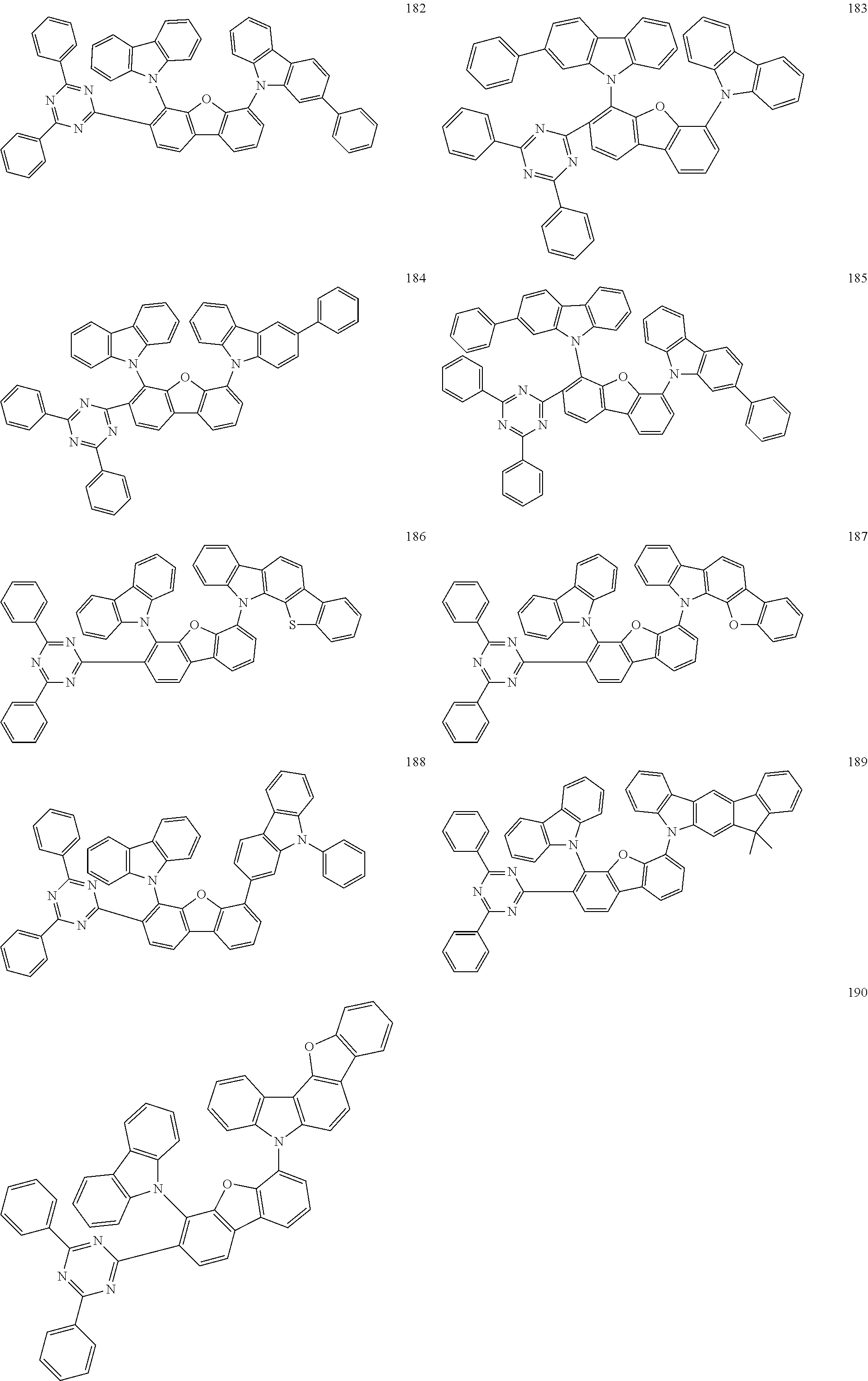

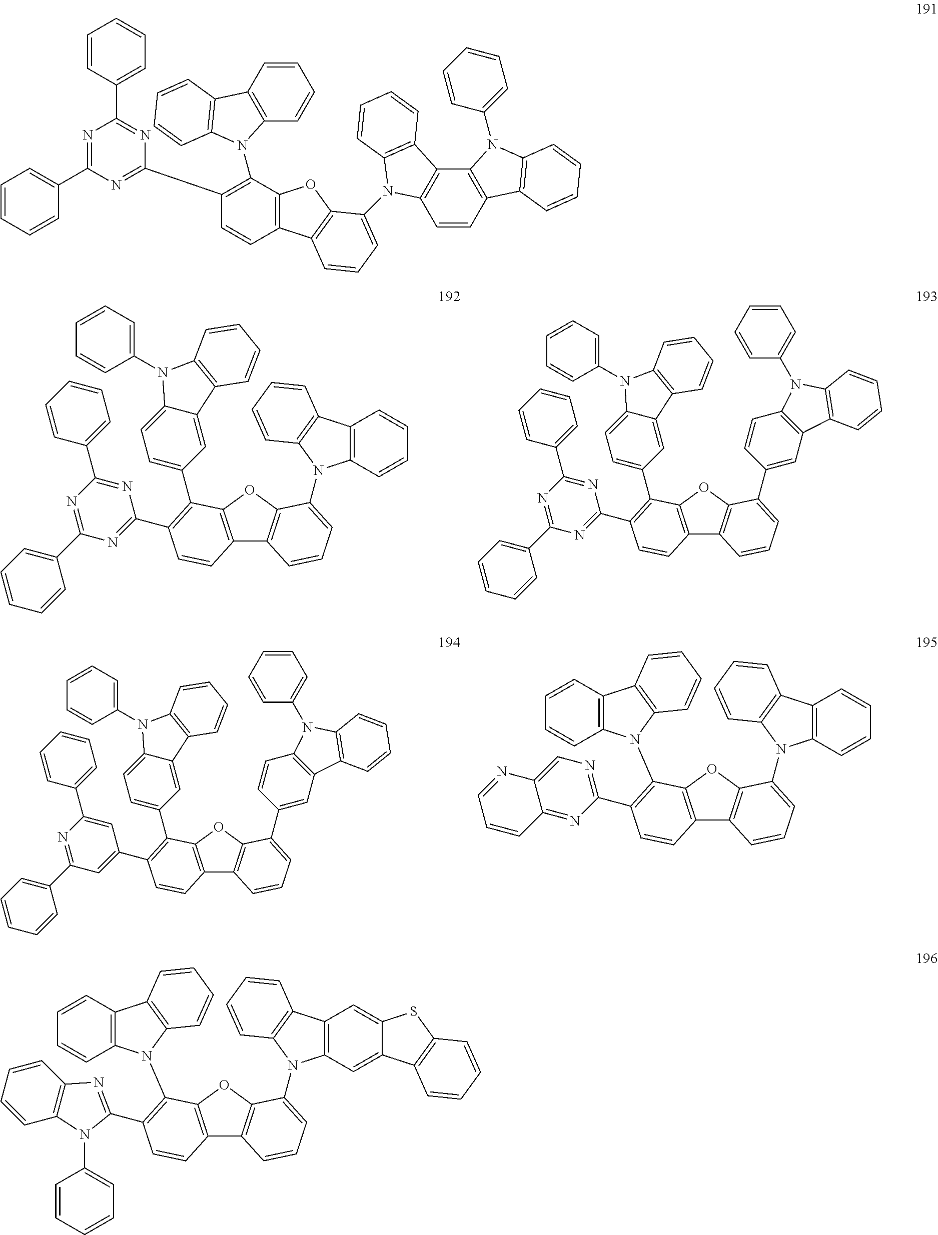

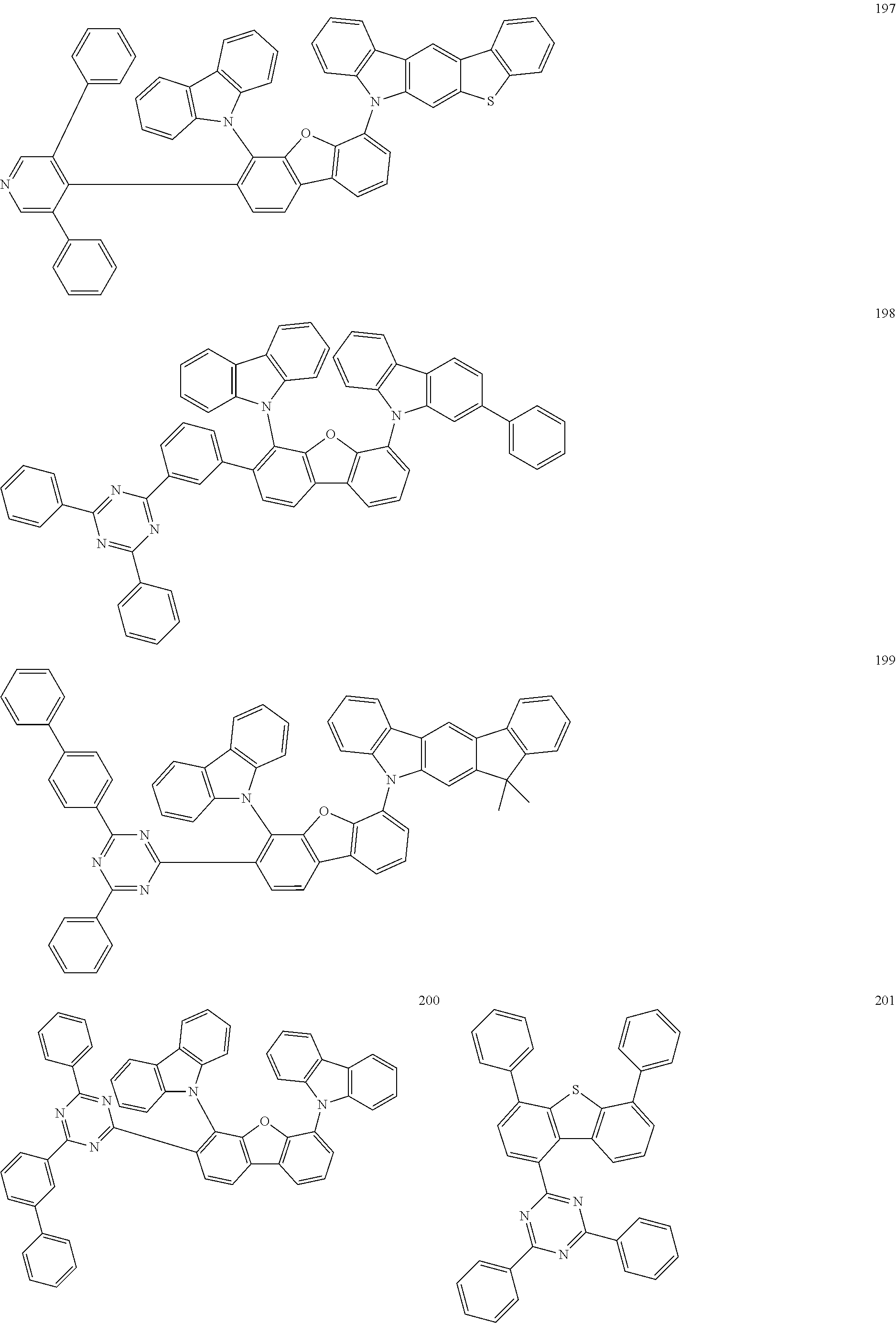

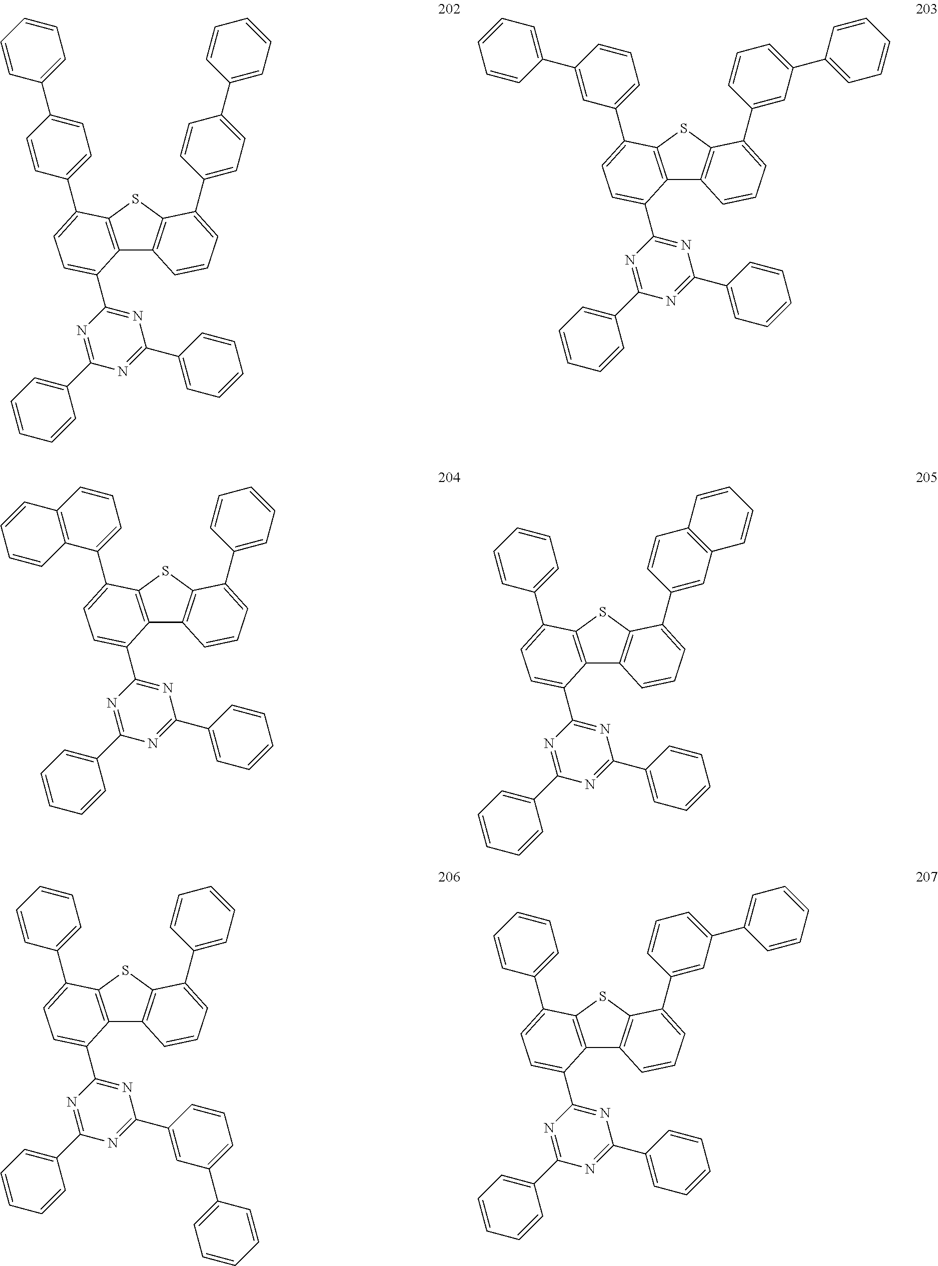

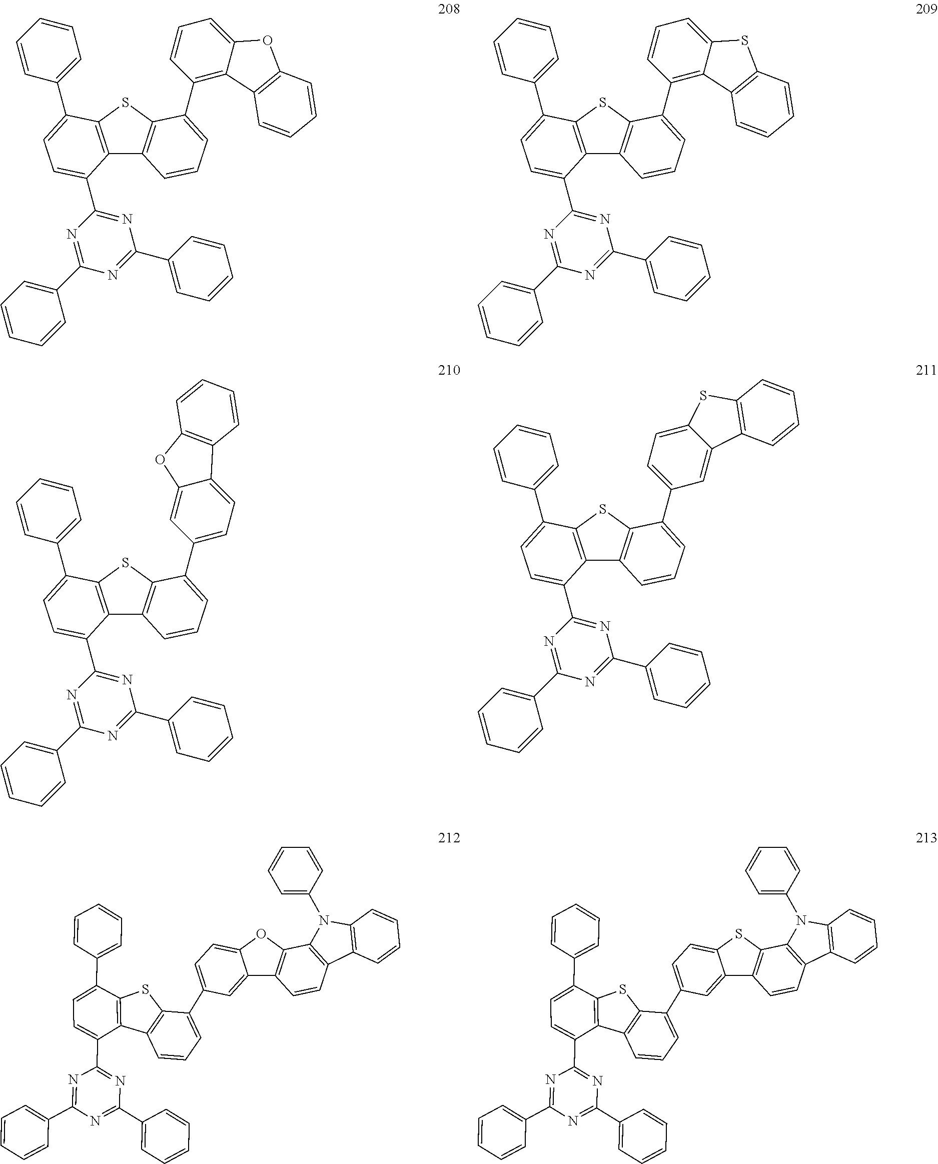

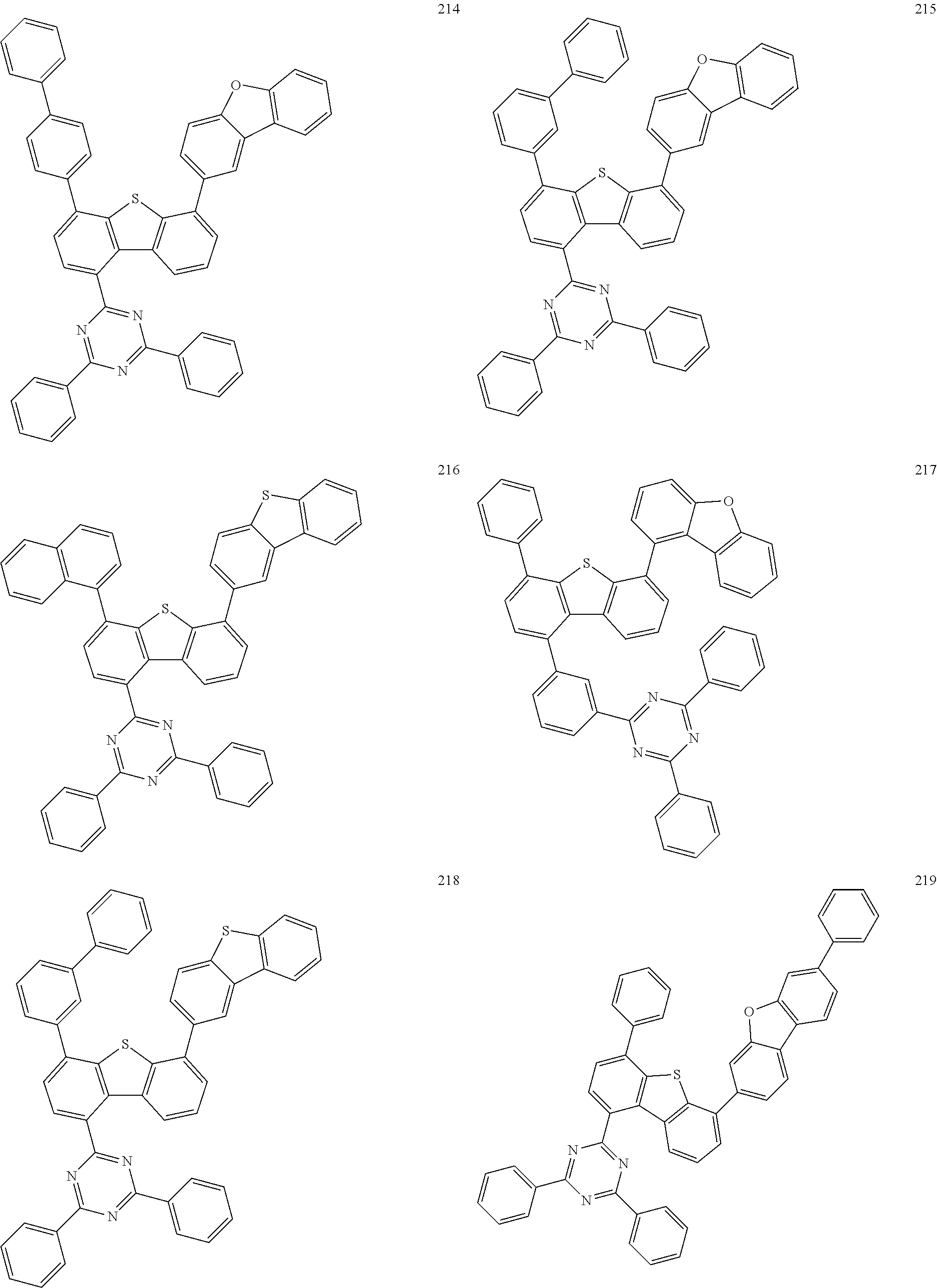

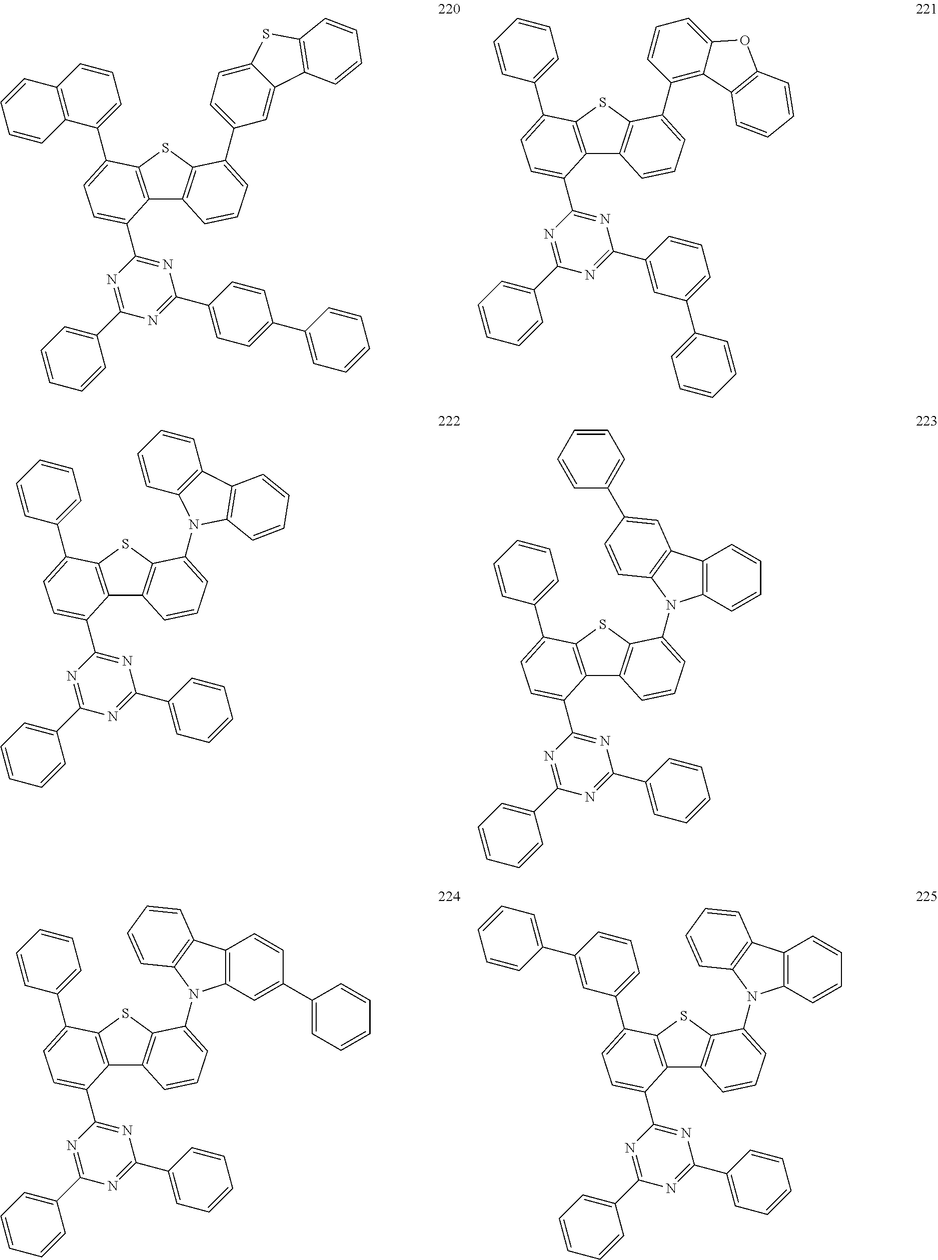

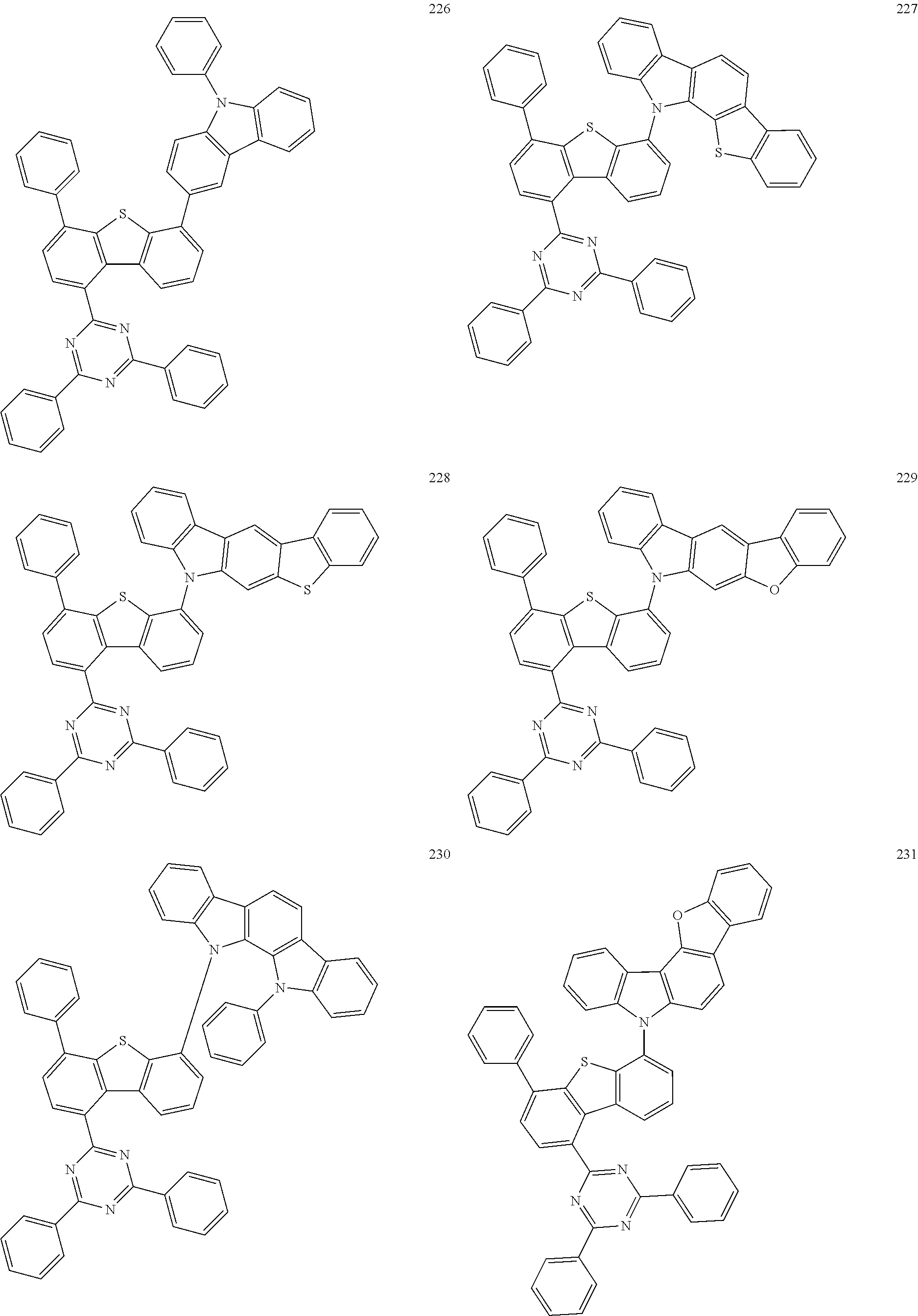

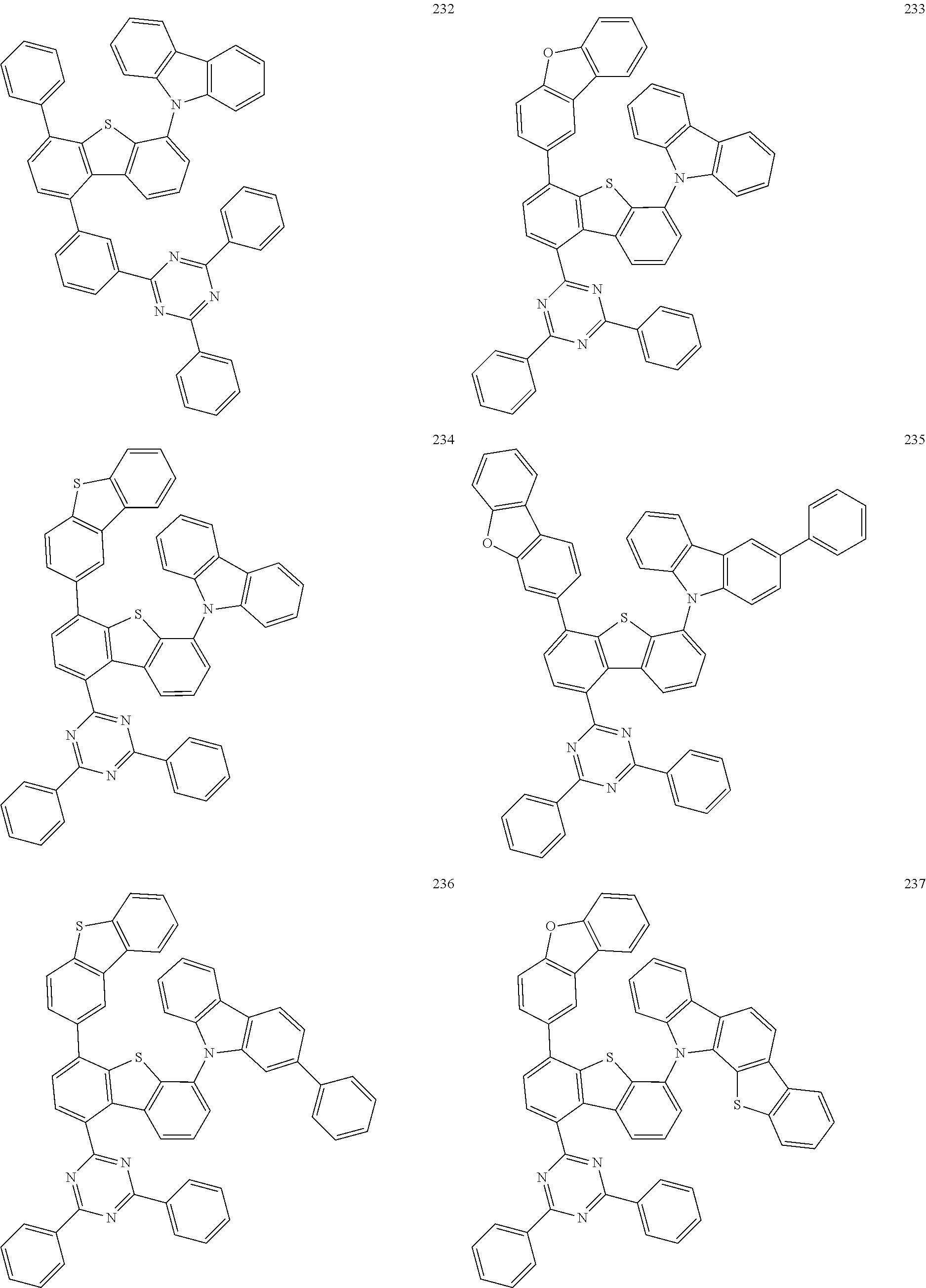

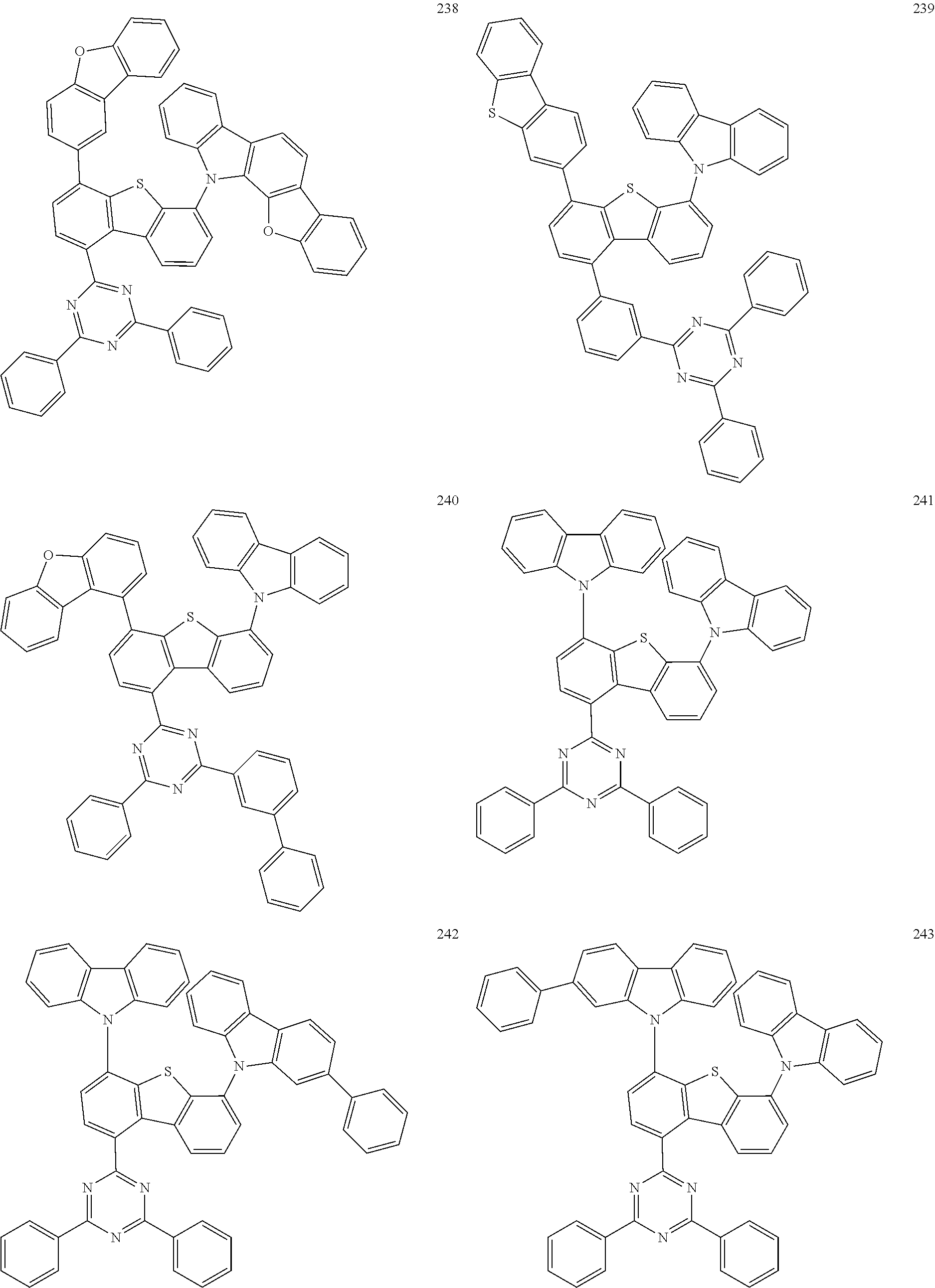

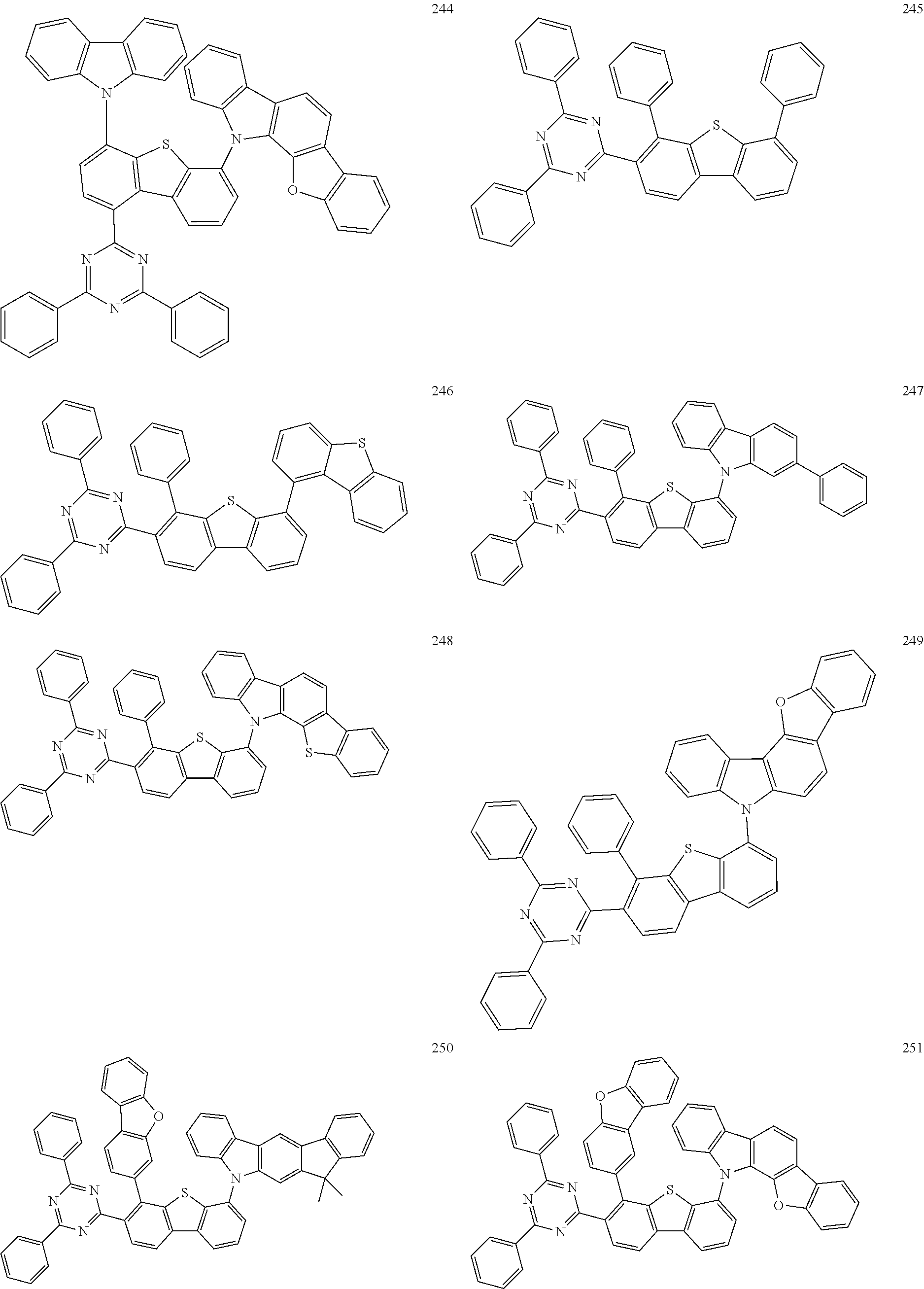

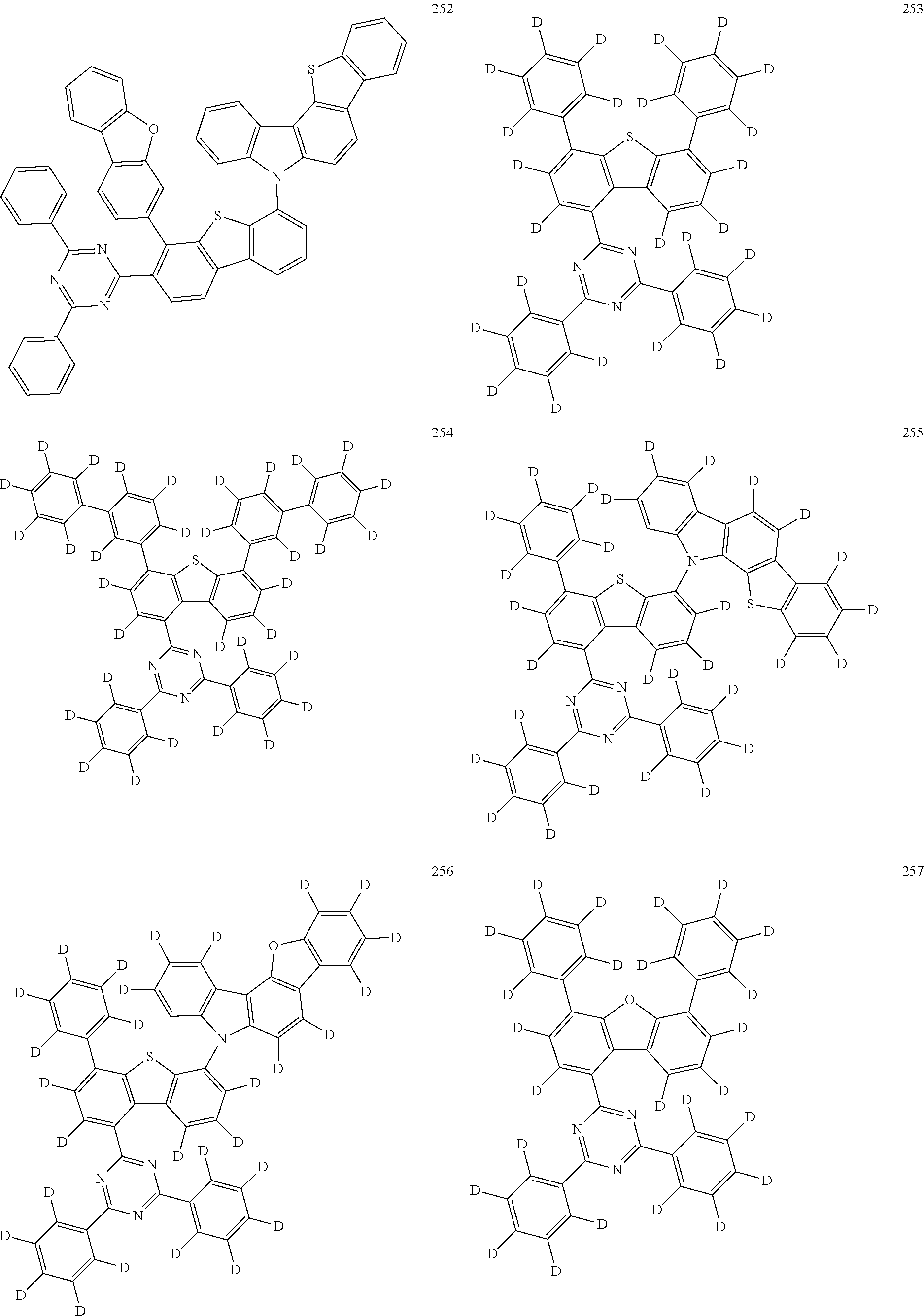

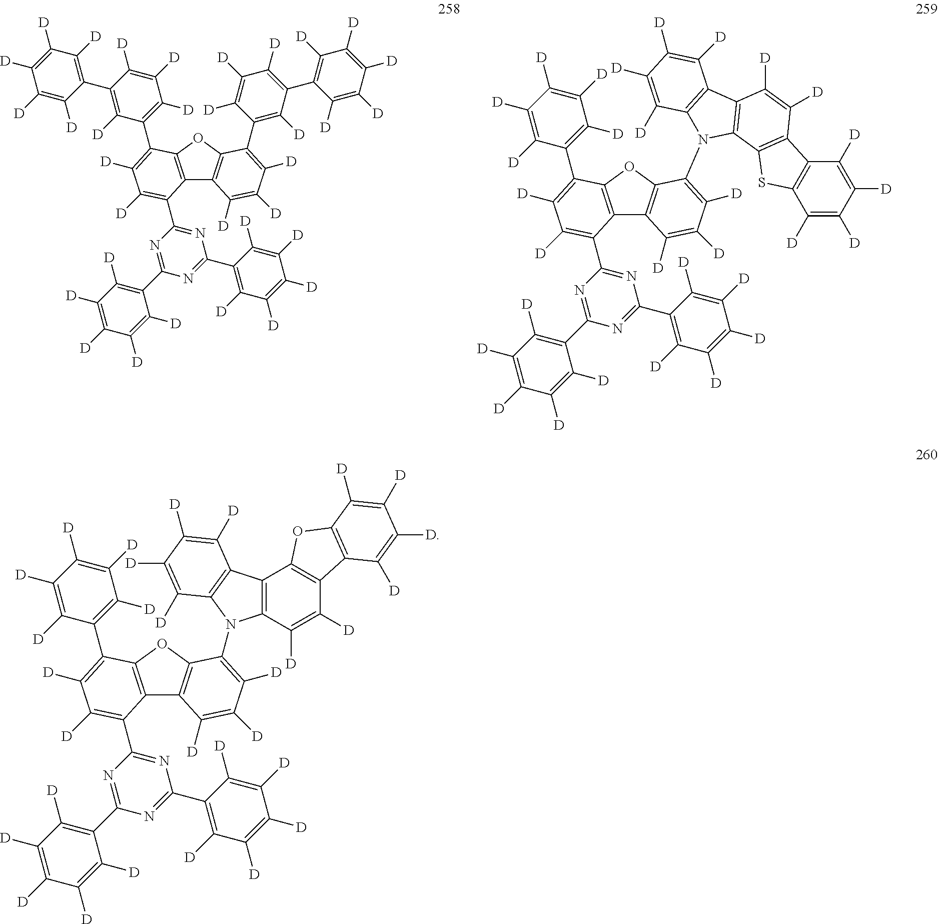

7. An organic light emitting device comprising:
a first electrode;
a second electrode provided opposite to the first electrode; and
one or more organic material layers provided between the first electrode and the second electrode,
wherein one or more layers of the organic material layers comprise the heterocyclic compound of claim 1.

8. The organic light emitting device of claim 7, wherein the organic material layer comprising the heterocyclic compound further comprises a heterocyclic compound represented by the following Chemical Formula A: 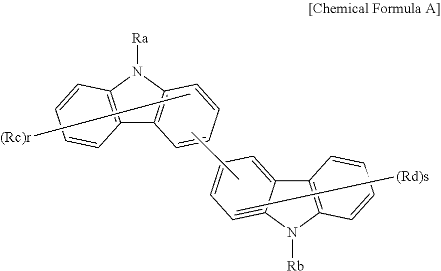
in Chemical Formula A,
Rc and Rd are the same as or different from each other, and each independently selected from the group consisting of hydrogen; deuterium; a halogen group; —CN; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiR201R202R203; —P(═O)R201R202; and —NR201R202, or two or more groups adjacent to each other bond to each other to form a substituted or unsubstituted C6 to C60 aromatic hydrocarbon ring or a substituted or unsubstituted C2 to C60 heteroring;
Ra and Rb are the same as or different from each other, and each independently a substituted or unsubstituted C6 to C60 aryl group; —SiR201R202R203; —P(═O)R201R202; or a substituted or unsubstituted C2 to C60 heteroaryl group;
R201, R202 and R203 are the same as or different from each other, and each independently hydrogen; deuterium; —CN; a substituted or unsubstituted C1 to C60 alkyl group; a substituted or unsubstituted C6 to C60 aryl group; or a substituted or unsubstituted C2 to C60 heteroaryl group;
r and s are an integer of 0 to 7; and
when r and s are 2 or greater, substituents in the parentheses are the same as or different from each other.

9. The organic light emitting device of claim 8, wherein the heterocyclic compound represented by Chemical Formula A is any one selected from among the following compounds:

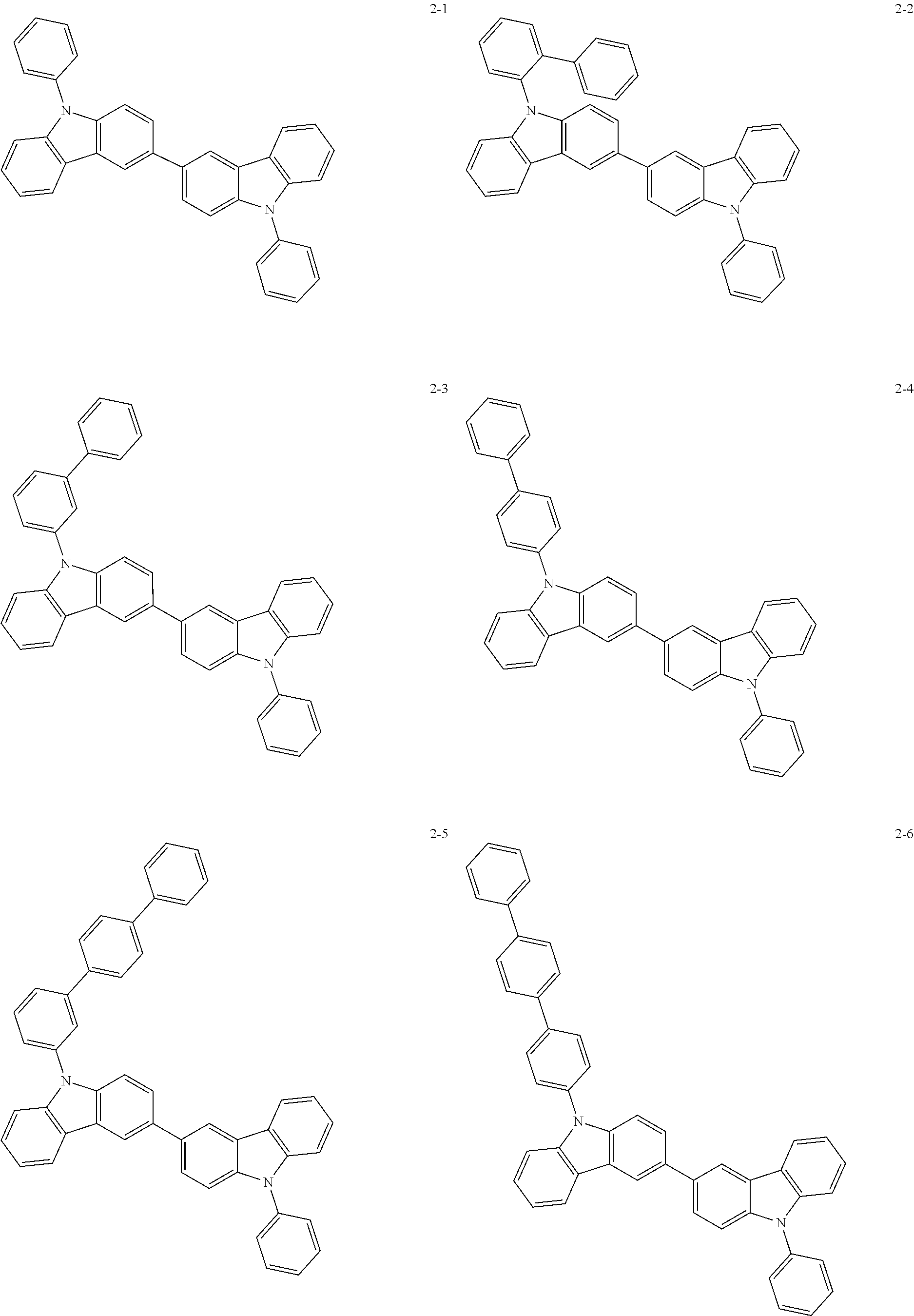 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

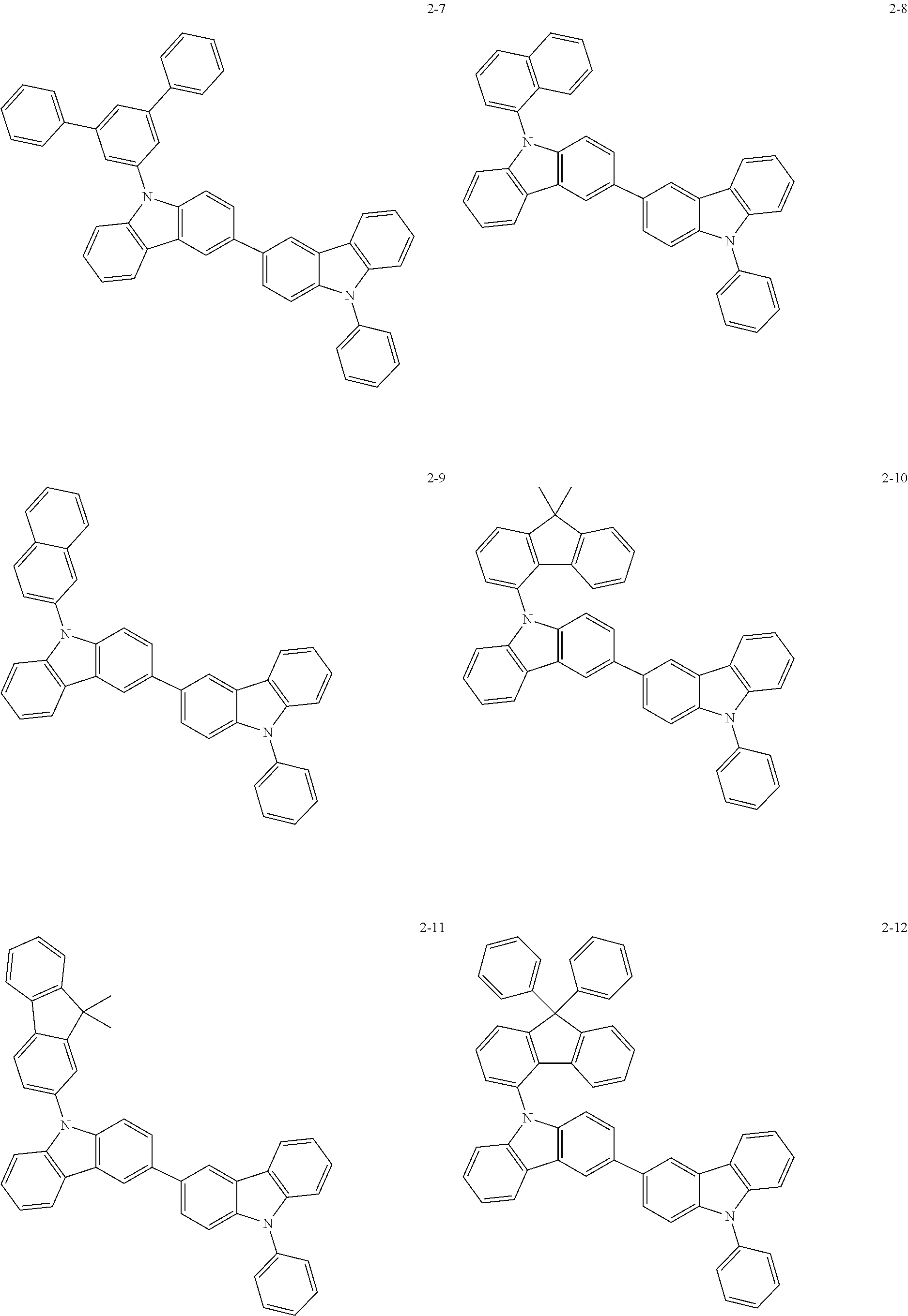 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

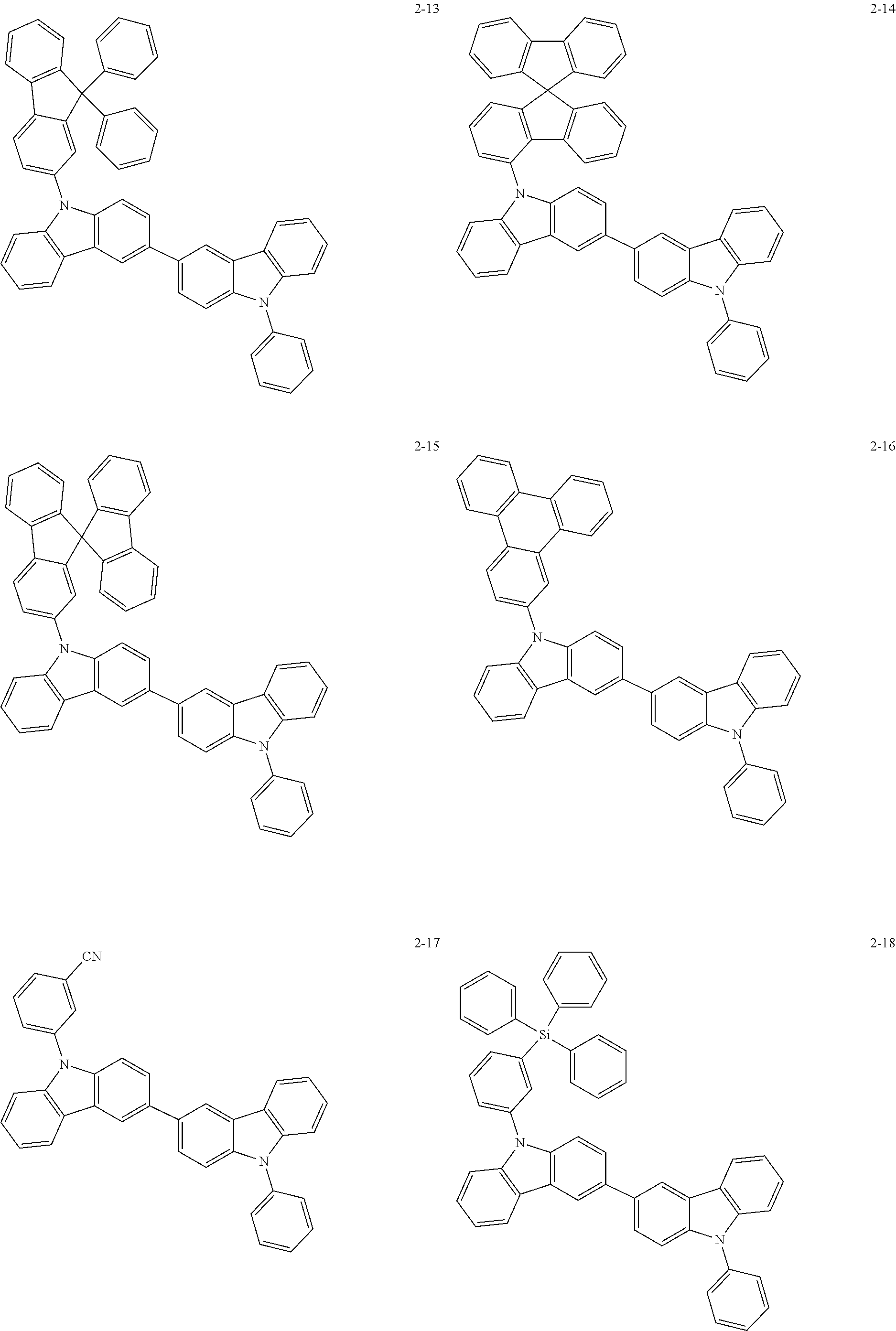

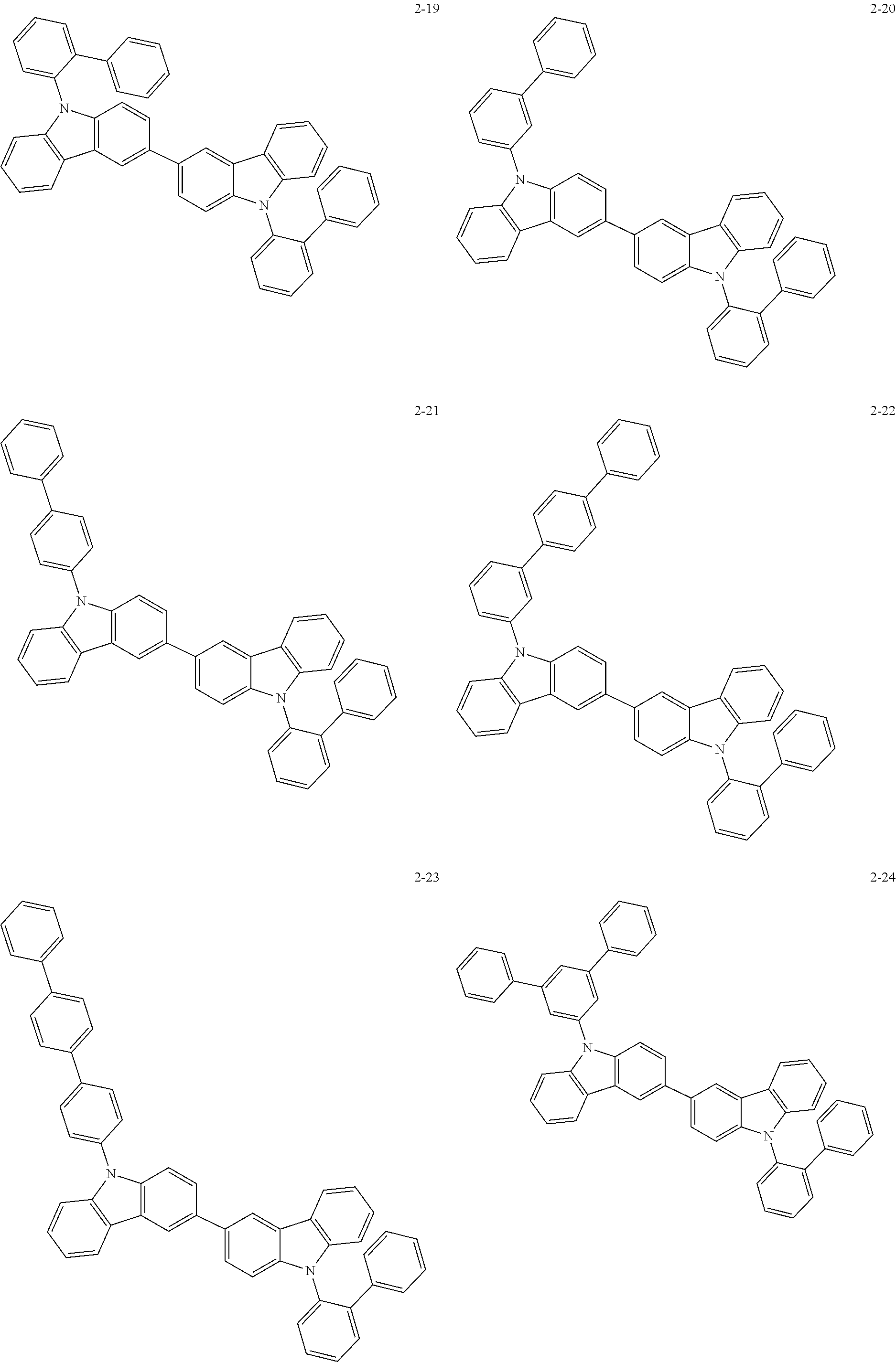

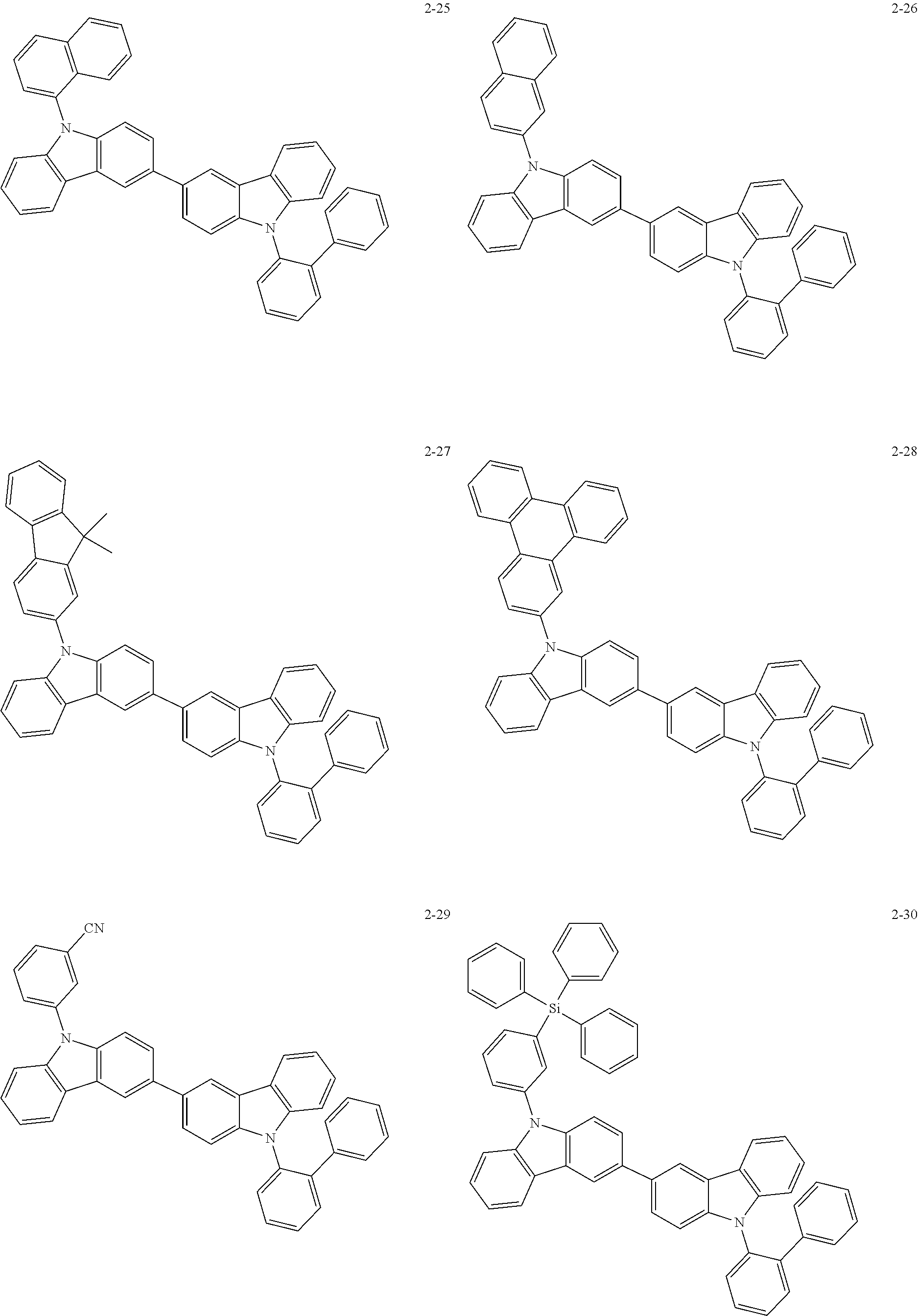

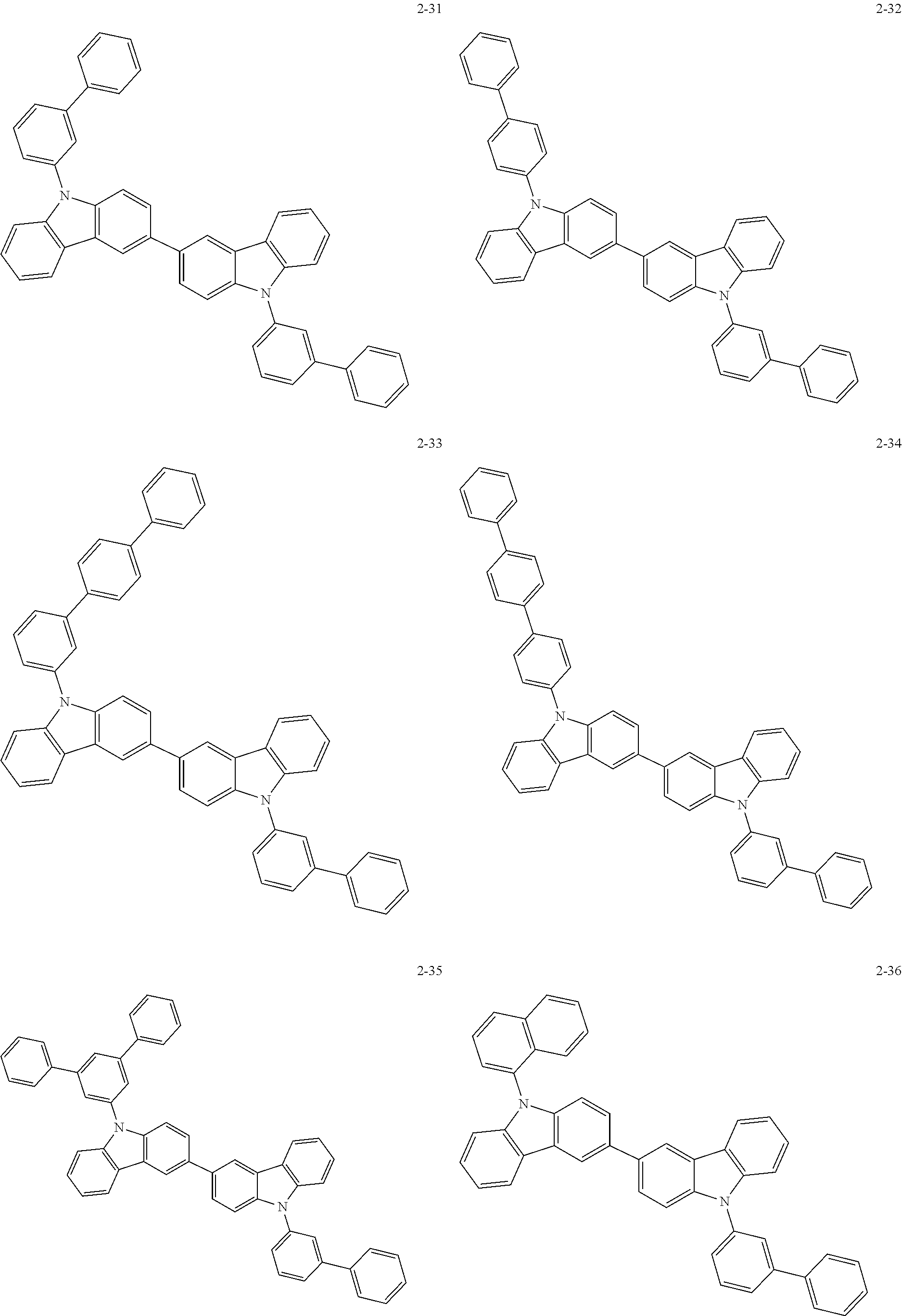

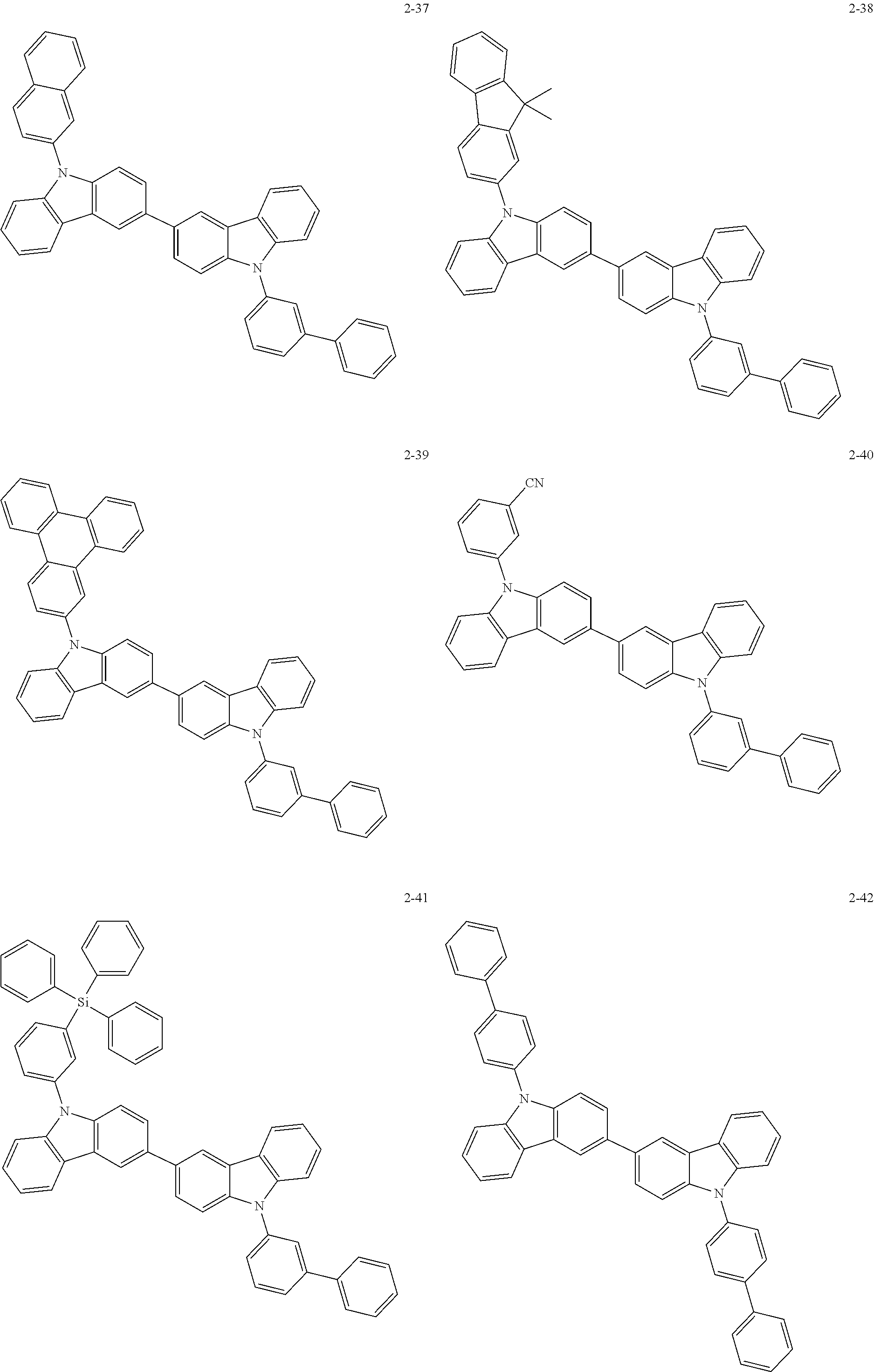

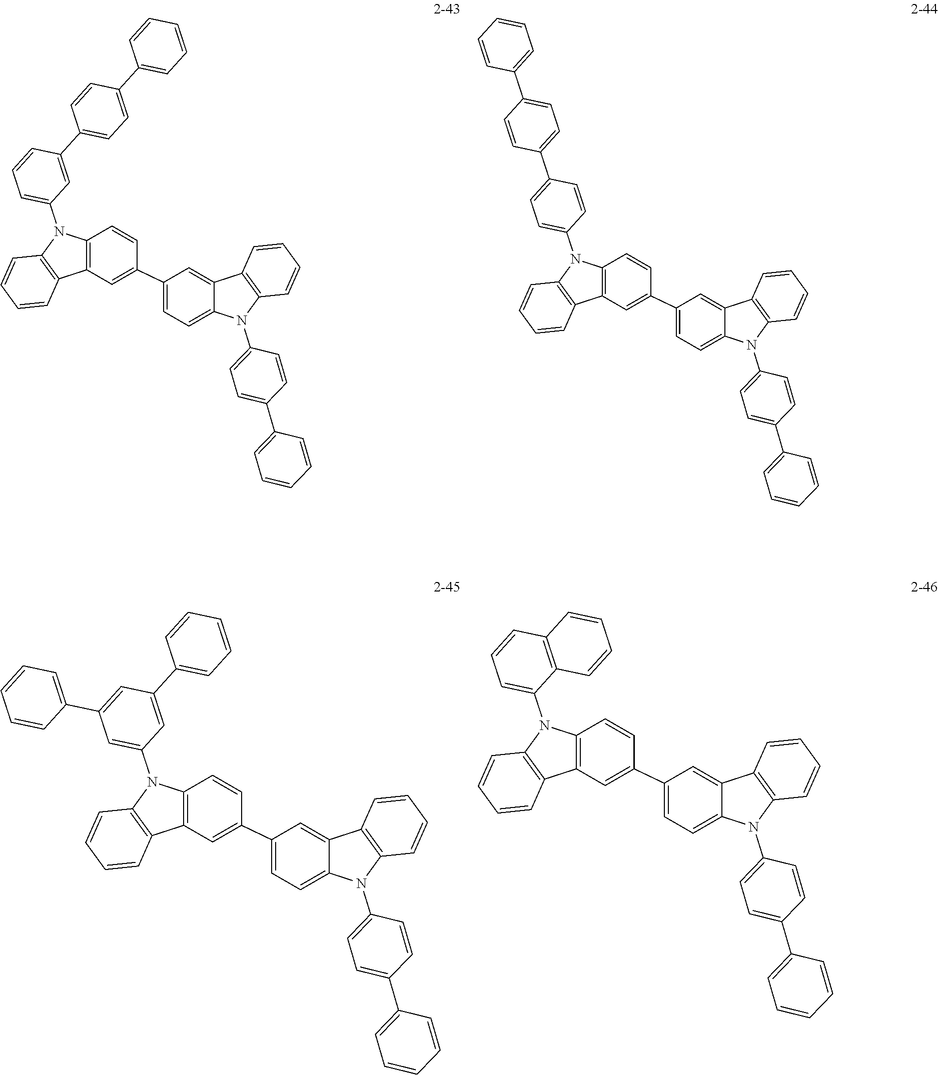 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2ccc(-n3c4ccccc4c4cc(-c5ccc6c(c5)c5ccccc5n6-c5cccc6ccccc56)ccc43)cc2)cc1]

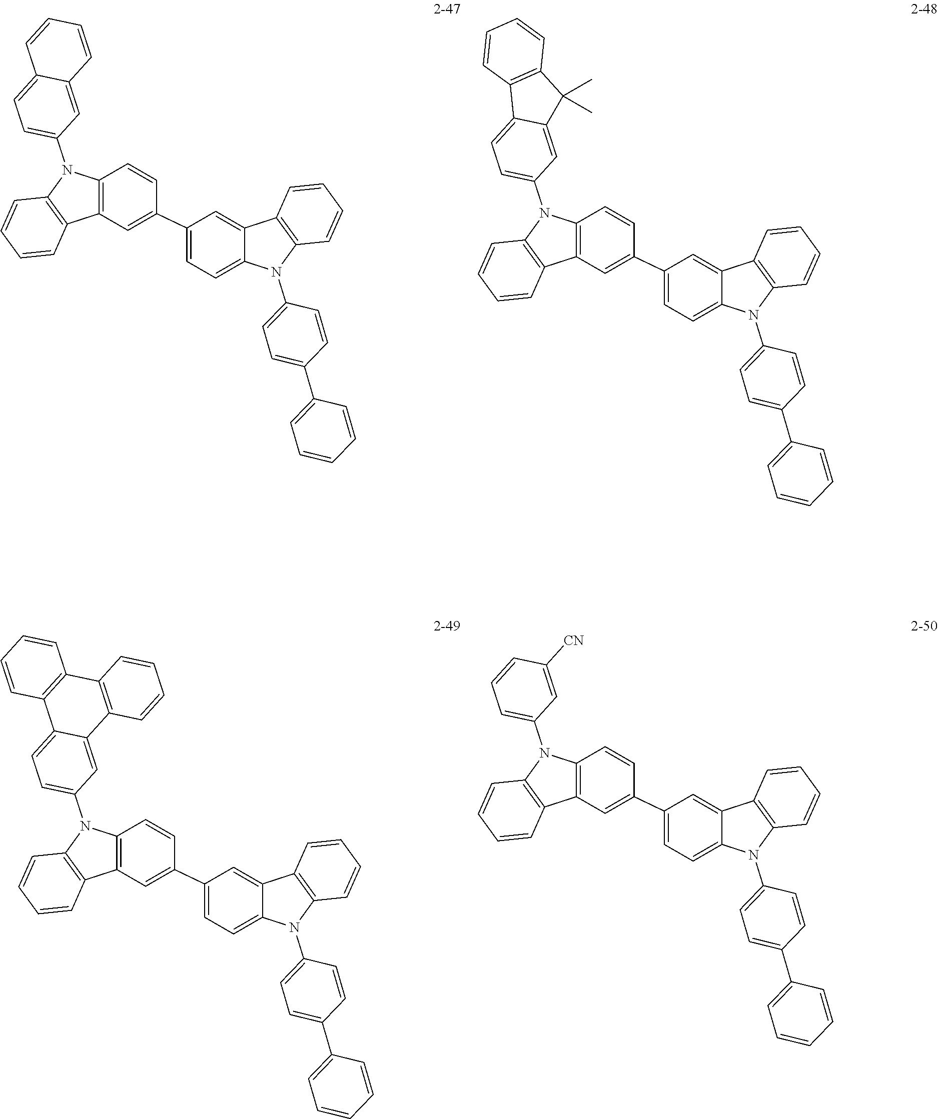 [SMILES: c1ccc(-c2ccc(-n3c4ccccc4c4cc(-c5ccc6c(c5)c5ccccc5n6-c5ccc6ccccc6c5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4ccc(-c5ccccc5)cc4)ccc32)c1]

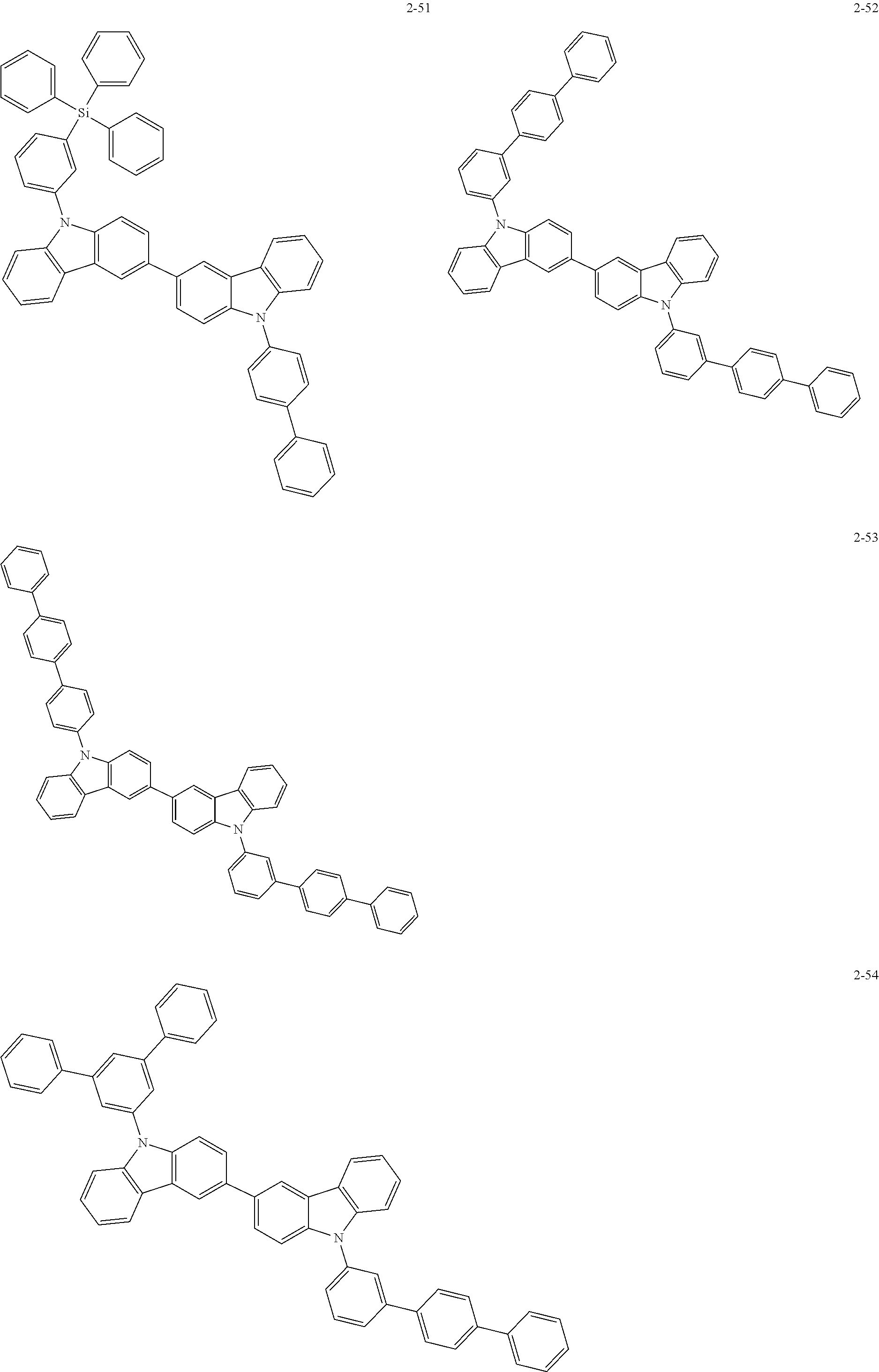 [SMILES: c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1 ; c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1]

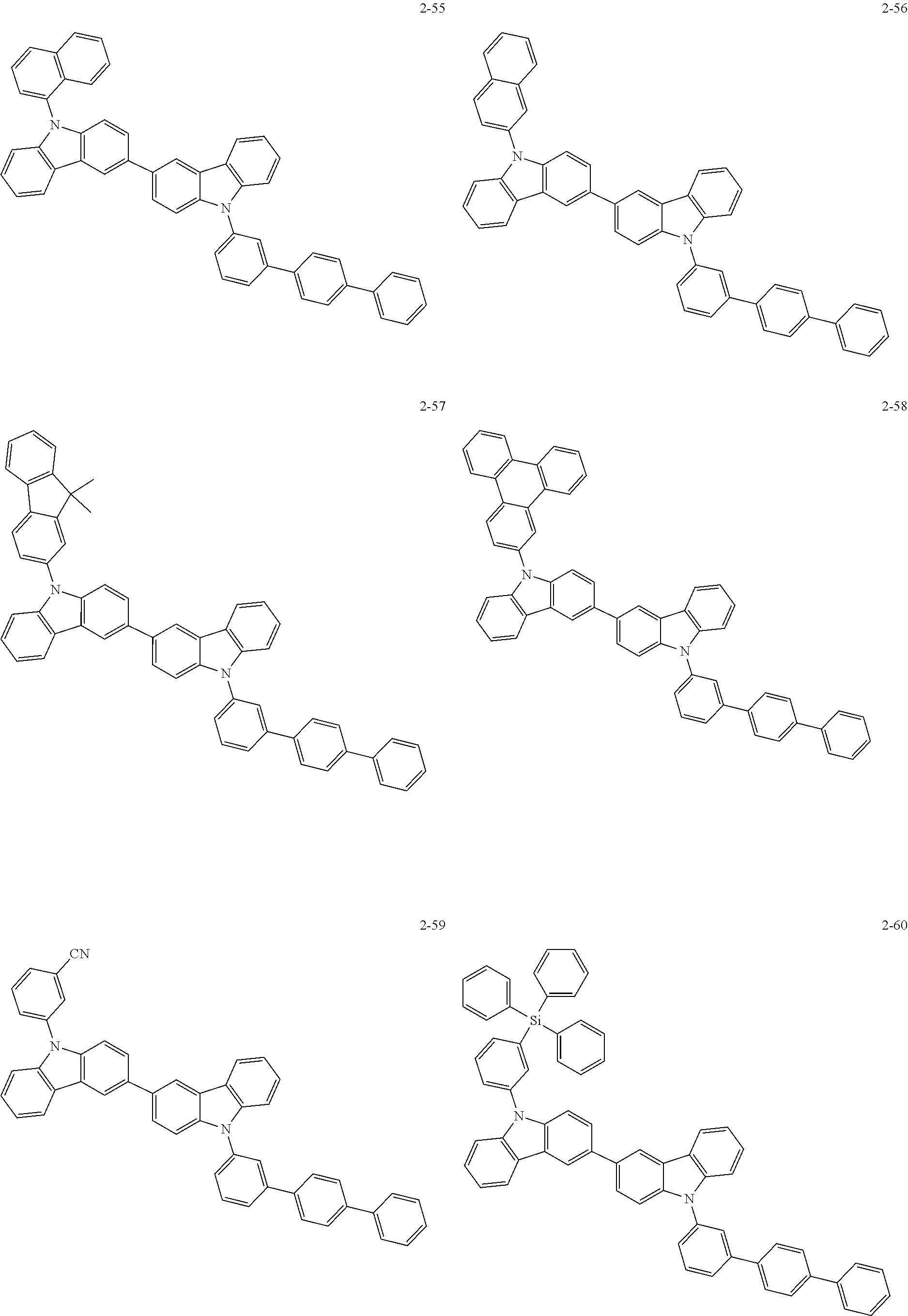

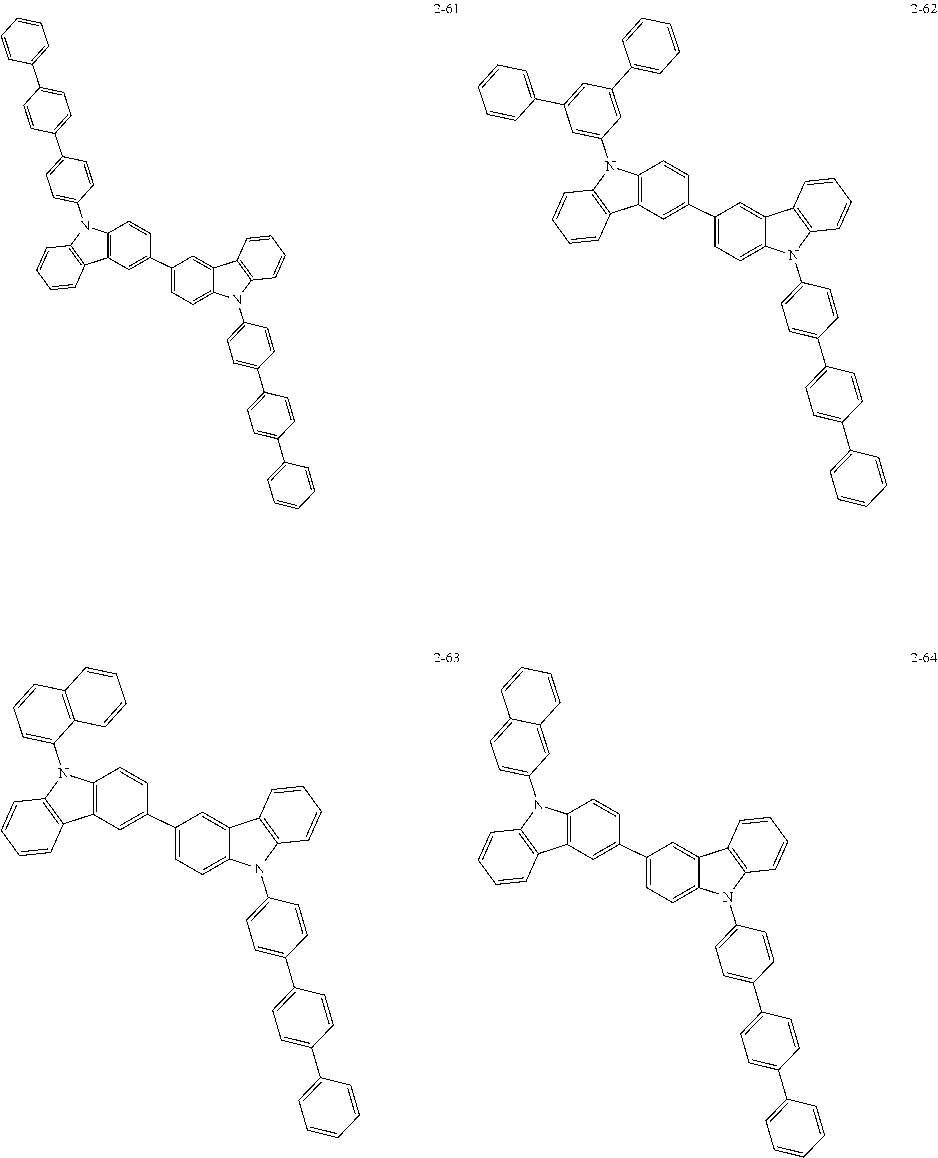 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

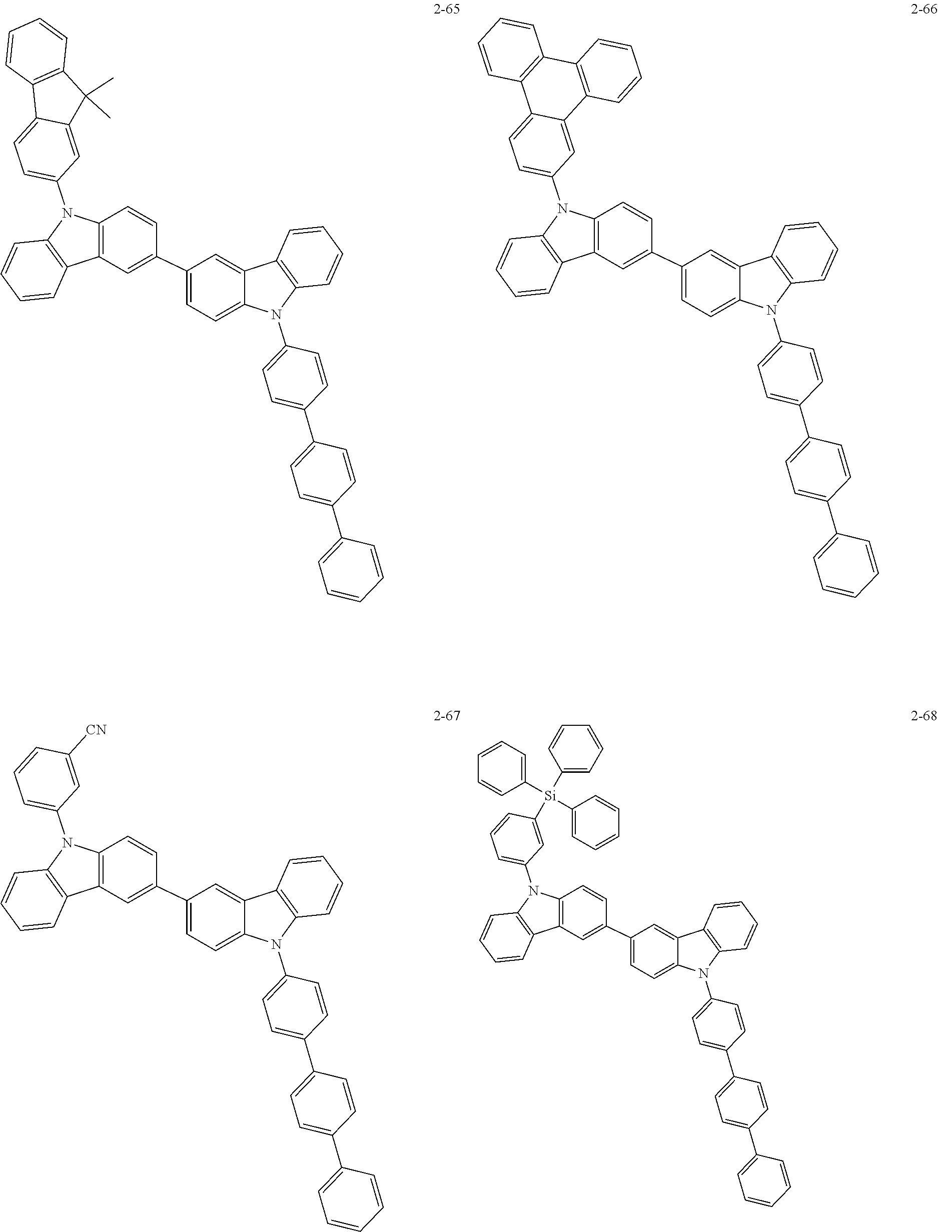 [SMILES: CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1 ; N#Cc1cccc(-n2c3ccccc3c3cc(-c4ccc5c(c4)c4ccccc4n5-c4ccc(-c5ccc(-c6ccccc6)cc5)cc4)ccc32)c1 ; c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1]

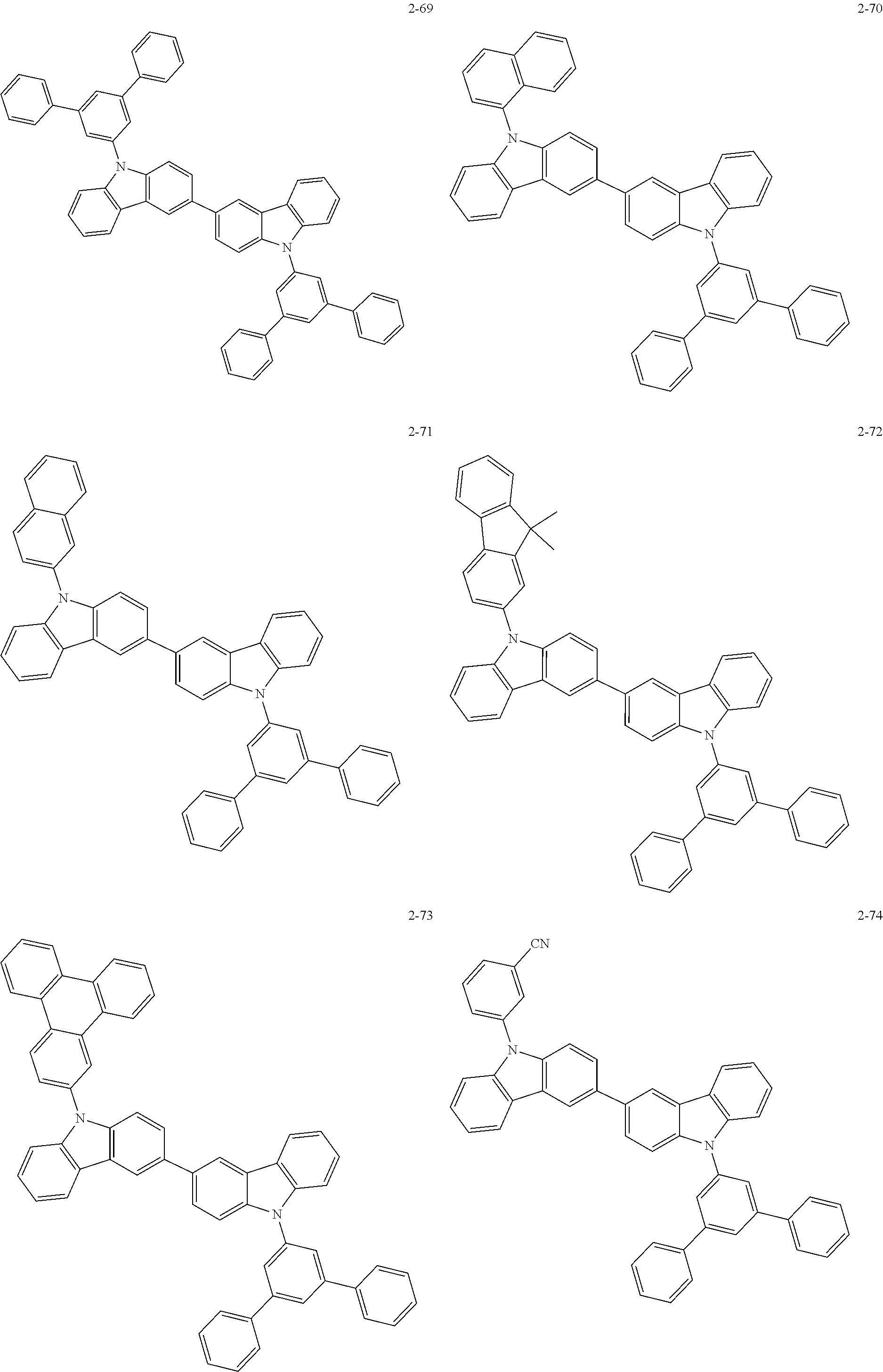

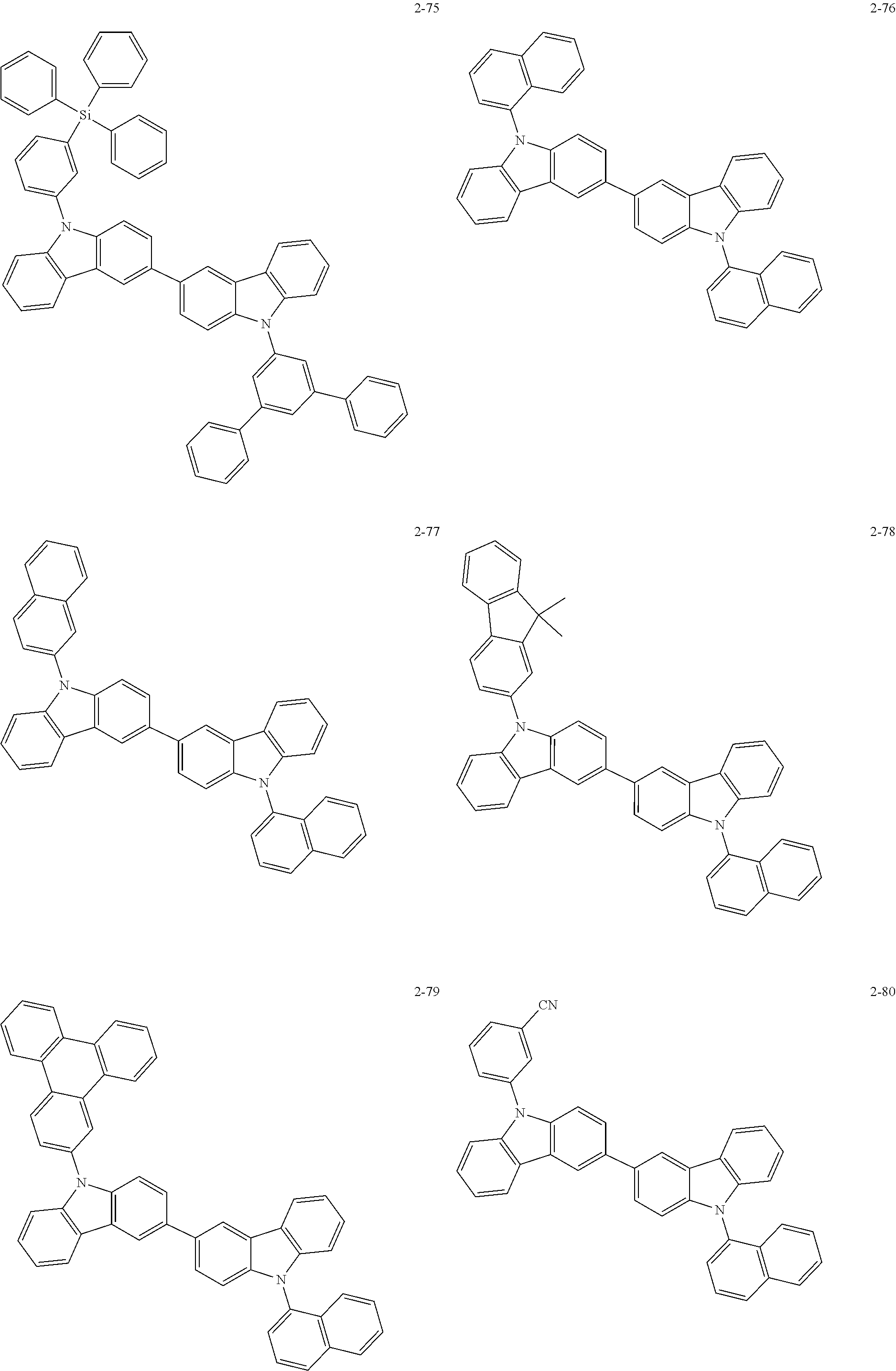

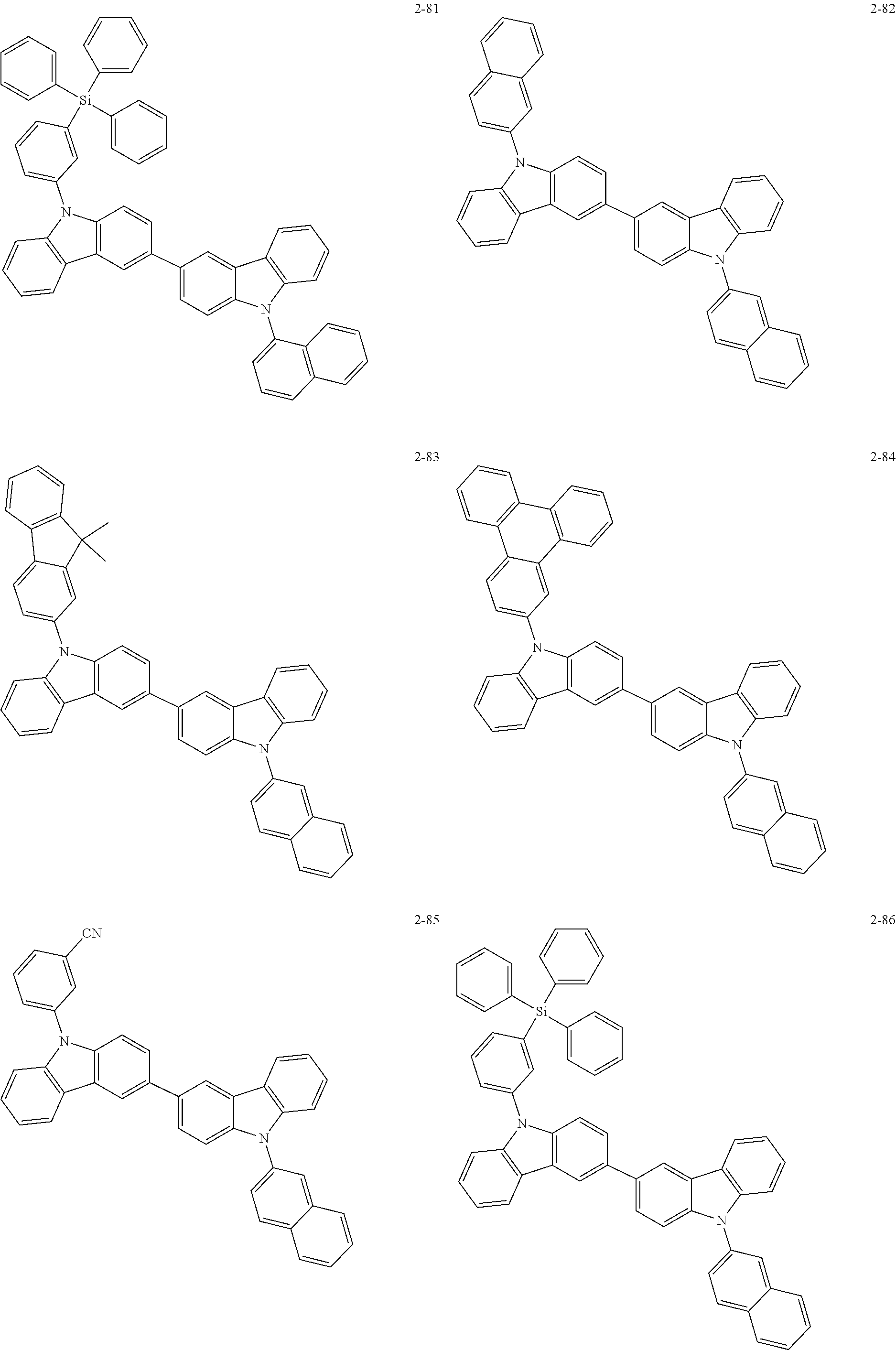

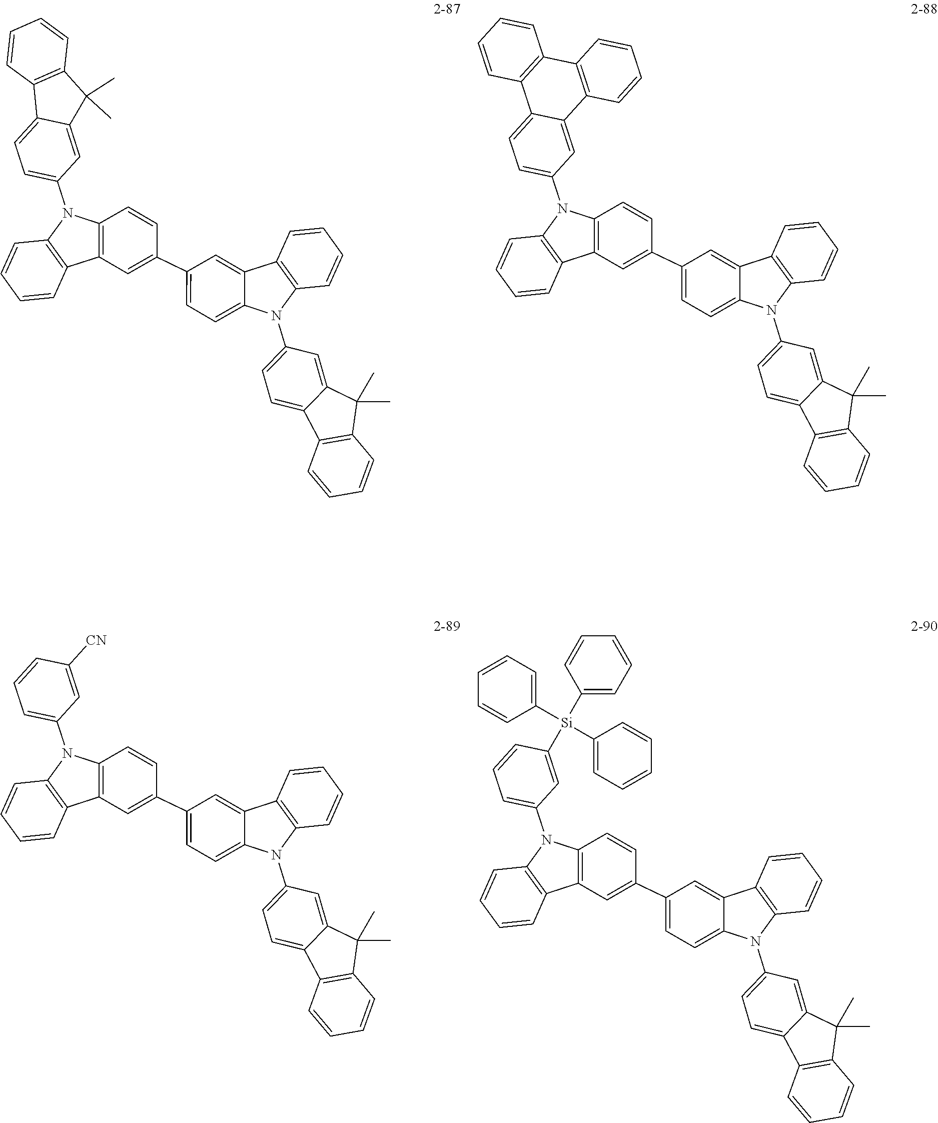 [SMILES: CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5ccccc5n6-c5ccc6c7ccccc7c7ccccc7c6c5)ccc43)cc21 ; CC1(C)c2ccccc2-c2ccc(-n3c4ccccc4c4cc(-c5ccc6c(c5)c5ccccc5n6-c5cccc(C#N)c5)ccc43)cc21 ; CC1(C)c2ccccc2-c2ccc(-n3c4ccccc4c4cc(-c5ccc6c(c5)c5ccccc5n6-c5cccc([Si](c6ccccc6)(c6ccccc6)c6ccccc6)c5)ccc43)cc21]

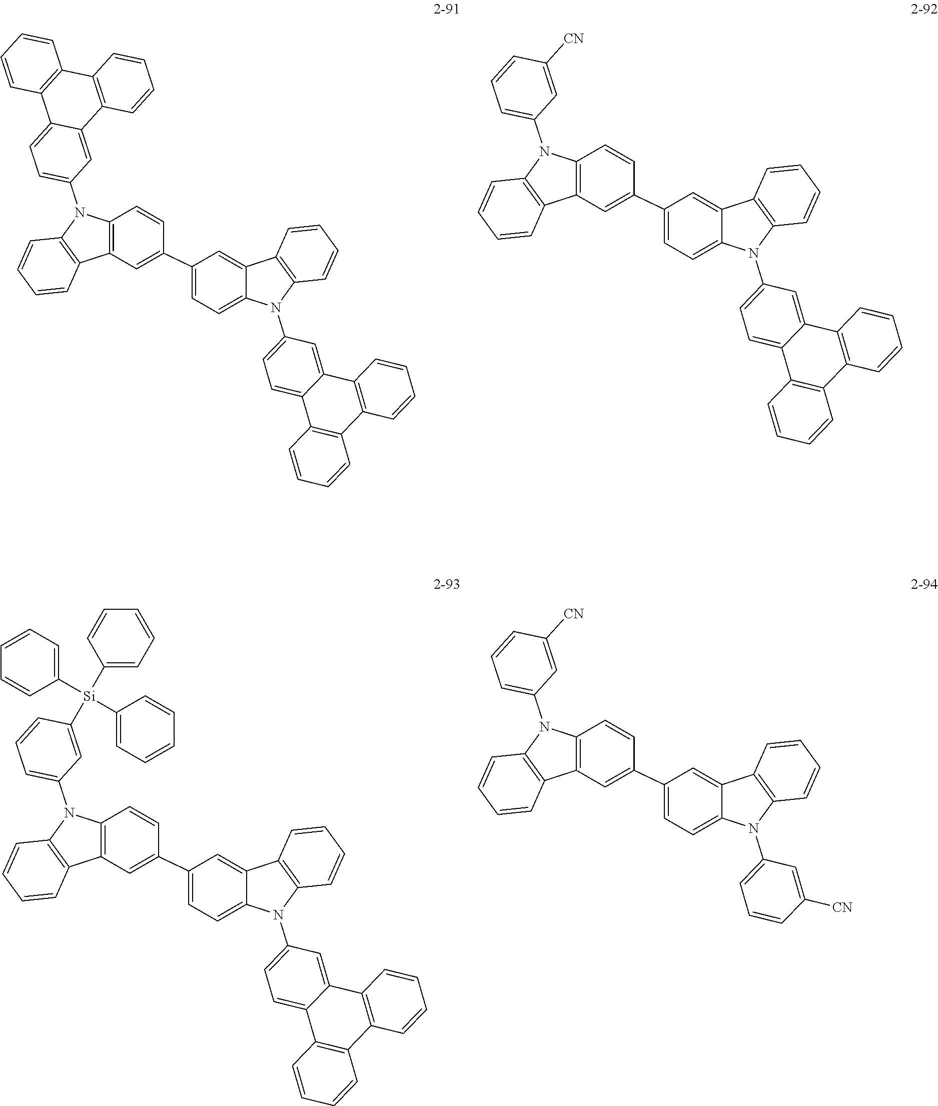 [SMILES: c1ccc2c(c1)c1ccccc1c1cc(-n3c4ccccc4c4cc(-c5ccc6c(c5)c5ccccc5n6-c5ccc6c7ccccc7c7ccccc7c6c5)ccc43)ccc21 ; N#Cc1cccc(-n2c3ccccc3c3cc(-c4ccc5c(c4)c4ccccc4n5-c4ccc5c6ccccc6c6ccccc6c5c4)ccc32)c1 ; c1ccc([Si](c2ccccc2)(c2ccccc2)c2cccc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cccc(C#N)c4)ccc32)c1]

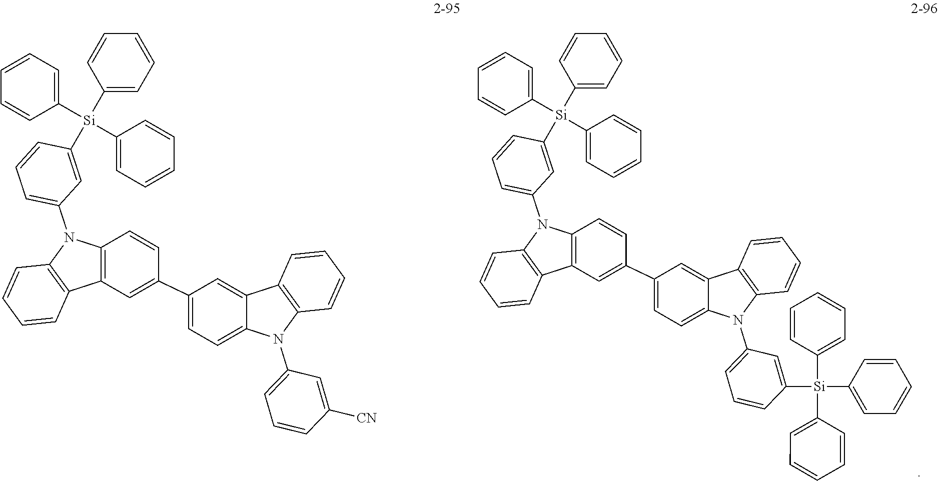 [SMILES: N#Cc1cccc(-n2c3ccccc3c3cc(-c4ccc5c(c4)c4ccccc4n5-c4cccc([Si](c5ccccc5)(c5ccccc5)c5ccccc5)c4)ccc32)c1 ; c1ccc([Si](c2ccccc2)(c2ccccc2)c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1]

10. The organic light emitting device of claim 8, wherein Rc and Rd are hydrogen.

11. The organic light emitting device of claim 7, wherein the organic material layer comprises a light emitting layer, and the light emitting layer comprises the heterocyclic compound of Chemical Formula 1.

12. The organic light emitting device of claim 7, wherein the organic material layer comprises a light emitting layer, the light emitting layer comprises a host material, and the host material comprises the heterocyclic compound of Chemical Formula 1.

13. The organic light emitting device of claim 7, further comprising one, two or more layers selected from the group consisting of a light emitting layer, a hole injection layer, a hole transport layer, an electron injection layer, an electron transport layer, an electron blocking layer and a hole blocking layer.

14. A composition for an organic material layer of an organic light emitting device, the composition comprising:
the heterocyclic compound of claim 1; and
a heterocyclic compound represented by the following Chemical Formula A: 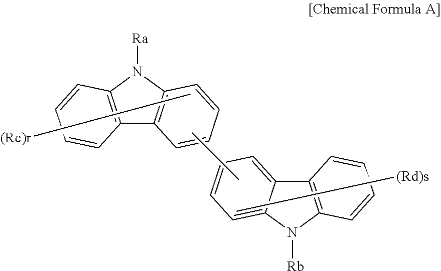
wherein, in Chemical Formula A,
Rc and Rd are the same as or different from each other, and each independently selected from the group consisting of hydrogen; deuterium; a halogen group; —CN; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiR201R202R203; —P(═O)R201R202; and —NR201R202, or two or more groups adjacent to each other bond to each other to form a substituted or unsubstituted C6 to C60 aromatic hydrocarbon ring or a substituted or unsubstituted C2 to C60 heteroring;
Ra and Rb are the same as or different from each other, and each independently a substituted or unsubstituted C6 to C60 aryl group; —SiR201R202R203; —P(═O)R201R202; or a substituted or unsubstituted C2 to C60 heteroaryl group;
R201, R202 and R203 are the same as or different from each other, and each independently hydrogen; deuterium; —CN; a substituted or unsubstituted C1 to C60 alkyl group; a substituted or unsubstituted C6 to C60 aryl group; or a substituted or unsubstituted C2 to C60 heteroaryl group;
r and s are an integer of 0 to 7; and
when r and s are 2 or greater, substituents in the parentheses are the same as or different from each other.

15. The composition for an organic material layer of an organic light emitting device of claim 14, wherein the heterocyclic compound: the heterocyclic compound represented by Chemical Formula A have a weight ratio of 1:10 to 10:1 in the composition.

16. A method for manufacturing an organic light emitting device, the method comprising:
preparing a substrate;
forming a first electrode on the substrate;
forming one or more organic material layers on the first electrode; and
forming a second electrode on the organic material layer,
wherein the forming of organic material layers comprises forming one or more organic material layers using the composition for an organic material layer of claim 14.

17. The method for manufacturing an organic light emitting device of claim 16, wherein the forming of organic material layers is forming using a thermal vacuum deposition method after pre-mixing the heterocyclic compound of Chemical Formula 1 and the heterocyclic compound of Chemical Formula A.
